(19) United States
(12) Patent Application Publication
(10) Pub. No.: US 2022/0202948 A1
(43) Pub. Date: Jun. 30, 2022

(54) Agent for Eliminating Pluripotent Stem Cells

(71) Applicant: Sumitomo Dainippon Pharma Co., Ltd., Osaka-shi, Osaka (JP)

(72) Inventors: Atsushi Suwa, Konohana-ku, Osaka-shi, Osaka (JP); Ayaka Fujiki, Chuo-ku, Kobe-shi, Hyogo (JP); Makiko Tsujiuchi, Konohana-ku, Osaka-shi, Osaka (JP)

(73) Assignee: Sumitomo Dainippon Pharma Co., Ltd., Osaka-shi, Osaka (JP)

(21) Appl. No.: 17/430,312

(22) PCT Filed: Feb. 12, 2020
(86) PCT No.: PCT/JP2020/005369

§ 371 Date: Aug. 11, 2021

(30) Foreign Application Priority Data
Feb. 13, 2019 (JP) 2019-023965

(51) Int. Cl. A61K 47/68; A61K 35/545

(52) U.S. Cl. CPC A61K 47/6849; A61K 35/545; A61K 47/6803

(57) ABSTRACT

An agent for eliminating a pluripotent stem cell, the agent comprising an antibody-drug conjugate that releases a compound represented by formula (1-1):

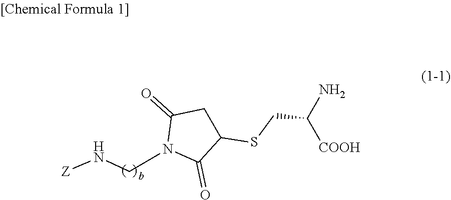

wherein
b represents an integer of 1 to 5; and
Z represents a group represented by formula (Z-1) or formula (Z-2),
   or a salt thereof.

### TECHNICAL FIELD

The present invention relates to agent for eliminating pluripotent stem cell.

### BACKGROUND ART

Induced pluripotent stem cells (iPS cells), one type of pluripotent stem cells, are cells possessing replication competence and differentiation potential in combination, and when iPS cells are directly transplanted in an organism, the iPS cells form a tumor called teratoma if being contaminated with undifferentiated iPS cells (Non Patent Literature 1). Teratoma is different from what is called cancer (malignant tumor); however, in development of a cell product from iPS cells as a starting material, the safety and efficacy of the product may be deteriorated if iPS cells remain in the final product and teratoma is formed from the cells. For this reason, it is crucial in development of a cell product derived from iPS cells that undifferentiated iPS cells, which possess teratomagenic potential, are not present in the product.

Currently, detection of iPS cells with high sensitivity has become feasible; however, such detection suffers from technical limitations, and particularly for cell products composed of numerous cells, the presence of a trace number of iPS cells is not completely denied in some cases. In view of this, various techniques have been developed, as approaches in an aspect of production, for the purpose of reducing the probability of the presence of a trace number of iPS cells to enhance safety by performing treatment to eliminate iPS cells for a product intermediate or final product. Examples thereof include a method of eliminating iPS cells by using a substance that induces cell death.

As substances capable of inducing cell death to iPS cells, for example, a fusion protein of a glycoprotein that recognizes iPS cells and a toxin (Non Patent Literature 2), an antibody that recognizes iPS cells to induce cell death (Non Patent Literature 3), a compound that inhibits fatty acid desaturation (Patent Literature 1 and Non Patent Literature 4), and an antibody-drug conjugate that selectively recognizes iPS cells to induce cell death (Non Patent Literature 5) are known. However, the hemiasterlin derivatives and antibody-drug conjugates thereof according to the present invention are not known as such substances. The fusion protein of a glycoprotein that recognizes iPS cells and a toxin and the antibody for eliminating iPS cells effectively act on iPS cells in a monolayer under plate culture; when the fusion protein or antibody is added to cell clusters without any vascular system, however, the efficiency to permeate into the inside of cell clusters is expected to be very poor because of the character of being a macromolecule with high molecular weight.

Antibody-drug conjugates are conjugates, for which an antibody is used as a target recognition molecule, formed by conjugating the antibody and a drug directly or via an appropriate linker. Such antibody-drug conjugates have a characteristic to eliminate target cells in a cell-selective manner through delivering the drug to target cells via an antibody that binds to an antigen expressed on the target cells.

For example, Adcetris, which is obtained by binding monomethyl auristatin to an anti-CD30 antibody, has been reported to induce cell death selectively to remaining iPS cells (Patent Literature 2 and Non Patent Literature 5).

In antibody-drug conjugates, drugs that exhibit membrane permeability cause cell damage also to cells expressing no antigen through the occurrence of drug diffusion from around target cells, which is called bystander effect. For example, Adcetris has been reported to have bystander effect (Non Patent Literature 6).

Hemiasterlin is a naturally occurring compound having a tripeptide structure, isolated from marine sponges, and is involved in microtubule depolymerization and mitotic arrest in cells (Non Patent Literature 7).

Several groups have so far conducted structural modification of hemiasterlin derivatives, and have reported structure-activity relationship (Patent Literatures 3 to 7 and Non Patent Literatures 8 to 11). As such, hemiasterlin derivatives exhibiting strong cytotoxicity (cellular toxicity) based on antimitotic effects have been found.

In addition, antibody-drug conjugates containing hemiasterlin derivatives, exhibiting cytotoxic activity in antigen-expressing cells, have also been reported (Patent Literatures 6 and 8 to 12).

Further, several other groups have reported that an antibody-drug conjugate formed by directly conjugating a cysteine residue of an antibody and a drug releases the Cys-drug moiety of the antibody-drug conjugate in cells through metabolism of the antibody (Non Patent Literature 12).

### CITATION LIST

### Patent Literature

Patent Literature 1: JP 2015-525207
Patent Literature 2: International Publication No. WO 2016/072519
Patent Literature 3: International Publication No. WO 2004/026293
Patent Literature 4: International Publication No. WO 96/33211
Patent Literature 5: U.S. Pat. No. 7,579,323
Patent Literature 6: International Publication No. WO 2014/144871
Patent Literature 7: International Publication No. WO 2003/082268
Patent Literature 8: International Publication No. WO 2016/123582
Patent Literature 9: International Publication No. WO 2015/095952
Patent Literature 10: International Publication No. WO 2015/095953
Patent Literature 11: International Publication No. WO 2013/173393
Patent Literature 12: International Publication No. WO 2014/057436

### Non Patent Literature

Non Patent Literature 1: Plos One, 9, 1-11 (2014).
Non Patent Literature 2: Stem Cell Reports, 4(5), 811-820 (2015).
Non Patent Literature 3: Journal of Biological Chemistry, 290, 20071-20085 (2015).
Non Patent Literature 4: Cell Stem Cell, 12, 167-179 (2013).
Non Patent Literature 5: Sci. Rep., 8, 3726 (2018).
Non Patent Literature 6: Clin. Cancer. Res., 16, 888-897 (2010).
Non Patent Literature 7: Tetrahedron Lett., 35, 4453-4456 (1994).
Non Patent Literature 8: Bioorg. Med. Chem. Lett., 14, 4353-4358 (2004).
Non Patent Literature 9: J. Med. Chem., 47, 4774-4786 (2004).
Non Patent Literature 10: Bioorg. Med. Chem. Lett., 14, 5317-5322 (2004).
Non Patent Literature 11: J. Nat. Prod., 66, 183-199 (2003).
Non Patent Literature 12: Bioconjugate Chem. 17, 114-124 (2006).

### SUMMARY OF INVENTION

### Technical Problem

An object of the present invention is to provide such an antibody-drug conjugate containing a hemiasterlin derivative that a compound produced from the antibody-drug conjugate provides cell damage specifically to pluripotent stem cells while suppressing cell damage to differentiated cells.

### Solution to Problem

As a result of diligent studies, the present inventors have found that a compound represented by formula (1-1), formula (1-2) or formula (1-3) exhibits strong cytotoxic activity to iPS cells compared with cytotoxicity to differentiated cells, thereby completing the present invention.

That is, the present invention is as follows:

[Item 1]

An agent for eliminating a pluripotent stem cell, the agent comprising:

an antibody-drug conjugate that releases a compound represented by formula (1-1):

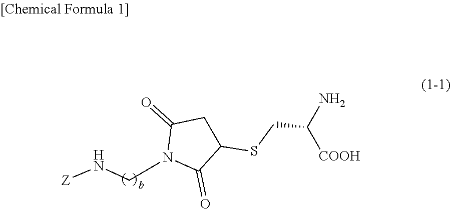

wherein

b represents an integer of 1 to 5; and

Z is a group represented by formula (Z-1) or formula (Z-2):

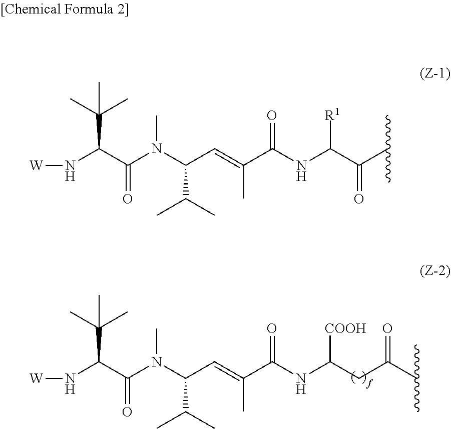

where
W represents a group represented by formula (W-1) or formula (W-2):

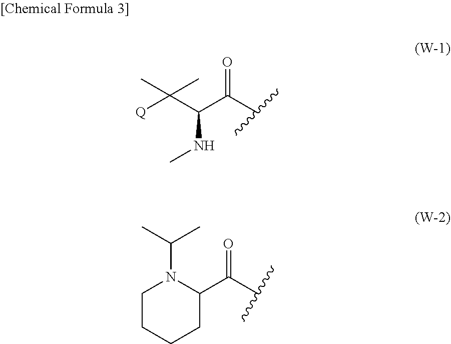

where
   Q represents a group represented by formula (Q-1) or formula (Q-2):

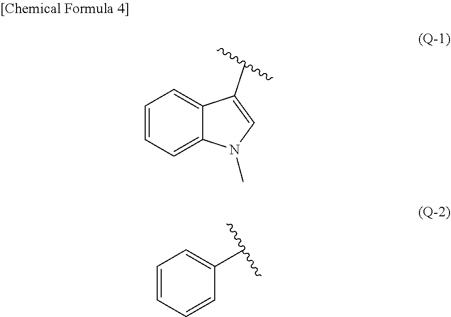 [SMILES: *c1cn(C)c2ccccc12 ; *c1ccccc1]

f represents 1 or 2;
R¹ represents —(CH₂)ᵤ—COOH; and
u represents 1 or 2,
   or a salt thereof.

[Item 2]

An agent for eliminating a pluripotent stem cell, the agent comprising:

an antibody-drug conjugate that releases a compound represented by formula (1-2):

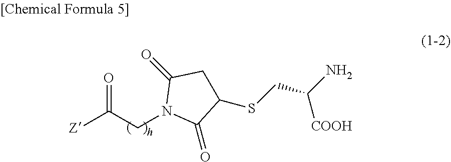

wherein

h represents an integer of 1 to 5; and

Z′ is a group represented by formula (Z-3) or formula (Z-4):

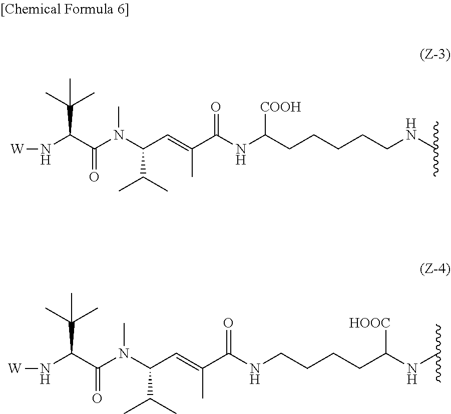

where
W represents a group represented by formula (W-1) or formula (W-2):

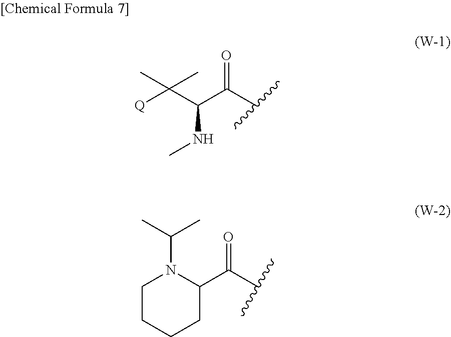

where
   Q represents a group represented by formula (Q-1) or formula (Q-2):

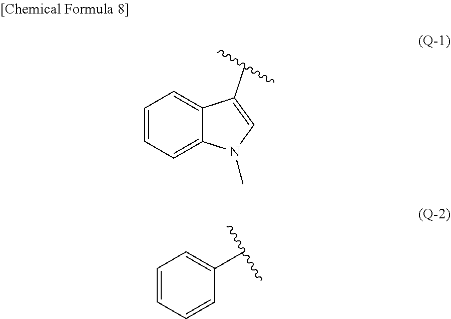 [SMILES: *c1cn(C)c2ccccc12 ; *c1ccccc1]

or a salt thereof.

[Item 3]

An agent for eliminating a pluripotent stem cell, the agent comprising:

an antibody-drug conjugate that releases a compound represented by formula (1-3):

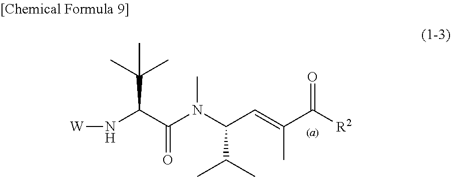

wherein

R² represents a glutamic acid residue (Glu), an aspartic acid residue (Asp) or a lysine residue (Lys);

an N-terminal nitrogen atom of R² forms an amide bond together with carbonyl (a); and

W is a group represented by formula (W-1) or formula (W-2):

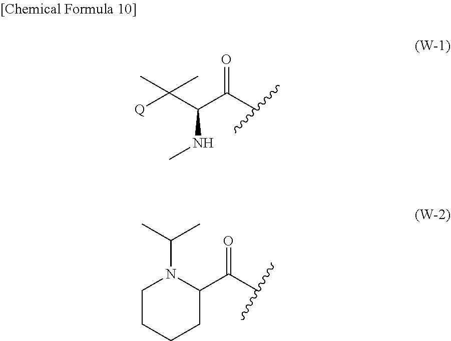

where
Q represents a group represented by formula (Q-1) or formula (Q-2):

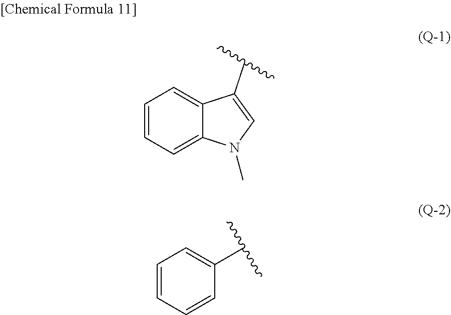 [SMILES: *c1cn(C)c2ccccc12 ; *c1ccccc1]

or a salt thereof.

[Item 4]

An agent for eliminating a pluripotent stem cell, the agent comprising:

an antibody-drug conjugate that releases the compound according to any one of items 1 to 3, wherein

W is a group represented by formula (W-1), or a salt thereof.

[Item 5]

An agent for eliminating a pluripotent stem cell, the agent comprising:

an antibody-drug conjugate that releases the compound according to item 1 or 4 selected from the following compounds:

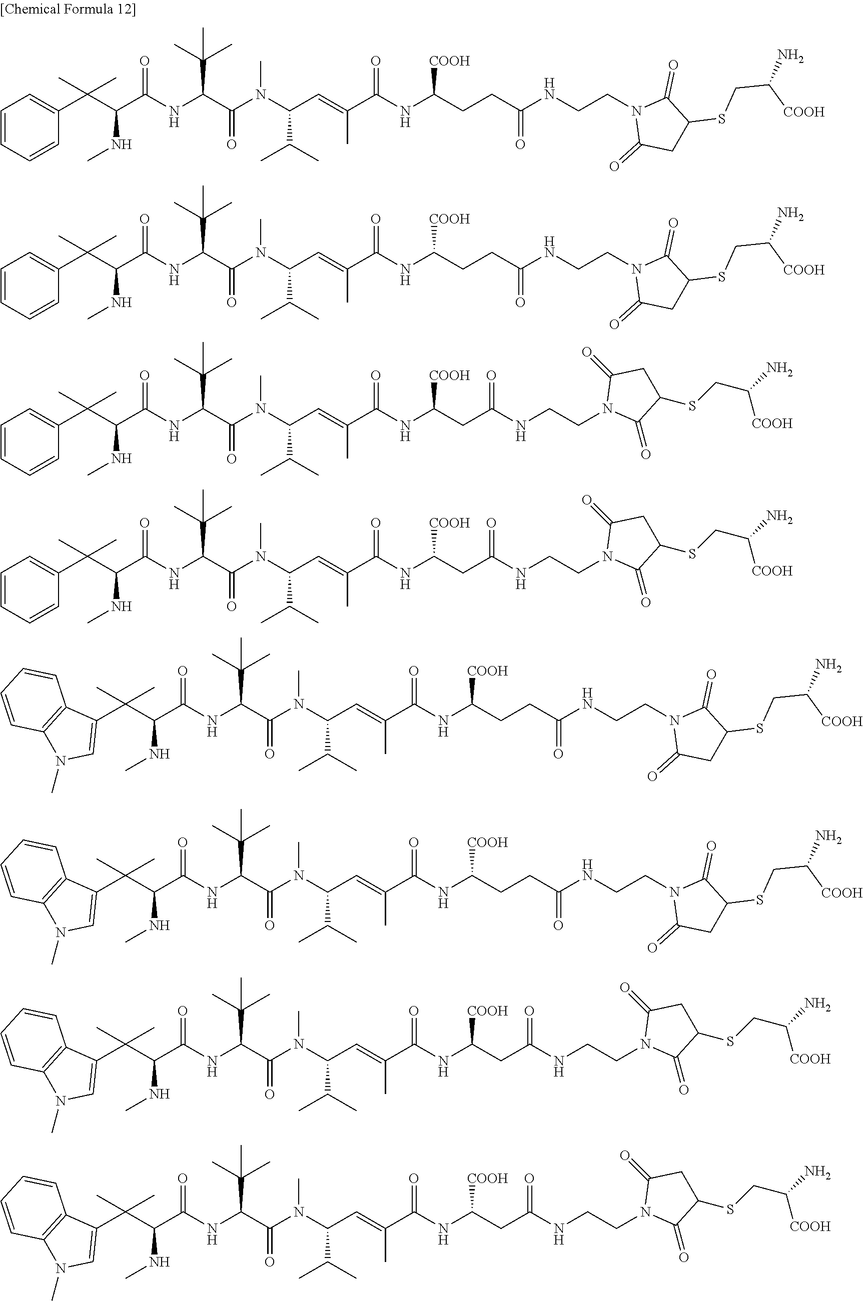

or a salt thereof.

[Item 6]

An agent for eliminating a pluripotent stem cell, the agent comprising:

an antibody-drug conjugate that releases the compound according to item 2 or 4 selected from the following compounds:

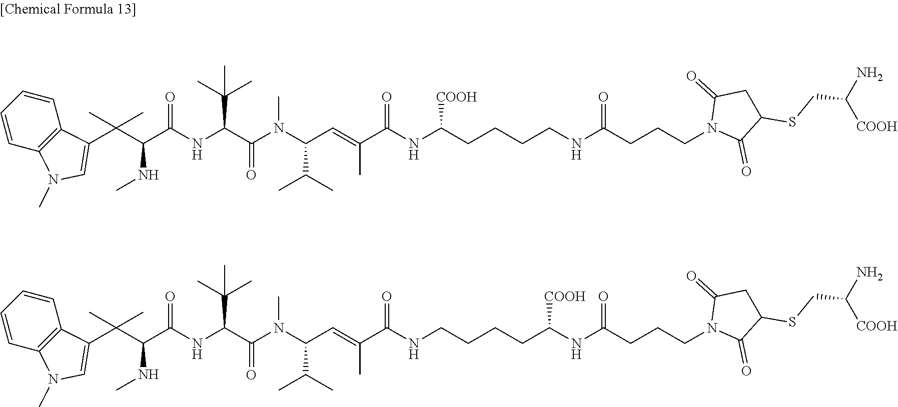 [SMILES: CN[C@H](C(=O)N[C@H](C(=O)N(C)[C@H](/C=C(\C)C(=O)N[C@@H](CCCCNC(=O)CCCN1C(=O)CC(SC[C@H](N)C(=O)O)C1=O)C(=O)O)C(C)C)C(C)(C)C)C(C)(C)c1cn(C)c2ccccc12 ; CN[C@H](C(=O)N[C@H](C(=O)N(C)[C@H](/C=C(\C)C(=O)NCCCC[C@@H](NC(=O)CCCN1C(=O)CC(SC[C@H](N)C(=O)O)C1=O)C(=O)O)C(C)C)C(C)(C)C)C(C)(C)c1cn(C)c2ccccc12]

or a salt thereof.

[Item 7]

An agent for eliminating a pluripotent stem cell, the agent comprising:

an antibody-drug conjugate that releases the compound according to item 3 or 4 selected from the following compounds:

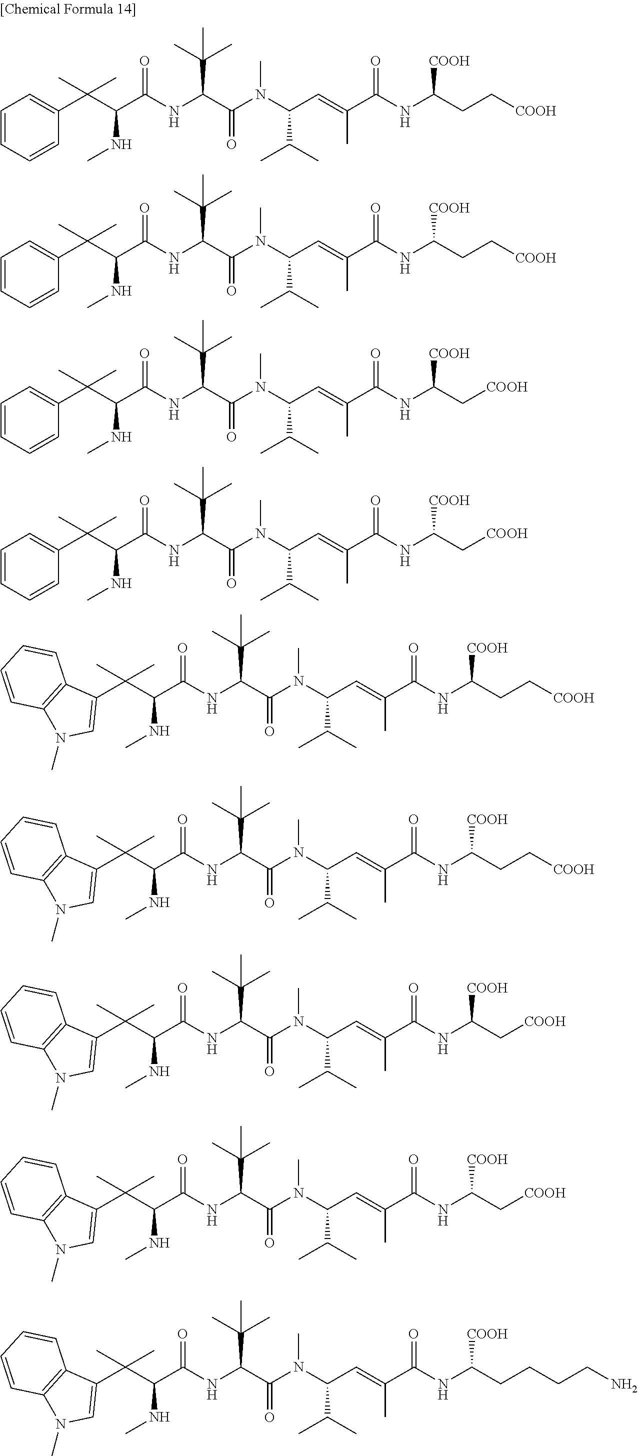

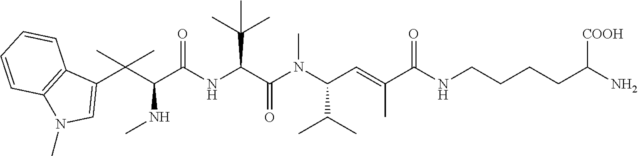 [SMILES: CN[C@H](C(=O)N[C@H](C(=O)N(C)[C@H](/C=C(\C)C(=O)NCCCCC(N)C(=O)O)C(C)C)C(C)(C)C)C(C)(C)c1cn(C)c2ccccc12]

or a salt thereof.

[Item 8]

An agent for eliminating a pluripotent stem cell, the agent comprising:

an antibody-drug conjugate represented by formula (2-1):

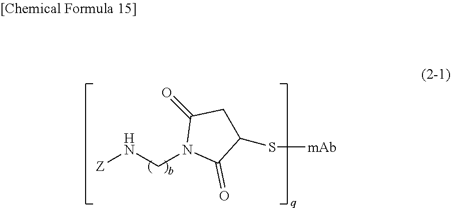

wherein

mAb represents an antibody recognizing an antigen expressed on a surface of a pluripotent stem cell;

q represents an integer of 1 to 20;

b represents an integer of 1 to 5; and

Z is a group represented by formula (Z-1) or formula (Z-2):

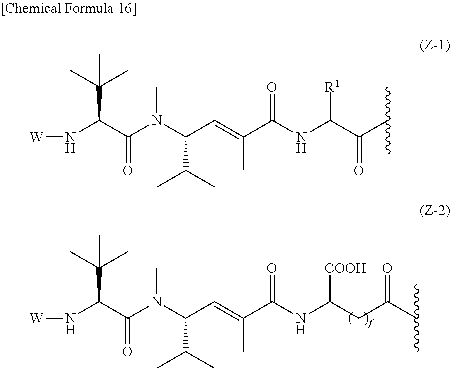

where
W represents a group represented by formula (W-1) or formula (W-2):

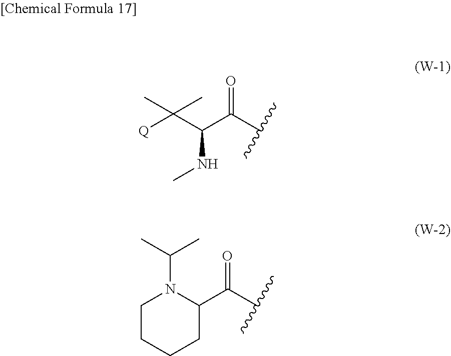

where
   Q represents a group represented by formula (Q-1) or formula (Q-2):

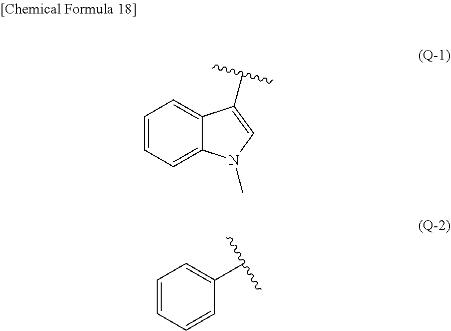 [SMILES: *c1cn(C)c2ccccc12 ; *c1ccccc1]

f represents 1 or 2;
R¹ represents —(CH₂)ᵤ—COOH; and
u represents 1 or 2,
   or a salt thereof.

[Item 9]

An agent for eliminating a pluripotent stem cell, the agent comprising:

an antibody-drug conjugate represented by formula (2-2):

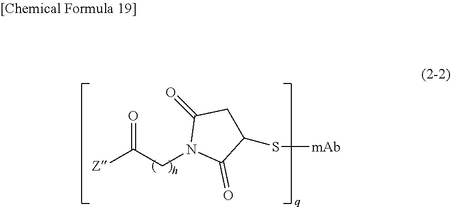

wherein

mAb represents an antibody recognizing an antigen expressed on a surface of a pluripotent stem cell;

q represents an integer of 1 to 20;

h represents an integer of 1 to 5;

Z″ is a group represented by formula (Z-5), formula (Z-6), formula (Z-7), formula (Z-8) or formula (Z-9):

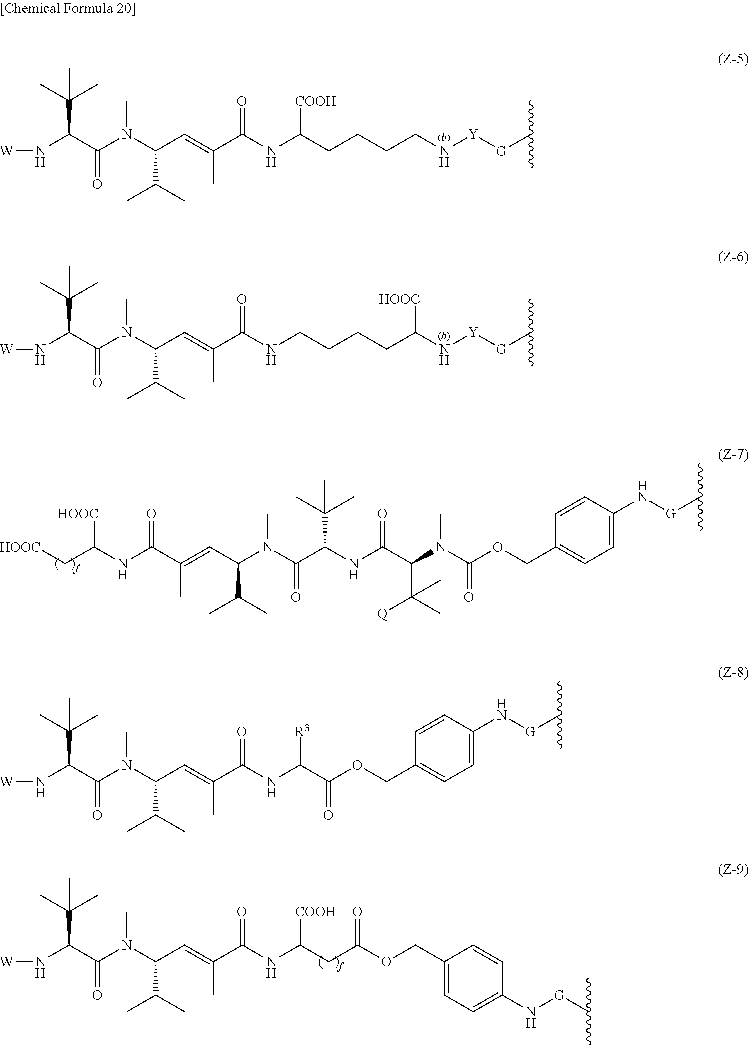

where
W represents a group represented by formula (W-1) or formula (W-2):

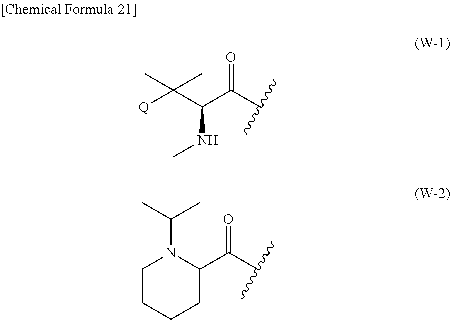

where
   Q represents a group represented by formula (Q-1) or formula (Q-2):

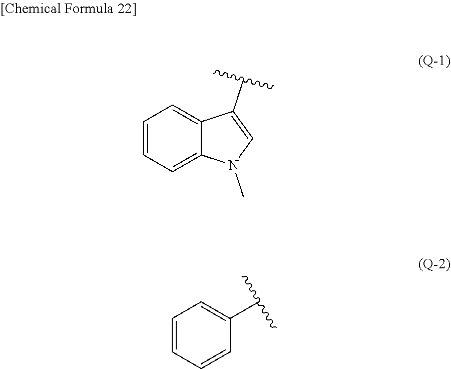 [SMILES: *c1cn(C)c2ccccc12 ; *c1ccccc1]

Y represents a single bond or a group represented by formula (Y-1):

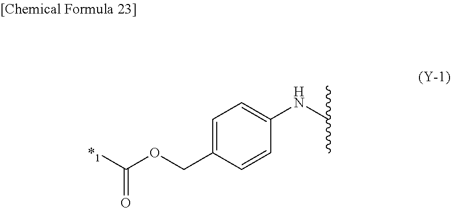

terminus *1 of the group represented by formula (Y-1) represents forming a bond together with amine (b);
G represents a single bond, *2-Gly-, *2-Gly-Gly-, *2-Lys-, *2-Lys-Phe-, *2-Lys-Val-, *2-Lys-Ala-, *2-Cit-Val-, *2-Cit-Phe-, *2-Cit-Leu-, *2-Arg-Phe-, *2-Cit-Ile-, *2-Cit-Trp-, *2-Lys-Phe-Phe-, *2-Lys-Phe-Ala-, *2-Lys-Phe-Gly-, *2-Asn-, *2-Asn-Ala-, *2-Asn-Ala-Ala-, *2-Asn-Ala-Thr-, *2-Asn-Ala-Pro-, *2-Asn-Ala-Val-, *2-Asn-Ala-Phe-, *2-Asn-Ala-Tyr-, *2-Asn-Ala-Leu-, *2-Asn-Ala-Gly-, *2-Asn-Thr-Ala-, *2-Asn-Thr-Pro-, *2-Asn-Thr-Thr-, *2-Gly-Phe-Gly-Gly-, *2-Gly-Leu-Phe-Gly- or *2-Leu-Ala-Leu-Ala-;
terminus *2 of G represents bonding to Y or —NH—;
   f represents 1 or 2;
R³ represents —(CH₂)ᵤ—COOH; and
u represents 1 or 2,
   or a salt thereof.

[Item 10]

An agent for eliminating a pluripotent stem cell, the agent comprising:

the antibody-drug conjugate according to item 9, wherein

Z″ is a group represented by formula (Z-5) or formula (Z-6);

Y is a single bond; and

G is a single bond,

or a salt thereof.

[Item 11]

An agent for eliminating a pluripotent stem cell, the agent comprising:

the antibody-drug conjugate according to item 9, wherein

Z″ is a group represented by formula (Z-5) or formula (Z-6);

Y is a group represented by formula (Y-1); and

G is *2-Cit-Val-, *2-Asn-Ala-, *2-Asn-Ala-Ala- or

### *2-Asn-Ala-Pro-,

or a salt thereof.

[Item 12]

An agent for eliminating a pluripotent stem cell, the agent comprising:

the antibody-drug conjugate according to item 9, wherein

Z″ is a group represented by formula (Z-7), formula (Z-8) or formula (Z-9); and

G is *2-Cit-Val-, *2-Asn-Ala-, *2-Asn-Ala-Ala- or *2-Asn-Ala-Pro-,

or a salt thereof.

[Item 13]

An agent for eliminating a pluripotent stem cell, the agent comprising:

the antibody-drug conjugate according to any one of items 8 to 12, wherein

W is a group represented by formula (W-1),

or a salt thereof.

[Item 14]

An agent for eliminating a pluripotent stem cell, the agent comprising:

the antibody-drug conjugate according to any one of items 8 to 13, wherein

mAb is an anti-CD30 antibody, an anti-TRA1-60 antibody, an anti-TRA1-81 antibody, an anti-SSEA3 antibody, an anti-SSEA4 antibody or an anti-rBC2LCN antibody,

or a salt thereof.

[Item 15]

An agent for eliminating a pluripotent stem cell, the agent comprising:

the antibody-drug conjugate according to any one of items 8 to 13, wherein

mAb is an anti-CD30 antibody,

or a salt thereof.

[Item 16]

An agent for eliminating a pluripotent stem cell, the agent comprising:

the antibody-drug conjugate according to any one of items 8 to 15, wherein

q is an integer of 1 to 8,

or a salt thereof.

[Item 17]

An agent for eliminating a pluripotent stem cell, the agent comprising:

an antibody-drug conjugate produced from a compound represented by formula (3-1):

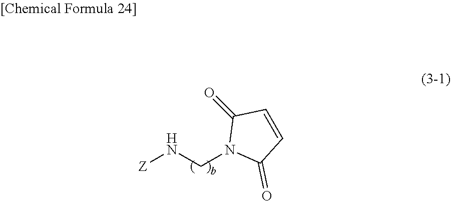

wherein

b represents an integer of 1 to 5; and

Z is a group represented by formula (Z-1) or formula (Z-2):

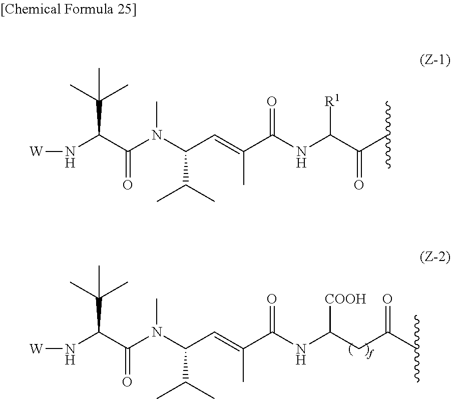

where
W represents a group represented by formula (W-1) or formula (W-2):

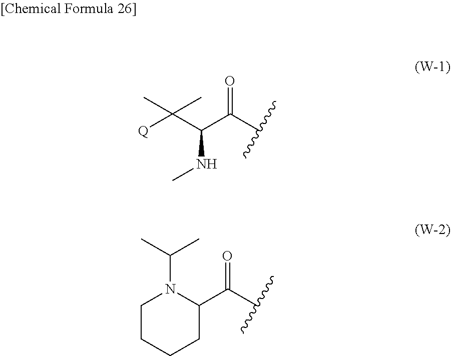

where
   Q represents a group represented by formula (Q-1) or formula (Q-2):

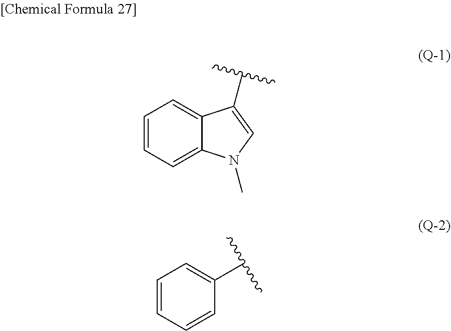 [SMILES: *c1cn(C)c2ccccc12 ; *c1ccccc1]

f represents 1 or 2;
R¹ represents —(CH₂)ᵤ—COOH; and
u represents 1 or 2,
   or a salt thereof.

[Item 18]

An agent for eliminating a pluripotent stem cell, the agent comprising:

an antibody-drug conjugate produced from a compound represented by formula (3-2):

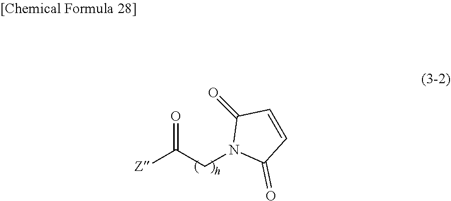

wherein

h represents an integer of 1 to 5; and

Z″ is a group represented by formula (Z-5), formula (Z-6), formula (Z-7), formula (Z-8) or formula (Z-9):

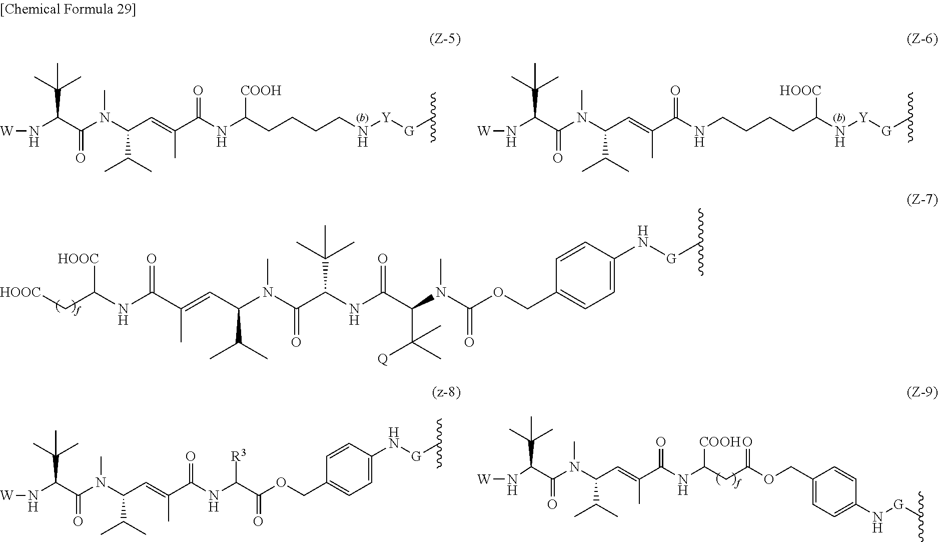

where
W represents a group represented by formula (W-1) or formula (W-2):

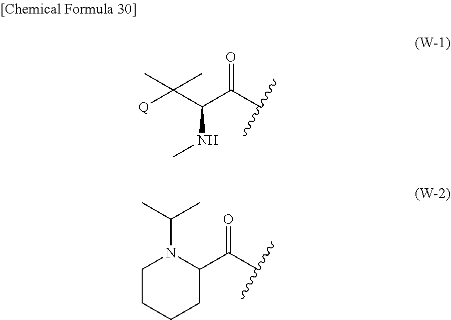

where
   Q represents a group represented by formula (Q-1) or formula (Q-2):

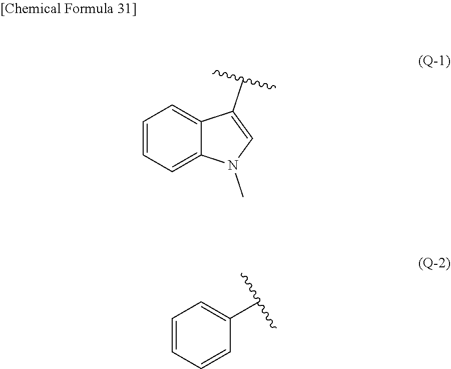 [SMILES: *c1cn(C)c2ccccc12 ; *c1ccccc1]

Y represents a single bond or a group represented by formula (Y-1):

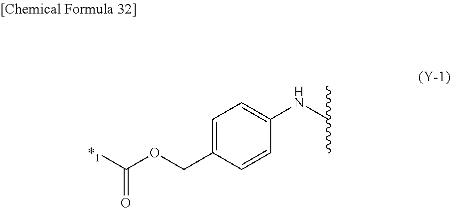

terminus *1 of the group represented by formula (Y-1) represents forming a bond together with amine (b);
G represents a single bond, *2-Gly-, *2-Gly-Gly-, *2-Lys-, *2-Lys-Phe-, *2-Lys-Val-, *2-Lys-Ala-, *2-Cit-Val-, *2-Cit-Phe-, *2-Cit-Leu-, *2-Arg-Phe-, *2-Cit-Ile-, *2-Cit-Trp-, *2-Lys-Phe-Phe-, *2-Lys-Phe-Ala-, *2-Lys-Phe-Gly-, *2-Asn-, *2-Asn-Ala-, *2-Asn-Ala-Ala-, *2-Asn-Ala-Thr-, *2-Asn-Ala-Pro-, *2-Asn-Ala-Val-, *2-Asn-Ala-Phe-, *2-Asn-Ala-Tyr-, *2-Asn-Ala-Leu-, *2-Asn-Ala-Gly-, *2-Asn-Thr-Ala-, *2-Asn-Thr-Pro-, *2-Asn-Thr-Thr-, *2-Gly-Phe-Gly-Gly-, *2-Gly-Leu-Phe-Gly- or *2-Leu-Ala-Leu-Ala-;
terminus *2 of G represents bonding to Y or —NH—;
f represents 1 or 2;
R³ represents —(CH₂)ᵤ—COOH; and
u represents 1 or 2,
   or a salt thereof.

[Item 19]

An agent for eliminating a pluripotent stem cell, the agent comprising:

an antibody-drug conjugate produced from the compound according to item 18, wherein

Z″ is a group represented by formula (Z-5) or formula (Z-6);

Y is a single bond; and

G is a single bond,

or a salt thereof.

[Item 20]

An agent for eliminating a pluripotent stem cell, the agent comprising:

an antibody-drug conjugate produced from the compound according to item 18, wherein

Z″ is a group represented by formula (Z-5) or formula (Z-6);

Y is a group represented by formula (Y-1); and

G is *2-Cit-Val-, *2-Asn-Ala-, *2-Asn-Ala-Ala- or *2-Asn-Ala-Pro-,

or a salt thereof.

[Item 21]

An agent for eliminating a pluripotent stem cell, the agent comprising:

an antibody-drug conjugate produced from the compound according to item 18, wherein

Z″ is a group represented by formula (Z-7), formula (Z-8) or formula (Z-9); and

G is *2-Cit-Val-, *2-Asn-Ala-, *2-Asn-Ala-Ala- or *2-Asn-Ala-Pro-,

or a salt thereof.

[Item 22]

An agent for eliminating a pluripotent stem cell, the agent comprising:

an antibody-drug conjugate produced from the compound according to any one of items 17 to 21, wherein

W is a group represented by formula (W-1),

or a salt thereof.

[Item 23]

A killing agent for a pluripotent stem cell, the killing agent comprising:

the antibody-drug conjugate according to any one of items 1 to 22 or a salt thereof.

[Item 24]

A reducer for a pluripotent stem cell, the reducer comprising: the antibody-drug conjugate according to any one of items 1 to 22 or a salt thereof.

[Item 25]

The agent for eliminating a pluripotent stem cell according to any one of items 1 to 22, wherein the pluripotent stem cell is an ES cell or an iPS cell.

[Item 26]

The agent for eliminating a pluripotent stem cell according to any one of items 1 to 22, wherein the pluripotent stem cell is an iPS cell.

[Item 27]

A method for eliminating a pluripotent stem cell, comprising:

a step of adding the antibody-drug conjugate according to any one of items 1 to 22 or a salt thereof to a culture solution containing a pluripotent stem cell.

[Item 28]

A method for eliminating a pluripotent stem cell, comprising:

a step of adding the antibody-drug conjugate according to any one of items 1 to 22 or a salt thereof to a culture solution containing a cell cluster produced by culturing a pluripotent stem cell.

[Item 29]

The method for eliminating a pluripotent stem cell according to item 27 or 28, wherein the pluripotent stem cell is an iPS cell.

[Item 30]

Use of the antibody-drug conjugate according to any one of items 1 to 22 or a salt thereof for producing a cell population including a differentiated cell derived from an iPS cell with substantially no iPS cell.

[Item 31]

A method for producing a cell population including a differentiated cell derived from an iPS cell with substantially no iPS cell, comprising:

a step of contacting the antibody-drug conjugate according to any one of items 1 to 22 or a salt thereof and a cell population including a differentiated cell derived from an iPS cell.

[Item 32]

A method for producing a cell population including a differentiated cell derived from an iPS cell with substantially no iPS cell, comprising:

(1) a step of performing induction of differentiation into a differentiated cell for a cell population including an iPS cell; and

(2) a step of contacting the cell population including a differentiated cell obtained in the step (1) with the antibody-drug conjugate according to any one of items 1 to 22 or a salt thereof.

[Item 33]

A cell population including a differentiated cell derived from an iPS cell with substantially no iPS cell, wherein the cell population is produced by the method according to item 31 or 32.

[Item 34]

The cell population according to item 33, wherein the differentiated cell is a cell for transplantation.

[Item 35]

A pharmaceutical composition comprising, as an active ingredient:

a differentiated cell included in the cell population according to item 33 or 34.

### Advantageous Effects of Invention

The agent for eliminating a pluripotent stem cell according to the present invention can efficiently eliminate pluripotent stem cells from cellular medicines derived from pluripotent stem cells. Especially, the agent for eliminating a pluripotent stem cell according to the present invention can selectively eliminate pluripotent stem cells from a cell population including a differentiated cell, the cell population produced by culturing a pluripotent stem cell.

### BRIEF DESCRIPTION OF DRAWINGS

FIG. 1 shows activities of monomethyl auristatin (MMAE), hemiasterlin and Example 1 to inhibit polymerization of porcine tubulins.

FIG. 2 shows cytotoxic activities of Example ADC1 and Example ADC23 to iPS cells.

FIG. 3 shows cytotoxic activities of Example ADC1 and Example ADC23 to differentiated cells.

### DESCRIPTION OF EMBODIMENTS

In the present specification, the “C₁₋₆ alkyl group” means a linear or branched saturated hydrocarbon group having 1 to 6 carbon atoms. Examples of the “C₁₋₆ alkyl group” preferably include a “C₁₋₄ alkyl group”, more preferably include a “C₁₋₃ alkyl group”, further preferably include a methyl group, an ethyl group, a propyl group or an isopropyl group, and particularly preferably include a methyl group or an ethyl group.

Specific examples of the “C₁₋₃ alkyl group” include a methyl group, an ethyl group, a propyl group and an isopropyl group. Specific examples of the “C₁₋₄ alkyl group” include a butyl group, a 1,1-dimethylethyl group, a 1-methylpropyl group and a 2-methylpropyl group in addition to those mentioned as the specific examples of the “C₁₋₃ alkyl group”. Specific examples of the “C₁₋₆ alkyl group” include a pentyl group, a 3-methylbutyl group, a 2-methylbutyl group, a 2,2-dimethylpropyl group, a 1-ethylpropyl group, a 1,1-dimethylpropyl group, a hexyl group, a 4-methylpentyl group, a 3-methylpentyl group, a 2-methylpentyl group, a 1-methylpentyl group, a 3,3-dimethylbutyl group, a 2,2-dimethylbutyl group, a 1,1-dimethylbutyl group and a 1,2-dimethylbutyl group in addition to those mentioned as the specific examples of the “C₁₋₄ alkyl group”.

In the present specification, examples of the “halogen atom” include a fluorine atom, a chlorine atom, a bromine atom or an iodine atom. Preferably, examples thereof include a fluorine atom or a chlorine atom, and more preferably, examples thereof include a fluorine atom.

<Hemiasterlin Derivative>

A compound represented by formula (1-1), formula (1-2) or formula (1-3), and a salt thereof (hereinafter, may be referred to as the “hemiasterlin derivative according to the present invention”) is as follows:

(1) Compound Represented by Formula (1-1) and Salt Thereof

Among hemiasterlin derivatives according to the present invention, a compound represented by the following formula (1-1) and a salt thereof will be described.

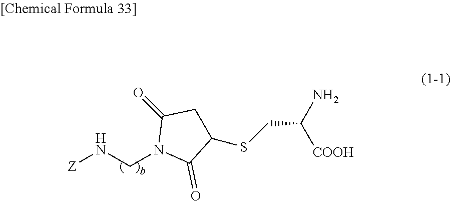

In the formula, b represents an integer of 1 to 5. That is, b is 1, 2, 3, 4 or 5. Examples of one aspect of b include an integer of 1 to 4; examples of another aspect thereof include an integer of 1 to 3; and examples of another aspect thereof include 2 or 3.

In the formula, Z represents a group represented by formula (Z-1) or formula (Z-2):

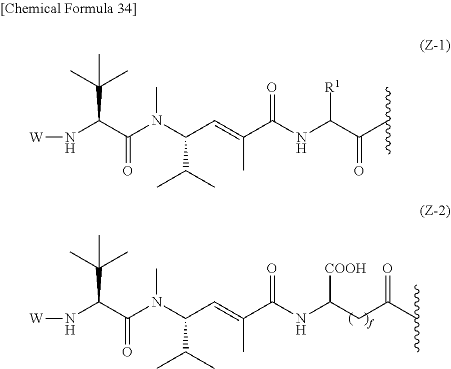

The configuration of each of the carbon atom to which substituent R¹ is bonding in formula (Z-1) and the carbon atom to which the carboxyl group (—COOH) is bonding in formula (Z-2) may be an S-form or an R-form.

In formula (Z-1), R¹ represents —(CH₂)ᵤ—COOH. Here, u is 1 or 2. Examples of one aspect of u include 1, and examples of another aspect thereof include 2.

In formula (Z-2), f represents 1 or 2. Examples of one aspect of f include 1, and examples of another aspect thereof include 2.

In formula (Z-1) or formula (Z-2), W represents a group represented by formula (W-1) or formula (W-2):

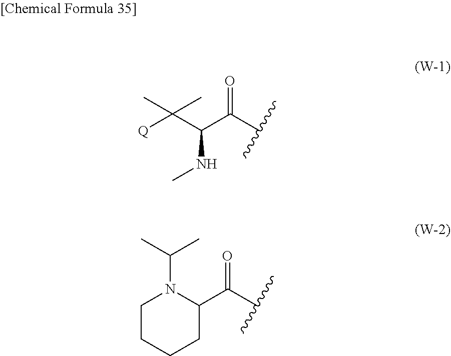

Examples of one aspect of W include a group represented by formula (W-1), and examples of another aspect thereof include a group represented by formula (W-2).

In formula (W-1), Q represents a group represented by formula (Q-1) or formula (Q-2):

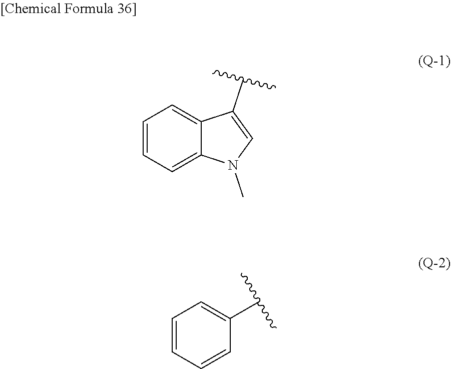 [SMILES: *c1cn(C)c2ccccc12 ; *c1ccccc1]

Examples of one aspect of Q include a group represented by formula (Q-1), and examples of another aspect thereof include a group represented by formula (Q-2).

In the present specification, a hydrogen atom may be ¹H or ²H(D). That is, for example, a deuterated product in which one or two or more ¹H of the compound represented by formula (1-1) are converted into ²H(D) is also encompassed in the compound represented by formula (1-1).

Examples of one aspect of the hemiasterlin derivative according to the present invention include the following (1-1-A).

### (1-1-A)

A compound, wherein, in formula (1-1),

b is 2, 3 or 4;

Z is a group represented by formula (Z-1) or formula (Z-2);

W is a group represented by formula (W-1);

Q is a group represented by formula (Q-1);

R¹ is —(CH₂)ᵤ—COOH;

u is an integer of 1 or 2; and

f is an integer of 1 or 2,

or a salt thereof.

Examples of one aspect of the hemiasterlin derivative according to the present invention include the following (1-1-B).

### (1-1-B)

A compound, wherein, in formula (1-1),

b is 2, 3 or 4;

Z is a group represented by formula (Z-1) or formula (Z-2);

W is a group represented by formula (W-1);

Q is a group represented by formula (Q-2);

R¹ is —(CH₂)ᵤ—COOH;

u is an integer of 1 or 2; and

f is an integer of 1 or 2,

or a salt thereof.

(2) Compound Represented by Formula (1-2) and Salt Thereof

Among hemiasterlin derivatives according to the present invention, a compound represented by the following formula (1-2) and a salt thereof will be described.

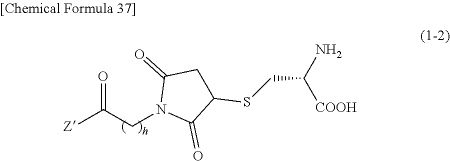

In the formula, h represents an integer of 1 to 5. That is, h is 1, 2, 3, 4 or 5. Examples of one aspect of h include an integer of 1 to 4; examples of another aspect thereof include an integer of 1 to 3; and examples of another aspect thereof include 3.

In the formula, Z′ represents a group represented by formula (Z-3) or formula (Z-4):

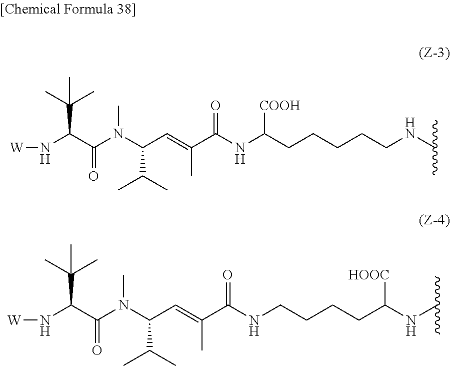

Examples of one aspect of Z′ include a group represented by formula (Z-3), and examples of another aspect thereof include a group represented by formula (Z-4). The configuration of the carbon atom to which the carboxyl group (—COOH) is bonding in formula (Z-3) and formula (Z-4) may be an S-form or an R-form.

In formula (Z-3) or formula (Z-4), W represents a group represented by formula (W-1) or formula (W-2):

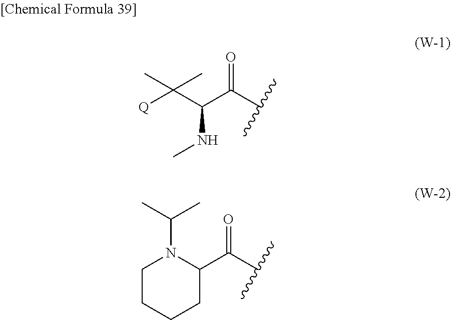

Examples of one aspect of W include a group represented by formula (W-1), and examples of another aspect thereof include a group represented by formula (W-2).

In formula (W-1), Q represents a group represented by formula (Q-1) or formula (Q-2):

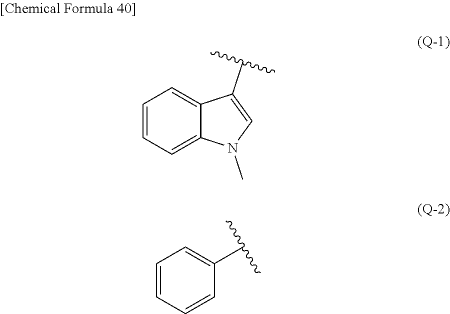 [SMILES: *c1cn(C)c2ccccc12 ; *c1ccccc1]

Examples of one aspect of Q include a group represented by formula (Q-1), and examples of another aspect thereof include a group represented by formula (Q-2).

Examples of one aspect of the hemiasterlin derivative according to the present invention include the following (1-2-A).

### (1-2-A)

A compound, wherein, in formula (1-2),

h is 2, 3, 4 or 5;

Z′ is a group represented by formula (Z-3);

W is a group represented by formula (W-1); and

Q is a group represented by formula (Q-1) or formula (Q-2),

or a salt thereof.

Examples of one aspect of the hemiasterlin derivative according to the present invention include the following (1-2-B).

### (1-2-B)

A compound, wherein, in formula (1-2),

h is 2, 3, 4 or 5;

Z′ is a group represented by formula (Z-4);

W is a group represented by formula (W-1); and

Q is a group represented by formula (Q-1) or formula (Q-2),

or a salt thereof.

(3) Compound Represented by Formula (1-3) and Salt Thereof

Among hemiasterlin derivatives according to the present invention, a compound represented by the following formula (1-3) and a salt thereof will be described.

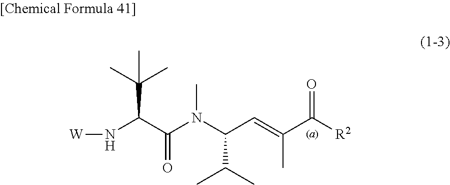

In the formula, R² represents a glutamic acid residue, an aspartic acid residue or a lysine residue, and examples thereof preferably include a glutamic acid residue or an aspartic acid residue. In the present specification, except when it is particularly necessary to make distinction, the three letter abbreviated notations shown below may be used for representing both α-amino acids and corresponding amino acid residues. In addition, the optical activity of the α-amino acids may include any of DL form, D form and L form unless otherwise specified. For example, “glutamic acid” or “Glu” represents DL-glutamic acid or a residue thereof, D-glutamic acid or a residue thereof, or L-glutamic acid or a residue thereof.

Asp: aspartic acid, Glu: glutamic acid, Lys: lysine

The N-terminal nitrogen atom of R² forms an amide bond together with carbonyl group (a). “The N-terminal nitrogen atom of R² forms an amide bond together with carbonyl (a)” means that, for example, when R² is Asp, nitrogen atom (b) of Asp and carbonyl group (a) are linked to form an amide bond, as represented by the following formula:

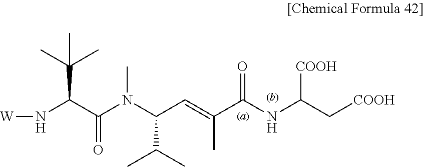

In the formula, W represents a group represented by formula (W-1) or formula (W-2):

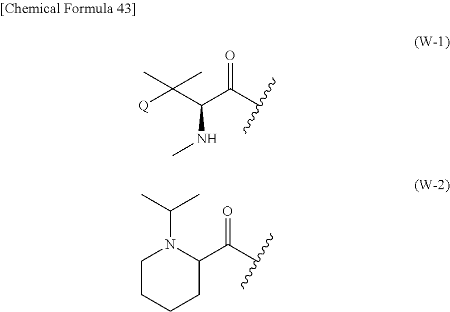

Examples of one aspect of W include a group represented by formula (W-1), and examples of another aspect thereof include a group represented by formula (W-2).

In formula (W-1), Q represents a group represented by formula (Q-1) or formula (Q-2):

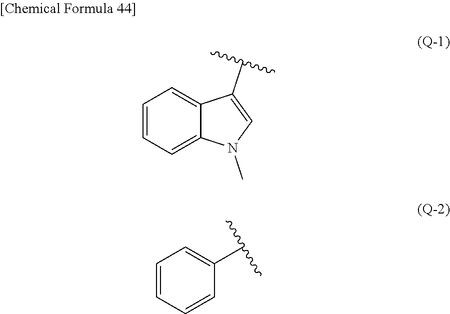 [SMILES: *c1cn(C)c2ccccc12 ; *c1ccccc1]

Examples of one aspect of Q include a group represented by formula (Q-1), and examples of another aspect thereof include a group represented by formula (Q-2).

Examples of one aspect of the hemiasterlin derivative according to the present invention include the following (1-3-A).

### (1-3-A)

A compound, wherein, in formula (1-3),

R² is a glutamic acid residue;

W is a group represented by formula (W-1); and

Q is a group represented by formula (Q-1) or formula (Q-2),

or a salt thereof.

Examples of one aspect of the hemiasterlin derivative according to the present invention include the following (1-3-B).

### (1-3-B)

A compound, wherein, in formula (1-3),

R² is an aspartic acid residue;

W is a group represented by formula (W-1); and

Q is a group represented by formula (Q-1) or formula (Q-2),

or a salt thereof.

Examples of one aspect of the hemiasterlin derivative according to the present invention include the following (1-3-C).

### (1-3-C)

A compound, wherein, in formula (1-3),

R² is a lysine residue;

W is a group represented by formula (W-1); and

Q is a group represented by formula (Q-1) or formula (Q-2),

or a salt thereof.

<Antibody-Drug Conjugate>

An antibody-drug conjugate represented by formula (2-1) or formula (2-2), or a salt thereof (hereinafter, may be referred to as the “antibody-drug conjugate according to the present invention”) is, as shown below, a conjugate in which the antibody moiety derived from an antibody molecule and a drug moiety derived from a drug molecule are covalently bonded. In the present specification, the “antibody-drug conjugate” may be referred to as “ADC”.

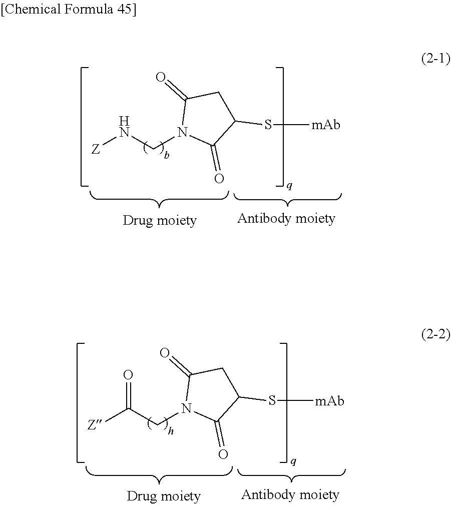

In the antibody-drug conjugate according to the present invention, a compound having a hemiasterlin-derived backbone represented by Z or Z″ is bonded to an antibody via a linker having succinimide structure. The drug moiety of the antibody-drug conjugate according to the present invention, having a part or the whole of the structure of the hemiasterlin derivative according to the present invention, refers to a structure of the antibody-drug conjugate excluding the antibody. The antibody-drug conjugate releases the compound derived from the drug moiety through undergoing metabolism, and the compound to be released may be a part or the whole of the drug moiety. That is, the compound to be released may be the hemiasterlin derivative according to the present invention, or a compound having a hemiasterlin-derived backbone, which is the partial structure of the hemiasterlin derivative according to the present invention. In the present specification, the compound to be released from the antibody-drug conjugate may be referred to as the “compound derived from the drug moiety”.

In the formulas, q indicates the drug antibody ratio (alternatively, referred to as DAR) in the antibody-drug conjugate molecules. Drug antibody ratio q means the number of drug molecules per antibody molecule in one molecule of the antibody-drug conjugate, that is, per antibody-drug conjugate molecule. Note that antibody-drug conjugates obtained through chemical synthesis are often a mixture of a plurality of antibody-drug conjugate molecules that may have different drug antibody ratio q. In the present specification, the overall drug antibody ratio in such a mixture of antibody-drug conjugates (that is, the average value of drug antibody ratio q of each antibody-drug conjugate) is referred to as the “average drug antibody ratio” or “average DAR”.

q represents an integer of 1 to 20. That is, q is 1, 2, 3, 4, 5, 6, 7, 8, 9, 10, 11, 12, 13, 14, 15, 16, 17, 18, 19 or 20. Examples of one aspect of q include an integer of 1 to 10; examples of another aspect thereof include an integer of 11 to 20; examples of another aspect thereof include an integer of 1 to 8; examples of another aspect thereof include an integer of 4 to 8; and examples of another aspect thereof include 8.

Examples of one aspect of the average DAR include 1 to 20; examples of another aspect thereof include 1 to 10; and examples of another aspect thereof include 10 to 20. Examples of another aspect thereof include 1 to 8, 1 to 2, 2 to 3, 3 to 4, 4 to 5, 6 to 7, 7 to 8, 8 to 9, 9 to 10, 5 to 15. It is possible to determine the average DAR by methods conventionally used to determine the average DAR, such as ultraviolet-visible-near-infrared spectroscopy, SDS-PAGE, mass spectrometry, ELISA (enzyme-linked immunosorbent assay) and HPLC (high performance liquid chromatography). It is possible to separate, purify and characterize an antibody-drug conjugate of a particular DAR from a mixture of a plurality of antibody-drug conjugates having different DARs by methods such as hydrophobic interaction chromatography (HIC) HPLC, reversed phase HPLC and electrophoresis.

mAb represents an “antibody”. Here, it is sufficient that the “antibody” be an antibody including at least a heavy chain variable domain and a light chain variable domain, and it may be a complete antibody or a fragment of a complete antibody that is an antigen-binding fragment having an antigen-recognition site. The complete antibody has two full length light chains and two full length heavy chains, and respective light chains and heavy chains are linked by disulfide bonds. The complete antibody includes IgA, IgD, IgE, IgM and IgG, and IgG includes IgG₁, IgG₂, IgG₃ and IgG₄ as subtypes. In addition, it is preferable that the antibody be a monoclonal antibody. The antibody moiety and the drug moiety are linked via a sulfhydryl group obtained by reducing a disulfide bond in the antibody.

It is desirable in the present specification that the antibody and mAb be an antibody that recognizes an antigen expressed on surfaces of pluripotent stem cells, specifically, ES cells or iPS cells. It is preferable that the antigen present on surfaces of pluripotent stem cells be an antigen specific to pluripotent stem cells, wherein the antigen is not expressed on differentiated cells or the expression level is low on differentiated cells. For example, an antigen whose expression level on pluripotent stem cells is 10 times or more, preferably 100 times or more, further preferably 1000 times or more the expression level on differentiated cells may be selected. Examples of the antigen expressed on surfaces of pluripotent stem cells such as ES cells or iPS cells include, but are not limited to, CD30, TRA1-60, TRA1-81, SSEA3, SSEA4 and rBC2LCN. Examples of one aspect of mAb include an anti-CD30 antibody, anti-TRA1-60 antibody, anti-TRA1-81 antibody, anti-SSEA3 antibody, anti-SSEA4 antibody or anti-rBC2LCN antibody, and examples of another aspect thereof include an anti-CD30 antibody.

Examples of the anti-CD30 antibody include brentuximab and iratumumab. Commercial products may be used for the anti-TRA1-60 antibody, anti-TRA1-81 antibody, anti-SSEA3 antibody, anti-SSEA4 antibody and anti-rBC2LCN antibody.

The antibody moiety of the antibody-drug conjugate according to the present invention may be an antibody against an antigen expressed on cell surfaces of pluripotent stem cells such as ES cells or iPS cells. Examples of one aspect of mAb include brentuximab or iratumumab, and preferably include brentuximab. Commercial products may be used for the antibodies specifically mentioned here, or the antibodies may be produced by known methods.

The antibody moiety of the antibody-drug conjugate according to the present invention may be an antibody against an antigen expressed on cell surfaces of pluripotent stem cells such as ES cells or iPS cells. Examples of mAb include an anti-CD30 antibody, anti-TRA1-60 antibody, anti-TRA1-81 antibody, anti-SSEA3 antibody, anti-SSEA4 antibody or anti-rBC2LCN antibody. Examples of another aspect of mAb include an anti-CD30 antibody.

Z and b in formula (2-1) are as defined for those symbols in formula (1-1).

Z″ in formula (2-2) represents a group represented by formula (Z-5), formula (Z-6), formula (Z-7), formula (Z-8) or formula (Z-9):

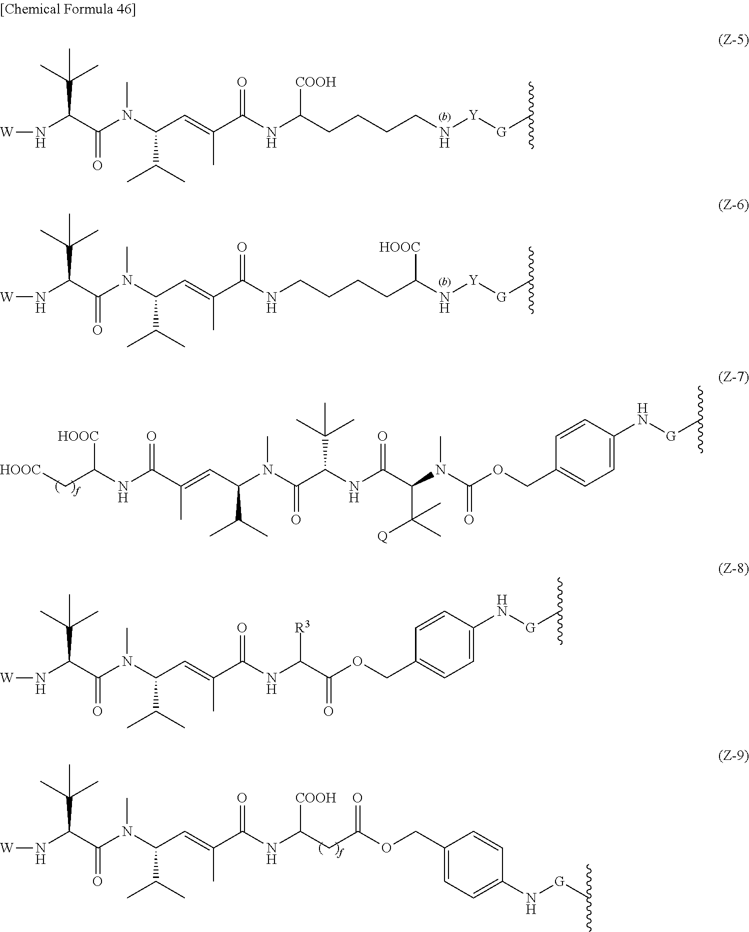

In the formula, G is a single bond, *²-Gly-, *²-Gly-Gly-, *²-Lys-, *2-Lys-Phe-, *²-Lys-Val-, *²-Lys-Ala-, *²-Cit-Val-, *²-Cit-Phe-, *²-Cit-Leu-, *²-Arg-Phe-, *²-Cit-Ile-, *²-Cit-Trp-, *²-Lys-Phe-Phe-, *²-Lys-Phe-Ala-, *²-Lys-Phe-Gly-, *²-Asn-, *²-Asn-Ala-, *²-Asn-Ala-Ala-, *²-Asn-Ala-Thr-, *²-Asn-Ala-Pro-, *²-Asn-Ala-Val-, *²-Asn-Ala-Phe-, *²-Asn-Ala-Tyr-, *²-Asn-Ala-Leu-, *²-Asn-Ala-Gly-, *²-Asn-Thr-Ala-, *²-Asn-Thr-Pro-, *²-Asn-Thr-Thr-, *²-Gly-Phe-Gly-Gly-, *²-Gly-Leu-Phe-Gly- or *²-Leu-Ala-Leu-Ala-, where terminus *² of G represents bonding to Y or —NH—. Examples of one aspect of G include a single bond, *²-Asn-Ala-, *²-Asn-Ala-Ala-, *²-Asn-Ala-Pro- or *²-Cit-Val-; examples of another aspect thereof include a single bond; and examples of another aspect thereof include *²-Asn-Ala-, *²-Asn-Ala-Ala-, *²-Asn-Ala-Pro- or *²-Cit-Val-, and preferably include *²-Cit-Val-.

In the formula, Y represents a single bond or a group represented by formula (Y-1):

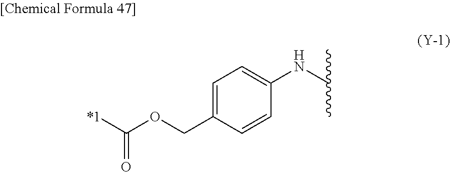

Examples of one aspect of Y include a single bond, and examples of another aspect thereof include a group represented by formula (Y-1).

Terminus *1 in formula (Y-1) forms a bond together with amine (b).

In formula (Z-5), formula (Z-6), formula (Z-8) or formula (Z-9), W represents a group represented by formula (W-1) or formula (W-2):

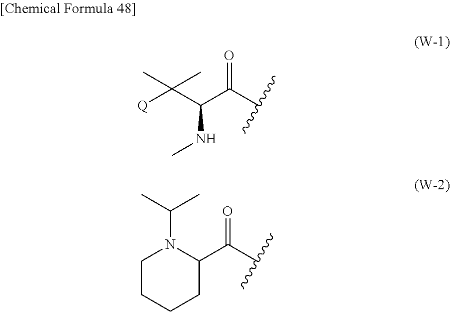

Examples of one aspect of W include a group represented by formula (W-1), and examples of another aspect thereof include a group represented by formula (W-2).

In formula (W-1) and formula (Z-7), Q represents a group represented by formula (Q-1) or formula (Q-2):

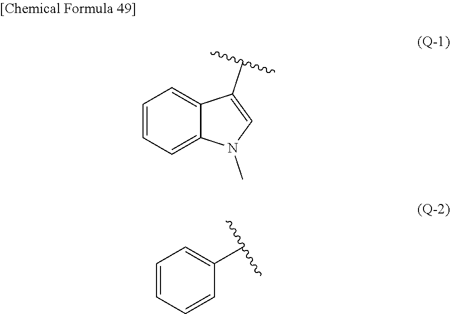 [SMILES: *c1cn(C)c2ccccc12 ; *c1ccccc1]

Examples of one aspect of Q include a group represented by formula (Q-1), and examples of another aspect thereof include a group represented by formula (Q-2).

In formula (Z-7) and formula (Z-9), f represents 1 or 2. Examples of one aspect of f include 1, and examples of another aspect thereof include 2.

In formula (Z-8), R³ represents —(CH₂)ᵤ—COOH. Here, u is 1 or 2.

In an organism, the antibody-drug conjugate according to the present invention, wherein, in the formula, G is *²-Gly-, *²-Gly-Gly-, *²-Lys-, *2-Lys-Phe-, *²-Lys-Val-, *²-Lys-Ala-, *²-Cit-Val-, *²-Cit-Phe-, *²-Cit-Leu-, *²-Arg-Phe-, *²-Cit-Ile-, *²-Cit-Trp-, *²-Lys-Phe-Phe-, *²-Lys-Phe-Ala-, *²-Lys-Phe-Gly-, *²-Asn-, *²-Asn-Ala-, *²-Asn-Ala-Ala-, *²-Asn-Ala-Thr-, *²-Asn-Ala-Pro-, *²-Asn-Ala-Val-, *²-Asn-Ala-Phe-, *²-Asn-Ala-Tyr-, *²-Asn-Ala-Leu-, *²-Asn-Ala-Gly-, *²-Asn-Thr-Ala-, *²-Asn-Thr-Pro-, *²-Asn-Thr-Thr-, *²-Gly-Phe-Gly-Gly-, *²-Gly-Leu-Phe-Gly- or *²-Leu-Ala-Leu-Ala-, and Y is represented by formula (Y-1), is assumed to be cleaved at the bond of Y-G in formula (Z-5) and formula (Z-6) or at the bond of —NH-G in formula (Z-7), formula (Z-8) and formula (Z-9) at first, and then cleaved at the bond between the drug and Y, thereby releasing the hemiasterlin derivative represented by formula (1-1), formula (1-2) or formula (1-3).

The G-Y bond or the G-NH bond is cleaved by intracellular peptidase, protease (for example, lysosomal protease or endosomal protease) or the like, which is present in an intracellular environment (for example, in lysosome, endosome or caveola).

As a protease present in an intracellular environment, for example, cathepsin B is known. Cleavage of the G-Y bond or the G-NH bond by cathepsin B is described in Dubowchik G. M., et al, 1998, Bioorg. Med. Chem. Lett., 8: 3341-3346 and the like. Specific examples of Y-G cleaved by cathepsin B include Y-Lys-Phe, Y-Lys-Val, Y-Lys-Ala, Y-Lys-Phe-Phe, Y-Lys-Phe-Ala, Y-Lys-Phe-Gly, Y-Lys, Y-Cit-Val, Y-Cit-Phe, Y-Cit-Leu, Y-Cit-Ile, Y-Cit-Trp and Y-Arg-Phe.

As another protease present in an intracellular environment, for example, asparagine endopeptidase is known. Cleavage of the G-Y bond or the G-NH bond by asparagine endopeptidase is described in Dando M. P., et al, 1999, Biochem. J. 339: 743-749 and the like. Specific examples of the G-Y bond or the G-NH bond cleaved by asparagine endopeptidase include Y-Asn-Ala-Ala, Y-Asn-Ala-Thr, Y-Asn-Ala-Val, Y-Asn-Ala-Pro, Y-Asn-Ala-Phe, Y-Asn-Ala-Tyr, Y-Asn-Ala-Leu and Y-Asn-Ala-Gly.

Putative mechanisms in which a drug is released from the antibody-drug conjugate according to the present invention, wherein G is *²-Gly-, *²-Gly-Gly-, *²-Lys-, *2-Lys-Phe-, *²-Lys-Val-, *²-Lys-Ala-, *²-Cit-Val-, *²-Cit-Phe-, *²-Cit-Leu-, *²-Arg-Phe-, *²-Cit-Ile-, *²-Cit-Trp-, *²-Lys-Phe-Phe-, *²-Lys-Phe-Ala-, *²-Lys-Phe-Gly-, *²-Asn-, *²-Asn-Ala-, *²-Asn-Ala-Ala-, *²-Asn-Ala-Thr-, *²-Asn-Ala-Pro-, *²-Asn-Ala-Val-, *²-Asn-Ala-Phe-, *²-Asn-Ala-Tyr-, *²-Asn-Ala-Leu-, *²-Asn-Ala-Gly-, *²-Asn-Thr-Ala-, *²-Asn-Thr-Pro-, *²-Asn-Thr-Thr-, *²-Gly-Phe-Gly-Gly-, *²-Gly-Leu-Phe-Gly- or *²-Leu-Ala-Leu-Ala-, and terminus *² of G bonds to a group represented by formula (Y-1) or —NH—, in an intracellular environment, are exemplified with formula (2-2), wherein Z″ is formula (Z-7). These drug release mechanisms are inferred from drug release mechanisms for antibody-drug conjugates described in Toki et al., 2002, J. Org. Chem. 67, 1866-1872. and the like.

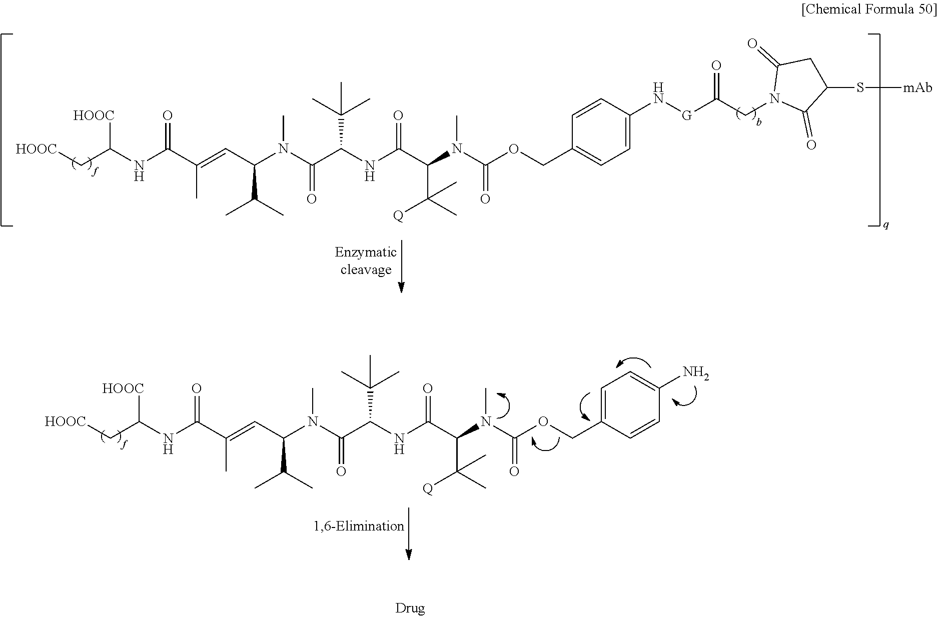

After the antibody-drug conjugate according to the present invention is taken up by pluripotent stem cells such as ES cells or iPS cells, the antibody is metabolized in the cells, and the compound derived from the drug moiety or the compound corresponding to a structure including a part of the antibody (antibody fragment) and the drug moiety may be released. In Doronina S. O. et al., 2006, Bioconjugate Chem. 17: 114-124, for example, it is disclosed that a Cys-drug moiety of an antibody-drug conjugate is released in cells through metabolism of the antibody. Examples of the Cys-bonded drug moiety that is released through the same mechanism in the antibody-drug conjugate according to the present invention include a compound represented by formula (1-1) or formula (1-2).

In the present specification, “release a compound” means that an antibody-drug conjugate releases a compound derived from the drug moiety into cells through undergoing metabolism of the antibody moiety by protease in the cells. The compound released exhibits pharmacological activity, that is, cytotoxic activity in cells, and induces cell death. An “antibody-drug conjugate that releases a compound” means an antibody-drug conjugate that can release a compound derived from the drug moiety through undergoing metabolism of the antibody moiety in cells.

The antibody-drug conjugate that releases the compound may be appropriately designed and produced by a technique well known to a person having ordinary skill in the art. For example, Antibody-Drug Conjugates (edited by Laurent Ducry, published by Humana Press, 2013) describes linkers that link an antibody and a drug and binding modes thereof, and discloses that antibody-drug conjugates designed and produced in such a manner release an intended compound through chemical reaction or enzymatic reaction.

Examples of one aspect of the antibody-drug conjugate according to the present invention include the following (2-1-A).

### (2-1-A)

An antibody-drug conjugate, wherein, in formula (2-1),

mAb is an antibody recognizing an antigen expressed on a surface of an iPS cell;

q is an integer of 1 to 8;

b is 2, 3, 4 or 5;

Z is a group represented by formula (Z-1) or formula (Z-2);

W is a group represented by formula (W-1);

Q is a group represented by formula (Q-1) or formula (Q-2);

f represents 1 or 2;

R¹ represents —(CH₂)ᵤ—COOH; and

u is 1 or 2,

or a salt thereof.

Examples of one aspect of the antibody-drug conjugate according to the present invention include the following (2-1-B).

### (2-1-B)

An antibody-drug conjugate, wherein, in formula (2-1),

mAb is an anti-CD30 antibody;

q is an integer of 1 to 8;

b is 2, 3, 4 or 5;

Z is a group represented by formula (Z-1) or formula (Z-2);

W is a group represented by formula (W-1);

Q is a group represented by formula (Q-1) or formula (Q-2);

f represents 1 or 2;

R¹ represents —(CH₂)ᵤ—COOH; and

u is 1 or 2,

or a salt thereof.

Examples of one aspect of the antibody-drug conjugate according to the present invention include the following (2-2-A).

### (2-2-A)

An antibody-drug conjugate, wherein, in formula (2-2),

mAb is an antibody recognizing an antigen expressed on a surface of an iPS cell;

q is an integer of 1 to 8;

h is 2, 3, 4 or 5;

Z″ is a group represented by formula (Z-5) or formula (Z-6);

G is a single bond;

Y is a single bond;

W is a group represented by formula (W-1); and

Q is a group represented by formula (Q-1) or formula (Q-2),

or a salt thereof.

Examples of one aspect of the antibody-drug conjugate according to the present invention include the following (2-2-B).

### (2-2-B)

An antibody-drug conjugate, wherein, in formula (2-2),

mAb is an anti-CD30 antibody;

q is an integer of 1 to 8;

h is 2, 3, 4 or 5;

Z″ is a group represented by formula (Z-5) or formula (Z-6);

G is a single bond;

Y is a single bond;

W is a group represented by formula (W-1); and

Q is a group represented by formula (Q-1) or formula (Q-2),

or a salt thereof.

Examples of one aspect of the antibody-drug conjugate according to the present invention include the following (2-2-C).

### (2-2-C)

An antibody-drug conjugate, wherein, in formula (2-2),

mAb is an antibody recognizing an antigen expressed on a surface of an iPS cell;

q is an integer of 1 to 8;

h is 2, 3, 4 or 5;

Z″ is a group represented by formula (Z-5) or formula (Z-6);

G is *²-Cit-Val-;

*² represents bonding between the G terminus and Y;

Y is a group represented by formula (Y-1);

W is a group represented by formula (W-1); and

Q is a group represented by formula (Q-1) or formula (Q-2),

or a salt thereof.

Examples of one aspect of the antibody-drug conjugate according to the present invention include the following (2-2-D).

### (2-2-D)

An antibody-drug conjugate, wherein, in formula (2-2),

mAb is an anti-CD30 antibody;

q is an integer of 1 to 8;

h is 2, 3, 4 or 5;

Z″ is a group represented by formula (Z-5) or formula (Z-6);

G is *²-Cit-Val-;

*² represents bonding between the G terminus and Y;

Y is a group represented by formula (Y-1);

W is a group represented by formula (W-1); and

Q is a group represented by formula (Q-1) or formula (Q-2),

or a salt thereof.

Examples of one aspect of the antibody-drug conjugate according to the present invention include the following (2-2-E).

### (2-2-E)

An antibody-drug conjugate, wherein, in formula (2-2),

mAb is an antibody recognizing an antigen expressed on a surface of an iPS cell;

q is an integer of 1 to 8;

h is 2, 3, 4 or 5;

Z″ is a group represented by formula (Z-7);

G is *²-Cit-Val-;

*² represents bonding between the G terminus and —NH—;

Q is a group represented by formula (Q-1) or formula (Q-2); and

f is 1 or 2,

or a salt thereof.

Examples of one aspect of the antibody-drug conjugate according to the present invention include the following (2-2-F).

### (2-2-F)

An antibody-drug conjugate, wherein, in formula (2-2),

mAb is an anti-CD30 antibody;

q is an integer of 1 to 8;

h is 2, 3, 4 or 5;

Z″ is a group represented by formula (Z-7);

G is *²-Cit-Val-;

*² represents bonding between the G terminus and —NH—;

Q is a group represented by formula (Q-1) or formula (Q-2); and

f is 1 or 2,

or a salt thereof.

Examples of one aspect of the antibody-drug conjugate according to the present invention include the following (2-2-G).

### (2-2-G)

An antibody-drug conjugate, wherein, in formula (2-2),

mAb is an antibody recognizing an antigen expressed on a surface of an iPS cell;

q is an integer of 1 to 8;

h is 2, 3, 4 or 5;

Z″ is a group represented by formula (Z-8);

G is *²-Asn-Ala-, *²-Asn-Ala-Ala-, *²-Asn-Ala-Pro- or *²-Cit-Val-;

*² represents bonding between the G terminus and —NH—;

R³ represents —(CH₂)ᵤ—COOH;

u represents 1 or 2;

W is a group represented by formula (W-1); and

Q is a group represented by formula (Q-1) or formula (Q-2),

or a salt thereof.

Examples of one aspect of the antibody-drug conjugate according to the present invention include the following (2-2-H).

### (2-2-H)

An antibody-drug conjugate, wherein, in formula (2-2),

mAb is an anti-CD30 antibody;

q is an integer of 1 to 8;

h is 2, 3, 4 or 5;

Z″ is a group represented by formula (Z-8);

G is *²-Asn-Ala-, *²-Asn-Ala-Ala-, *²-Asn-Ala-Pro- or *²-Cit-Val-;

*² represents bonding between the G terminus and —NH—;

R³ represents —(CH₂)ᵤ—COOH;

u represents 1 or 2;

W is a group represented by formula (W-1); and

Q is a group represented by formula (Q-1) or formula (Q-2),

or a salt thereof.

Examples of one aspect of the antibody-drug conjugate according to the present invention include the following (2-2-I).

### (2-2-I)

An antibody-drug conjugate, wherein, in formula (2-2),

mAb is an antibody recognizing an antigen expressed on a surface of an iPS cell;

q is an integer of 1 to 8;

h is 2, 3, 4 or 5;

Z″ is a group represented by formula (Z-9);

G is *²-Asn-Ala-, *²-Asn-Ala-Ala-, *²-Asn-Ala-Pro- or *²-Cit-Val-;

*² represents bonding between the G terminus and —NH—;

f is 1 or 2;

W is a group represented by formula (W-1); and

Q is a group represented by formula (Q-1) or formula (Q-2),

or a salt thereof.

Examples of one aspect of the antibody-drug conjugate according to the present invention include the following (2-2-J).

### (2-2-J)

An antibody-drug conjugate, wherein, in formula (2-2),

mAb is an anti-CD30 antibody;

q is an integer of 1 to 8;

h is 2, 3, 4 or 5;

Z″ is a group represented by formula (Z-9);

G is *²-Asn-Ala-, *²-Asn-Ala-Ala-, *²-Asn-Ala-Pro- or *²-Cit-Val-;

*² represents bonding between the G terminus and —NH—;

f is 1 or 2;

W is a group represented by formula (W-1); and

Q is a group represented by formula (Q-1) or formula (Q-2),

or a salt thereof.

<ADC Intermediate>

A synthetic intermediate for synthesizing the antibody-drug conjugate according to the present invention (hereinafter, may be referred to as the “ADC intermediate according to the present invention”) is a compound represented by the following formula (3-1) or formula (3-2), or a salt thereof.

(1) Compound Represented by Formula (3-1) or Salt Thereof

Among ADC intermediates according to the present invention, a compound represented by the following formula (3-1) or a salt thereof will be described.

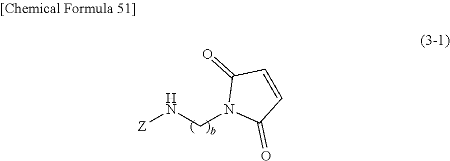

Z and b in formula (3-1) are as defined for those symbols in formula (2-1).

(2) Compound Represented by Formula (3-2) or Salt Thereof

Among ADC intermediates according to the present invention, a compound represented by the following formula (3-2) or a salt thereof will be described.

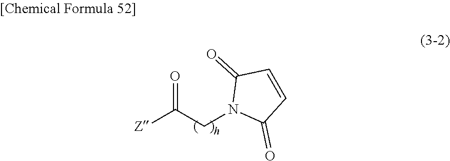

Z″ and h in formula (3-2) are as defined for those symbols in formula (2-2).

Examples of one aspect of the ADC intermediate according to the present invention include the following (3-1-A).

### (3-1-A)

A compound, wherein, in formula (3-1),

b is 2, 3, 4 or 5;

Z is a group represented by formula (Z-1) or formula (Z-2);

W is a group represented by formula (W-1);

Q is a group represented by formula (Q-1);

f represents 1 or 2;

R¹ represents —(CH₂)ᵤ—COOH; and

u is 1 or 2,

or a salt thereof.

Examples of one aspect of the ADC intermediate according to the present invention include the following (3-1-B).

### (3-1-B)

A compound, wherein, in formula (3-1),

b is 2, 3, 4 or 5;

Z is a group represented by formula (Z-1) or formula (Z-2);

W is a group represented by formula (W-1);

Q is a group represented by formula (Q-2);

f represents 1 or 2;

R¹ represents —(CH₂)ᵤ—COOH; and

u is 1 or 2,

or a salt thereof.

Examples of one aspect of the ADC intermediate according to the present invention include the following (3-2-A).

### (3-2-A)

A compound, wherein, in formula (3-2),

h is 2, 3, 4 or 5;

Z″ is a group represented by formula (Z-5) or formula (Z-6);

G is a single bond;

Y is a single bond;

W is a group represented by formula (W-1); and

Q is a group represented by formula (Q-1),

or a salt thereof.

Examples of one aspect of the ADC intermediate according to the present invention include the following (3-2-B).

### (3-2-B)

A compound, wherein, in formula (3-2),

h is 2, 3, 4 or 5;

Z″ is a group represented by formula (Z-5) or formula (Z-6);

G is a single bond;

Y is a single bond;

W is a group represented by formula (W-1); and

Q is a group represented by formula (Q-2),

or a salt thereof.

Examples of one aspect of the ADC intermediate according to the present invention include the following (3-2-C).

### (3-2-C)

A compound, wherein, in formula (3-2),

h is 2, 3, 4 or 5;

Z″ is a group represented by formula (Z-5) or formula (Z-6);

G is *²-Cit-Val-;

*² represents bonding between the G terminus and Y;

Y is a group represented by formula (Y-1);

W is a group represented by formula (W-1); and

Q is a group represented by formula (Q-1),

or a salt thereof.

Examples of one aspect of the ADC intermediate according to the present invention include the following (3-2-D).

### (3-2-D)

A compound, wherein, in formula (3-2),

h is 2, 3, 4 or 5;

Z″ is a group represented by formula (Z-5) or formula (Z-6);

G is *²-Cit-Val-;

*² represents bonding between the G terminus and Y;

Y is a group represented by formula (Y-1);

W is a group represented by formula (W-1); and

Q is a group represented by formula (Q-2),

or a salt thereof.

Examples of one aspect of the ADC intermediate according to the present invention include the following (3-2-E).

### (3-2-E)

A compound, wherein, in formula (3-2),

h is 2, 3, 4 or 5;

Z″ is a group represented by formula (Z-7);

G is *²-Cit-Val-;

*² represents bonding between the G terminus and —NH—;

Q is a group represented by formula (Q-1); and

f is 1 or 2,

or a salt thereof.

Examples of one aspect of the ADC intermediate according to the present invention include the following (3-2-F).

### (3-2-F)

A compound, wherein, in formula (3-2),

h is 2, 3, 4 or 5;

Z″ is a group represented by formula (Z-7);

G is *²-Cit-Val-;

*² represents bonding between the G terminus and —NH—;

Q is a group represented by formula (Q-2); and

f is 1 or 2,

or a salt thereof.

Examples of one aspect of the ADC intermediate according to the present invention include the following (3-2-G).

### (3-2-G)

A compound, wherein, in formula (3-2),

h is 2, 3, 4 or 5;

Z″ is a group represented by formula (Z-8);

G is *²-Asn-Ala-, *²-Asn-Ala-Ala-, *²-Asn-Ala-Pro- or *²-Cit-Val-;

*² represents bonding between the G terminus and —NH—;

R³ represents —(CH₂)ᵤ—COOH;

u represents 1 or 2;

W is a group represented by formula (W-1); and

Q is a group represented by formula (Q-1),

or a salt thereof.

Examples of one aspect of the ADC intermediate according to the present invention include the following (3-2-H).

### (3-2-H)

A compound, wherein, in formula (3-2),

h is 2, 3, 4 or 5;

Z″ is a group represented by formula (Z-8);

G is *²-Asn-Ala-, *²-Asn-Ala-Ala-, *²-Asn-Ala-Pro- or *²-Cit-Val-;

*² represents bonding between the G terminus and —NH—;

R³ represents —(CH₂)ᵤ—COOH;

u represents 1 or 2;

W is a group represented by formula (W-1); and

Q is a group represented by formula (Q-2),

or a salt thereof.

Examples of one aspect of the ADC intermediate according to the present invention include the following (3-2-I).

### (3-2-I)

A compound, wherein, in formula (3-2),

h is 2, 3, 4 or 5;

Z″ is a group represented by formula (Z-9);

G is *²-Asn-Ala-, *²-Asn-Ala-Ala-, *²-Asn-Ala-Pro- or *²-Cit-Val-; *² represents bonding between the G terminus and —NH—;

f is 1 or 2;

W is a group represented by formula (W-1); and

Q is a group represented by formula (Q-1),

or a salt thereof.

Examples of one aspect of the ADC intermediate according to the present invention include the following (3-2-J).

### (3-2-J)

An antibody-drug conjugate, wherein, in formula (3-2),

h is 2, 3, 4 or 5;

Z″ is a group represented by formula (Z-9);

G is *²-Asn-Ala-, *²-Asn-Ala-Ala-, *²-Asn-Ala-Pro- or *²-Cit-Val-;

*² represents bonding between the G terminus and —NH—;

f is 1 or 2;

W is a group represented by formula (W-1); and

Q is a group represented by formula (Q-2),

or a salt thereof.

The “salt” is a suitable salt of the hemiasterlin derivative according to the present invention and is acceptable as a pharmaceutical raw material, and is preferably a common non-toxic salt. For the “salt”, for example, in addition to acid addition salts such as organic acid salts (for example, acetate, trifluoroacetate, maleate, fumarate, citrate, tartrate, methanesulfonate, benzenesulfonate, formate, p-toluenesulfonate or the like) and inorganic acid salts (for example, hydrochloride, hydrobromide, hydroiodide, sulfate, nitrate, phosphateor the like); salts with amino acids (for example, arginine, aspartic acid, glutamic acid or the like); metal salts such as alkali metal salts (for example, sodium salt, potassium salt or the like) and alkaline earth metal salts (for example, calcium salt, magnesium salt or the like); ammonium salts; or organic base salts (for example, trimethylamine salt, triethylamine salt, pyridine salt, picoline salt, dicyclohexylamine salt, N,N′-dibenzylethylenediamine salt or the like), a person having ordinary skill in the art may select appropriate salts as appropriate.

In general, it is possible to carry out production and analysis of the antibody-drug conjugate by an arbitrary technique known to a person having ordinary skill in the art. Examples of such a method include the method described in Antibody-Drug Conjugates (edited by Laurent Ducry, published by Humana Press, 2013).

The antibody-drug conjugate according to the present invention may be formed by, for example, reducing a disulfide bond in the antibody into a sulfhydryl group and allowing this sulfhydryl group to react with an ADC intermediate.

When it is desired to acquire a salt of the hemiasterlin derivative, antibody-drug conjugate or ADC intermediate according to the present invention, if the target compound is obtained in the form of salt, that compound may be purified as is, and if the target compound is obtained in the free form, that compound may be dissolved or suspended in an appropriate organic solvent or buffer solution, to which an acid or base is added to form a salt by a conventional method.

The hemiasterlin derivative, antibody-drug conjugate and ADC intermediate according to the present invention may be present in the form of hydrates and/or solvates (ethanolate and the like) with various solvents, and these hydrates and/or solvates are also included in the hemiasterlin derivative, antibody-drug conjugate and ADC intermediate according to the present invention. Furthermore, all modes of crystal forms of the hemiasterlin derivative, antibody-drug conjugate and ADC intermediate according to the present invention are also included in the present invention.

Among the hemiasterlin derivative, antibody-drug conjugate and ADC intermediate according to the present invention, some may have optical isomers based on the optically active center, atropisomers based on axial or planar chirality caused by restraint of intramolecular rotation, and all of the other stereoisomers, tautomers and geometrical isomers, and all possible isomers including the above are encompassed within the scope of the present invention.

In particular, optical isomers and atropisomers may be obtained as racemate, and when optically active starting materials or intermediates are used, optically active substances may be obtained. If necessary, at an appropriate stage in the following production methods, corresponding raw material, intermediate or racemate, the final product, may be optically resolved into optical enantiomers physically or chemically through known separation methods such as a method using an optically active column and fractional crystallization method. Specifically, for example, in diastereomer method, two diastereomers are formed from racemate through a reaction using an optically active resolving agent. In general, these different diastereomers have different physical properties, and thus, can be optically resolved by known methods such as fractional crystallization.

Production methods for the hemiasterlin derivative according to the present invention will be mentioned below. The hemiasterlin derivative according to the present invention represented by formula (1-1), formula (1-2), formula (1-3), formula (3-1) or formula (3-2) may be produced by, for example, the following production method A to L.

Production Method A

When Z is a group represented by formula (Z-1); W is a group represented by formula (W-1); and Q is a group represented by formula (Q-1), the compound represented by formula (1-1) or formula (3-1) may be produced by, for example, the following production method:

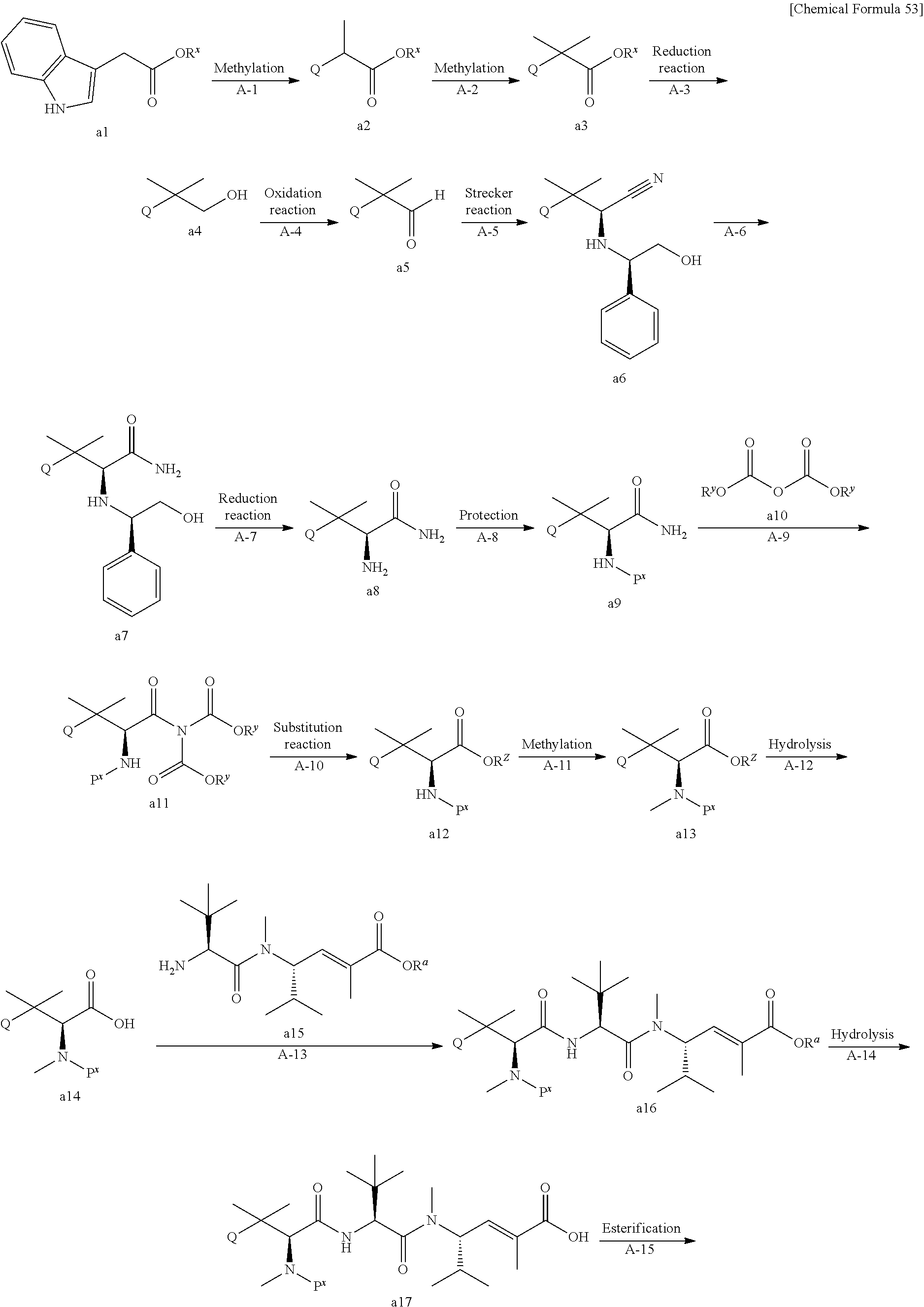

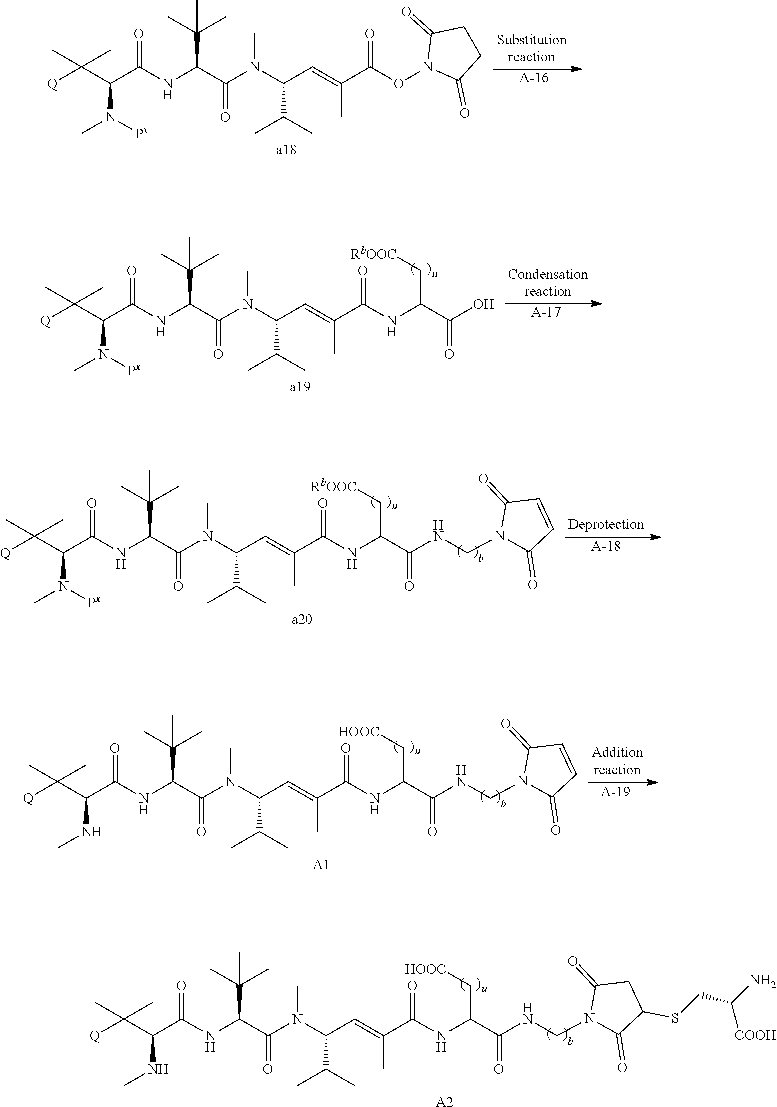

wherein, u and b are as defined in item 1 or item 17; R^{a}, R^{b}, R^{x}, R^{y} and R^{z} each independently represent a C₁₋₆ alkyl group or a benzyl group; and P^{X} represents a protecting group for the amino group.

As the above protecting group for the amino group, represented by P^{X}, the protecting groups described in Protective Groups in Organic Synthesis (authored by Theodora W. Greene, Peter G. M. Wuts, issued by John Wiley & Sons, Inc., 1999) and the like may be used. As the above protecting group for the amino group, represented by P^{X}, the protecting groups described in Protective Groups in Organic Synthesis (authored by Theodora W. Greene, Peter G. M. Wuts, issued by John Wiley & Sons, Inc., 1999) and the like may be used.

Compound a1 may be produced by the method described in, for example, J. Med. Chem., 2007, 50, 4329-4339 and the like, or may be purchased as a commercial product. Compound a15 may be produced by the method described in, for example, Tetrahedron Lett., 1997, 38, 317-320 and the like, or may be purchased as a commercial product.

[A-1 Step]

Compound a2 may be produced by allowing compound a1 to react with various methylating reagents in an appropriate solvent in the presence of an appropriate base. Examples of the methylating reagent include methyl halide, and preferably include methyl iodide, methyl bromide and methyl chloride. Examples of the base preferably include potassium hexamethyldisilazide. Examples of the solvent preferably include tetrahydrofuran. The reaction time is normally 5 minutes to 48 hours, and is preferably 10 minutes to 2 hours. The reaction temperature is normally −78° C. to 100° C., and is preferably −78° C. to 10° C. This step may be carried out in accordance with the method described in J. Nat. Prod. 2003, 66, 183-199 and the like.

[A-2 Step]

Compound a3 may be produced from compound a2 in accordance with the method described in the above A-1 step.

[A-3 Step]

Compound a4 may be produced by allowing compound a3 to react with an appropriate reducing agent in an appropriate solvent. The reducing agent is selected from reducing agents used in usual organic synthesis reactions as appropriate, and examples thereof preferably include diisobutylaluminum hydride. Examples of the solvent preferably include diethyl ether. The reaction time is normally 5 minutes to 48 hours, and is preferably 10 minutes to 24 hours. The reaction temperature is normally −78° C. to 100° C., and is preferably −78° C. to 50° C. This step may be carried out in accordance with the method described in J. Nat. Prod. 2003, 66, 183-199 and the like.

[A-4 Step]

Compound a5 may be produced by oxidizing compound a4 using an appropriate oxidizing agent in an appropriate solvent. The oxidizing agent may be selected from oxidizing agents used in usual organic synthesis reactions as appropriate, and examples thereof preferably include tetrapropylammonium perruthenate. Examples of the solvent preferably include dichloromethane. The reaction time is normally 5 minutes to 72 hours, and is preferably 30 minutes to 24 hours. The reaction temperature is normally −78° C. to 100° C., and is preferably −78° C. to 50° C. This step may be carried out in accordance with the method described in J. Nat. Prod. 2003, 66, 183-199 and the like.

[A-5 Step]

Compound a6 may be produced by α-aminocyanating the aldehyde of the compound a5 in an appropriate solvent. Examples of the solvent preferably include toluene and dichloromethane. The reaction time is normally 5 minutes to 96 hours, and is preferably 24 hours to 72 hours. The reaction temperature is normally 0° C. to 200° C., and is preferably 0° C. to 100° C. This step may be carried out in accordance with the method described in Org. Lett. 2002, 4, 695-697 and the like.

[A-6 Step]

Compound a7 may be produced from compound a6 by using an appropriate oxidizing agent in an appropriate solvent in the presence of or in the absence of an appropriate base. The oxidizing agent may be selected from oxidizing agents used in usual organic synthesis reactions as appropriate, and examples thereof preferably include hydrogen peroxide. Examples of the base preferably include potassium carbonate. Examples of the solvent preferably include methanol. The reaction time is normally 5 minutes to 72 hours, and is preferably 30 minutes to 24 hours. The reaction temperature is normally 0° C. to 200° C., and is preferably 0° C. to 60° C. This step may be carried out in accordance with the method described in J. Org. Chem. 2001, 66, 7355-7364 and the like.

[A-7 Step]

Compound a8 may be produced by reducing compound a7 using a reducing agent in an appropriate solvent in the presence of an appropriate catalyst. The reducing agent may be selected from reducing agents used in usual organic synthesis reactions as appropriate, and examples thereof preferably include hydrogen, formate such as ammonium formate, or hydrazine. Examples of the catalyst include transition metals such as palladium, nickel, rhodium, cobalt and platinum, salts thereof or complexes thereof, or supports such as polymer having the above transition metals supported thereon. Examples of the solvent preferably include ethanol or methanol. The reaction time is normally 5 minutes to 72 hours, and is preferably 30 minutes to 24 hours. The reaction temperature is normally 0° C. to 200° C., and is preferably 0° C. to 100° C. This step may be carried out in accordance with the method described in J. Org. Chem. 2001, 66, 7355-7364 and the like.

[A-8 Step]

Compound a9 may be produced by protecting the amino group of compound a8 with protecting group P^{X}. This step may be carried out in accordance with the method described in Protective Groups in Organic Synthesis (authored by Theodora W. Greene, Peter G. M. Wuts, issued by John Wiley & Sons, Inc., 1999) and the like.

[A-9 Step]

Compound a11 may be produced by allowing compound a9 to react with various acylating reagents (for example, compound a10) in an appropriate solvent in the presence of or in the absence of an appropriate base. Examples of the acylating reagent include carboxylic halide and carboxylic anhydride, and preferably include di-tert-butyl dicarbonate. Examples of the base preferably include diisopropylethylamine. Examples of the solvent preferably include chloroform. The reaction time is normally 5 minutes to 72 hours, and is preferably 30 minutes to 24 hours. The reaction temperature is normally 0° C. to 200° C., and is preferably 0° C. to 50° C.

[A-10 Step]

Compound a12 may be produced by allowing compound a11 to react with an appropriate alkali metal alkoxide in an appropriate solvent. The alkali metal alkoxide may be selected from alkali metal alkoxides used in usual organic synthesis reactions as appropriate, and examples thereof preferably include lithium methoxide or lithium ethoxide. Examples of the solvent preferably include methanol or ethanol. The reaction time is normally 5 minutes to 72 hours, and is preferably 30 minutes to 6 hours. The reaction temperature is normally −78° C. to 200° C., and is preferably −78° C. to 50° C.

[A-11 Step]

Compound a13 may be produced by allowing compound a12 to react with various methylating reagents in an appropriate solvent in the presence of an appropriate base. Examples of the methylating reagent include methyl halide, and preferably include methyl iodide, methyl bromide and methyl chloride. Examples of the base preferably include sodium hydride. Examples of the solvent preferably include tetrahydrofuran. The reaction time is normally 5 minutes to 48 hours, and is preferably 10 minutes to 2 hours. The reaction temperature is normally −78° C. to 100° C., and is preferably −78° C. to 10° C.

[A-12 Step]

Compound a14 may be produced by hydrolyzing the ester of compound a13, in an appropriate solvent in the presence of an appropriate base. Examples of the base preferably include lithium hydroxide. Examples of the solvent preferably include water or methanol. The reaction time is normally 5 minutes to 72 hours, and is preferably 30 minutes to 24 hours. The reaction temperature is normally 0° C. to 200° C., and is preferably 0° C. to 100° C.

[A-13 Step]

Compound a16 may be produced by condensing compound a14 and compound a15 using various condensing agents in an appropriate solvent in the presence of an appropriate base. As the condensing agent, various condensing agents used in usual organic synthesis reactions may be used, and examples thereof preferably include (1-ethyl-3-(3-dimethylaminopropyl)carbodiimide or bromotripyrrolidinophosphonium hexafluorophosphate. In addition a carbonyl activating reagent such as 1-hydroxybenzotriazole may be used together as necessary, in order to improve efficiency of the condensation reaction. Examples of the base preferably include diisopropylethylamine. Examples of the solvent preferably include N,N-dimethylformamide. The reaction time is normally 5 minutes to 72 hours, and is preferably 30 minutes to 24 hours. The reaction temperature is normally −78° C. to 200° C., and is preferably 0° C. to 100° C. This step may be carried out in accordance with the method described in Tetrahedron Lett., 1997, 38, 317-320 and the like.

[A-14 Step]

Compound a17 may be produced by hydrolyzing the ester of compound a16, in accordance with the method described in the above A-12 step. This step may be carried out in accordance with the method described in Tetrahedron Lett., 1997, 38, 317-320 and the like.

[A-15 Step]

Compound a18 may be produced by allowing compound a17 to react with N-hydroxysuccinimide using various condensing agents in an appropriate solvent in the presence of an appropriate base. As the condensing agent, various condensing agents used in usual organic synthesis reactions may be used, and examples thereof preferably include (1-ethyl-3-(3-dimethylaminopropyl)carbodiimide, or bromotripyrrolidinophosphonium hexafluorophosphate. In addition, a carbonyl activating reagent such as 1-hydroxybenzotriazole may be used together as necessary, in order to improve efficiency of the reaction. Examples of the base preferably include diisopropylethylamine. Examples of the solvent preferably include N,N-dimethylformamide. The reaction time is normally 5 minutes to 72 hours, and is preferably 30 minutes to 24 hours. The reaction temperature is normally −78° C. to 200° C., and is preferably 0° C. to 100° C.

[A-16 Step]

Compound a19 may be produced by allowing compound a18 to react with an ester of an amino acid in an appropriate solvent in the presence of an appropriate base. Examples of the base preferably include diisopropylethylamine. Examples of the solvent preferably include N,N-dimethylformamide. The reaction time is normally 5 minutes to 72 hours, and is preferably 30 minutes to 24 hours. The reaction temperature is normally 0° C. to 200° C., and is preferably 0° C. to 100° C.

[A-17 Step]

Compound a20 may be produced by condensing compound a19 and an aminoalkylmaleimide compound in accordance with the method described in the above A-13 step.

[A-18 Step]

Compound A1 may be produced by deprotection of the protecting group P^{X} for the amino group of compound a20 and hydrolysis of the ester (—COOR^{b}). This step may be carried out in accordance with the method described in Protective Groups in Organic Synthesis (authored by Theodora W. Greene, Peter G. M. Wuts, issued by John Wiley & Sons, Inc., 1999) and the like.

[A-19 Step]

Compound A2 may be produced by allowing compound A1 to react together with cysteine in an appropriate solvent. Examples of the solvent preferably include water, dimethylsulfoxide and N,N-dimethylformamide. The reaction time is normally 5 minutes to 72 hours, and is preferably 30 minutes to 24 hours. The reaction temperature is normally 0° C. to about 200° C., and is preferably 0° C. to 40° C.

Production Method B

When Z is a group represented by formula (Z-1); W is a group represented by formula (W-1); and Q is a group represented by formula (Q-2), the compound represented by formula (1-1) or formula (3-1) may be produced by, for example, the following production method:

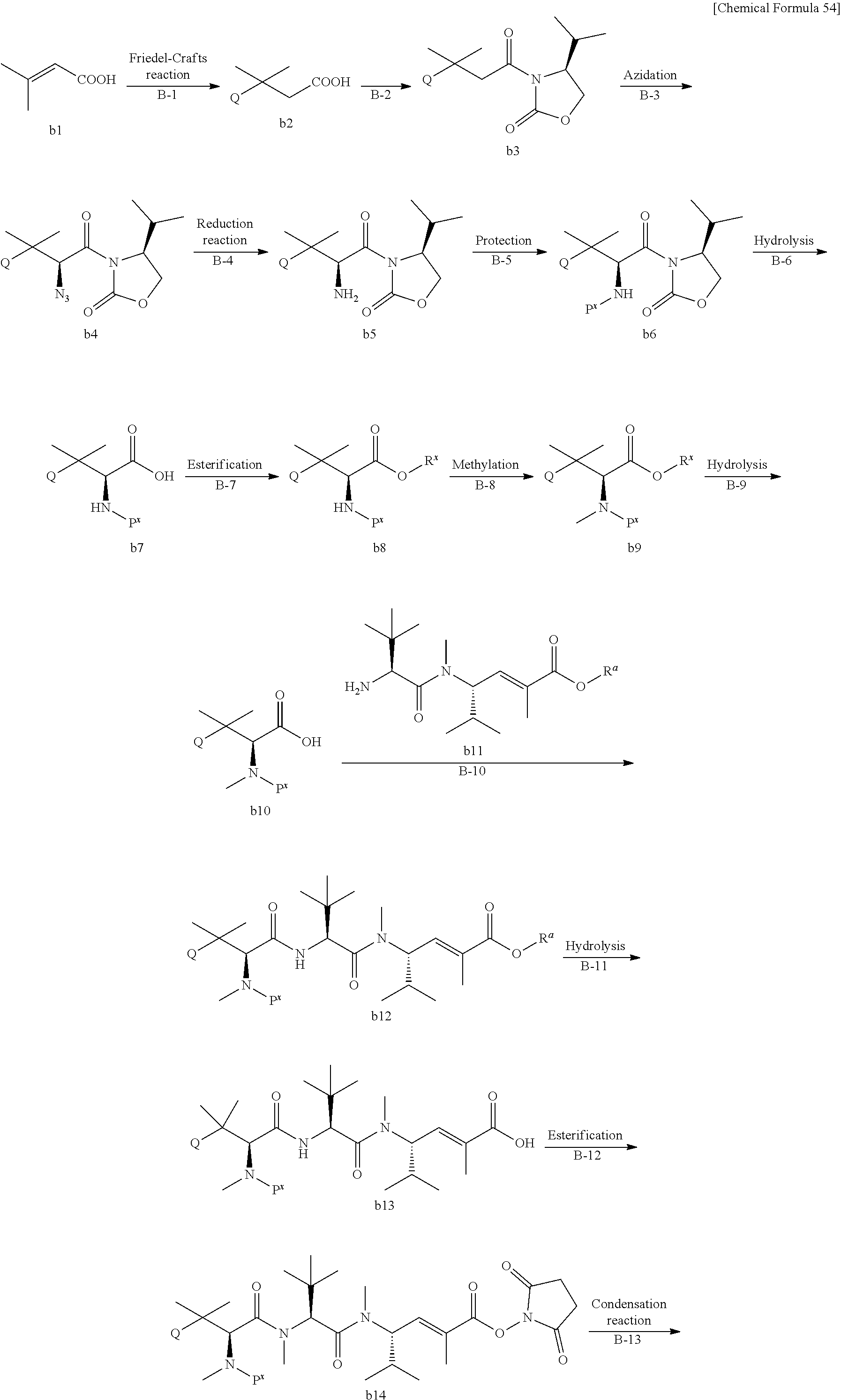

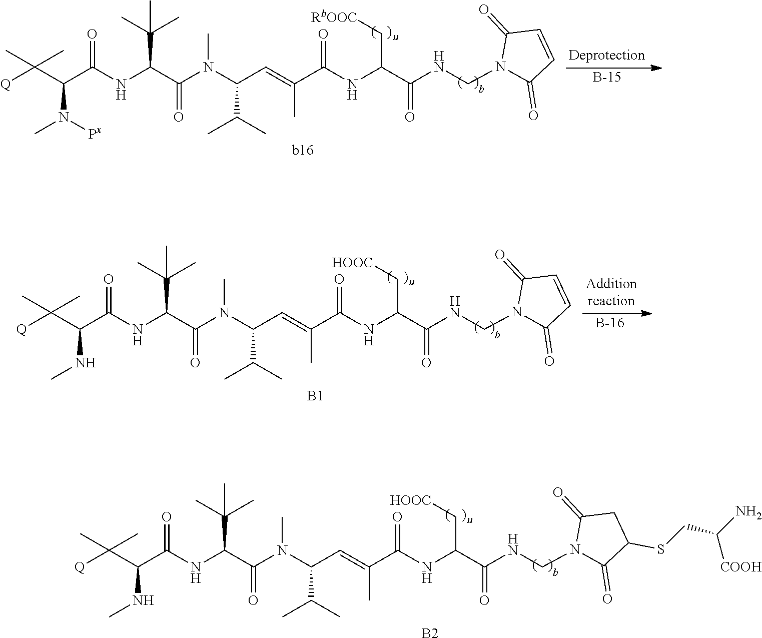

wherein, u or b is as defined in item 1 or item 17;
R^{a}, R^{b} and R^{x} each represent a C₁₋₆ alkyl group or a benzyl group; and
P^{X} means a protecting group for the amino group.

Compound b1 may be, for example, purchased as a commercial product. Compound b11 may be produced by the method described in, for example, Tetrahedron Lett., 1997, 38, 317-320 and the like, or may be purchased as a commercial product.

[B-1 Step]

Compound b2 may be produced by allowing compound b1 to react with benzene in the presence of various Lewis acids. Examples of the Lewis acid include boron halide, aluminum halide, gallium halide, iron halide and titanium halide, and preferably include aluminum chloride and iron chloride. The reaction time is normally 5 minutes to 48 hours, and is preferably 30 minutes to 4 hours. The reaction temperature is normally −78° C. to 200° C., and is preferably 50° C. to 150° C. This step may be carried out in accordance with the method described in J. Nat. Prod. 2003, 66, 183-199 and the like.

[B-2 Step]

Compound b3 may be produced by allowing compound b2 to react with various carboxylic halides and then to react with an alkali metallized 4-alkyl-2-oxazolidinone in an appropriate solvent in the presence of an appropriate base. Examples of the base preferably include triethylamine or diisopropylethylamine. Examples of the solvent preferably include tetrahydrofuran. Examples of the carboxylic halide include carboxylic chloride, and preferably include pivaloyl chloride. Examples of the alkali metallized 4-alkyl-2-oxazolidinone include 4-alkyl-2-oxazolidinone lithium and 4-alkyl-2-oxazolidinone sodium, and preferably include 4-isopropyl-2-oxazolidinone lithium. The reaction time is normally 5 minutes to 48 hours, and is preferably 10 minutes to 24 hours. The reaction temperature is normally −78° C. to 100° C., and is preferably −78° C. to 50° C. This step may be carried out in accordance with the method described in J. Nat. Prod. 2003, 66, 183-199 and the like.

[B-3 Step]

Compound b4 may be produced by allowing compound b3 to react with various azidating reagents in an appropriate solvent in the presence of an appropriate base. Examples of the azidating reagent include sodium azide, trimethylsilyl azide and diphenylphosphoryl azide, and preferably include trimethylsilyl azide. Examples of the base preferably include potassium hexamethyldisilazide. Examples of the solvent preferably include tetrahydrofuran. The reaction time is normally 5 minutes to 72 hours, and is preferably 30 minutes to 24 hours. The reaction temperature is normally −78° C. to 200° C., and is preferably −78° C. to 75° C. This step may be carried out in accordance with the method described in J. Nat. Prod. 2003, 66, 183-199 and the like.

[B-4 Step]

Compound b5 may be produced from compound b4 in accordance with the method described in the above A-7 step.

[B-5 Step]

Compound b6 may be produced from compound b5 in accordance with the method described in the above A-8 step.

[B-6 Step]

Compound b7 may be produced from compound b6 by using an appropriate oxidizing agent in an appropriate solvent in the presence of an appropriate base. Examples of the base preferably include lithium hydroxide. Examples of the solvent preferably include methanol, tetrahydrofuran or water. The oxidizing agent may be selected from oxidizing agents used in usual organic synthesis reactions as appropriate, and examples thereof preferably include hydrogen peroxide. The reaction time is normally 5 minutes to 72 hours, and is preferably 30 minutes to 24 hours. The reaction temperature is normally 0° C. to 200° C., and is preferably 0° C. to 60° C. This step may be carried out in accordance with the method described in J. Nat. Prod. 2003, 66, 183-199 and the like.

[B-7 Step]

Compound b8 may be produced by allowing compound b7 to react with various alkylating reagents in an appropriate solvent in the presence of an appropriate base. Examples of the alkylating reagent include alkyl halide, and preferably include alkyl iodide, alkyl bromide and alkyl chloride. Examples of the base preferably include sodium carbonate and potassium carbonate. Examples of the solvent preferably include N,N-dimethylformamide. The reaction time is normally 5 minutes to 48 hours, and is preferably 10 minutes to 2 hours. The reaction temperature is normally −78° C. to 100° C., and is preferably −10° C. to 25° C. This step may be carried out in accordance with the method described in Protective Groups in Organic Synthesis (authored by Theodora W. Greene, Peter G. M. Wuts, issued by John Wiley & Sons, Inc., 1999) and the like.

[B-8 Step]

Compound b9 may be produced from compound b8 in accordance with the method described in the above A-11 step.

[B-9 Step]

Compound b10 may be produced from compound b9 in accordance with the method described in the above A-12 step.

[B-10 Step]

Compound b12 may be produced from compound b10 and compound b11 in accordance with the method described in the above A-13 step.

[B-11 Step]

Compound b13 may be produced by hydrolyzing the ester of compound b12 in accordance with the method described in the above A-12 step.

[B-12 Step]

Compound b14 may be produced from compound b13 in accordance with the method described in the above A-15 step.

[B-13 Step]

Compound b15 may be produced from compound b14 in accordance with the method described in the above A-16 step.

[B-14 Step]

Compound b16 may be produced from compound b15 in accordance with the method described in the above A-17 step.

[B-15 Step]

Compound B1 may be produced from compound b16 in accordance with the method described in the above A-18 step.

[B-16 Step]

Compound B2 may be produced from compound B1 in accordance with the method described in the above A-19 step.

Production Method C

When Z is a group represented by formula (Z-2); and W is a group represented by formula (W-1), the compound represented by formula (1-1) or formula (3-1) may be produced by, for example, the following production method:

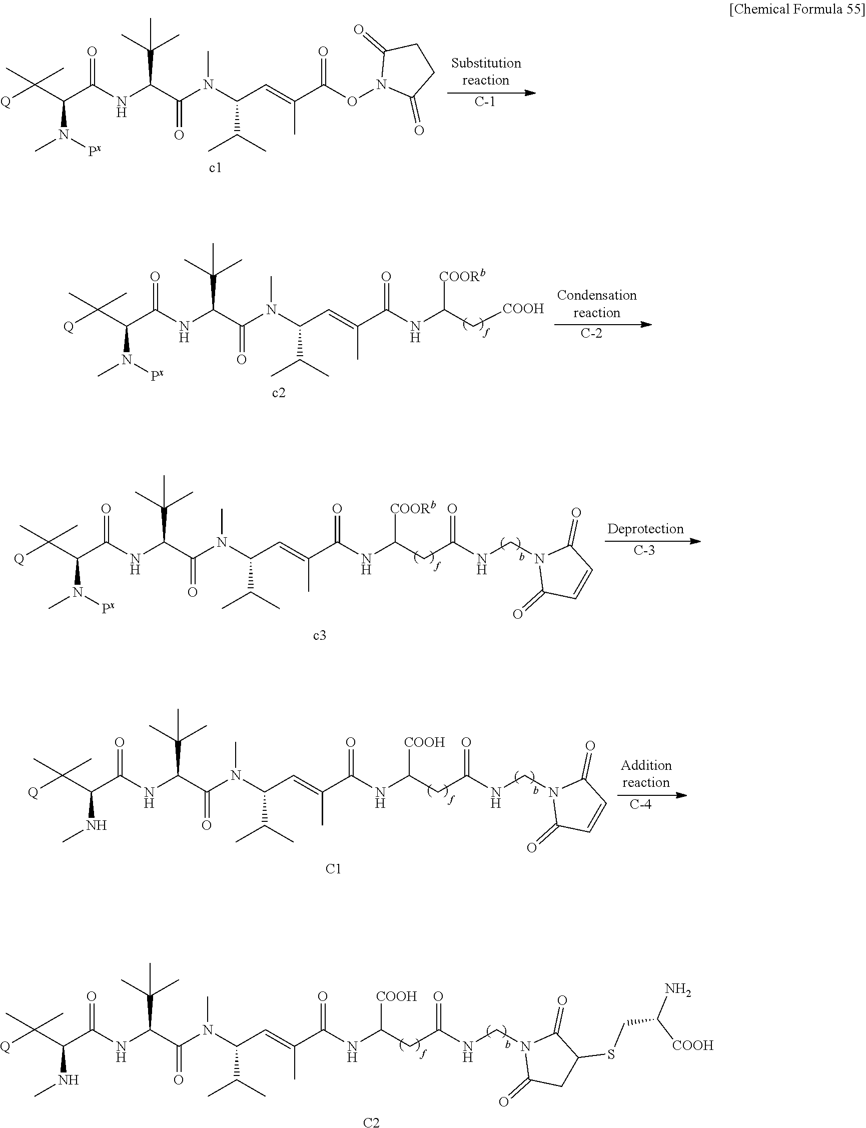

wherein, Q, f and b are as defined in item 1 or item 17; and R^{b} and P^{X} are as defined above.

Compound c1 represents compound a18 of Production Method A or compound b14 of Production Method B.

[C-1 Step]

Compound c2 may be produced from compound c1 in accordance with the method described in the above A-16 step.

[C-2 Step]

Compound c3 may be produced from compound c2 in accordance with the method described in the above A-17 step.

[C-3 Step]

Compound C1 may be produced from compound c3 in accordance with the method described in the above A-18 step.

[C-4 Step]

Compound C2 may be produced from compound c4 in accordance with the method described in the above A-19 step.

Production Method D

When R² is a lysine (Lys) residue; Z″ is a group represented by formula (Z-3) or formula (Z-5); Y is a single bond; G is a single bond; and W is a group represented by formula (W-1), the compound represented by formula (1-2), formula (1-3) or formula (3-2) may be produced by, for example, the following production method:

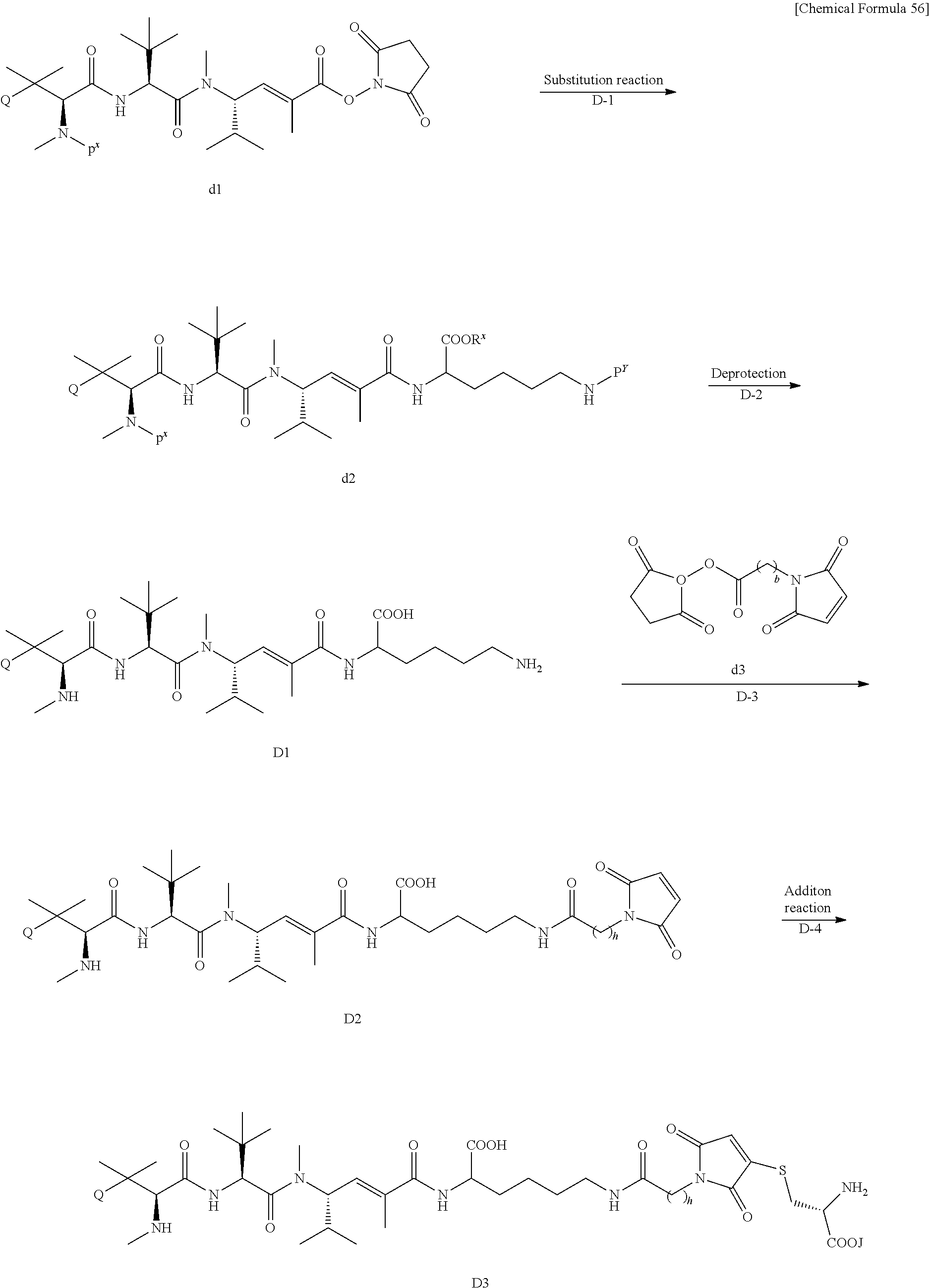

wherein, Q and h are as defined in item 2, item 3 or item 18; R^{x} and P^{X} are as defined above; and P^{Y} means a protecting group for the amino group.

Compound d1 represents compound a18 of Production Method A or compound b14 of Production Method B. Compound d3 may be, for example, purchased as a commercial product.

[D-1 Step]

Compound d2 may be produced from compound d1 in accordance with the method described in the above A-16 step.

[D-2 Step]

Compound D1 may be produced from compound d2 in accordance with the method described in the above A-18 step.

[D-3 Step]

Compound D2 may be produced from compound D1 and compound d3 in accordance with the method described in the above A-16 step.

[D-4 Step]

Compound D3 may be produced from compound D2 in accordance with the method described in the above A-19 step.

Production Method E

When R² is a lysine (Lys) residue; Z″ is a group represented by formula (Z-4) or formula (Z-6); Y is a single bond; G is a single bond; and W is a group represented by formula (W-1), the compound represented by formula (1-2), formula (1-3) or formula (3-2) may be produced by, for example, the following production method:

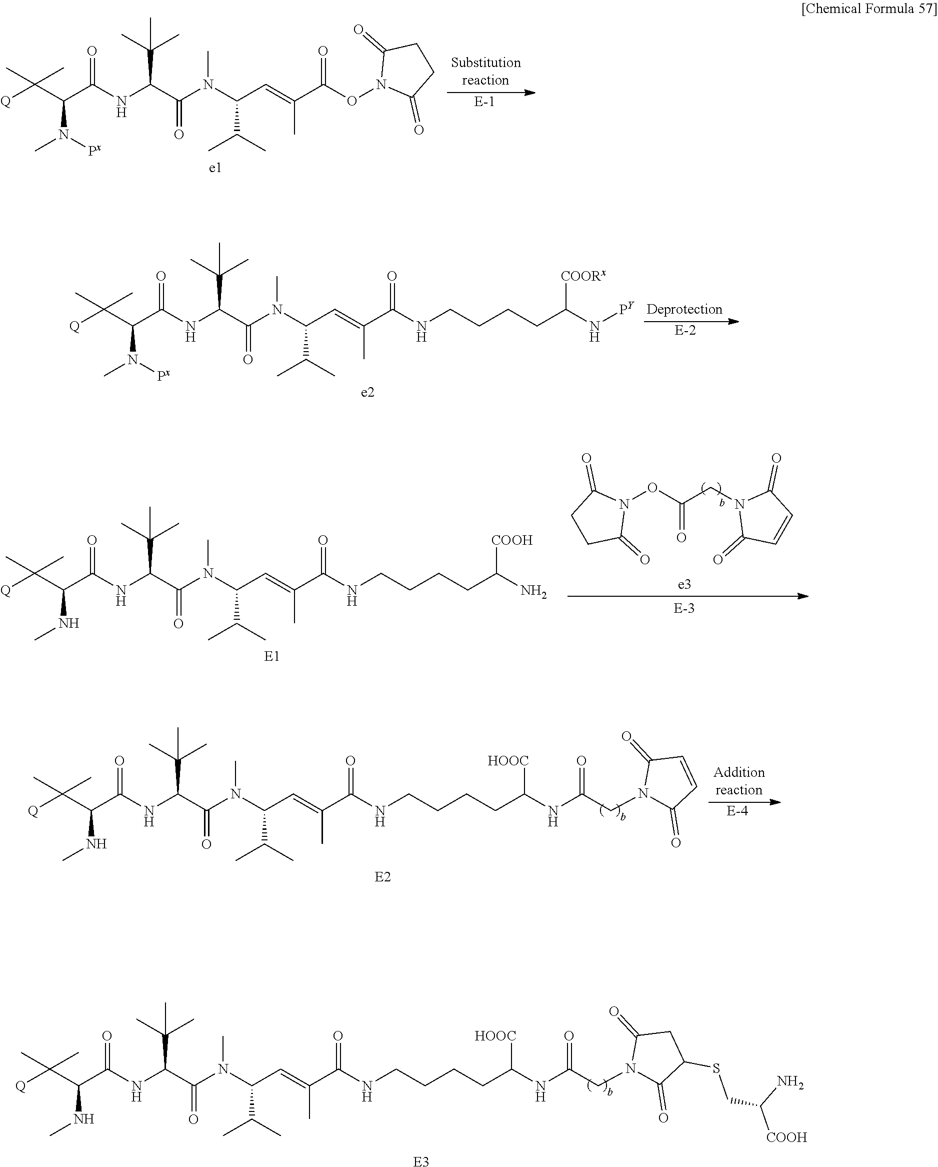

wherein, Q and h are as defined in item 2, item 3 or item 18; and R^{x}, P^{X} and P^{Y} are as defined above.

Compound e1 represents compound a18 of Production Method A or compound b14 of Production Method B. Compound e3 may be, for example, purchased as a commercial product.

[E-1 Step]

Compound e2 may be produced from compound e1 in accordance with the method described in the above A-16 step.

[E-2 Step]

Compound E1 may be produced from compound e2 in accordance with the method described in the above A-18 step.

[E-3 Step]

Compound E2 may be produced from compound E1 and compound e3 in accordance with the method described in the above A-16 step.

[E-4 Step]

Compound E3 may be produced from compound E2 in accordance with the method described in the above A-19 step.

Production Method F

When R² is an aspartic acid (Asp) residue or a glutamic acid (Glu) residue; and W is a group represented by formula (W-1), the compound represented by formula (1-3) may be produced by, for example, the following production method:

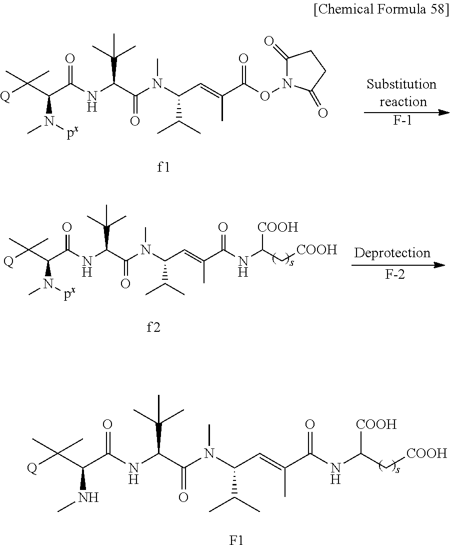

wherein, Q is as defined in item 3; s represents 1 or 2; and P^{X} is as defined above.

Compound f1 represents compound a18 of Production Method A or compound b14 of Production Method B.

[F-1 Step]

Compound f2 may be produced from compound f1 in accordance with the method described in the above A-16 step.

[F-2 Step]

Compound F1 may be produced from compound f2 in accordance with the method described in the above A-18 step.

Production Method G

When Z″ is a group represented by formula (Z-7); W is a group represented by formula (W-1); and G is a group represented as an amino acid or a peptide, the compound represented by formula (3-2) may be produced by, for example, the following production method:

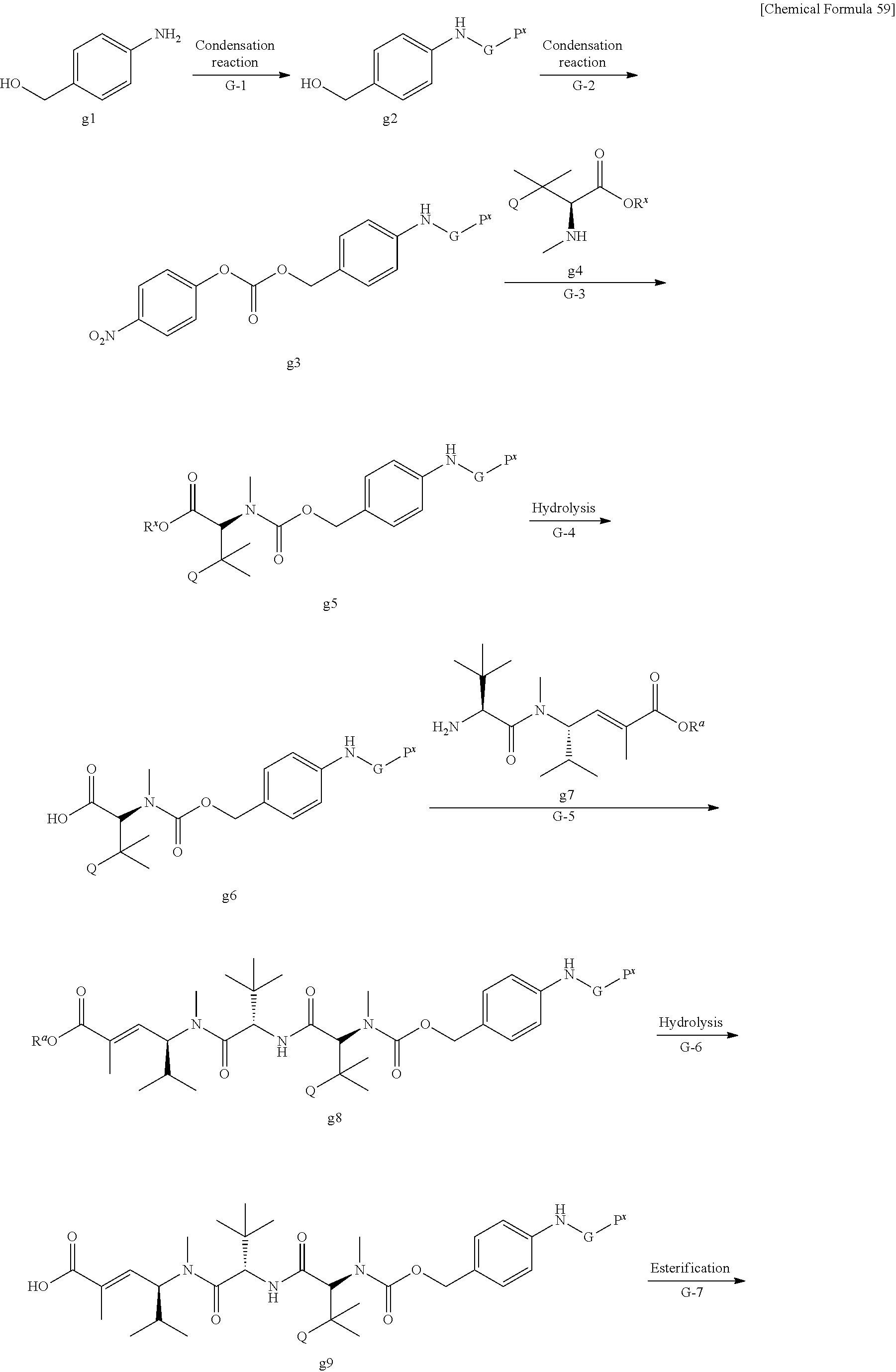

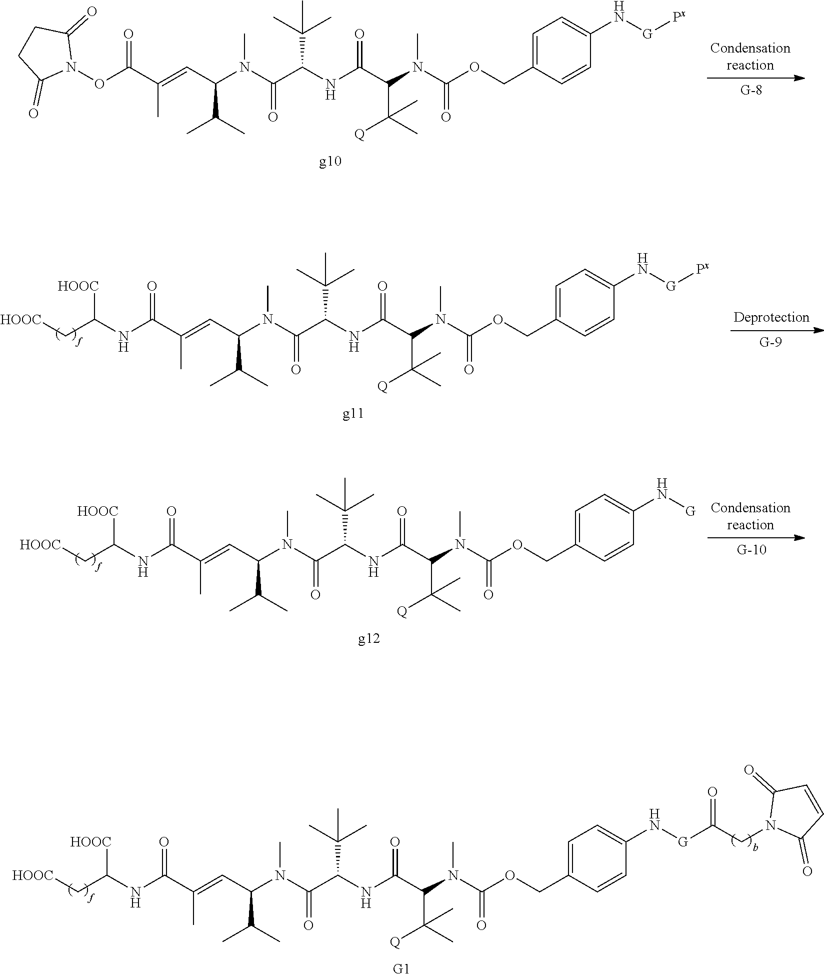

wherein, Q, f, G, and h are as defined in item 18; and R^{a}, R^{x} and P^{X} are as defined above.

Compound g1 may be, for example, purchased as a commercial product. Compound g4 may be produced by the methods described in, for example, J. Nat. Prod. 2003, 66, 183-199; J. Med. Chem., 2004, 47, 4774-4786: and the like, or may be purchased as a commercial product. Compound g7 may be produced by the method described in, for example, Tetrahedron Lett., 1997, 38, 317-320 and the like, or may be purchased as a commercial product.

[G-1 Step]

Compound g2 may be produced by condensing compound g1 and an amino acid or a peptide which is a raw material for G group, using various condensing agents in an appropriate solvent in the presence of an appropriate base. As the condensing agent, various condensing agents used in usual organic synthesis reactions may be used, and examples thereof preferably include 1-ethoxycarbonyl-2-ethoxy-1,2-dihydroquinoline (EEDQ). In addition, a carbonyl activating reagent such as 1-hydroxybenzotriazole may be used together as necessary, in order to improve efficiency of the condensation reaction. Examples of the base preferably include diisopropylethylamine. Examples of the solvent preferably include dichloromethane. The reaction time is normally 5 minutes to 72 hours, and is preferably 30 minutes to 24 hours. The reaction temperature is normally −78° C. to 200° C., and is preferably 0° C. to 50° C. This step may be carried out in accordance with the method described in Bioconjugate Chem. 2002, 13, 855-869 and the like.

[G-2 Step]

Compound g3 may be produced by allowing compound g2 to react with various p-nitrophenyl carbonate esterifying reagents in an appropriate solvent in the presence of an appropriate base. Examples of the p-nitrophenyl carbonate esterifying reagent include 4-nitrophenyl chloroformate and bis(4-nitrophenyl)carbonate, and preferably include bis(4-nitrophenyl)carbonate. Examples of the base preferably include N,N-diisopropylethylamine. Examples of the solvent preferably include tetrahydrofuran, dichloromethane and N,N-dimethylformamide. The reaction time is normally 5 minutes to 72 hours, and is preferably 1 hour to 24 hours. The reaction temperature is normally −78° C. to 200° C., and is preferably 10° C. to 50° C. This step may be carried out in accordance with the methods described in Bioconjugate Chem. 2002, 13, 855-869, Bioconjugate Chem. 2015, 26, 650-659 and the like.

[G-3 Step]

Compound g5 may be produced by allowing compound g3 and compound g4 to react in an appropriate solvent in the presence of an appropriate base. In addition, a carbonyl activating reagent such as 1-hydroxy-7-benzotriazole may be used together as necessary, in order to improve efficiency of the condensation reaction. Examples of the base preferably include triethylamine, diisopropylethylamine and 2,6-lutidine. Examples of the solvent preferably include N,N-dimethylformamide. The reaction time is normally 5 minutes to 72 hours, and is preferably 1 hour to 24 hours. The reaction temperature is normally −78° C. to 200° C., and is preferably 10° C. to 50° C. This step may be carried out in accordance with the methods described in Bioconjugate Chem. 2002, 13, 855-869, Bioconjugate Chem. 2015, 26, 650-659 and the like.

[G-4 Step]

Compound g6 may be produced by hydrolyzing the ester of compound g5, in accordance with the method described in the above A-12 step.

[G-5 Step]

Compound g8 may be produced from compound g6 and compound g7 in accordance with the method described in the above A-13 step.

[G-6 Step]

Compound g9 may be produced by hydrolyzing the ester of compound g8, in accordance with the method described in the above A-12 step.

[G-7 Step]

Compound g10 may be produced from compound g9 in accordance with the method described in the above A-15 step.

[G-8 Step]

Compound g11 may be produced from compound g10 in accordance with the method described in the above A-16 step.

[G-9 Step]

Compound g12 may be produced from compound g11 in accordance with the method described in the above A-17 step.

[G-10 Step]

Compound G1 may be produced from compound g12 in accordance with the method described in the above A-13 step or A-16 step.

Production Method H

When Z″ is a group represented by formula (Z-8); and G is a group represented as an amino acid or a peptide, the compound represented by formula (3-2) may be produced by, for example, the following production method:

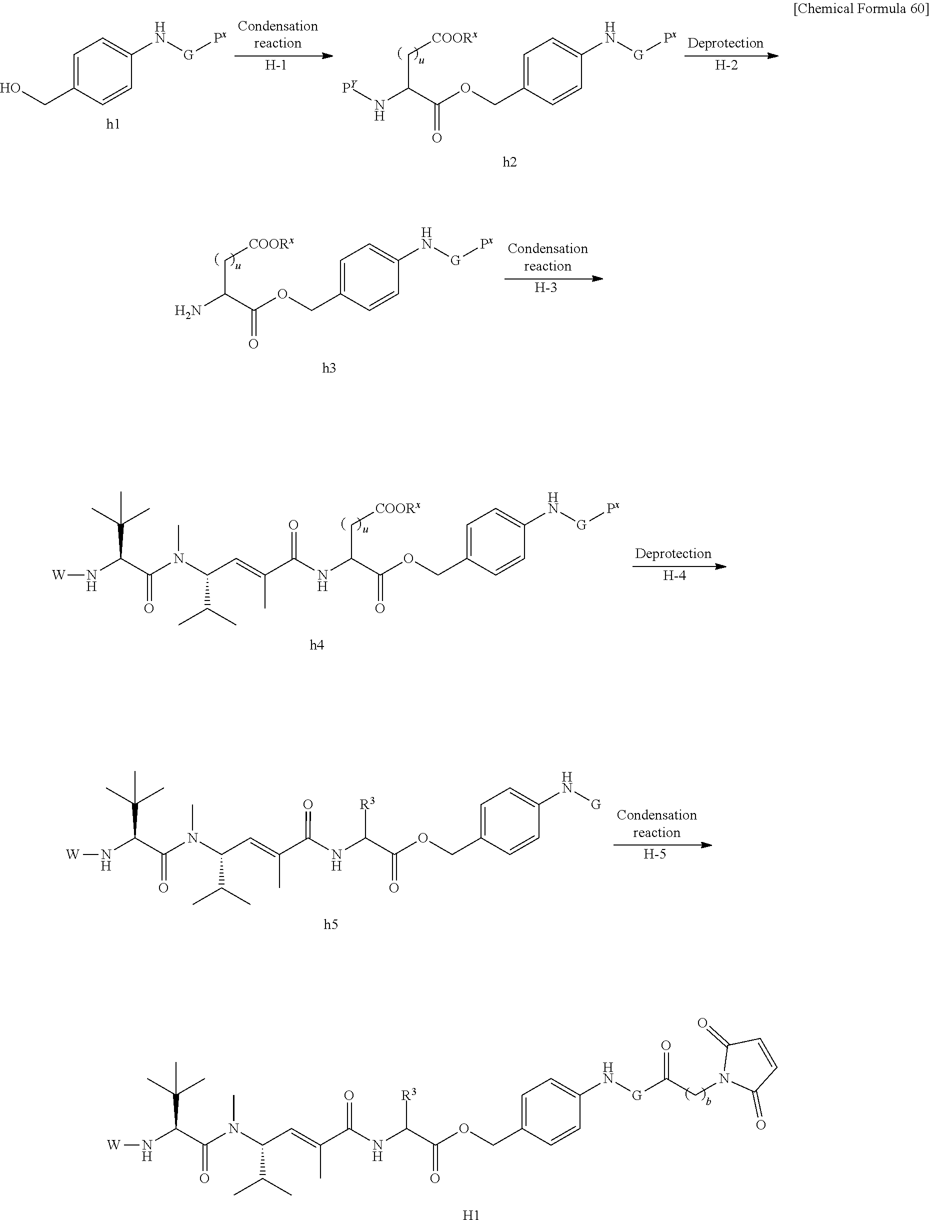

wherein, W, R³, u, G and h are as described in item 18; and R^{X}, P^{X} and P^{Y} are as defined above.

Compound h1 represents compound g2 of Production Method G.

[H-1 Step]

Compound h2 may be produced by subjecting compound h1 and a glutamic acid derivative or aspartic acid derivative to condensation reaction in an appropriate solvent in the presence of an appropriate sulfonylating reagent and imidazole. Examples of the sulfonylating reagent include methylsulfonyl chloride and p-toluenesulfonyl chloride, and preferably include p-toluenesulfonyl chloride. Examples of the imidazole include imidazole and 1-methylimidazole, and preferably include 1-methylimidazole. Examples of the solvent preferably include acetonitrile. The reaction time is normally 5 minutes to 72 hours, and is preferably 30 minutes to 24 hours. The reaction temperature is normally −78° C. to 200° C., and is preferably 0° C. to 50° C.

[H-2 Step]

Compound h3 may be produced from compound h2 in accordance with the method described in the above A-18 step.

[H-3 Step]

Compound h4 may be produced from compound h3 in accordance with the method described in the above A-13 step or A-16 step.

[H-4 Step]

Compound h5 may be produced from compound h4 in accordance with the method described in the above A-18 step.

[H-5 Step]

Compound H1 may be produced from compound h5 in accordance with the method described in the above A-13 step or A-16 step.

Production Method I

When Z″ is a group represented by formula (Z-9); and G is a group represented as an amino acid or a peptide, the compound represented by formula (3-2) may be produced by, for example, the following production method:

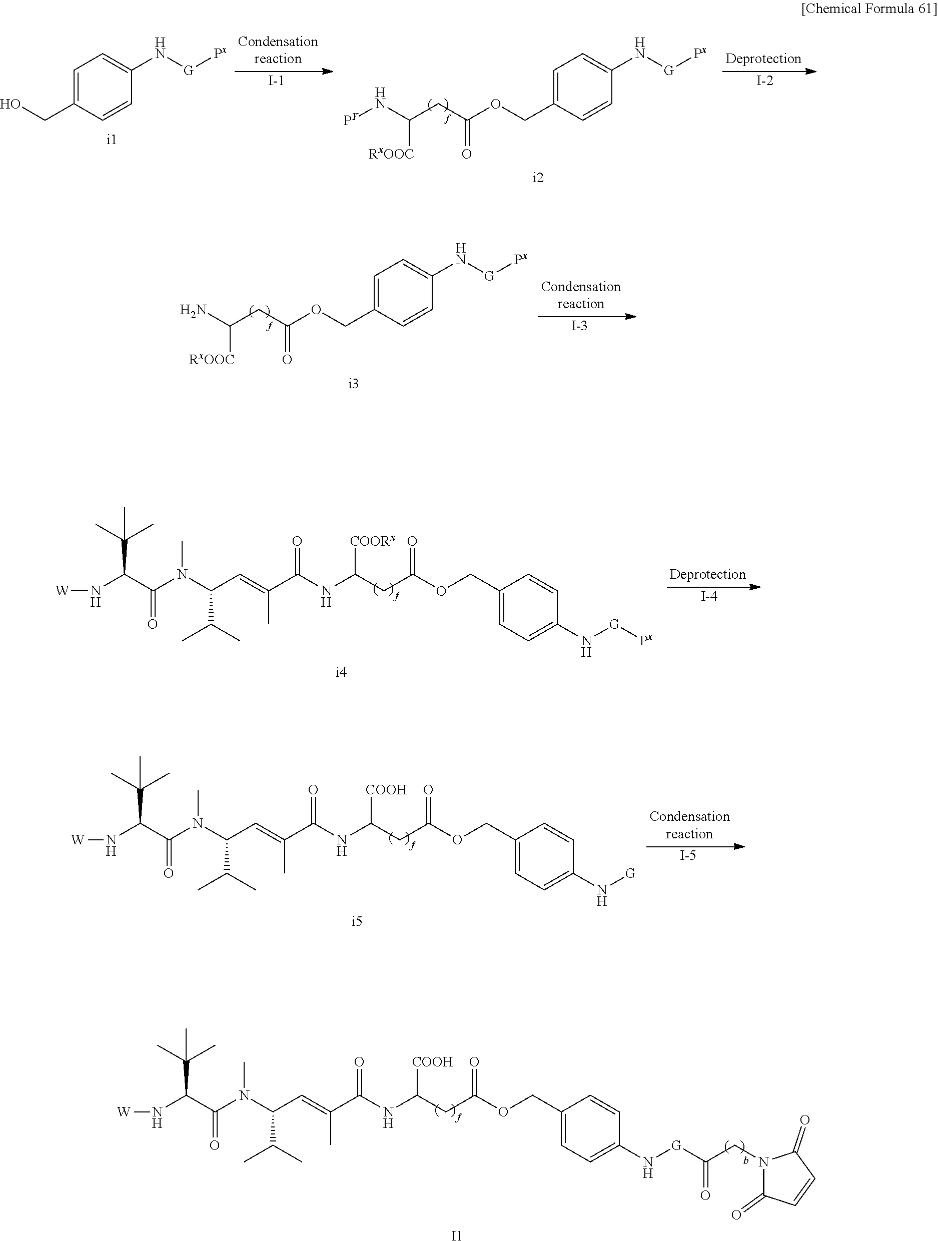

wherein, W, f, G and h are as described in item 18; and R^{X}, P^{X} and P^{Y} are as defined above.

Compound i1 represents compound g2 of Production Method G.

[I-1 Step]

Compound i2 may be produced from compound i1 in accordance with the method described in the above H-1 step.

[I-2 Step]

Compound i3 may be produced from compound i2 in accordance with the method described in the above A-18 step.

[I-3 Step]

Compound i4 may be produced from compound i3 in accordance with the method described in the above A-13 step or A-16 step.

[I-4 Step]

Compound i5 may be produced from compound i4 in accordance with the method described in the above A-18 step.

[I-5 Step]

Compound I1 may be produced from compound i5 in accordance with the method described in the above A-13 step or A-16 step.

Production Method J

When Z″ is a group represented by formula (Z-5); Y is a group represented by formula (Y-1); and G is a group represented as an amino acid or a peptide, the compound represented by formula (3-2) may be produced by, for example, the following production method:

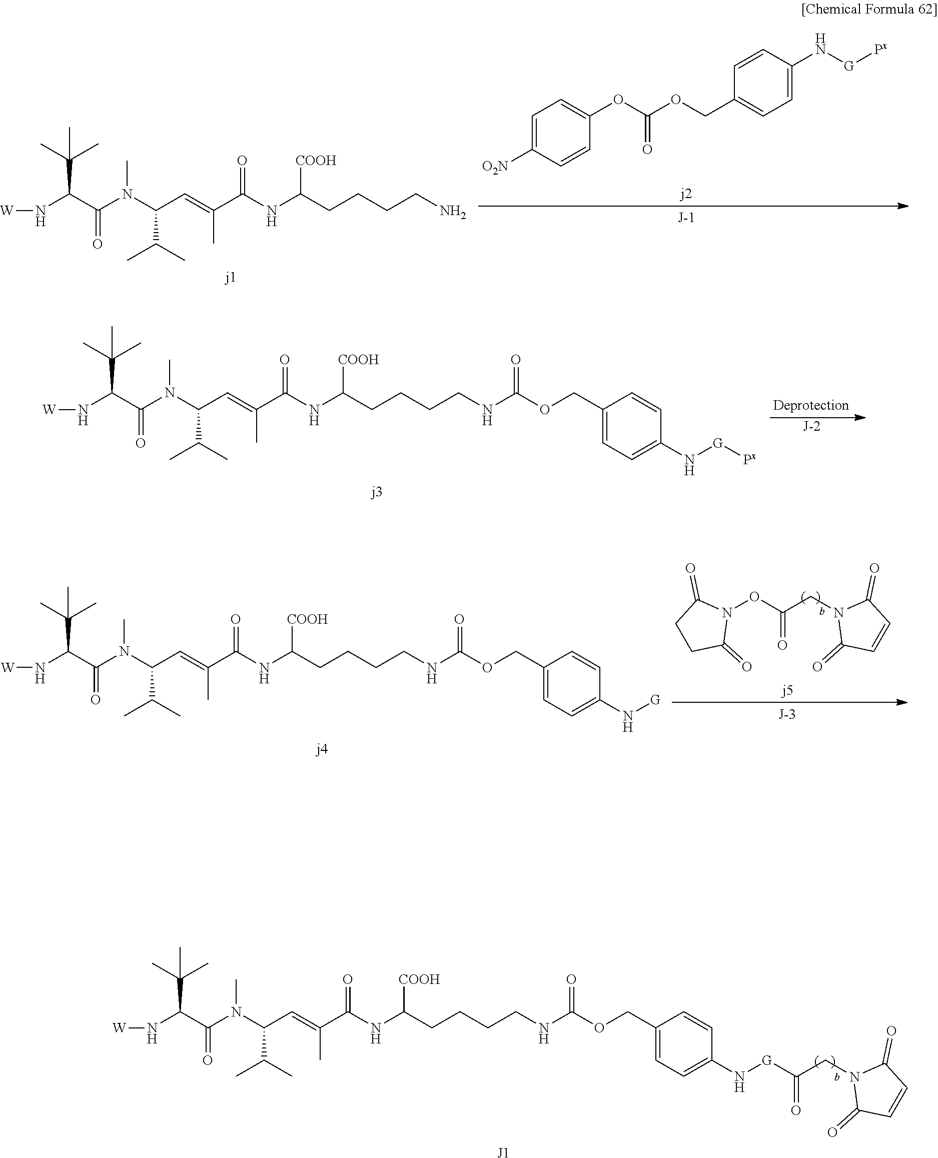

wherein, W, G and h are as described in item 18; and P^{X} is as defined above.

Compound j1 represents compound D1 of Production Method D or compound L5 of Production Method L. Compound j2 represents compound g3 of Production Method G. Compound j5 may be, for example, purchased as a commercial product.

[J-1 Step]

Compound j3 may be produced from compound j1 and compound j2 in accordance with the method described in the above G-3 step.

[J-2 Step]

Compound j4 may be produced from compound j3 in accordance with the method described in the above A-18 step.

[J-3 Step]

Compound J1 may be produced from compound j4 and compound j5 in accordance with the method described in the above A-16 step.

Production Method K

When Z″ is a group represented by formula (Z-6); Y is a group represented by formula (Y-1); and G is a group represented as an amino acid or a peptide, the compound represented by formula (3-2) may be produced by, for example, the following production method:

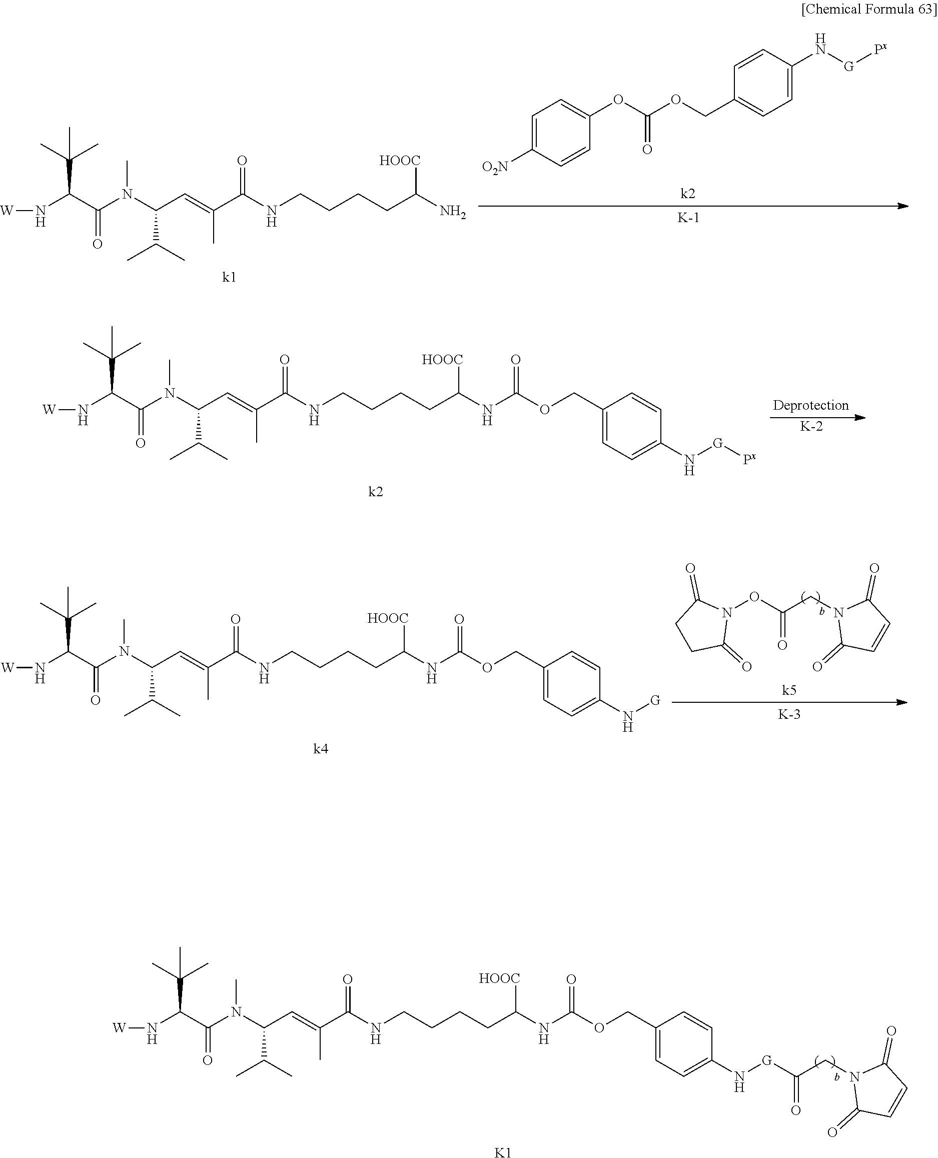

wherein, W, G and h are as described in item 18; and P^{X} is as defined above.

Compound k1 represents compound D1 of Production Method D or compound L5 of Production Method L. Compound k2 represents compound g3 of Production Method G. Compound k5 may be, for example, purchased as a commercial product.

[K-1 Step]

Compound k3 may be produced from compound k1 and compound k2 in accordance with the method described in the above G-3 step.

[K-2 Step]

Compound k4 may be produced from compound k3 in accordance with the method described in the above A-18 step.

[K-3 Step]

Compound K1 may be produced from compound k4 and compound k5 in accordance with the method described in the above A-16 step.

Production Method L

When W is a group represented by formula (W-2); Z is a group represented by formula (Z-1) or formula (Z-2); Z′ is a group represented by formula (Z-3) or formula (Z-4); and Z″ is a group represented by formula (Z-5), formula (Z-6), formula (Z-8) or formula (Z-9), compound 16 is a production intermediate of compound L1, L2, L3, L4 or L5 represented by formula (1-1), formula (1-2), formula (1-3), formula (3-1) or (3-2). Compound 16 may be produced by, for example, the following production method. Compound L1, L2, L3, L4 or L5 may be produced from compound 16 in accordance with the production method described in A-16 step to A-19 step of Production Method A.

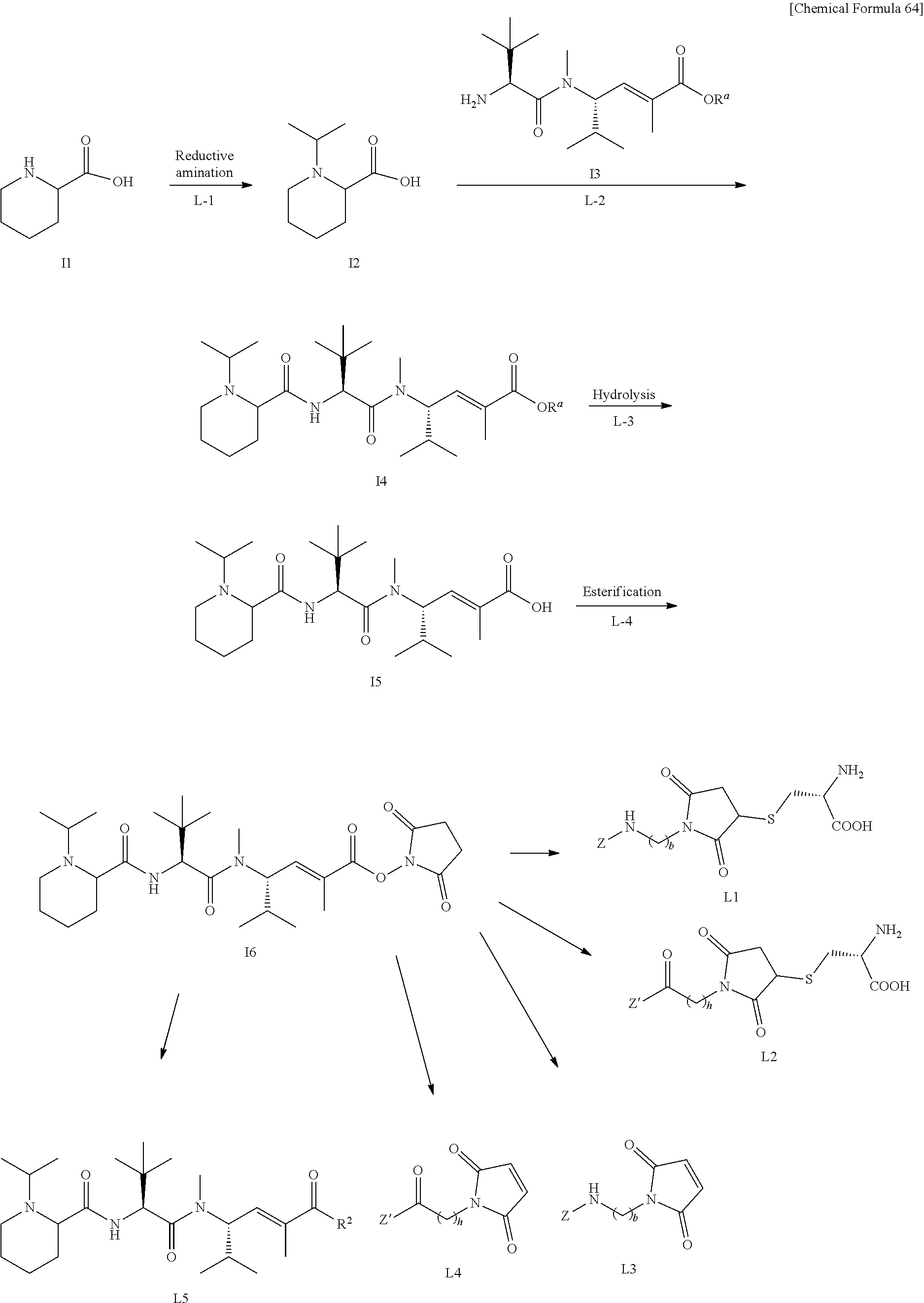

wherein, R², b and h are as defined in item 1, item 2, item 3, item 17 or item 18; and R^{a} represents a C₁₋₆ alkyl group.

Compound 11 may be, for example, purchased as a commercial product. Compound 13 may be produced by the method described in, for example, Tetrahedron Lett., 1997, 38, 317-320 and the like, or may be purchased as a commercial product.

[L-1 Step]

Compound 12 may be produced in accordance with the method described in, for example, International Publication No. WO 2003/082268 and the like.

[L-2 Step]

Compound 14 may be produced from compound 12 and compound 13 in accordance with the method described in the above A-13 step.

[L-3 Step]

Compound 15 may be produced from compound 14 in accordance with the method described in the above A-14 step.

[L-4 Step]

Compound 16 may be produced from compound 15 in accordance with the method described in the above A-15 step.

The antibody-drug conjugate of the present invention represented by formula (2-1) or (2-2) may be produced by, for example, the following production method M or production method N:

Production Method M

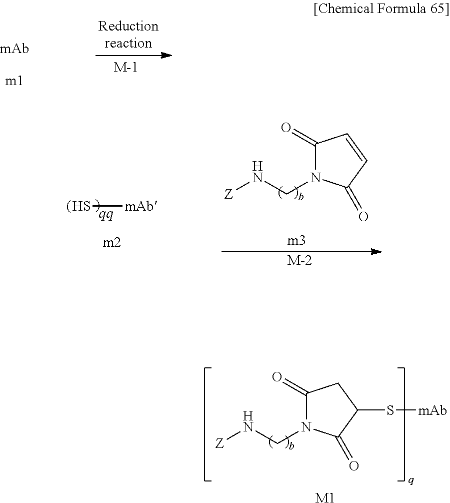

wherein, mAb, q, b and Z are as defined in item 8; mAb′ represents mAb in which a disulfide bond is reduced; and qq represents an integer of 1 to 20.

[M-1 Step]

Compound m2 may be produced by allowing compound m1 to react with an appropriate disulfide reducing agent in an appropriate buffer solution. Examples of the disulfide reducing agent include dithiothreitol, mercaptoethanol and tris(2-carboxyethyl)phosphine; and preferably include tris(2-carboxyethyl)phosphine. Examples of the buffer solution include Tris-HCl, PBS, HEPES, acetate buffers, borate buffers, phosphate buffers and carbonate buffers, and preferably include Tris-HCl and PBS. The pH upon reaction is normally 2 to 12, and is preferably 4 to 9. The reaction time is normally 5 minutes to 24 hours, and is preferably 5 minutes to 5 hours. The reaction temperature is normally −10° C. to 50° C., and is preferably 0° C. to 40° C.

[M-2 Step]

Compound M1 may be produced by allowing compound m2 and compound m3 to react in an appropriate buffer solution. Examples of the buffer solution include Tris-HCl, PBS, HEPES, acetate buffers, borate buffers, phosphate buffers and carbonate buffers, and preferably include Tris-HCl and PBS. The pH upon reaction is normally 2 to 12, and is preferably 4 to 9. The reaction time is normally 5 minutes to 72 hours, and is preferably 30 minutes to 24 hours. The reaction temperature is normally −78° C. to 200° C., and is preferably 0° C. to 25° C.

Production Method N

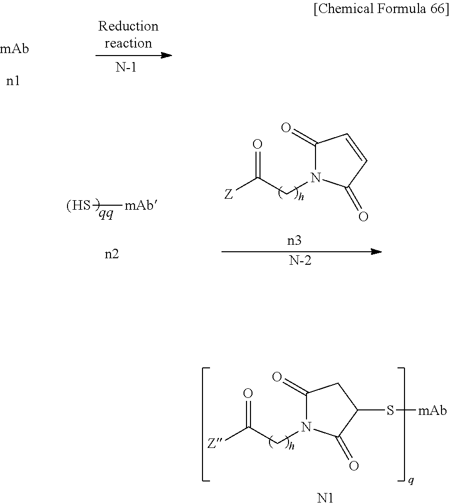

wherein, mAb, q, h and Z″ are as defined in item 9; mAb′ represents mAb in which a disulfide bond is reduced; and qq represents an integer of 1 to 20.

[N-1 Step]

Compound n2 may be produced from compound n1 in accordance with the method described in the above M-1 step.

[N-2 Step]

Compound N1 may be produced from compound n2 and compound n3 in accordance with the method described in the above M-2 step.

The production methods for the hemiasterlin derivative and antibody-drug conjugate according to the present invention have been shown in the above. However, the hemiasterlin derivative and antibody-drug conjugate according to the present invention may also be produced even by a method other than those, for example, by appropriately combining methods known to a person having ordinary skill in the art.

Appropriate bases used in each step of the above production methods should be selected as appropriate depending on reactions, types of raw material compounds and the like, and examples thereof include alkali bicarbonates such as sodium bicarbonate and potassium bicarbonate; alkali carbonates such as sodium carbonate and potassium carbonate; metal hydrides such as sodium hydride and potassium hydride; alkali metal hydroxides such as sodium hydroxide and potassium hydroxide; alkali metal alkoxides such as sodium methoxide and sodium t-butoxide; organometallic bases such as butyllithium and lithium diisopropylamide; and organic bases such as triethylamine, diisopropylethylamine, pyridine, 4-dimethylaminopyridine (DMAP) and 1,8-diazabicyclo[5.4.0]-7-undecene (DBU).

Appropriate solvents used in each step of the above production methods should be selected as appropriate depending on reactions, types of raw material compounds and the like, and examples thereof include alcohols such as methanol, ethanol and isopropanol; ketones such as acetone and methyl ketone; halogenated hydrocarbons such as methylene chloride and chloroform; ethers such as tetrahydrofuran (THF) and dioxane; aromatic hydrocarbons such as toluene and benzene; aliphatic hydrocarbons such as hexane and heptane; esters such as ethyl acetate and propyl acetate; amides such as N,N-dimethylformamide (DMF) and N-methyl-2-pyrrolidone; sulfoxides such as dimethylsulfoxide (DMSO); nitriles such as acetonitrile; distilled water; and the like, and one of these solvents may be used singly, or two or more of them may be mixed for use. In addition, depending on the type of reactions, organic bases such as triethylamine, diisopropylethylamine and pyridine may be used as the solvent.

The hemiasterlin derivative and antibody-drug according to the present invention may be separated and purified by methods known to a person having ordinary skill in the art. Examples thereof include extraction, partitioning, reprecipitation, column chromatography (for example, silica gel column chromatography, ion exchange column chromatography or preparative liquid chromatography) or recrystallization.

As the recrystallization solvent, for example, alcohol solvents such as methanol, ethanol and 2-propanol; ether solvents such as diethyl ether; ester solvents such as ethyl acetate; aromatic hydrocarbon solvents such as benzene and toluene; ketone solvents such as acetone; halogenated solvents such as dichloromethane and chloroform; hydrocarbon solvents such as hexane; aprotic solvents such as dimethylformamide acetonitrile; water; or mixed solvents thereof may be used.

As other purification method, the method described in The Experimental Chemistry (edited by The Chemical Society of Japan, Maruzen), vol. 1 and the like may be used. In addition, determination of the molecular structure of the hemiasterlin derivative and antibody-drug conjugate according to the present invention may be readily carried out by spectroscopic approaches such as nuclear magnetic resonance, infrared absorption technique and circular dichroism spectroscopy, or mass spectrometry, with reference to the structure derived from their respective raw material compounds.

In addition, intermediates or final products in the above production methods may also be derivatized into other compounds included in the present invention by converting their functional groups as appropriate, in particular, by extending various side chains using an amino group, hydroxy group, carbonyl group, halogen atom or the like as the basis, and upon this, by carrying out protection and deprotection of the above functional groups as necessary. The conversion of functional groups and extension of side chains may be carried out by general methods that are conventionally performed (for example, see Comprehensive Organic Transformations, R. C. Larock, John Wiley & Sons Inc. (1999) and the like).

The hemiasterlin derivative and antibody-drug conjugate according to the present invention may have asymmetry or may have a substituent having an asymmetric carbon, and optical isomers are present in such compounds. Optical isomers may be produced in accordance with conventional methods. Examples of the production method include a method of using a raw material having an asymmetric point or a method of introducing asymmetry in the midway stage. For example, in the case of optical isomers, optical isomers may be obtained by using optically active raw materials or by carrying out optical resolution or the like at an appropriate stage during the production process. When the hemiasterlin derivative and antibody-drug conjugate according to the present invention have a basic functional group, examples of the optical resolution method include a diastereomer method, in which a salt is formed using an optically active acid (for example, monocarboxylic acids such as mandelic acid, N-benzyloxyalanine and lactic acid; dicarboxylic acids such as tartaric acid, o-diisopropylidene tartaric acid and malic acid; sulfonic acids such as camphorsulfonic acid and bromocamphorsulfonic acid) in an inert solvent (for example, an alcohol solvent such as methanol, ethanol and 2-propanol; an ether solvent such as diethyl ether; an ester solvent such as ethyl acetate; a hydrocarbon solvent such as toluene; an aprotic solvent such as acetonitrile; or a mixed solvent of two or more selected from the above solvents). When the hemiasterlin derivative or synthetic intermediate thereof according to the present invention has an acidic functional group such as a carboxyl, optical resolution can also be carried out by using an optically active amine (for example, an organic amine such as 1-phenylethylamine, quinine, quinidine, cinchonidine, cinchonine and strychnine) to form a salt.

Examples of the temperature at which the salt is formed include the range from −50° C. to the boiling point of the solvent, preferably include the range from 0° C. to the boiling point, and more preferably include the range from room temperature to the boiling point of the solvent. In order to improve optical purity, it is desirable that the temperature be once raised to the vicinity of the boiling point of the solvent. Upon separating the precipitated salt by filtration, the yield may be improved by cooling as necessary. Examples of the amount of the optically active acid or amine to be used include the range of about 0.5 to about 2.0 equivalent to the substrate, and preferably include the range around 1 equivalent. As necessary, an optically active salt with high purity can be obtained by recrystallizing a crystal in an inert solvent (for example, an alcohol solvent such as methanol, ethanol and 2-propanol; an ether solvent such as diethyl ether; an ester solvent such as ethyl acetate; a hydrocarbon solvent such as toluene; an aprotic solvent such as acetonitrile; or a mixed solvent of two or more selected from the above solvents). In addition, a free form may be obtained by treating a salt that has been optically resolved with an acid or base through a conventional method, as necessary.

Among the raw materials or intermediates in the production methods described above, those, for which the production method was not described, are either commercially available compounds or may be synthesized from commercially available compounds by methods known to a person having ordinary skill in the art or methods equivalent thereto.

The pluripotent stem cells in the present invention are not limited in any way as long as they are stem cells possessing pluripotency that allows differentiation into any cell present in organisms and proliferative capacity in combination. Pluripotent stem cells may be induced, for example, from fertilized ova, cloned embryos, germline stem cells, interstitial stem cells or somatic cells. Examples of pluripotent stem cells can include embryonic stem cells (ES cells), embryonic germ cells (EG cells) and induced pluripotent stem cells (iPS cells). Multi-lineage differentiating stress enduring cell (Muse cells) obtained from mesenchymal stem cells (MSC) and spermatogonial stem cells (germline stem cells: GS cells) produced from germ cells (for example, testis) are also encompassed in pluripotent stem cells. ES cells were established in 1981 for the first time, and have been applied even to production of knockout mice since 1989. By 1998, human embryonic stem cells had been established, and are now increasingly used even for regenerative medicine. ES cells can be produced by culturing an inner cell mass on feeder cells or in a culture medium containing LIF (leukemia inhibitory factor). Production methods for ES cells are described, for example, in WO 96/22362, WO 02/101057, U.S. Pat. Nos. 5,843,780, 6,200,806 and 6,280,718. ES cells may be obtained from appropriate institutions, or may be purchased as a commercial product. For example, KhES-1, KhES-2 and KhES-3, which are human ES cells, are available from Institute for Frontier Medical Sciences, Kyoto University. An Rx::GFP cell line (derived from the KhES-1 cell line), which is human ES cells, is available from Institute of Physical and Chemical Research. An EB5 cell line and D3 cell line, which are mouse ES cells, are available from Institute of Physical and Chemical Research and ATCC, respectively.

Nuclear transfer embryonic stem cells (ntES cells), one type of ES cells, can be established from a cloned embryo made by transplanting a somatic nucleus into an ovum removed of its nucleus. EG cells can be produced by culturing primordial germ cells in a culture medium containing mSCF, LIF and bFGF (Cell, 70:841-847, 1992).

Induced pluripotent stem cells (iPS cells) in the present invention are cells for which pluripotency has been induced by reprogramming somatic cells, for example, by a known method (Cell 126, p 663-676, 2006, Cell 131, p 861-872, 2007, Science 318, p 1917-1920, 2007, Nat Biotechnol 26, p 101-106, 2008). Specific examples thereof include cells obtained by reprogramming differentiated somatic cells such as fibroblasts and peripheral blood mononuclear cells with any combination of multiple genes selected from a group of reprogramming genes including Oct3/4, Klf4, Klf1, Klf2, Klf5, Sox2, Sox1, Sox3, Sox15, Sox17, Sox18, c-Myc, N-Myc, L-Myc, TERT, SV40 Large T antigen, Glis1, Nanog, Sa114, lin28 and Esrrb. A combination including at least one, two or three reprogramming factors is suitable, and a combination of four or more reprogramming factors is preferred. Examples of preferred combination of reprogramming factors can include (1) Oct3/4, Sox2, Klf4 and Myc (c-Myc or L-Myc) and (2) Oct3/4, Sox2, Klf4, Lin28 and L-Myc.

These reprogramming factors may be introduced in the form of protein into cells with a method such as lipofection, fusion with cell-penetrating peptide and microinjection, or may be introduced in the form of nucleic acid (DNA/RNA) into cells with a method such as lipofection, liposomes, microinjection, a virus, a plasmid vector, an episomal vector and an artificial chromosome vector. Examples of the virus vector include a lentivirus vector, a retrovirus vector, an adenovirus vector, an adeno-associated virus vector and a Sendai virus vector. As the plasmid vector, a commonly available plasmid for mammalian cells may be used, into which regulatory sequences such as a promoter, an enhancer, a ribosome-binding sequence and terminator are commonly incorporated in order to enhance the expression efficiencies of reprogramming factors, and a factor such as EBNA-1 is incorporated in some cases in order to enhance the self-replication efficiency of the plasmid. Not only by a method of production through direct reprogramming by gene expression, iPS cells can be induced from somatic cells, for example, by addition of a compound (Science 341, p 651-654, 2013, WO 2010/068955).

Further, an established iPS cell can also be obtained, and, for example, an iPS cell line established in Center for iPS Cell Research and Application, Kyoto University (CiRA) is available from Kyoto University and iPS PORTAL, Inc.

Somatic cells for use as a starting material in producing iPS cells may be any type of cells except germ cells, and examples thereof include fibroblasts, epithelial cells, mucosal epithelial cells, exocrine gland epithelial cells, hormone-secreting cells, alveolar cells, neurons, pigment cells, hematopoietic cells (for example, peripheral blood mononuclear cells (PBMC), T cells, cord blood cells), mesenchymal stem cells, liver cells, pancreatic cells, intestinal epithelial cells and smooth muscle cells, and precursor cells thereof. There is no limitation to the degree of differentiation of tissue and the age of an animal for collection, and any of the mentioned cells may be used as a somatic cell material in the present invention.

The iPS cells for use in the present invention are, for example iPS cells of a mammal (for example, human, monkey, pig, rabbit, rat or mouse), preferably iPS cells of a rodent (for example, mouse or rat) or a primate (for example, human or monkey), and more preferably human iPS cells. The iPS cells for use in the present invention include iPS cells genetically modified by an approach of, for example, genome editing.

ES cells are one type of stem cells possessing pluripotency like iPS cells, and known to be expressing the same antigen as iPS cells on their cell surfaces (Cell, 131:861-872, 2007). Hence, the antibody-drug conjugate according to the present invention can induce cell death not only to iPS cells but also to ES cells. That is, the antibody-drug conjugate according to the present invention is capable of selectively eliminating ES cells from a cell population including a differentiated cell derived from an ES cell. Because pluripotent stem cells are also expressing a common antigen with iPS cells on their cell surfaces, the antibody-drug conjugate according to the present invention can induce cell death to pluripotent stem cells, and is capable of selectively eliminating pluripotent stem cells from a cell population including a differentiated cell derived from a pluripotent stem cell.

In the present specification, the “agent for eliminating a pluripotent stem cell” means an agent that induces the cell death of a pluripotent stem cell to eliminate the pluripotent stem cell. Pluripotent stem cells can be completely or partially eliminated from a cell population including a differentiated cell derived from a pluripotent stem cell by allowing the agent for eliminating a pluripotent stem cell to act thereon to induce the cell death of the pluripotent stem cell.

In the present specification, a “killing agent for a pluripotent stem cell” means an agent that induces the cell death of a pluripotent stem cell to kill the pluripotent stem cell. In the present specification, a “reducer for a pluripotent stem cell” means an agent that induces the cell death of a pluripotent stem cell to reduce the number of pluripotent stem cells or the proportion thereof in a cell population.

The antibody-drug conjugate is delivered specifically into particular antigen-expressing cells through uptake into cells utilizing antibody-antigen reaction, and then undergoes metabolism by an enzyme in cells through the mechanisms mentioned above to release the compound derived from the drug moiety from the antibody-drug conjugate, thereby successfully exerting drug efficacy only in the particular antigen-expressing cells. That is, the antibody moiety of the antibody-drug conjugate according to the present invention against an antigen expressed on cell surfaces of pluripotent stem cells recognizes the pluripotent stem cells and can be taken up specifically by the pluripotent stem cells, and hence the antibody-drug conjugate according to the present invention can be expected to exert cellular toxicity to pluripotent stem cells by releasing the compound derived from the drug moiety in the cells and in contrast exhibit low cellular toxicity to differentiated cells, which do not express the antigen on their cell surfaces.

On the other hand, the antibody-drug conjugate is considered to be broken down by protease or the like contained in a culture medium before being delivered to intended cells and release the compound derived from the drug moiety in the culture medium to disadvantageously damage cells in a non-selective manner. In case of conventional antibody-drug conjugates, cell membrane permeability of the drug moiety is high, and therefore, the compound derived from the drug moiety released into a culture medium is also passively diffused into and taken up by differentiated cells. As a result, unintentional exposure is caused, which is unfavorable because damage to differentiated cells tends to occur.

In contrast, the drug moiety of the antibody-drug conjugate according to the present invention, that is, the hemiasterlin derivative has low cell membrane permeability, and thus, even if the compound derived from the drug moiety is released in a culture medium before reaching pluripotent stem cells such as iPS cells, the drug is unlikely to be passively diffused into and taken up by differentiated cells, thus, it can be expected that damage to differentiated cells is small.

Furthermore, when a compound with low membrane permeability is used as the drug moiety to be conjugated with an antibody, the compound derived from the drug moiety released in intended pluripotent stem cells is hindered from flowing outside the cells via the cell membrane, and therefore, the compound derived from the drug moiety can remain in the intended cells for a long period of time and it is expected that satisfactory cell-eliminating effect is exerted.

That is, because the antibody-drug conjugate according to the present invention has a hemiasterlin derivative with low cell membrane permeability as the drug moiety, it is expected to exhibit cellular toxicity specifically to pluripotent stem cells, and also to have small influence on differentiated cells.

Since the antibody-drug conjugate according to the present invention is taken up by pluripotent stem cells such as ES cells and iPS cells and the drug released in the cells exhibits cellular toxicity to induce the growth inhibition and cell death of the pluripotent stem cells, the antibody-drug conjugate according to the present invention can effectively eliminate remaining pluripotent stem cells from a cell population including a differentiated cell derived from a pluripotent stem cell. Further, since the antibody-drug conjugate according to the present invention induces cell death in a manner specific to iPS cells, the antibody-drug conjugate according to the present invention allows efficient elimination of iPS cells with toxicity to differentiated cells reduced.

Examples of the cell cluster in the present invention include a cell laminate produced by laminating two or more monolayers of cells or newly forming a cell layer on monolayered cells, a cell aggregate produced by aggregating cells, a cell assembly obtained by three-dimensionally laminating cells by using a device such as a 3D bioprinter, and an organoid formed through self-assembly in three-dimensional culture. Each of these cell clusters, in which cells provide a culture scaffold by adhering to each other to retain the structure, may be in a state in which a scaffold material such as hydrogel is contained in the cell cluster. Hydrogel, a substance that can contain a large amount of water, can allow substances necessary for survival such as oxygen, water and nutrients, and waste products to readily diffuse and move. A biocompatible substance is typically used, and examples thereof include gelatin hydrogel.

The cell population including differentiated cells derived from iPS cells, to which the present invention is to be applied, is a cell population that results from induction of differentiation of iPS cells and serves as an active ingredient of cellular medicines, including products for regenerative medicine, or a production intermediate thereof, and examples thereof include plate-cultured cells including a colony, suspension-cultured cells as single cells, and the above-defined cell cluster.

Examples of cells obtained by induction of differentiation of iPS cells include, but are not limited to, cells constituting tissues of the hair, eye (retina, cornea), nerve tissue (brain, spinal cord, peripheral nerve), heart, bone (cartilage), lung, kidney, pancreas, intestinal tract, blood vessel, blood, muscle, meniscus, Achilles tendon, liver, fat (breast), skin and esophagus, or stem cells/precursor cells of the cells.

The method of inducing differentiation from iPS cells into tissues in the step of inducing differentiation of a cell population including iPS cells into differentiated cells may be any method capable of inducing differentiation, and is not limited in any way. Examples of the method of inducing differentiation from iPS cells into tissues include a method of inducing differentiation of neural progenitor cells by culturing iPS cells in a serum-free culture medium in the presence of a BMP inhibitor and an activin/TGFβ family inhibitor.

In the present invention, a “cell population including a differentiated cell derived from an iPS cell with substantially no iPS cell” means a cell population that includes a differentiated cell derived from an iPS cell and does not include such a number of iPS cells that the iPS cells cause formation of teratoma in an organism. Whether any iPS cell remains may be detected by a method well known to a person having ordinary skill in the art, and the proportion of iPS cells to the total number of cells can also be quantified. Examples of the method for quantifying iPS cells include, but are not particularly limited to, a method of measuring the expression level of a marker molecule expressed on the surface or inside of cells. Examples of the marker molecule expressed on cell surfaces include TRA1-60 and SSEA4, and examples of the marker molecule expressed in the inside of cells (in nuclei) include NANOG, OCT4 and LIN28A. Examples of measurement methods for these marker molecules include flow cytometry for expression markers on cell surfaces, and immunostaining and reverse transcription polymerase chain reaction (RT-PCR) for expression markers in cell nuclei. Examples of one aspect of the cell population including a differentiated cell derived from an iPS cell with substantially no iPS cell include a cell population including a differentiated cell derived from an iPS cell in which the proportion of the number of iPS cells to the total number of the cells is less than 1%; examples of another aspect thereof include a cell population including a differentiated cell derived from an iPS cell in which the proportion of the number of iPS cells to the total number of the cells is less than 0.1%; and examples of another aspect thereof include a cell population including a differentiated cell derived from an iPS cell in which the proportion of the number of iPS cells to the total number of the cells is less than 0.01%.

In order to eliminate undifferentiated cells retaining pluripotency or cells with resistance to differentiation, specifically, remaining iPS cells or iPS-like cells from a cell population resulting from induction of differentiation of iPS cells, the antibody-drug conjugate according to the present invention can be allowed to act on the cell population. In the step of contacting a cell population including a differentiated cell derived from an iPS cell with the antibody-drug conjugate according to the present invention, it is preferred as a method for contacting the cell population with the antibody-drug conjugate to directly contact the antibody-drug conjugate according to the present invention with the cells or cell population. Specifically, a liquid containing the antibody-drug conjugate (a solution or a suspension), or the antibody-drug conjugate itself can be added to a culture solution for the cells or cell population, and a method of adding a concentrate of the antibody-drug conjugate to a culture solution is commonly used. Although the solvent used for the concentrate of the antibody-drug conjugate may be any solvent that can dissolve the antibody-drug conjugate therein, phosphate buffered saline, which has relatively high dissolvability irrespective of physical properties of antibody-drug conjugates and have low toxicity to cells, is often used. The concentration of the antibody-drug conjugate in the concentrate is, for example, in the range from 0.01 μg/mL to 10 mg/mL, and is, in a preferred mode, in the range from 0.1 μg/mL to 1 mg/mL. Alternatively, for a culture solution containing cells or cell population, the antibody-drug conjugate according to the present invention may also be contacted with the cells or cell population by exchanging the culture solution with a culture solution containing a required amount of the antibody-drug conjugate according to the present invention.

The time to contact the antibody-drug conjugate according to the present invention with the cell population is not limited in any way as long as differentiated cells can survive, and normally is in the range from 1 hour to 96 hours, and preferably in the range from 24 hours to 96 hours.

The temperature in contacting the antibody-drug conjugate according to the present invention with the cell population is not limited in any way as long as differentiated cells can survive at the temperature, and is normally in the range from 4° C. to 40° C., and preferably in the range from 20° C. to 37° C.

A common culture medium for cell culture or a minimal essential culture medium prepared with a buffer may be used for the culture medium for use in contacting the antibody-drug conjugate according to the present invention and the cell population, and a culture medium for induction of differentiation of cells is preferably used. The minimal essential culture medium is not limited in any way as long as the minimal essential culture medium is a culture medium applicable to culture of animal cells such as BME medium, BGJb medium, CMRL1066 medium, Glasgow MEM medium, Improved MEM Zinc Option medium, IMDM medium, Medium 199 medium, Eagle MEM medium, αMEM medium, DMEM medium, Ham's medium, RPMI 1640 medium and Fischer's medium, and mixed culture media of them.

In the present specification, a “cell for transplantation” means a cell to be used for administration into the living body of a human or an animal other than human in regenerative medicine or the like. The cell for transplantation may be a cell that repairs the function of a tissue or organ, or a cell that prevents or treats a disease or damage. Examples of the method for administering the cell for transplantation include, but are not particularly limited to, a method of surgically transplanting to an affected part.

In the present invention, the pharmaceutical composition comprising, as an active ingredient, a cell included in an iPS cell-derived cell population with substantially no iPS cell may be used, for example, by administering to a human or an animal other than human in regenerative medicine or the like (preferably, transplantation). The pharmaceutical composition appropriately contains a carrier and/or additive.

### EXAMPLES

Hereinafter, the present invention will be explained further specifically with reference to Reference Examples, Examples and Test Examples, but the present invention is not limited to them, of course. Note that the names of compounds shown in the following Reference Examples and Examples do not necessarily follow the IUPAC nomenclature of chemistry.

Compounds of Reference Examples and Examples may be obtained as an acid addition salt such as a TFA salt, depending on a method of treatment after the reaction and the like.

In order to simplify description of the specification, abbreviations as shown below may be used in Reference Examples, Examples and the tables in Examples. As abbreviations used for substituents, Me represents a methyl group, Et represents an ethyl group, Boc represents a tert-butoxycarbonyl group, Fmoc represents a 9-fluorenylmethyloxycarbonyl group, trt represents a trityl group, Ph represents a phenyl group. DMSO represents dimethyl sulfoxide, TFA represents trifluoroacetic acid, THF represents tetrahydrofuran, TCEP represents tris(2-carboxyethyl)phosphine, Tris-HCl represents trishydroxymethylaminomethane hydrochloride, PBS represents phosphate buffered saline, HEPES represents 2-[4-(2-hydroxyethyl)piperazin-1-yl]ethanesulfonic acid, and PIPES represents piperazine-1,4-bis(2-ethanesulfonic acid). For symbols used for NMR, s means a singlet, d means a doublet, dd means a doublet of doublets, t means a triplet, q means a quartet, m means a multiplet, br means broad, brs means a broad singlet, brd means a broad doublet, brm means a broad multiplet, and J means the binding constant.

High Performance Liquid Chromatography-Mass Spectrometer; measurement conditions for LCMS are as follows, and the observed value of mass spectrometry [MS (m/z)] is shown as [M+nH]ⁿ⁺/n, [M+Na]⁺ or [M−nH]ⁿ⁻/n, and the retention time is shown as Rt (min). Note that, for each found value, the measurement conditions used for the measurement are denoted by A to D or F to H.

Measurement Condition A

### Detection Equipment: Shimadzu LCMS-IT-TOF

Column: Phenomenex Kinetex (1.7 μm C18.50 mm×2.10 mm)
Solvents: solution A: 0.1% HCOOH/CH₃CN, solution B: 0.1% HCOOH/H₂O

### Gradient Condition:

0.0 min; A/B=1:99

0.0 to 1.4 min; Linear gradient from 1% to 95% A

1.4 to 1.6 min; A/B=95:5

1.6 to 2.0 min; A/B=1:99

Flow Rate: 1.2 mL/min

### UV: 220/254 nm

### Column Temperature: 40° C.

Measurement Condition B

### Detection Equipment: Shimadzu LCMS-IT-TOF

Column: Phenomenex Kinetex (1.7 μm C18, 50 mm×2.10 mm)
Solvents: solution A: 0.1% HCOOH/CH₃CN, solution B: 0.1% HCOOH/H₂O

### Gradient Condition:

0.0 min; A/B=10:90

0.0 to 1.4 min; Linear gradient from 10% to 90% A

1.4 to 1.6 min; A/B=90:10

1.6 to 2.0 min; A/B=10:90

Flow Rate: 1.2 mL/min

### UV: 220/254 nm

### Column Temperature: 40° C.

Measurement Condition C

### Detection Equipment: Shimadzu LCMS-IT-TOF

Column: Phenomenex Kinetex (1.7 μm C8, 50 mm×2.10 mm)
Solvents: solution A: 0.1% HCOOH/CH₃CN, solution B: 0.1% HCOOH/H₂O

### Gradient Condition:

0.0 min; A/B=1:99

0.0 to 1.4 min; Linear gradient from 1% to 95% A

1.4 to 1.6 min; A/B=95:5

1.6 to 2.0 min; A/B=1:99

Flow Rate: 1.2 mL/min

### UV: 220/254 nm

### Column Temperature: 40° C.

Measurement Condition D

### Detection Equipment: Shimadzu LCMS-IT-TOF

Column: Phenomenex Kinetex (1.7 μm C8, 50 mm×2.10 mm)
Solvents: solution A: 0.1% HCOOH/CH₃CN, solution B: 0.1% HCOOH/H₂O

### Gradient Condition:

0.0 min; A/B=10:90

0.0 to 1.4 min; Linear gradient from 10% to 90% A

1.4 to 1.6 min; A/B=90:10

1.6 to 2.0 min; A/B=10:90

Flow Rate: 1.2 mL/min

### UV: 220/254 nm

### Column Temperature: 40° C.

Measurement Condition E

Detection Equipment: Perkin-Elmer Sciex API 150EX Mass spectrometer

### HPLC: Shimadzu LC 10ATVP

Column: Shiseido CAPCELL PAK C18 ACR (S-5 μm, 4.6 mm×50 mm)
Solvents: solution A: 0.035% TFA/CH₃CN, solution B: 0.05% TFA/H₂O
Gradient Condition: 0.0 to 0.5 min solution A 10%, 0.5 to 4.8 min solution A Linear gradient from 10% to 99% A, 4.8 to 5.0 min
Flow Rate: 3.5 mL/min solution A 99%

### UV: 220/254 nm

### Column Temperature: 25° C.

Measurement Condition F

Detection Equipment: ACQUITY (registered Trademark) SQdetecter (Waters Corporation)
HPLC: ACQUITY (registered Trademark) system
Column: Waters ACQUITY UPLC (registered Trademark) BEH C18 (1.7 μm, 2.1 mm×30 mm)
Solvents: solution A: 0.06% formic acid/CH₃CN, solution B: 0.06% formic acid/H₂O
Gradient Condition: 0.0 to 1.3 min Linear gradient from 2% to 96% A
Flow Rate: 0.8 mL/min

### UV: 220/254 nm

### Column Temperature: 25° C.

Measurement Condition G

### Detection Equipment: Shimadzu LCMS-IT-TOF

Column: Phenomenex Kinetex (1.7 μm, C8.50 mm×2.10 mm)
Solvents: solution A: 0.1% HCOOH/CH₃CN, solution B: 0.1% HCOOH/H₂O

### Gradient Condition:

0.0 min; A/B=10:90

0.0 to 1.4 min; Linear gradient from 10% to 95% A

1.4 to 1.6 min; A/B=95:5

1.6 to 2.0 min; A/B=10:90

Flow Rate: 1.2 mL/min

### UV: 220/254 nm

### Column Temperature: 40° C.

Measurement Condition H

### Detection Equipment: Shimadzu LCMS-IT-TOF

Column: Phenomenex Kinetex (1.7 μm C8, 50 mm×2.10 mm)
Solvents: solution A: 0.1% HCOOH/CH₃CN, solution B: 0.1% HCOOH/H₂O

### Gradient Condition:

0.0 min; A/B=40:60

0.0 to 1.4 min; Linear gradient from 40% to 95% A

1.4 to 1.6 min; A/B=95:5

1.6 to 2.0 min; A/B=5:95

Flow Rate: 1.2 mL/min

### UV: 220/254 nm

### Column Temperature: 40° C.

High Performance Liquid Chromatography; measurement conditions for determining the average Drug Antibody Ratio (average DAR) are as follows, and the retention time is shown as Rt (min). Note that, for each found value, the measurement conditions used for the measurement are denoted by E or I.

Measurement Condition I

HPLC: Shimadzu LC-10A series
Column: nonporous TSKgel Butyl-NPR column (Tosoh Bioscience, 2.5 μm, 35 mm×4.6 mm)
Solvents: solution A: 1.5 mol/L ammonium sulfate, 25 mmol/L aqueous sodium phosphate solution (pH 6.95), solution B: 25% isopropanol/25 mmol/L aqueous sodium phosphate solution (pH 6.95)

### Gradient Condition:

0.0 min; A/B=100:0

0.0 to 12.0 min; Linear gradient from 0% to 100% B

12.1 to 18.0 min; A/B=100:0

Flow Rate: 0.8 mL/min

### UV: 230 nm

### Column Temperature: 25° C.

Measurement Condition J

HPLC: Shimadzu LC-10A series
Column: nonporous TSKgel Butyl-NPR column (Tosoh Bioscience, 2.5 μm, 35 mm×4.6 mm)
Solvents: solution A: 1.5 mol/L ammonium sulfate, 25 mmol/L aqueous sodium phosphate solution (pH 6.95), solution B: 25% isopropanol/25 mmol/L aqueous sodium phosphate solution (pH 6.95)

Gradient Condition:
0.0 min; A/B=100:0
0.0 to 24.0 min; Linear gradient from 0% to 100% B
24.1 to 60.0 min; A/B=100:0
   Flow Rate: 0.8 mL/min

### UV: 230 nm

### Column Temperature: 25° C.

### Reference Example 1

### N-(tert-butoxycarbonyl)-N,β,β,1-tetramethyl-L-tryptophyl-N-{(3S,4E)-6-[(2,5-dioxopyrrolidin-1-yl)oxy]-2,5-dimethyl-6-oxohex-4-en-3-yl}-N, 3-dimethyl-L-valinamide

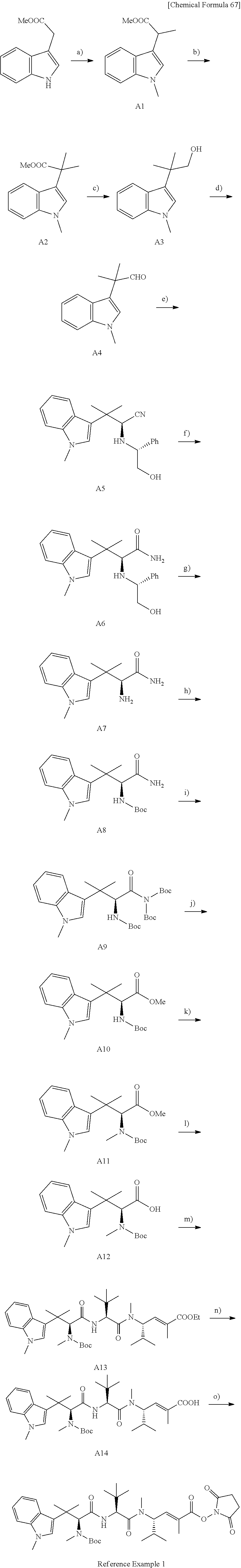

### a) Production of methyl 2-(1-methyl-1H-indol-3-yl)propanoate (Compound A1)

Under nitrogen atmosphere, to a solution of indole-3-acetic acid methyl ester (3.8 g) in tetrahydrofuran (87 mL) at −78° C., potassium hexamethyldisilazide (1 mol/L tetrahydrofuran solution, 65.5 mL) was added dropwise, and the resultant mixture was then stirred at 0° C. for 2 hours. After cooling the reaction solution to −78° C., methyl iodide (23 g) was added dropwise thereto, and the reaction solution was then stirred at 0° C. for 3 hours. After the reaction ended, water was added and the resultant mixture was extracted with diethyl ether. The organic layer was washed with saturated brine, followed by drying over anhydrous magnesium sulfate, and the solvent was distilled off under reduced pressure. The residue was purified by silica gel column chromatography (eluting solvent; hexane:ethyl acetate) to give compound A1 (3.95 g).

¹H-NMR (400 MHz, CDCl₃):1.60 (3H, d, J=7.1 Hz), 3.67 (3H, s), 3.76 (3H, s), 4.02 (1H, q, J=7.1 Hz), 7.00 (1H, s), 7.12 (1H, t, J=7.8 Hz), 7.23 (1H, t, J=7.8 Hz), 7.29 (1H, d, J=7.8 Hz), 7.66 (1H, d, J=7.8 Hz).

### b) Production of methyl 2-methyl-2-(1-methyl-1H-indol-3-yl) propanoate (Compound A2)

Under nitrogen atmosphere, to a solution of compound A1 (3.94 g) in tetrahydrofuran (200 mL) at −78° C., potassium hexamethyldisilazide (1 mol/L tetrahydrofuran solution, 27.7 mL) was added dropwise, and the resultant mixture was then stirred at 0° C. for 2 hours. After cooling the reaction solution to −78° C., methyl iodide (15.4 g) was added dropwise thereto, and the reaction solution was then stirred at 0° C. for 3 hours. After the reaction ended, water was added and the resultant mixture was extracted with diethyl ether. The organic layer was washed with saturated brine, followed by drying over anhydrous magnesium sulfate, and the solvent was distilled off under reduced pressure. The residue was purified by silica gel column chromatography (eluting solvent; hexane:ethyl acetate) to give compound A2 (3.59 g).

¹H-NMR (400 MHz, CDCl₃):1.66 (6H, s), 3.61 (3H, s), 3.73 (3H, s), 6.91 (1H, s), 7.06 (1H, t, J=8.0 Hz), 7.19 (1H, t, J=8.0 Hz), 7.27 (1H, d, J=8.0 Hz), 7.61 (1H, d, J=7.9 Hz).

### c) Production of 2-methyl-2-(1-methyl-1H-indol-3-yl) propan-1-ol (Compound A3)

Under nitrogen atmosphere, to a solution of compound A2 (3.59 g) in diethyl ether (169 mL) and dichloromethane (47 mL) at −78° C., diisobutylaluminum hydride (1 mol/L n-hexane solution, 38.8 mL) was added dropwise, and the resultant mixture was then stirred at 0° C. for 1 hour. After the reaction ended, water was added, and then, to the reaction mixture at 25° C., a saturated aqueous solution of potassium sodium tartrate was added, and the resultant mixture was then extracted with diethyl ether. The organic layer was washed with saturated brine, followed by drying over anhydrous magnesium sulfate, and the solvent was distilled off under reduced pressure. The residue was purified by silica gel column chromatography (eluting solvent; hexane:ethyl acetate) to give compound A3 (3.14 g).

¹H-NMR (400 MHz, CDCl₃):1.42 (6H, s), 3.74 (3H, s), 3.77 (2H, s), 6.87 (1H, s), 7.07 (1H, t, J=7.9 Hz), 7.20 (1H, t, J=7.9 Hz), 7.29 (1H, d, J=8.0 Hz), 7.75 (1H, d, J=8.0 Hz).

### d) Production of 2-methyl-2-(1-methyl-1H-indol-3-yl)propanal (Compound A4)

Under nitrogen atmosphere, a mixed solution of compound A3 (3.14 g), tetrapropylammonium perruthenate (271 mg), N-methylmorpholine-N-oxide (3.26 g) and molecular sieve 4A (7.7 g) in dichloromethane (110 mL) was stirred at 25° C. for 1 hour. After the reaction ended, the reaction solution was filtered through celite and the solvent was then distilled off, and the residue was purified by silica gel column chromatography (eluting solvent; hexane:ethyl acetate) to give compound A4 (2.4 g).

¹H-NMR (400 MHz, CDCl₃):1.53 (6H, s), 3.77 (3H, s), 6.94 (1H, s), 7.07 (1H, t, J=8.0 Hz), 7.22 (1H, t, J=8.0 Hz), 7.30 (1H, d, J=8.0 Hz), 7.53 (1H, d, J=8.0 Hz), 9.47 (1H, s).

### e) Production of (2S)-2-{[(1R)-2-hydroxy-1-phenylethyl]amino}-3-methyl-3-(1-methyl-1H-indol-3-yl)butanenitrile (Compound A5)

Under nitrogen atmosphere, a solution of compound A4 (2.4 g) and (R)-(−)-2-phenylglycinol (1.63 g) in toluene (47 mL) was subjected to heating reflux for 1.5 hours, and after distilling off water with a Dean-Stark apparatus, the solvent was distilled off. Under nitrogen atmosphere, dichloromethane (69 mL) at 0° C. was added to the residue and trimethylsilyl cyanide (2.36 g) was then added, and the resultant mixture was stirred at 25° C. for 96 hours. To the reaction solution, tetra-n-butylammonium fluoride (1 mol/L tetrahydrofuran solution, 1 mL) was added, and after stirring the solution for further 30 minutes, water was added and the resultant mixture was extracted with chloroform. The organic layer was dried over anhydrous sodium sulfate, and the solvent was distilled off under reduced pressure. The residue was purified by silica gel column chromatography (eluting solvent; hexane:ethyl acetate) to give compound A5 (2.74 g).

¹H-NMR (400 MHz, CDCl₃):1.64 (3H, s), 1.65 (3H, s), 3.49-3.55 (1H, m), 3.73 (1H, dd, J=10.9, 4.2 Hz), 3.79 (1H, s), 3.80 (3H, s), 4.05 (1H, dd, J=7.9, 3.6 Hz), 6.96-7.00 (2H, m), 7.11 (2H, d, J=8.0 Hz), 7.21-7.40 (6H, m).

### f) Production of Nα-[(1R)-2-hydroxy-1-phenylethyl]-β,β,1-trimethyl-L-tryptophanamide (Compound A6)

To a suspension of compound A5 (2.74 g), dimethyl sulfoxide (6.16 g) and potassium carbonate (10.9 g) in methanol (50 mL) and water (2.1 mL), a 30% aqueous hydrogen peroxide solution (8.94 mL) was added at 0° C., and the resultant mixture was stirred at 45° C. for 1.5 hours. After the reaction ended, a saturated aqueous sodium thiosulfate solution was added, and the resultant mixture was extracted with ethyl acetate. The organic layer was washed with water and then dried over anhydrous sodium sulfate, and the solvent was distilled off under reduced pressure. The residue was purified by silica gel column chromatography (eluting solvent; chloroform:methanol) to give compound A6 (2.32 g).

¹H-NMR (400 MHz, CDCl₃):1.49 (3H, s), 1.51 (3H, s), 2.06-2.14 (1H, br), 2.37 (1H, dd, J=6.0, 6.0 Hz), 3.44-3.50 (1H, m), 3.50-3.54 (1H, m), 3.56-3.63 (m, 2H), 3.75 (3H, s), 5.52 (1H, brs), 6.14 (1H, brs), 6.71-6.73 (2H, m), 6.81-6.85 (2H, m), 6.97-7.00 (2H, m), 7.10-7.18 (2H, m), 7.24-7.28 (2H, m).

### g) Production of β,β,1-trimethyl-L-tryptophanamide (Compound A7)

To a solution of compound A6 (2.32 g) in methanol (65 mL), palladium hydroxide/carbon (2.8 g) was added, and the resultant mixture was stirred at room temperature for 3 hours under hydrogen atmosphere. The reaction solution was filtered through celite and the solvent was then distilled off, and the residue was purified by silica gel column chromatography (eluting solvent; chloroform:methanol) to give compound A 7 (1.27 g).

¹H-NMR (400 MHz, DMSO-d6):1.24 (2H, brs), 1.28 (3H, s), 1.42 (3H, s), 3.68 (1H, s), 3.71 (3H, s), 6.93-7.00 (2H, m), 7.06 (1H, s), 7.11 (1H, t, J=7.7 Hz), 7.29 (1H, brs), 7.36 (1H, d, J=8.3 Hz), 7.88 (1H, d, J=8.2 Hz).

### h) Production of Na-(tert-butoxycarbonyl)-β,β,1-trimethyl-L-tryptophanamide (Compound A8)

A mixed solution of compound A7 (1.27 g), sodium bicarbonate (522 mg), di-tert-butyl dicarbonate (1.35 g), tetrahydrofuran (13 mL), chloroform (13 mL) and water (6.5 mL) was stirred at 25° C. for 16 hours. After the reaction ended, water was added and the resultant mixture was extracted with chloroform. The organic layer was dried over anhydrous sodium sulfate, and the solvent was distilled off under reduced pressure. The residue was purified by silica gel column chromatography (eluting solvent; chloroform:methanol) to give compound A8 (1.80 g).

¹H-NMR (400 MHz, CDCl₃):1.33 (3H, s), 1.47 (9H, s), 1.50 (3H, s), 3.73 (3H, d, J=1.3 Hz), 4.51 (1H, brs), 4.86 (1H, brs), 5.02 (1H, brd, J=8.2 Hz), 5.59 (1H, brd, J=6.4 Hz), 6.83 (1H, d, J=1.8 Hz), 7.15 (1H, t, J=7.3 Hz), 7.21-7.25 (1H, m), 7.30 (1H, d, J=8.2 Hz), 8.05 (1H, brd, J=7.3 Hz).

LC-MS: 346 (M+H)⁺ (1.211 min, Measurement Condition A)

### i) Production of N,N,Nα-tris(tert-butoxycarbonyl)-β,β,1-trimethyl-L-tryptophanamide (Compound A9)

A mixed solution of compound A8 (1.79 g), di-tert-butyl dicarbonate (2.8 g), N,N-diisopropylethylamine (2.68 g), 4-dimethylaminopyridine (0.19 g) and chloroform (20 mL) was stirred at 25° C. for 2.5 hours. After the reaction ended, water was added and the resultant mixture was extracted with chloroform. The organic layer was washed with saturated brine and then dried over anhydrous sodium sulfate, and the solvent was distilled off under reduced pressure. The residue was purified by silica gel column chromatography (eluting solvent; hexane:ethyl acetate) to give compound A9 (1.99 g).

¹H-NMR (400 MHz, CDCl₃):1.08-1.58 (33H, m), 3.70 (3H, s), 4.67-4.90 (0.2H, m), 5.25-5.45 (0.8H, m), 6.00-6.03 (1H, m), 6.81-6.87 (1H, m), 7.04-7.09 (1H, m), 7.13-7.18 (1H. m), 7.21-7.27 (1H, m), 7.91-7.94 (1H, m).

LC-MS: 546 (M+H)⁺ (1.630 min, Measurement Condition A)

### j) Production of methyl N-(tert-butoxycarbonyl)-β,β,1-trimethyl-L-tryptophanate (Compound A10)

Under nitrogen atmosphere, to a solution of compound A9 (2.29 g) in methanol (21 mL), lithium methoxide (176 mg) was added at 0° C., and the resultant mixture was then stirred at 25° C. for 2 hours. After the reaction ended, a saturated aqueous ammonium chloride solution was added, and the reaction mixture was extracted with ethyl acetate. The organic layer was washed with saturated brine and then dried over anhydrous sodium sulfate, and the solvent was distilled off under reduced pressure. The residue was purified by silica gel column chromatography (eluting solvent; hexane:ethyl acetate) to give compound A10 (927 mg).

¹H-NMR (400 MHz, CDCl₃):1.17-1.59 (15H, m), 3.45 and 3.58 (3H, 2brs), 3.71 (3H, s), 4.56-4.73 (1.2H, m), 5.06 (0.8H, brd, J=7.3 Hz), 6.81-6.82 (1H, m), 7.05-7.10 (1H, m), 7.16-7.21 (1H, m), 7.24-7.29 (1H, m), 7.73-7.80 (1H, m).

LC-MS: 361 (M+H)⁺ (1.379 min, Measurement Condition A).

k) Production of methyl N-(tert-butoxycarbonyl)-N,β,β,1-tetramethyl-L-tryptophanate (Compound A11)

Under nitrogen atmosphere, to a solution of compound A10 (927 mg) in N,N-dimethylformamide (13 mL), sodium hydride 60% dispersion (168 mg) was added at 0° C., and the resultant mixture was then stirred at 25° C. for 15 minutes. After cooling the reaction suspension to 0° C., methyl iodide (1.1 g) was added thereto, and the reaction solution was then stirred at 25° C. for 1 hour. After the reaction ended, water was added and the resultant mixture was extracted with ethyl acetate. The organic layer was dried over anhydrous sodium sulfate, and the solvent was distilled off under reduced pressure. The residue was purified by silica gel column chromatography (eluting solvent; hexane:ethyl acetate) to give compound A11 (915 mg).

¹H-NMR (400 MHz, CDCl₃):1.42 (9H, s), 1.52 and 1.64 (6H, 2s), 2.80 and 2.86 (3H, 2s), 3.46 (3H, s), 3.71 (3H, s), 5.27 and 5.52 (1H, 2s), 6.85 (1H, s), 7.07-7.27 (3H, m), 7.78 and 7.92 (1H, 2d, J=7.88 Hz).

LC-MS: 397 (M+Na)⁺ (1.406 min, Measurement Condition B)

### l) Production of N-(tert-butoxycarbonyl)-N,β,β,1-tetramethyl-L-tryptophan (Compound A12)

To a solution of compound A11 (639 mg) in water (11 mL)-methanol (44 mL), 1 mol/L lithium hydroxide (13.5 mL) was added, and the resultant mixture was stirred at 60° C. for 24 hours. After the reaction ended, a 1 mol/L aqueous oxalic acid solution was added to change the pH of the reaction solution to 4, and water was then added and the resultant mixture was extracted with chloroform. The organic layer was washed with saturated brine and then dried over anhydrous sodium sulfate, and the solvent was distilled off under reduced pressure. The residue was purified by silica gel column chromatography (eluting solvent; chloroform:methanol) to give compound A12 (610 mg).

¹H-NMR (400 MHz, CDCl₃):1.43 (9H, s), 1.53 (3H, s), 1.63 (3H, s), 2.76 and 2.89 (3H, 2s), 3.71 (3H, s), 5.36 and 5.44 (1H, 2s), 6.85 and 6.87 (1H, 2s), 7.02-7.11 (1H, m), 7.18 (1H, t, J=7.3 Hz), 7.24-7.27 (1H, m), 7.81 and 7.96 (1H, 2d, J=7.9 Hz).

LC-MS: 361 (M+H)⁺, 359 (M−H)⁻ (1.300 min, Measurement Condition A).

### m) Production of N-(tert-butoxycarbonyl)-N,β,β,1-tetramethyl-L-tryptophyl-N-[(3S,4E)-6-ethoxy-2,5-dimethyl-6-oxohex-4-en-3-yl]-N,3-dimethyl-L-valinamide (Compound A13)

A mixed solution of compound A12 (500 mg), ethyl (2E,4S)-2,5-dimethyl-4-[methyl(3-methyl-L-valyl)amino]hex-2-enoate (520 mg), 1-ethyl-3-(3-dimethylaminopropyl)carbodiimide hydrochloride (399 mg), 1-hydroxy-1H-benzotriazole monohydrate (425 mg) and N,N-dimethylformamide (5 mL) was stirred at 25° C. for 16 hours. After the reaction ended, water was added and the resultant mixture was extracted with chloroform. The organic layer was washed with saturated brine and then dried over anhydrous sodium sulfate, and the solvent was distilled off under reduced pressure. The residue was purified by silica gel column chromatography (eluting solvent; hexane:ethyl acetate) to give compound A13 (759 mg).

LC-MS: 655 (M+H)⁺ (1.714 min, Measurement Condition A)

### n) Production of N-(tert-butoxycarbonyl)-N,β,β,1-tetramethyl-L-tryptophyl-N-[(3S,4E)-5-carboxy-2-methylhex-4-en-3-yl]-N,3-dimethyl-L-valinamide (Compound A14)

To a solution of compound A13 (127 mg) in water (1.55 mL)-methanol (4.65 mL), 1 mol/L lithium hydroxide (1.65 mL) was added, and the resultant mixture was stirred at 25° C. for 24 hours. After the reaction ended, a 1 mol/L aqueous oxalic acid solution was added to change the pH of the reaction solution to 4, and water was then added and the resultant mixture was extracted with chloroform. The organic layer was washed with saturated brine and then dried over anhydrous sodium sulfate, and the solvent was distilled off under reduced pressure. The residue was purified by silica gel column chromatography (eluting solvent; chloroform:methanol) to give compound A14 (93 mg).

LC-MS: 627 (M+H)⁺ (1.508 min, Measurement Condition A)

o) Production of N-(tert-butoxycarbonyl)-N,β,β,1-tetramethyl-L-tryptophyl-N-{(3S,4E)-6-[(2,5-dioxopyrrolidin-1-yl)oxy]-2,5-dimethyl-6-oxohex-4-en-3-yl}-N, 3-dimethyl-L-valinamide (Reference Example 1)

A mixed solution of compound A14 (185 mg), N-hydroxysuccinimide (97 mg), bromotripyrrolidinophosphonium hexafluorophosphate (391 mg), 4-dimethylaminopyridine (102 mg), N,N-diisopropylethylamine (108 mg) and N,N-dimethylformamide (2.8 mL) was stirred at 25° C. for 4 hours. After the reaction ended, water was added and the resultant mixture was extracted with ethyl acetate. The organic layer was washed with water and then dried over anhydrous sodium sulfate, and the solvent was distilled off under reduced pressure. The residue was purified by silica gel column chromatography (eluting solvent; chloroform:methanol) to give Reference Example 1 (166 mg).

¹H-NMR (400 MHz, CDCl₃):8.27 and 7.96 (1H, 2d, J=7.9 Hz), 7.16-7.04 (4H, m), 6.88 (1H, d, J=9.1 Hz), 6.17 and 6.09 (1H, 2d, J=8.5 Hz), 5.96 and 5.66 (1H, 2s), 5.07 (1H, t, J=9.3 Hz), 4.45 and 3.87 (1H, 2d, J=8.6 Hz), 3.74 and 3.73 (3H, 2s), 2.99 (3H, s), 2.95 (3H, s), 2.83 (4H, brs), 1.97 (3H, s), 1.92-1.86 (1H, m), 1.57-1.42 (14H, m), 0.89 (3H, d, J=6.1 Hz), 0.83-0.80 (3H, m), 0.48 and 0.41 (9H, 2s).

LC-MS: 724 (M+H)⁺ (1.573 min, Measurement Condition A)

### Reference Example 2

### (6S,9S,12S,13E,17R)-9-tert-Butyl-17-(3-{[2-(2,5-dioxo-2,5-dihydro-1H-pyrrol-1-yl)ethyl]amino}-3-oxopropyl)-2,2,5,11,14-pentamethyl-6-[2-(1-methyl-1H-indol-3-yl)propan-2-yl]-4,7,10,15-tetraoxo-12-(propan-2-yl)-3-oxa-5,8,11,16-tetraazaoctadec-13-en-18-oic acid tert-butyl ester

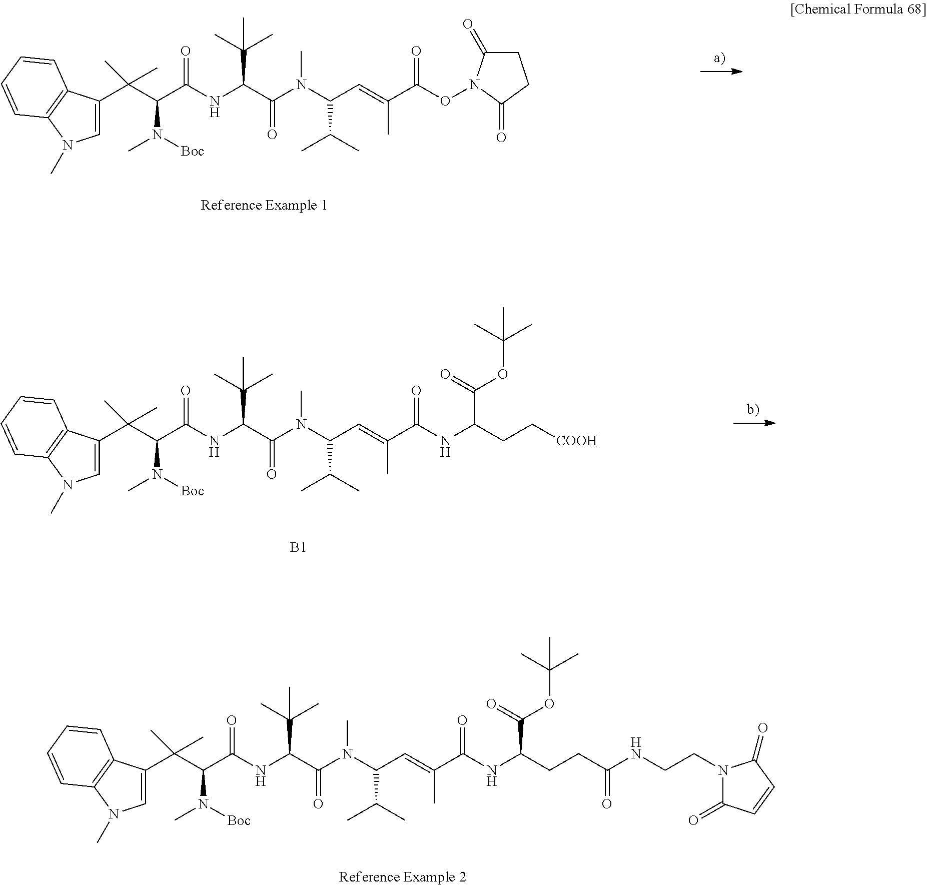

### a) Production of (6S,9S,12S,13E,17R)-17-(tert-butoxycarbonyl)-9-tert-butyl-2,2,5,11,14-pentamethyl-6-[2-(1-methyl-1H-indol-3-yl)propan-2-yl]-4,7,10,15-tetra oxo-12-(propan-2-yl)-3-oxa-5,8,11,16-tetraazaicos-13-en-20-oic acid (Compound B1)

A mixed solution of Reference Example 1 (30 mg), D-glutamic acid α-tert-butyl ester hydrochloride (10.7 mg), N,N-diisopropylethylamine (49.7 mg) and N,N-dimethylformamide (1.0 mL) was stirred at 25° C. for 3 hours. After the reaction ended, water was added, and the resultant mixture was extracted with chloroform. The organic layer was washed with water and then dried over anhydrous sodium sulfate, and the solvent was distilled off under reduced pressure. The residue was purified by silica gel column chromatography (eluting solvent; chloroform:methanol) to give compound B1 (14.2 mg).

LC-MS 834 (M+Na)⁺ (1.574 min, Measurement Condition D)

### b) Production of (6S,9S,12S,13E,17R)-9-tert-butyl-17-(3-{[2-(2,5-dioxo-2,5-dihydro-1H-pyrrol-1-yl)ethyl]amino}-3-oxopropyl)-2,2,5,11,14-pentamethyl-6-[2-(1-methyl-1H-indol-3-yl)propan-2-yl]-4,7,10,15-tetraoxo-12-(propan-2-yl)-3-oxa-5,8,11,16-tetraazaoctadec-13-en-18-oic acid tert-butyl ester (Reference Example 2)

A mixed solution of compound B1 (14 mg), N-(2-aminoethyl)maleimide hydrochloride (3.0 mg), 1-ethyl-3-(3-dimethylaminopropyl)carbodiimide hydrochloride (6.6 mg), 1-hydroxy-1H-benzotriazole monohydrate (5.2 mg), N,N-diisopropylethylamine (4.4 mg) and N,N-dimethylformamide (0.5 mL) was stirred at 25° C. for 2 hours. After the reaction ended, water was added, and the resultant mixture was extracted with ethyl acetate. The organic layer was washed with water and then dried over anhydrous sodium sulfate, and the solvent was distilled off under reduced pressure. The residue was purified by silica gel column chromatography (eluting solvent; chloroform:methanol) to give Reference Example 2 (16.6 mg).

LC-MS: 934 (M+H)⁺ (1.597 min, Measurement Condition D)

### Reference Example 3

### N-(tert-Butoxycarbonyl)-N,β,β,1-tetramethyl-L-tryptophyl-N-[(3S,4E)-6-{[(1R)-1,3-dicarboxypropyl]amino}-2,5-dimethyl-6-oxohex-4-en-3-yl]-N,3-dimethyl-L-valinamide

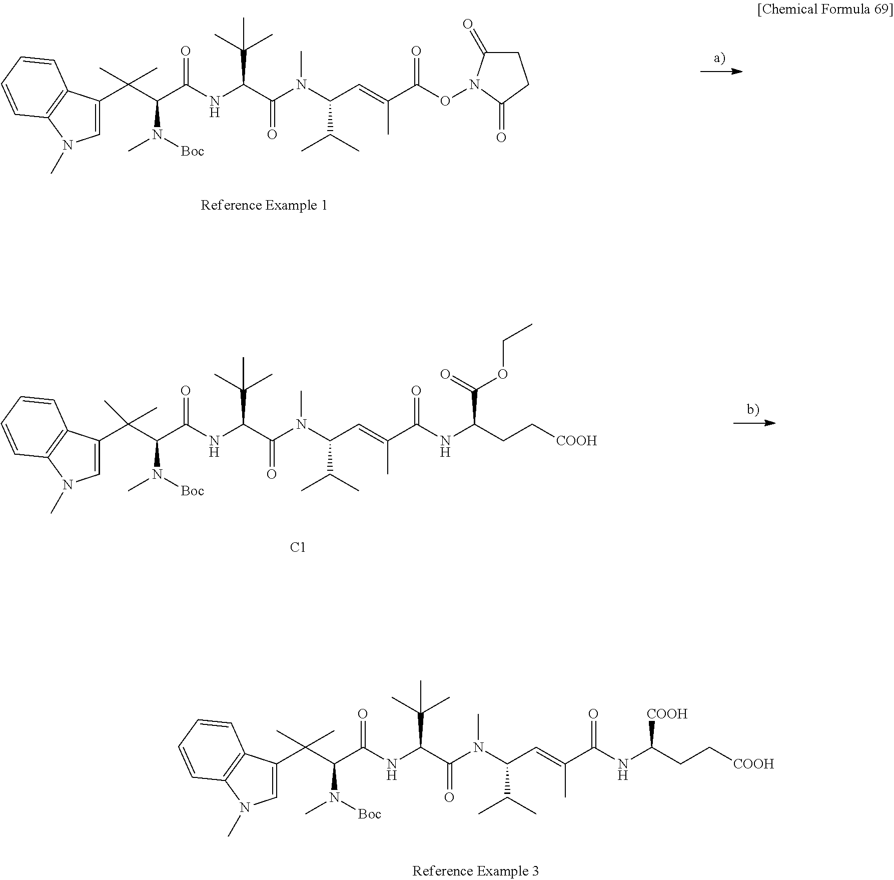 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)ON1C(=O)CCC1=O ; CCOC(=O)[C@@H](CCC(=O)O)NC(=O)/C(C)=C/[C@H](C(C)C)N(C)C(=O)[C@@H](NC(=O)[C@@H](N(C)C(=O)OC(C)(C)C)C(C)(C)c1cn(C)c2ccccc12)C(C)(C)C ; "C1" ; C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@H](CCC(=O)O)C(=O)O]

### a) Production of (6S,9S,12S,13E,17R)-9-tert-butyl-17-(ethoxycarbonyl)-2,2,5,11,14-pentamethyl-6-[2-(1-methyl-1H-indol-3-yl)propan-2-yl]-4,7,10,15-tetraoxo-12-(propan-2-yl)-3-oxa-5,8,11,16-tetraazaicos-13-en-20-oic acid (Compound C1)

A mixed solution of Reference Example 1 (160 mg), D-glutamic acid α-ethyl ester-trifluoroacetate (122 mg), N,N-diisopropylethylamine (100 mg) and N,N-dimethylformamide (2.2 mL) was stirred at 25° C. for 6 hours. After the reaction ended, a 1 mol/L aqueous oxalic acid solution was added to change the pH to 4, and the resultant mixture was extracted with chloroform. The organic layer was washed with water and saturated brine and then dried over anhydrous sodium sulfate, and the solvent was distilled off under reduced pressure. The residue was purified by silica gel column chromatography (eluting solvent; chloroform:methanol) to give compound C1 (155 mg).

¹H-NMR (400 MHz, CDCl₃):8.26 and 7.97 (1H, 2d, J=7.9 Hz), 7.32-7.05 (4H, m), 6.71 (1H, t, J=6.7 Hz), 6.45 (1H, d, J=8.6 Hz), 6.31-6.26 (1H, m), 5.95 and 5.63 (1H, 2s), 4.94-4.82 (1H, m), 4.64-4.59 (11H, m), 4.51 and 4.41 (11H, 2d, J=9.1 Hz), 4.21 (2H, q, J=7.3 Hz), 3.75 and 3.74 (3H, 2s), 3.00 (3H, s), 2.97 and 2.95 (3H, 2s),2.52-2.38 (2H, m), 2.29-2.20 (1H, m), 2.10-2.00 (1H, m), 1.98-1.90 (1H, m), 1.90 (3H, s), 1.57-1.45 (14H, m), 1.28 (3H, t, J=7.3 Hz), 0.88 (3H, d, J=6.1 Hz), 0.82 (3H, d, J=6.7 Hz), 0.53 and 0.46 (9H, 2s).

LC-MS: 784 (M+H)⁺, 782 (M−H)⁻ (1.472 min, Measurement Condition A)

### b) Production of N-(tert-butoxycarbonyl)-N,β,β,1-tetramethyl-L-tryptophyl-N-[(3S,4E)-6-{[(1R)-1,3-dicarboxypropyl]amino}-2,5-dimethyl-6-oxohex-4-en-3-yl]-N,3-dimethyl-L-valinamide (Reference Example 3)

To a solution of compound C1 (103 mg) in water (0.8 mL)-methanol (3.3 mL), 1 mol/L lithium hydroxide (1 mL) was added, and the resultant mixture was stirred at 25° C. for 16 hours. After the reaction ended, a 1 mol/L aqueous oxalic acid solution was added to change the pH of the reaction solution to 4, and then the reaction solution was purified by silica gel column chromatography (eluting solvent; chloroform:methanol) to give Reference Example 3 (100 mg).

LC-MS: 756 (M+H)⁺, 754 (M−H)⁻ (1.388 min, Measurement Condition A)

### Reference Example 4

### N-(tert-Butoxycarbonyl)-N,β,β-trimethyl-L-phenylalanyl-N-{(3S,4E)-6-[(2,5-dioxopyrrolidin-1-yl)oxy]-2,5-dimethyl-6-oxohex-4-en-3-yl}-N,3-dimethyl-L-valinamide

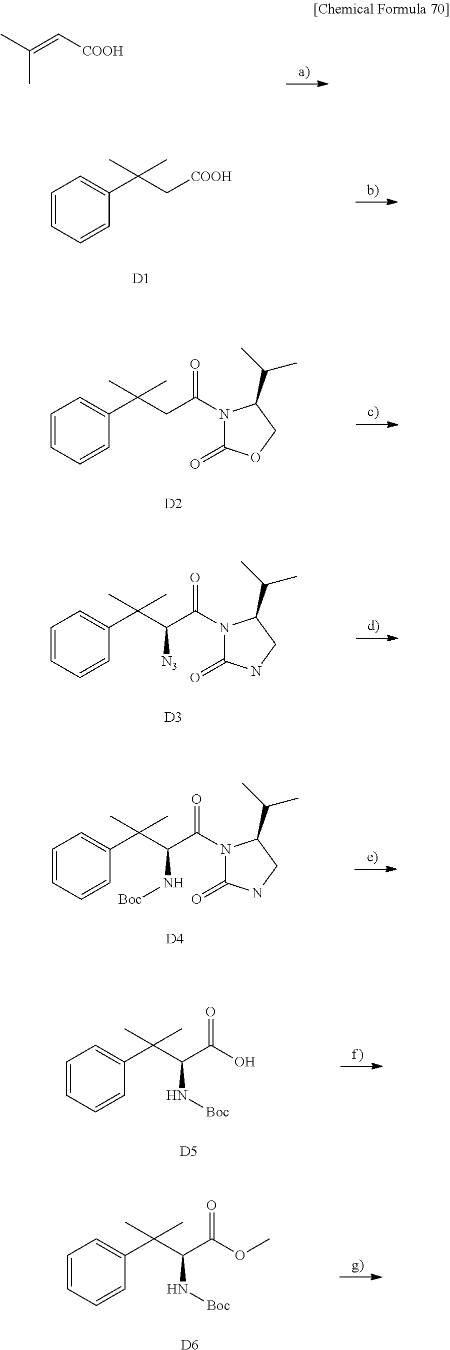

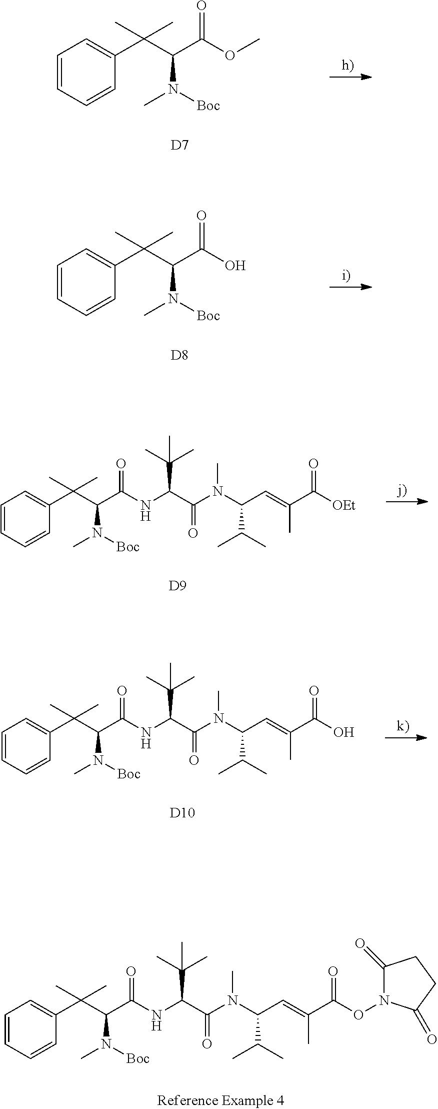

a) Production of 3-methyl-3-phenylbutanoic acid (Compound D1)

To a solution of 3-methyl-2-butenoic acid (15 g) in benzene (100 mL), aluminum chloride (24.1 g) was added at 10° C., and the resultant mixture was stirred for 30 minutes and then stirred at 40° C. for 1 hour. After cooling the reaction solution to 0° C., ice water was added, and the resultant mixture was extracted with tert-butyl methyl ether, concentrated to some extent, and the organic layer was extracted with a saturated aqueous sodium bicarbonate solution. The pH of the aqueous layer was changed to 2 with concentrated hydrochloric acid, and the resultant mixture was extracted with tert-butyl methyl ether. The organic layer was dried over anhydrous magnesium sulfate, and the solvent was distilled off under reduced pressure to give compound D1 (26.3 g).

¹H-NMR (400 MHz, CDCl₃):1.46 (6H, s), 2.65 (2H, s), 7.20 (1H, t, J=7.2 Hz), 7.31 (11H, t, J=7.2 Hz), 7.37 (2H, d, J=7.2 Hz).

### b) Production of (4S)-3-(3-methyl-3-phenylbutanoyl)-4-(propan-2-yl)-1,3-oxazolidin-2-one (Compound D2)

To a solution of compound D1 (17.2 g) in THF (900 mL), triethylamine (23.7 mL) and pivaloyl chloride (15.3 mL) was added at −78° C. After raising the temperature to 0° C., the resultant mixture was stirred for 1 hour. Separately, to a solution of (S)-isopropyloxazolidinone (19.5 g) in THF (760 mL), n-butyllithium (1.64 mol/L hexane solution, 89.8 mL) was added at −78° C., the resultant mixture was stirred for 30 minutes to prepare a lithium salt. The previous reaction solution was cooled to −78° C., the lithium salt was added dropwise, the resultant mixture was stirred for 1 hour, and the temperature was then raised to 0° C. After stirring the mixture for further 30 minutes, water was added, and the resultant mixture was extracted with tert-butyl methyl ether. The organic layer was washed with saturated brine and dried over anhydrous magnesium sulfate. The solvent was distilled off under reduced pressure, and the residue was purified by silica gel column chromatography (eluting solvent; hexane:tert-butyl methyl ether) to give compound D2 (27.0 g).

¹H-NMR (400 MHz, CDCl₃):0.723 (3H, d, J=6.8 Hz), 0.80 (3H, d, J=6.8 Hz), 1.49 (s, 6H), 2.13-2.18 (m, 1H), 3.36 (s, 3H), 3.99-4.09 (m, 2H), 4.20-4.23 (m, 1H), 7.16-7.20 (m, 1H), 7.28-7.32 (m, 2H), 7.38-7.40 (m, 2H).

c) Production of (4S)-3-[(2S)-2-azido-3-methyl-3-phenylbutanoyl]-4-(propan-2-yl)-1,3-oxazolidin-2-one (Compound D3)

A suspension of compound D2 (27.0 g) in THF (560 mL) was cooled to −78° C., potassium hexamethyldisilazide (1.06 mol/L tetrahydrofuran solution, 99.5 mL) was added, and the resultant mixture was stirred for 1.5 hours. A solution of 2,4,6-triisopropylbenzenesulfonyl azide (40 g) in THE (330 mL) at −78° C. was added, and after 10 minutes, acetic acid (24.5 mL) was added, the temperature was raised to 40° C., and the resultant mixture was stirred for 1 hour. Saturated brine was added, and the resultant mixture was extracted with tert-butyl methyl ether. The organic layer was washed with a saturated aqueous sodium bicarbonate solution and saturated brine, and the organic layer was then dried over anhydrous magnesium sulfate. The solvent was distilled off under reduced pressure, and the residue was purified by silica gel column chromatography (eluting solvent; hexane:chloroform) to give compound D3 (16.4 g).

¹H-NMR (400 MHz, CDCl₃):0.80 (3H, d, J=6.8 Hz), 0.84 (3H, d, J=7.2 Hz), 1.54 (3H, s), 1.56 (3H, s), 2.28-2.33 (1H, m), 3.54-3.59 (1H, m), 3.87-3.90 (1H, m), 3.95-3.98 (1H, m), 5.66 (1H, s), 7.23-7.420 (5H, m).

d) Production of tert-butyl {(2S)-3-methyl-1-oxo-1-[(4S)-2-oxo-4-(propan-2-yl)-1,3-oxazolidin-3-yl]-3-phenylbutan-2-yl}carbamate (Compound D4)

To a solution of compound D3 (16.4 g) in ethyl acetate (1200 mL), di-tert-butyl dicarbonate (24.0 g) and 10% Pd-C (11.6 g, 50% wet) were added, and the resultant mixture was stirred for 2 hours under hydrogen atmosphere. The reaction solution was filtered through celite, and was washed with ethyl acetate. The solvent was distilled off under reduced pressure, and the residue was purified by silica gel column chromatography (eluting solvent; hexane:tert-butyl methyl ether) to give compound D4 (16.1 g).

¹H-NMR (400 MHz, CDCl₃):0.77 (3H, d, J=6.8 Hz), 0.82 (3H, d, J=6.8 Hz), 1.42 (3H, s), 1.43 (9H, s), 1.48 (3H, s), 2.20-2.29 (1H, m), 3.45 (1H, t, J=8.8 Hz), 3.80-3.83 (1H, m), 3.89-3.92 (1H, dd, J=2.0 Hz, J=8.4 Hz), 5.16 (1H, brs), 6.13 (1H, d, J=9.6 Hz), 7.21-7.26 (1H, m), 7.29-7.33 (2H, m). 7.42 (2H, d, J=7.2 Hz).

### e) Production of N-(tert-butoxycarbonyl)-β,β-dimethyl-L-phenylalanine (Compound D5)

To a solution of compound D4 (16.1 g) in THE (468 mL) and water (117 mL), a 30% aqueous hydrogen peroxide solution (32.5 mL) and an aqueous lithium hydroxide solution (1 mol/L, 119 mL) were added at 0° C., the temperature was raised to 25° C., and the resultant mixture was stirred for 3 hours. An aqueous sodium bisulfate solution (1.5 mol/L, 470 mL) was added at 0° C., the temperature was raised to 25° C., and the resultant mixture was stirred for 1 hour. The pH was changed to 3 with an aqueous citric acid solution (1 mol/L), and the resultant mixture was extracted with tert-butyl methyl ether. The organic layer was washed with saturated brine, and the organic layer was dried over anhydrous magnesium sulfate. The solvent was distilled off under reduced pressure to give compound D5 (14.2 g).

¹H-NMR (400 MHz, CDCl₃):1.38 (9H, s), 1.44 (3H, s), 1.46 (3H, s), 4.56 (1H, brd, J=11.6 Hz), 4.94 (1H, brd, J=14.4 Hz), 7.21-7.38 (5H, m).

### f) Production of N-(tert-butoxycarbonyl)-β,β-dimethyl-L-phenylalanine methyl ester (Compound D6)

To a solution of compound D5 (14.2 g) in N,N-dimethylformamide (84 ml), sodium carbonate (8.44 g) and methyl iodide (9.91 mL) were added, and the resultant mixture was stirred at 25° C. for 15 hours. After cooling the mixture to 0° C., chilled water was added and the resultant mixture was extracted with tert-butyl methyl ether, and the organic layer thus obtained was washed with saturated brine and then dried over anhydrous magnesium sulfate. The solvent was distilled off under reduced pressure, and the residue was purified by silica gel column chromatography (eluting solvent; hexane:tert-butyl methyl ether) to give compound D6 (11.1 g).

¹H-NMR (400 MHz, CDCl₃):1.36 (9H, s), 1.37 (3H, s), 1.41 (3H, s), 3.48 (3H, brs), 4.49 (1H, brd, J=9.8 Hz), 4.98 (1H, brd, J=9.1 Hz), 7.18-7.22 (1H, m), 7.27-7.33 (4H, m).

### g) Production of N-(tert-butoxycarbonyl)-N,β,β-trimethyl-L-phenylalanine methyl ester (Compound D7)

By the same approach as Reference Example 1-k), from compound D6 (307 mg), compound D7 (245 mg) was obtained.

LC-MS: 344 (M+Na)⁺ (1.589 min, Measurement Condition C)

h) Production of N-(tert-butoxycarbonyl)-N,β,β-trimethyl-L-phenylalanine (Compound B8)

By the same approach as Reference Example 1-1), from compound D7 (235 mg), compound D8 (195 mg) was obtained.

LC-MS: 330 (M+Na)⁺ (1.420 min, Measurement Condition C)

### i) Production of N-(tert-butoxycarbonyl)-N,β,β-trimethyl-L-phenylalanyl-N-[(3S,4E)-6-ethoxy-2,5-dimethyl-6-oxohex-4-en-3-yl]-N,3-dimethyl-L-valinamide (Compound D9)

By the same approach as Reference Example 1-m), from compound D8 (195 mg), compound D9 (307 mg) was obtained.

LC-MS: 624 (M+Na)⁺ (1.797 min, Measurement Condition C)

### j) Production of N-(tert-butoxycarbonyl)-N,β,β-trimethyl-L-phenylalanyl-N-[(3S,4E)-5-carboxy-2-methylhex-4-en-3-yl]-N,3-dimethyl-L-valinamide (Compound D10)

By the same approach as Reference Example 1-n), from compound D9 (307 mg), compound D10 (286 mg) was obtained.

LC-MS: 596 (M+Na)⁺, 572 (M−H)⁻ (1.596 min, Measurement Condition C)

### k) Production of N-(tert-butoxycarbonyl)-N,β,β-trimethyl-L-phenylalanyl-N-{(3S,4E)-6-[(2,5-dioxopyrrolidin-1-yl)oxy]-2,5-dimethyl-6-oxohex-4-en-3-yl}-N,3-dimethyl-L-valinamide (Reference Example 4)

By the same approach as Reference Example 1-o), from compound D10 (286 mg), Reference Example 4 (227 mg) was obtained. LC-MS: 693 (M+Na)⁺ (1.658 min, Measurement Condition C)

### Reference Example 5

The compound shown in the following Table 1 was obtained in accordance with the method described in the literature (International Publication No. WO 2003/082268).

TABLE 1

| | | | LC-MS |
|---|---|---|---|
| Reference | | | Measurement |
| Example | Structural Formula | LC-MS/Rt (min) | Condition |
| 5 | 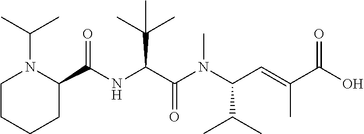 [SMILES: C/C(=C\[C@H](C(C)C)N(C)C(=O)[C@@H](NC(=O)[C@H]1CCCCN1C(C)C)C(C)(C)C)C(=O)O] | 438(M + H)⁺/0.886 | F |

### Reference Examples 6 and 7

The compounds shown in the following Table 2 were obtained through the same reaction and treatment as step m) of Reference Example 1 or step o) of Reference Example 1, using Reference Example 5.

TABLE 2

| | | | LC-MS |
|---|---|---|---|
| Reference | | | Measurement |
| Example | Structural Formula | LC-MS/Rt (min) | Condition |
| 6 | 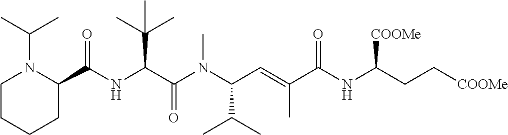 [SMILES: COC(=O)CC[C@@H](NC(=O)/C(C)=C/[C@H](C(C)C)N(C)C(=O)[C@@H](NC(=O)[C@H]1CCCCN1C(C)C)C(C)(C)C)C(=O)OC] | 595(M + H)⁺/0.698 | F |
| 7 | 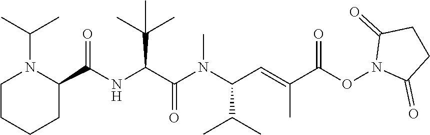 [SMILES: C/C(=C\[C@H](C(C)C)N(C)C(=O)[C@@H](NC(=O)[C@H]1CCCCN1C(C)C)C(C)(C)C)C(=O)ON1C(=O)CCC1=O] | 535(M + H)⁺/0.707 | F |

### Reference Examples 8 and 9

The compounds shown in the following Table 3 were obtained through the same reaction and treatment as m) step of Reference Example 1, using corresponding raw material compounds.

TABLE 3

| | | | LC-MS |
|---|---|---|---|
| Reference | | | Measurement |
| Example | Structural Formula | LC-MS/Rt (min) | Condition |
| 8 | 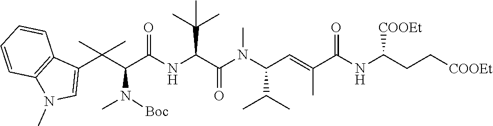 [SMILES: CCOC(=O)CC[C@H](NC(=O)/C(C)=C/[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)OCC] | 812(M + H)⁺/1.518 | G |
| 9 | 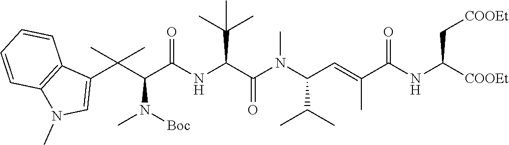 [SMILES: CCOC(=O)C[C@H](NC(=O)/C(C)=C/[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)OCC] | 798(M + H)⁺/1.33 | F |

### Reference Examples 10 to 35

The compounds shown in the following Table 4 were obtained through the same reaction and treatment as step a) of Reference Example 2, using corresponding raw material compounds.

TABLE 4

| | | | LC-MS |
|---|---|---|---|
| Reference | | | Measurement |
| Example | Structural Formula | LC-MS/Rt (min) | Condition |
| 10 | 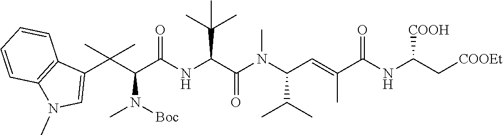 [SMILES: CCOC(=O)C[C@H](NC(=O)/C(C)=C/[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)O] | 770(M + H)⁺/1.22 | E |
| 11 | 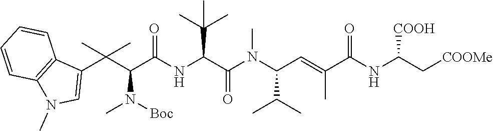 [SMILES: COC(=O)C[C@H](NC(=O)/C(C)=C/[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)O] | 756(M + H)⁺/1.518 | G |
| 12 | 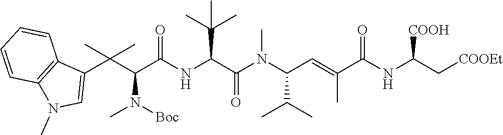 [SMILES: CCOC(=O)C[C@@H](NC(=O)/C(C)=C/[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)O] | 770(M + H)⁺/1.22 | E |
| 13 | 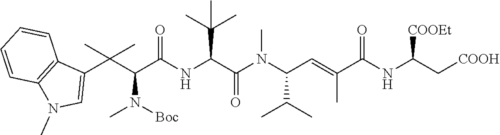 [SMILES: CCOC(=O)[C@@H](CC(=O)O)NC(=O)/C(C)=C/[C@H](C(C)C)N(C)C(=O)[C@@H](NC(=O)[C@@H](N(C)C(=O)OC(C)(C)C)C(C)(C)c1cn(C)c2ccccc12)C(C)(C)C] | 770(M + H)⁺/1.21 | E |
| 14 | 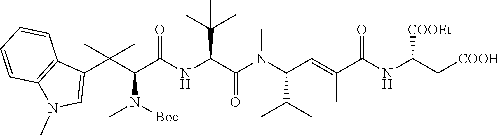 [SMILES: CCOC(=O)[C@H](CC(=O)O)NC(=O)/C(C)=C/[C@H](C(C)C)N(C)C(=O)[C@@H](NC(=O)[C@@H](N(C)C(=O)OC(C)(C)C)C(C)(C)c1cn(C)c2ccccc12)C(C)(C)C] | 770(M + H)⁺/1.21 | E |
| 15 | 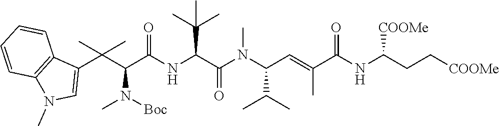 [SMILES: COC(=O)CC[C@H](NC(=O)/C(C)=C/[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)OC] | 806(M + Na)⁺/1.652 | G |
| 16 | 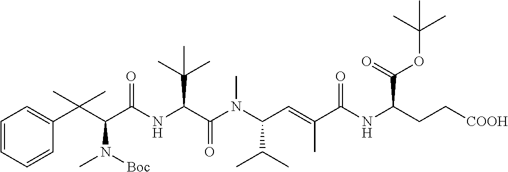 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CCC(=O)O)C(=O)OC(C)(C)C] | 759(M + H)⁺/1.427 | C |
| 17 | 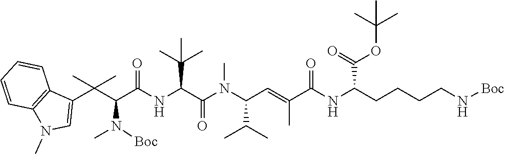 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@@H](CCCCNC(=O)OC(C)(C)C)C(=O)OC(C)(C)C] | 911(M + H)⁺/1.709 | G |
| 18 | 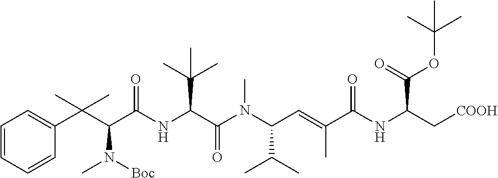 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CC(=O)O)C(=O)OC(C)(C)C] | 745(M + H)⁺/1.594 | G |
| 19 | 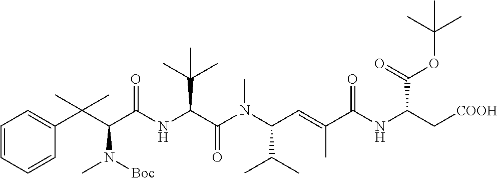 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@@H](CC(=O)O)C(=O)OC(C)(C)C] | 745(M + H)⁺/1.526 | G |
| 20 | 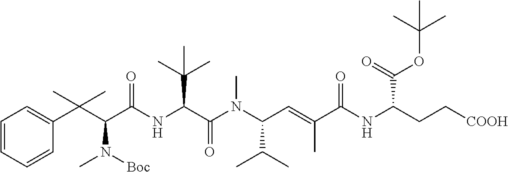 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@@H](CCC(=O)O)C(=O)OC(C)(C)C] | 759(M + H)⁺/1.693 | G |
| 21 | 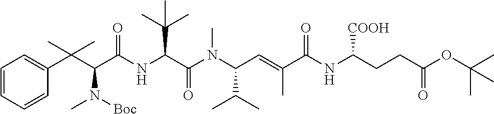 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@@H](CCC(=O)OC(C)(C)C)C(=O)O] | 759(M + H)⁺/1.732 | G |
| 22 | 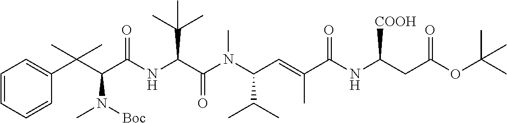 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CC(=O)OC(C)(C)C)C(=O)O] | 745(M + H)⁺/1.439 | G |
| 23 | 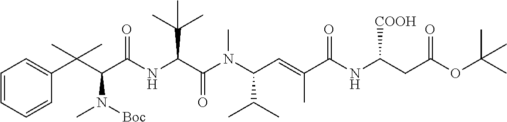 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@@H](CC(=O)OC(C)(C)C)C(=O)O] | 745(M + H)⁺/1.400 | G |
| 24 | 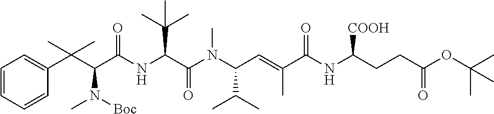 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CCC(=O)OC(C)(C)C)C(=O)O] | 759(M + H)⁺/1.413 | G |
| 25 | 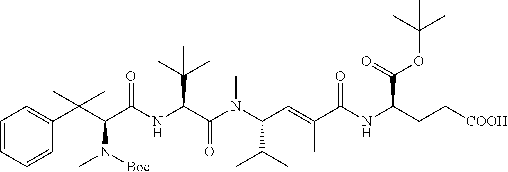 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CCC(=O)O)C(=O)OC(C)(C)C] | 759(M + H)⁺/1.475 | G |
| 26 | 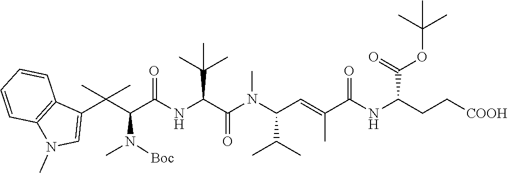 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@@H](CCC(=O)O)C(=O)OC(C)(C)C] | 812(M + H)⁺/1.560 | G |
| 27 | 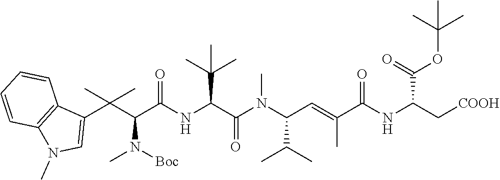 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@@H](CC(=O)O)C(=O)OC(C)(C)C] | 798(M + H)⁺/1.415 | G |
| 28 | 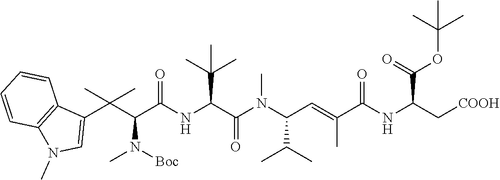 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@H](CC(=O)O)C(=O)OC(C)(C)C] | 798(M + H)⁺/1.474 | G |
| 29 | 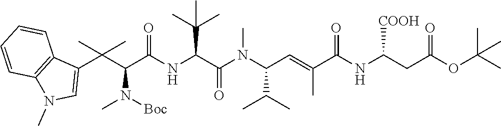 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@@H](CC(=O)OC(C)(C)C)C(=O)O] | 798(M + H)⁺/1.470 | G |
| 30 | 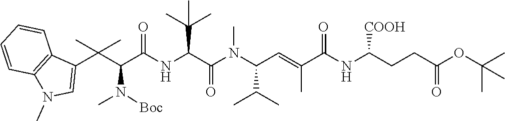 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@@H](CCC(=O)OC(C)(C)C)C(=O)O] | 812(M + H)⁺/1.460 | G |
| 31 | 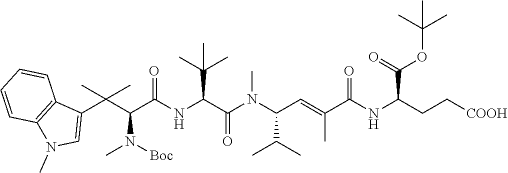 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@H](CCC(=O)O)C(=O)OC(C)(C)C] | 812(M + H)⁺/1.450 | G |
| 32 | 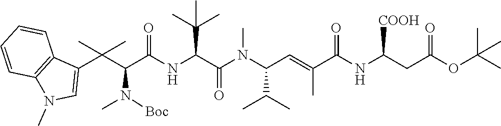 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@H](CC(=O)OC(C)(C)C)C(=O)O] | 798(M + H)⁺/1.420 | G |
| 33 | 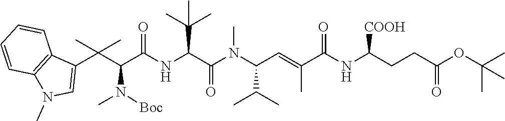 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@H](CCC(=O)OC(C)(C)C)C(=O)O] | 812(M + H)⁺/1.457 | G |
| 34 | 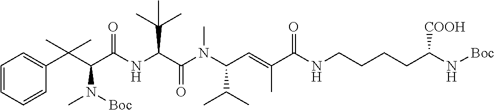 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)NCCCC[C@@H](NC(=O)OC(C)(C)C)C(=O)O] | 802(M + H)⁺/1.390 | G |
| 35 | 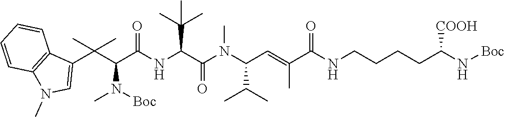 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)NCCCC[C@@H](NC(=O)OC(C)(C)C)C(=O)O] | 855(M + H)⁺/1.588 | G |

### Reference Examples 36 to 53

The compounds shown in the following Table 5 were obtained through the same reaction and treatment as step b) of Reference Example 2, using corresponding raw material compounds.

TABLE 5

| | | | LC-MS |
|---|---|---|---|
| Reference | | | Measurement |
| Example | Structural Formula | LC-MS/Rt (min) | Condition |
| 36 | 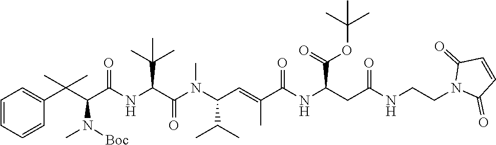 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CC(=O)NCCN1C(=O)C=CC1=O)C(=O)OC(C)(C)C] | 889(M + Na)⁺/1.498 | G |
| 37 | 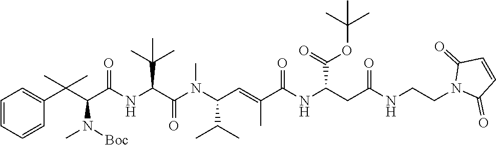 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@@H](CC(=O)NCCN1C(=O)C=CC1=O)C(=O)OC(C)(C)C] | 867(M + H)⁺/1.433 | G |
| 38 | 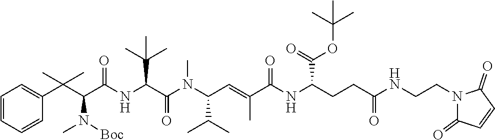 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@@H](CCC(=O)NCCN1C(=O)C=CC1=O)C(=O)OC(C)(C)C] | 881(M + H)⁺/1.701 | G |
| 39 | 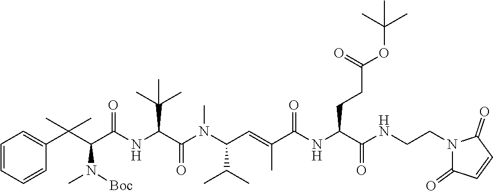 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@@H](CCC(=O)OC(C)(C)C)C(=O)NCCN1C(=O)C=CC1=O] | 881(M + H)⁺/1.170 | G |
| 40 | 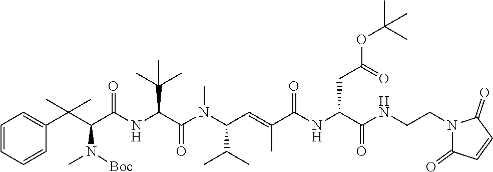 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CC(=O)OC(C)(C)C)C(=O)NCCN1C(=O)C=CC1=O] | 867(M + H)⁺/1.447 | G |
| 41 | 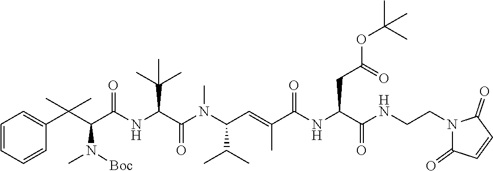 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@@H](CC(=O)OC(C)(C)C)C(=O)NCCN1C(=O)C=CC1=O] | 867(M + H)⁺/1.413 | G |
| 42 | 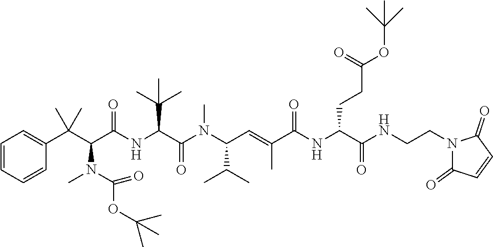 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CCC(=O)OC(C)(C)C)C(=O)NCCN1C(=O)C=CC1=O] | 881(M + H)⁺/1.422 | G |
| 43 | 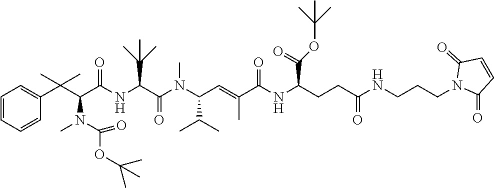 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CCC(=O)NCCCN1C(=O)C=CC1=O)C(=O)OC(C)(C)C] | 895(M + H)⁺/1.419 | G |
| 44 | 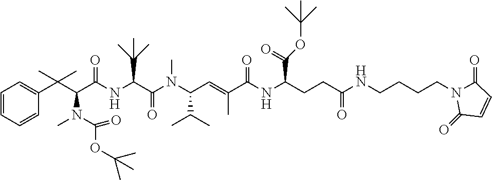 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CCC(=O)NCCCCN1C(=O)C=CC1=O)C(=O)OC(C)(C)C] | 909(M + H)⁺/1.475 | G |
| 45 | 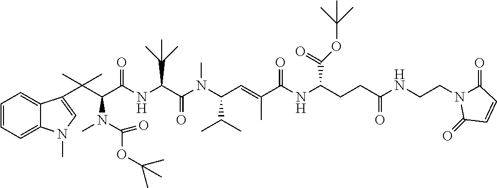 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@@H](CCC(=O)NCCN1C(=O)C=CC1=O)C(=O)OC(C)(C)C] | 934(M + H)⁺/1.441 | G |
| 46 | 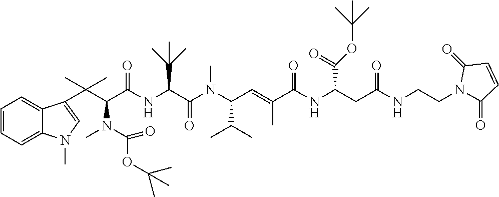 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@@H](CC(=O)NCCN1C(=O)C=CC1=O)C(=O)OC(C)(C)C] | 920(M + H)⁺/1.498 | G |
| 47 | 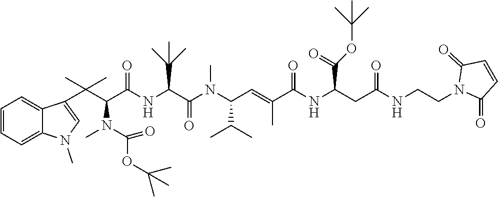 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@H](CC(=O)NCCN1C(=O)C=CC1=O)C(=O)OC(C)(C)C] | 920(M + H)⁺/1.443 | G |
| 48 | 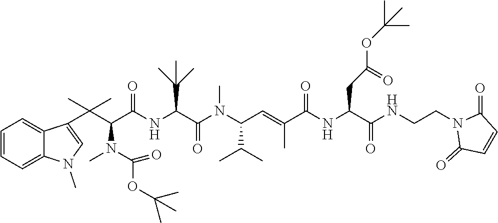 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@@H](CC(=O)OC(C)(C)C)C(=O)NCCN1C(=O)C=CC1=O] | 942(M + Na)⁺/1.440 | G |
| 49 | 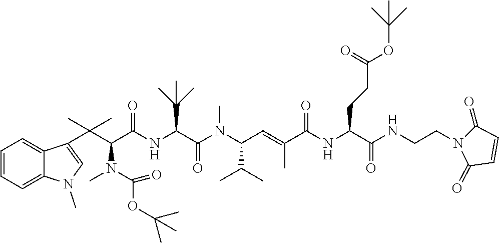 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@@H](CCC(=O)OC(C)(C)C)C(=O)NCCN1C(=O)C=CC1=O] | 934(M + H)⁺/1.475 | G |
| 50 | 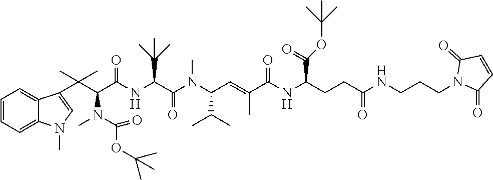 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@H](CCC(=O)NCCCN1C(=O)C=CC1=O)C(=O)OC(C)(C)C] | 970(M + Na)⁺/1.503 | G |
| 51 | 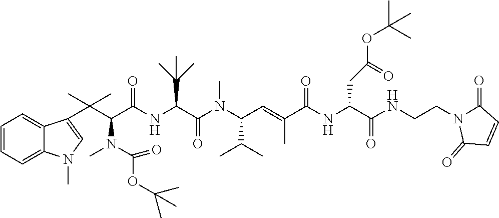 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@H](CC(=O)OC(C)(C)C)C(=O)NCCN1C(=O)C=CC1=O] | 920(M + H)⁺/1.459 | G |
| 52 | 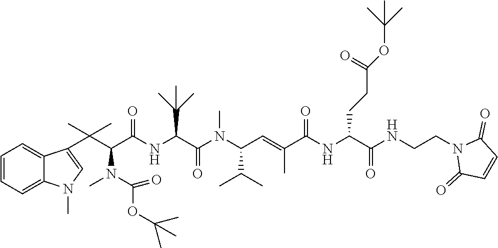 [SMILES: C/C(=C\[C@H](C(C)C)N(C)C(=O)[C@@H](NC(=O)[C@@H](N(C)C(=O)OC(C)(C)C)C(C)(C)c1cn(C)c2ccccc12)C(C)(C)C)C(=O)N[C@H](CCC(=O)OC(C)(C)C)C(=O)NCCN1C(=O)C=CC1=O] | 934(M + H)⁺/1.497 | G |
| 53 | 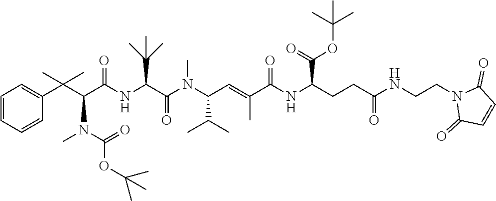 [SMILES: C/C(=C\[C@H](C(C)C)N(C)C(=O)[C@@H](NC(=O)[C@@H](N(C)C(=O)OC(C)(C)C)C(C)(C)c1ccccc1)C(C)(C)C)C(=O)N[C@H](CCC(=O)NCCN1C(=O)C=CC1=O)C(=O)OC(C)(C)C] | 881(M + H)⁺/1.373 | G |

### Reference Example 54

### Diethyl ((S,E)-4-((S)-2-amino-N,3,3-trimethylbutanamido)-2,5-dimethylhex-2-enoyl)-D-glutamate

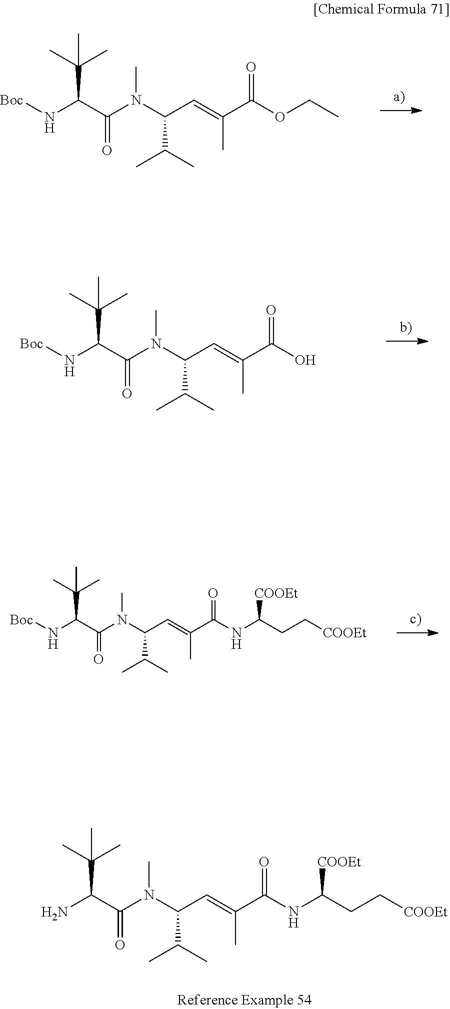 [SMILES: CCOC(=O)/C(C)=C/[C@H](C(C)C)N(C)C(=O)[C@@H](NC(=O)OC(C)(C)C)C(C)(C)C ; C/C(=C\[C@H](C(C)C)N(C)C(=O)[C@@H](NC(=O)OC(C)(C)C)C(C)(C)C)C(=O)O ; CCOC(=O)CC[C@@H](NC(=O)/C(C)=C/[C@H](C(C)C)N(C)C(=O)[C@@H](NC(=O)OC(C)(C)C)C(C)(C)C)C(=O)OCC ; CCOC(=O)CC[C@@H](NC(=O)/C(C)=C/[C@H](C(C)C)N(C)C(=O)[C@@H](N)C(C)(C)C)C(=O)OCC]

### a) Production of (S,E)-4-((S)-2-((tert-butoxycarbonyl)amino)-N,3,3-trimethylbutanamido)-2,5-dimethylhex-2-enoic acid

By the same approach as Reference Example 1-n), from ethyl (S,E)-4-((S)-2-((tert-butoxycarbonyl)amino)-N,3,3-trimethylbutanamido)-2,5-dimethylhex-2-enoate (1.64 g), (S,E)-4-((S)-2-((tert-butoxycarbonyl)amino)-N,3,3-trimethylbutanamido)-2,5-dimethylhex-2-enoic acid (1.55 g) was obtained.

LC-MS: 385 (M+H)⁺/0.986 min, Measurement Condition E

### b) Production of diethyl ((S,E)-4-((S)-2-((tert-butoxycarbonyl) amino)-N,3,3-trimethylbutanamido)-2,5-dimethylhex-2-enoyl)-D-glutamate

By the same approach as Reference Example 1-m), from (S,E)-4-((S)-2-((tert-butoxycarbonyl)amino)-N,3,3-trimethylbutanamido)-2,5-dimethylhex-2-enoic acid (376 mg), diethyl ((S,E)-4-((S)-2-((tert-butoxycarbonyl)amino)-N,3,3-trimethylbutanamido)-2,5-dimethylhex-2-enoyl)-D-glutamate (500 mg) was obtained.

### c) Production of diethyl ((S,E)-4-((S)-2-amino-N,3,3-trimethylbutanamido)-2,5-dimethylhex-2-enoyl)-D-glutamate (Reference Example 54)

By the same approach as Reference Example 103-a), from diethyl ((S,E)-4-((S)-2-((tert-butoxycarbonyl)amino)-N,3,3-trimethylbutanamido)-2,5-dimethylhex-2-enoyl)-D-glutamate (528 mg), Reference Example 54 (515 mg) was obtained.

LC-MS: 470 (M+H)+/1.223 min, Measurement Condition C

### Reference Example 55

### tert-Butyl N5-(2-(2,5-dioxo-2,5-dihydro-1H-pyrrol-1-yl)ethyl)-D-glutaminate

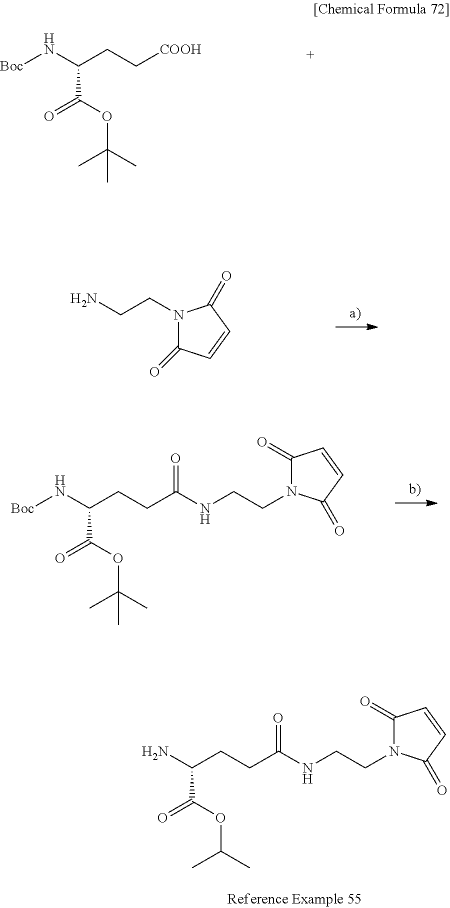 [SMILES: CC(C)(C)OC(=O)N[C@H](CCC(=O)O)C(=O)OC(C)(C)C ; NCCN1C(=O)C=CC1=O ; CC(C)(C)OC(=O)N[C@H](CCC(=O)NCCN1C(=O)C=CC1=O)C(=O)OC(C)(C)C ; CC(C)(C)OC(=O)[C@H](N)CCC(=O)NCCN1C(=O)C=CC1=O]

### a) Production of tert-butyl N2-(tert-butoxycarbonyl)-N5-(2-(2,5-dioxy-2,5-dihydro-1H-pyrrol-1-yl) ethyl)-D-glutaminate

A mixed solution of BOC-D-glutamic acid α-tert-butyl ester (2.061 g), 1-(2-amino-ethyl)-pyrrole-2,5-dione hydrochloride (1.20 g), 2-(1H-benzo[d][1,2,3]triazoll-1-yl)-1,1,3,3-tetramethylisouronium hexafluorophosphate (V) (3.87 g), N,N-diisopropylethylamine (3.47 mL) and N,N-dimethylformamide (10 mL) was stirred at room temperature for 1 hour. After the reaction ended, ethyl acetate was added, the organic layer was washed with a saturated aqueous sodium bicarbonate solution and saturated brine and then dried over anhydrous sodium sulfate, and the solvent was distilled off under reduced pressure. The residue was purified by silica gel chromatography (eluting solvent; hexane:ethyl acetate) to give tert-butyl N2-(tert-butoxycarbonyl)-N5-(2-(2,5-dioxy-2,5-dihydro-1H-pyrrol-1-yl) ethyl)-D-glutaminate (2.8 g).

LC-MS: 426 (M+H)⁺ (1.030 min, Measurement Condition F)

### b) Production of tert-butyl N5-(2-(2,5-dioxo-2,5-dihydro-1H-pyrrol-1-yl)ethyl)-D-glutaminate (Reference Example 5)

A mixed solution of tert-butyl N2-(tert-butoxycarbonyl)-N5-(2-(2,5-dioxy-2,5-dihydro-1H-pyrrol-1-yl) ethyl)-D-glutaminate (51.8 mg) and TFA (1 mL) was stirred at room temperature for 1 hour 20 minutes. The reaction solution was ice-cooled, and then concentrated under reduced pressure to give Reference Example 55. The compound was used for the subsequent reaction without purification.

LC-MS: 326 (M+H)⁺ (0.496 min, Measurement Condition F)

### Reference Example 56

### N5-(2-(2,5-Dioxo-2,5-dihydro-1H-pyrrol-1-yl)ethyl)-D-glutamine

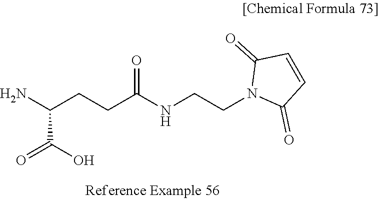 [SMILES: N[C@H](CCC(=O)NCCN1C(=O)C=CC1=O)C(=O)O]

A mixed solution of tert-butyl N2-(tert-butoxycarbonyl)-N5-(2-(2,5-dioxy-2,5-dihydro-1H-pyrrol-1-yl) ethyl)-D-glutaminate (64.8 mg) and TFA (1 mL) was stirred at room temperature for 17 hours. After the reaction ended, the resultant mixture was concentrated under reduced pressure to give Reference Example 56. The compound was used for the subsequent reaction without purification.

LC-MS: 270 (M+H)⁺ (0.254 min, Measurement Condition F)

### Reference Example 101

### tert-Butyl ((S)-3-methyl-1-(((S)-1-((4-((((4-nitrophenoxy)carbonyl)oxy) methyl)phenyl)amino)-1-oxo-5-ureidopentan-2-yl)amino)-1-oxobutan-2-yl)carbamate

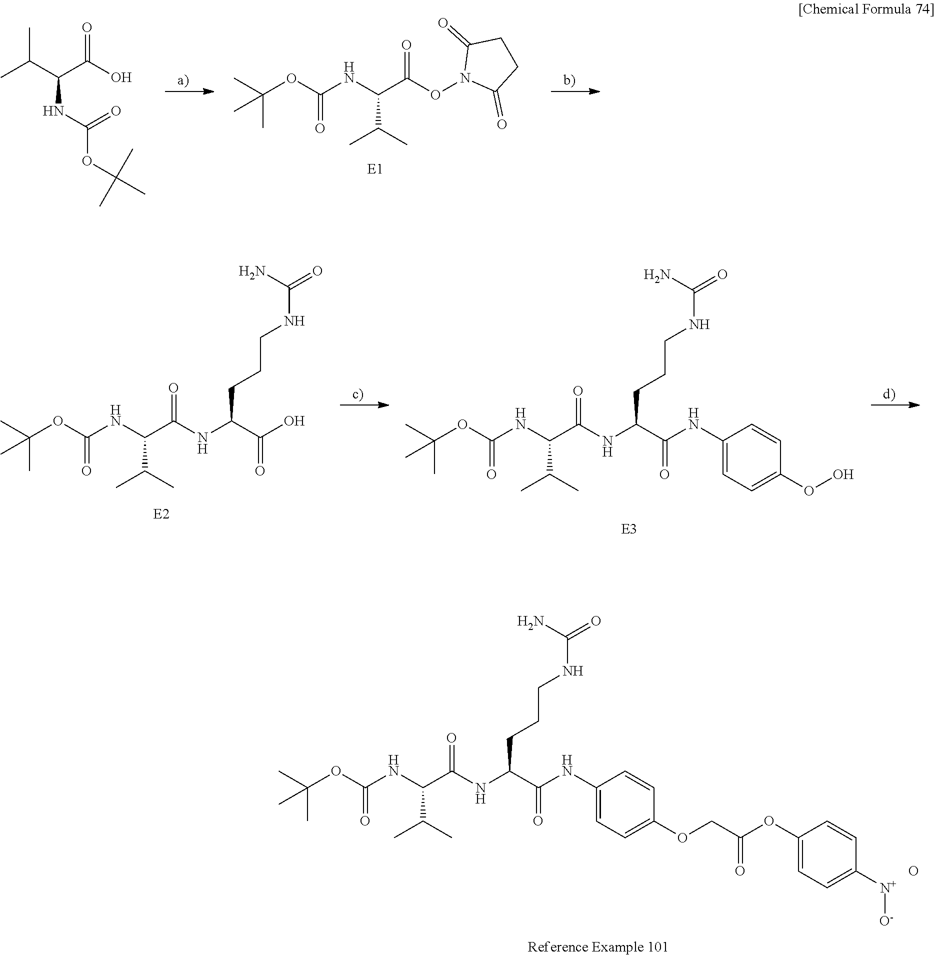

### a) Production of 2,5-dioxopyrrolidin-1-yl (tert-butoxycarbonyl)-L-valinate (Compound E1)

By the same approach as Reference Example 1-o), from (tert-butoxycarbonyl)-L-valine (3.72 g), compound E1 (4.9 g) was obtained.

1H-NMR (400 MHz, CDCl₃): 1.01 (3H, d, J=7.2 Hz), 1.05 (3H, d, J=6.8 Hz), 1.44 (9H, s), 2.28 (1H, m), 2.82 (4H, s), 4.58 (1H, m), 4.97 (1H, m)

### b) Production of (S)-2-((S)-2-((tert-butoxycarbonyl) amino)-3-methylbutanamido)-5-ureidopentanoic acid (Compound E2)

By the same approach as Reference Example 2-a), from 2,5-dioxopyrrolidin-1-yl (tert-butoxycarbonyl)-L-valinate (4.9 g), compound E2 (5.31 g) was obtained.

LC-MS: 375 (M+H)⁺/0.972 min, Measurement Condition C

### c) Production of tert-butyl ((S)-1-(((S)-1-((4-(hydroxymethyl) phenyl)amino)-1-oxo-5-ureidopentan-2-yl)amino)-3-methyl-1-oxobutan-2-yl)carbamate (Compound E3)

A mixed solution of compound E2 (701 mg), 4-aminobenzyl alcohol (461 mg), ethyl 2-ethoxyquinoline-1(2H)-carboxylate (926 mg), methanol (10 mL) and dichloromethane (20 mL) was stirred at room temperature for 24 hours under shading. After distilling off the solvent under reduced pressure, through purification by silica gel column chromatography (eluting solvent; chloroform:methanol), compound E3 (243 mg) was obtained.

LC-MS: 480 (M+H)⁺/1.583 min, Measurement Condition G

### d) Production of tert-butyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (Reference Example 101)

A mixed solution of compound E3 (2.0 g), bis(4-nitrophenyl)carbonate (3.81 g), N,N-diisopropylethylenediamine (2.179 mL) and N,N-dimethylformamide was stirred at room temperature for 2 hours. After distilling off the solvent under reduced pressure, the residue was purified by silica gel chromatography (eluting solvent; chloroform:methanol) to give Reference Example 101 (2.1 g).

LC-MS: 645 (M+H)⁺/1.225 min, Measurement Condition G

### Reference Example 102

### tert-Butyl((S)-3-methyl-1-(((S)-1-((4-((((4-nitrophenoxy)carbonyl)oxy) methyl)phenyl)amino)-1-oxopropan-2-yl)amino)-1-oxobutan-2-yl)carbamate

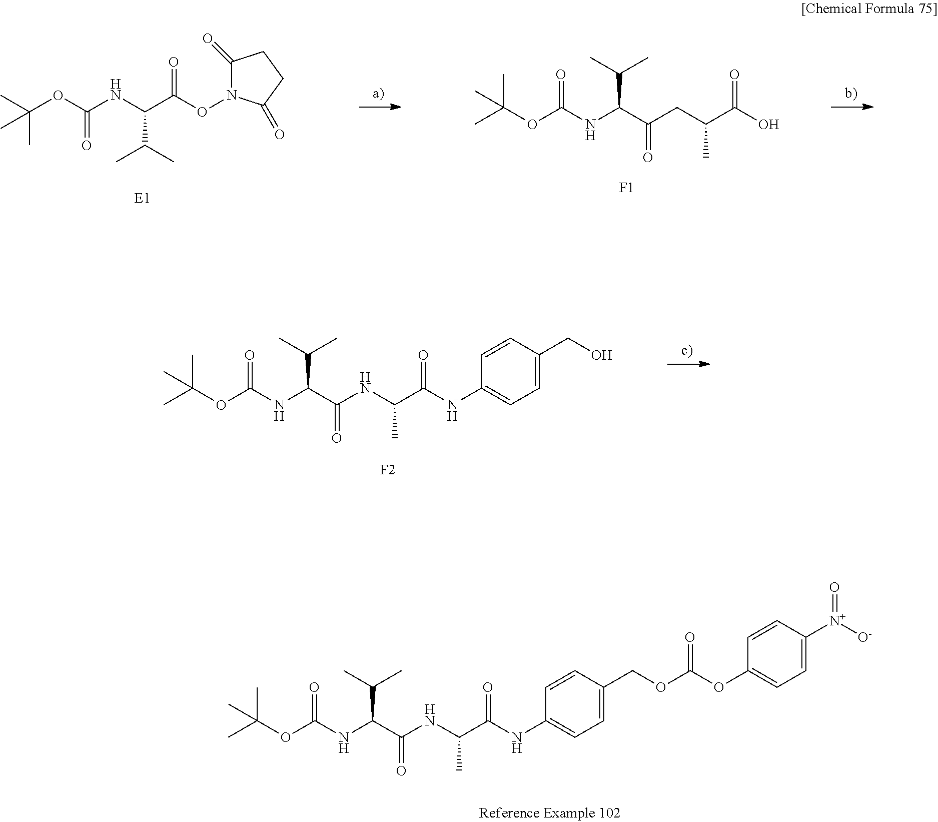

### Reference Example 102

### a) Production of (tert-butoxycarbonyl)-L-valyl-L-alanine (Compound F1)

By the same approach as Reference Example 2-a), from 2,5-dioxopyrrolidin-1-yl (tert-butoxycarbonyl)-L-valinate (1.0 g), compound F1 (676 mg) was obtained.

LC-MS: 278 (M−H)⁻/0.925 min, Measurement Condition G

### b) Production of tert-butyl((S)-1-(((S)-1-((4-(hydroxymethyl)phenyl)amino)-1-oxopropan-2-yl)amino)-3-methyl-1-oxobutan-2-yl)carbamate (Compound F2)

By the same approach as c) step of Reference Example 101-c), from compound F1 (676 mg), compound F2 (400 mg) was obtained.

LC-MS: 394 (M+H)⁺/0.974 min, Measurement Condition G

### c) Production of tert-butyl((S)-3-methyl-1-(((S)-1-((4-((((4-nitrophenoxy)carbonyl)oxy) methyl)phenyl)amino)-1-oxopropan-2-yl)amino)-1-oxobutan-2-yl)carbamate (Reference Example 44)

By the same approach as Reference Example 101-d), from compound F2 (400 mg), Reference Example 102 (567 mg) was obtained.

LC-MS: 559(M+H)⁺/1.217 min, Measurement Condition G

### Reference Example 103

### L-Valyl-N-{4-[(5S,8S,11S,12E,16R)-8-tert-butyl-16,18-dicarboxy-4,10,13-trimethyl-5-[2-(1-methyl-1H-indol-3-yl)propan-2-yl]-3,6,9,14-tetraoxo-11-(propan-2-yl)-2-oxa-4,7,10,15-tetraazaoctadec-12-en-1-yl]phenyl}-N⁵-carbamoyl-L-ornithine amide

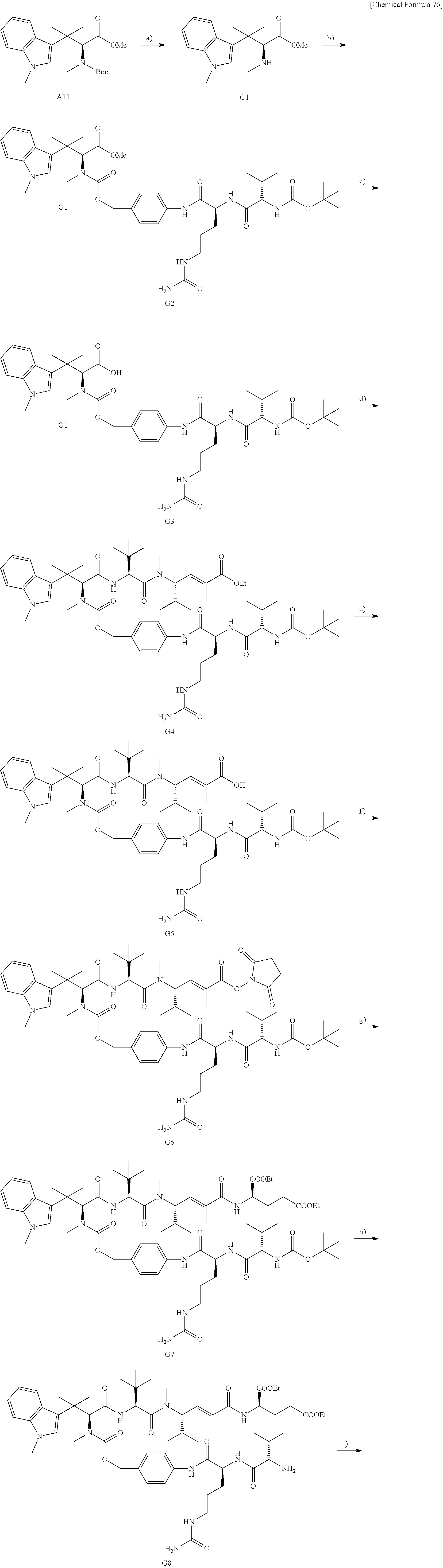

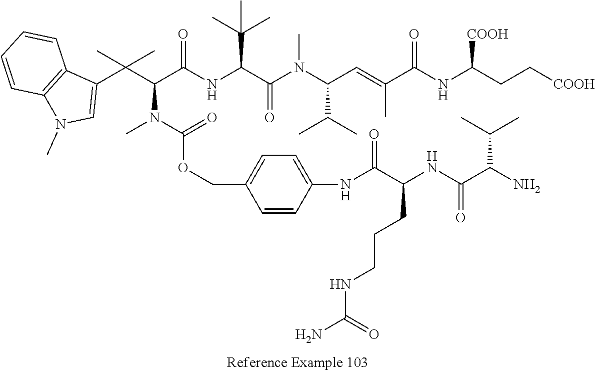

### a) Production of N,β,β,1-tetramethyl-L-tryptophan methyl ester (Compound G1)

To a solution compound A11 (402 mg) in chloroform (5 mL), trifluoroacetic acid (1 mL) was added, and the resultant mixture was stirred at 25° C. for 45 minutes. After the reaction ended, the reaction solution was purified by silica gel column chromatography (eluting solvent; methanol:chloroform) to give compound G1 (306 mg).

¹H-NMR (400 MHz, CDCl₃):1.47-1.48 (6H, m), 2.21 (3H, d, J=1.8 Hz), 3.61 (3H, d, J=2.3 Hz), 3.71 (1H, d, J=1.8 Hz), 3.72 (3H, d, J=1.8 Hz), 6.83 (1H, d, J=1.8 Hz), 7.08 (1H, t, J=8.2 Hz), 7.19 (1H, t, J=8.2 Hz), 7.27 (1H, d, J=8.2 Hz), 7.81 (1H, d, J=8.2 Hz).

LC-MS: 275 (M+H)⁺ (0.856 min, Measurement Condition D)

### b) Production of N-(tert-butoxycarbonyl)-L-valyl-N⁵-carbamoyl-N-{4-[({[(2S)-1-methoxy-3-methyl-3-(1-methyl-1H-indol-3-yl)-1-oxobutan-2-yl](methyl)carbamoyl}oxy)methyl]phenyl}-L-ornithine amide (Compound G2)

A mixed solution of compound G1 (306 mg), Reference Example 43 (101 mg), 2,6-lutidine (663 mg) and N,N-dimethylformamide (5.5 mL) was stirred at 45° C. for 8 hours. After the reaction ended, water was added and the resultant mixture was extracted with chloroform. The organic layer was washed with water and saturated sodium bicarbonate and then dried over anhydrous sodium sulfate, and the solvent was distilled off under reduced pressure. The residue was purified by silica gel column chromatography (eluting solvent; chloroform:methanol) to give compound G2 (516 mg).

LC-MS: 780 (M+H)⁺ (1.369 min, Measurement Condition D)

### c) Production of N-(tert-butoxycarbonyl)-L-valyl-N⁵-carbamoyl-N-{4-[({[(1S)-1-carboxy-2-methyl-2-(1-methyl-1H-indol-3-yl)propyl](methyl)carbamoyl}oxy) methyl]phenyl}-L-ornithine amide (Compound G3)

By the same approach as Reference Example 1-1), from compound G2 (516 mg), compound G3 (175 mg) was obtained.

LC-MS: 766 (M+H)⁺, 764 (M−H)⁻ (1.285 min, Measurement Condition D)

### d) Production of N-(tert-butoxycarbonyl)-L-valyl-N-{4-[(5S,8S,11S,12E)-8-tert-butyl-4,10,13-trimethyl-5-[2-(1-methyl-1H-indol-3-yl)propan-2-yl]-3,6,9,14-tetraoxo-11-(propan-2-yl)-2,15-dioxa-4,7,10-triazaheptadec-12-en-1-yl]phenyl}-N⁵-carbamoyl-L-ornithine amide (Compound G4)

By the same approach as Reference Example 1-m), from compound G3 (130 mg), compound G4 (146 mg) was obtained.

LC-MS: 1060 (M+H)⁺ (1.380 min, Measurement Condition D)

### e) Production of N-(tert-butoxycarbonyl)-L-valyl-N-{4-[(5S,8S,11S,12E)-8-tert-butyl-13-carboxy-4,10-dimethyl-5-[2-(1-methyl-1H-indol-3-yl)propan-2-yl]-3,6,9-trioxo-11-(propan-2-yl)-2-oxa-4,7,10-triazatetradec-12-en-1-yl]phenyl}-N⁵-carbamoyl-L-ornithine amide (Compound G5)

By the same approach as Reference Example 1-n), from compound G4 (148 mg), compound G5 (145 mg) was obtained.

LC-MS: 1032 (M+H)⁺ (1.231 min, Measurement Condition D)

### f) Production of N-(tert-butoxycarbonyl)-L-valyl-N-{4-[(5S,8S,11S,12E)-8-tert-butyl-14-[(2,5-dioxopyrrolidin-1-yl)oxy]-4,10,13-trimethyl-5-[2-(1-methyl-1H-indol-3-yl)propan-2-yl]-3,6,9,14-tetraoxo-11-(propan-2-yl)-2-oxa-4,7,10-triazatetradec-12-en-1-yl]phenyl}-N⁵-carbamoyl-L-ornithine amide (Compound G6)

By the same approach as Reference Example 1-o), from compound G5 (145 mg), compound G6 (139 mg) was obtained.

LC-MS: 1129 (M+H)⁺ (1.271 min, Measurement Condition D)

### g) Production of diethyl (2R)-2-{[(5S,8S,11S,12E)-1-(4-{[(2S)-2-({(2S)-2-[(tert-butoxycarbonyl)amino]-3-methylbutanoyl}amino)-5-(carbamoylamino)pentanoyl]amino}phenyl)-8-tert-butyl-4,10,13-trimethyl-5-[2-(1-methyl-1H-indol-3-yl) propan-2-yl]-3,6,9,14-tetraoxo-11-(propan-2-yl)-2-oxa-4,7,10-triazatetradec-12-en-14-yl]amino}pentanedioate (Compound G7)

By the same approach as Reference Example 3-a), from compound G6 (139 mg), compound G7 (154 mg) was obtained.

LC-MS: 1217 (M+H)⁺ (1.533 min, Measurement Condition D)

### h) Production of diethyl (2R)-2-{[(5S,8S,11S,12E)-1-(4-{[(2S)-2-{[(2S)-2-amino-3-methylbutanoyl]amino}-5-(carbamoylamino)pentanoyl]amino}phenyl)-8-tert-butyl-4,10,13-trimethyl-5-[2-(1-methyl-1H-indol-3-yl)propan-2-yl]-3,6,9,14-tetraoxo-11-(propan-2-yl)-2-oxa-4,7,10-triazatetradec-12-en-14-yl]amino}

pentanedioate (Compound G8) By the same approach as Reference Example 103-a), from compound G7 (92 mg), compound G8 (87 mg) was obtained.

LC-MS: 1117 (M+H)⁺ (1.279 min, Measurement Condition D)

### i) Production of L-valyl-N-{4-[(5S,8S,11S,12E,16R)-8-tert-butyl-16,18-dicarboxy-4,10,13-trimethyl-5-[2-(1-methyl-1H-indol-3-yl)propan-2-yl]-3,6,9,14-tetraoxo-11-(propan-2-yl)-2-oxa-4,7,10,15-tetraazaoctadec-12-en-1-yl]phenyl}-N⁵-carbamoyl-L-ornithine amide (Reference Example 103)

By carrying out synthesis by the same approach as Reference Example 1-n), and through purification by reversed phase column chromatography (eluting solvent; acetonitrile with 0.1% TFA:water), from compound G8 (87 mg), Reference Example 103 (79 mg) was obtained.

LC-MS: 1061 (M+H)⁺ (1.124 min, Measurement Condition D)

### Reference Example 104

### L-Prolyl-L-alanyl-N¹-(4-{[(N-{(2E,4S)-2,5-dimethyl-4-[methyl(N,β,β,1-tetramethyl-L-tryptophyl-3-methyl-L-valyl)amino]hex-2-enoyl}-L-α-glutamyl)oxy]methyl}phenyl)-L-aspartamide

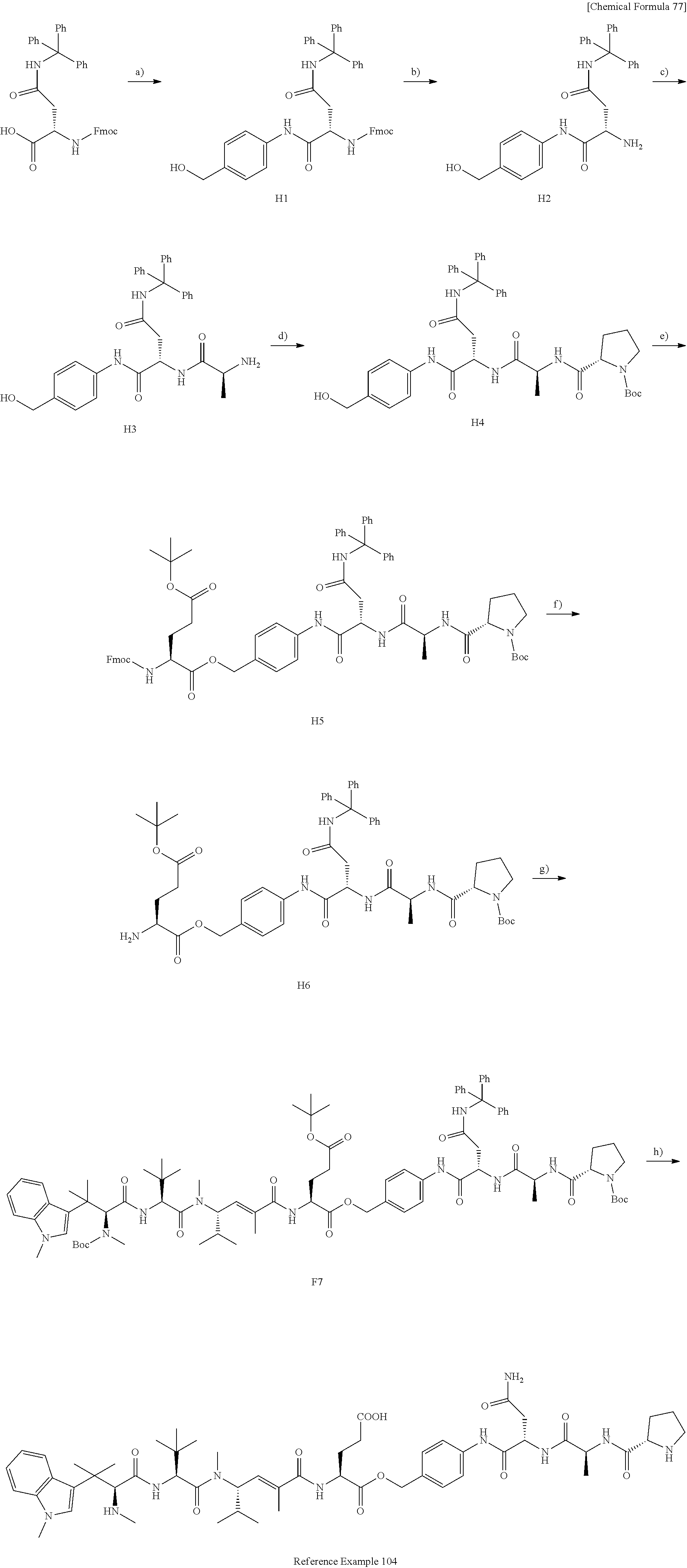

### a) Production of 9H-fluoren-9-ylmethyl[(2S)-1-{[4-(hydroxymethyl)phenyl]amino}-1,4-dioxo-4-(trithylamino)butan-2-yl]carbamate (Compound H1)

To a solution of N²-[(9H-fluoren-9-ylmethoxy)carbonyl]-N-trimethyl-L-asparagine (Fmoc-Asn(Trt)-OH, 18 g) and p-aminobenzyl alcohol (3.9 g) in THF (150 mL), 1-ethoxycarbonyl-2-ethoxy-1,2-dihydroquinoline (EEDQ) (8.6 g) was added, and the resultant mixture was stirred at room temperature overnight. To the residue obtained by distilling off the solvent under reduced pressure, ethyl acetate was added, and the resultant mixture was stirred at room temperature. The solid thus obtained was collected by filtration, washed with ethyl acetate and dried under reduced pressure. The same washing was carried out again to give compound H1 (19.2 g).

LC-MS: 702 (M+H)⁺ (3.36 min, Measurement Condition G)

### b) Production of N¹-[4-(hydroxymethyl)phenyl]-N⁴-trityl-L-aspartamide (Compound H2)

To compound H1 (3.0 g), a 30% piperidine-THF solution was added, and the resultant mixture was stirred at room temperature for 5 hours. To the residue obtained by concentrating the mixture under reduced pressure, diethyl ether was added, and the resultant mixture was washed and the solid was collected by filtration. The solid thus obtained was washed with diethyl ether and dried. The same washing was carried out again to give compound H2 (1.82 g).

LC-MS: 480 (M+H)⁺ (3.00 min, Measurement Condition F)

### c) Production of L-alanyl-N¹-[4-(hydroxymethyl)phenyl]-N⁴-trityl-L-aspartamide (Compound H3)

A solution of compound H2 (1.5 g), N-[(9H-fluoren-9-ylmethoxy)carbonyl]-L-alanine (Fmoc-Ala-OH, 1.23 g), 1-ethyl-3-(3-dimethylaminopropyl)carbodiimide hydrochloride (714 mg), 1-hydroxy-1H-benzotriazole monohydrate (505 mg) in N,N-dimethylformamide (15 mL) was stirred at room temperature overnight. After adding ethyl acetate, the resultant mixture was washed with a saturated aqueous ammonium chloride solution and saturated brine, and the organic solvent layer was collected and dried over sodium sulfate. After removing sodium sulfate, the amorphous product obtained through concentration was purified by short column chromatography (eluting solvent; methanol:chloroform). To the amorphous product thus obtained, a 30% piperidine-TIF solution was added, and the resultant mixture was stirred at room temperature for 5 hours. The residue obtained by concentrating the mixture under reduced pressure was purified by column chromatography (eluting solvent; methanol:chloroform) to give compound H3 (562 mg).

LC-MS: 551 (M+H)⁺ (2.89 min, Measurement Condition F)

### d) Production of 1-(tert-butoxycarbonyl)-L-prolyl-L-alanyl-N¹-[4-(hydroxymethyl)phenyl]-N⁴-trityl-L-aspartamide (Compound H4)

A solution of compound H3 (275 mg), 1-(tert-butoxycarbonyl)-L-proline (Boc-Pro-OH, 118 mg), 1-ethyl-3-(3-dimethylaminopropyl)carbodiimide hydrochloride (714 mg), 1-hydroxy-1H-benzotriazole monohydrate (505 mg) in N,N-dimethylformamide (15 mL) was stirred at room temperature overnight. After adding ethyl acetate, the resultant mixture was washed with a saturated aqueous ammonium chloride solution and saturated brine, and the organic solvent layer was collected and dried over sodium sulfate. After removing sodium sulfate, the amorphous product obtained through concentration was purified by column chromatography (eluting solvent; methanol:chloroform) to give compound H4 (321 mg).

LC-MS: 748 (M+H)⁺ (2.42 min, Measurement Condition G)

### e) Production of 1-(4-{[(2S)-2-{[(2S)-2-({[(2S)-1-(tert-butoxycarbonyl)pyrrolidin-2-yl]carbonyl}amino)propanoyl]amino}-4-oxo-4-(tritylamino)butanoyl]amino}benzyl)5-tert-butyl (2S)-2-{[(9H-fluoren-9-ylmethoxy)carbonyl]amino}pentanedioate (Compound H5)

Compound H4 (242 mg), (2S)-5-tert-butoxy-2-{[(9H-fluoren-9-ylmethoxy)carbonyl]amino}-5-oxopentanoic acid monohydrate (Fmoc-Glu(OtBu)-OH—H₂O, 150 mg), p-toluenesulfonyl chloride (TsCl) (65 mg) were dissolved in acetonitrile (5.0 mL), and the solution was cooled to 0° C. To that acetonitrile solution, 1-methylimidazole (0.06 mL) was added, and the solution was stirred overnight while bringing it back to room temperature. After adding ethyl acetate, the resultant mixture was washed with a saturated aqueous ammonium chloride solution and saturated brine, and the organic solvent layer was collected and dried over sodium sulfate. After removing sodium sulfate, the amorphous product obtained through concentration was purified by column chromatography (eluting solvent; methanol:chloroform) to give compound H5 (342 mg).

LC-MS: 1155 (M+H)⁺ (4.60 min, Measurement Condition G)

### f) Production of 1-(4-{[(2S)-2-{[(2S)-2-({[(2S)-1-(tert-butoxycarbonyl)pyrrolidin-2-yl]carbonyl}amino)propanoyl]amino}-4-oxo-4-(tritylamino)butanoyl]amino}benzyl)5-tert-butyl (2S)-2-aminopentanedioate (Compound H6)

By the same approach as Reference Example 104-b), from compound H5 (342 mg), compound H6 (110 mg) was obtained.

LC-MS: 933 (M+H)⁺ (2.23 min, Measurement Condition G)

g) Production of 1-(4-{[(2S)-2-{[(2S)-2-({[(2S)-1-(tert-butoxycarbonyl)pyrrolidin-2-yl]carbonyl}amino)propanoyl]amino}-4-oxo-4-(tritylamino)butanoyl]amino}benzyl)5-tert-butyl(2S)-2-{[(6S,9S,12S,13E)-9-tert-butyl-2,2,5,11,14-pentamethyl-6-[2-(1-methyl-1H-indol-3-yl)propan-2-yl]-4,7,10,15-tetraoxo-12-(propan-2-yl)-3-oxa-5,8,11-triazapentadec-13-en-15-yl]amino}pentanedioate (Compound H7)

Compound H6 (30 mg), compound A13 (22 mg), bromotripyrrolidinophosphonium hexafluorophosphate (20 mg), 4-dimethylaminopyridine (5 mg) were dissolved in N,N-dimethylformamide (5.0 mL), and the solution was cooled to 0° C. To that mixed solution, N-diisopropylethylamine (0.017 mL) was added dropwise, and the solution was stirred overnight while bringing it back to room temperature. Ethyl acetate was added, and the resultant mixture was washed with a saturated aqueous ammonium chloride solution and saturated brine, and dried over sodium sulfate. After removing sodium sulfate, the amorphous product obtained through concentration was purified by silica gel column chromatography (eluting solvent; methanol:chloroform) to give compound H7 (42 mg).

LC-MS: 1541 (M+H)⁺ (5.23 min, Measurement Condition G)

### h) Production of L-prolyl-L-alanyl-N¹-(4-{[(N-{(2E,4S)-2,5-dimethyl-4-[methyl(N,β,β,1-tetramethyl-L-tryptophyl-3-methyl-L-valyl)amino]hex-2-enoyl}-L-α-glutamyl)oxy]methyl}phenyl)-L-aspartamide (Reference Example 104)

By carrying out synthesis by the same approach as Reference Example 103-a), and through purification by reversed phase column chromatography (eluting solvent; acetonitrile with 0.1% TFA:water), from compound H7 (42 mg), Reference Example 104 (10.3 mg) was obtained.

LC-MS: 1043 (M+H)⁺ (2.79 min, Measurement Condition F)

### Reference Examples 105 to 116

The compounds shown in the following table 6 were obtained through the same reaction and treatment as Reference Example 103 and Reference Example 104, using corresponding raw material compounds.

TABLE 6

| | | | LC-MS |
|---|---|---|---|
| Reference | | | Measurement |
| Example | Structural Formula | LC-MS/Rt (min) | Condition |
| 105 | 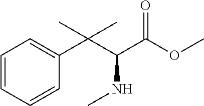 [SMILES: CN[C@H](C(=O)OC)C(C)(C)c1ccccc1] | 222(M + H)⁺/0.911 | C |
| 106 | 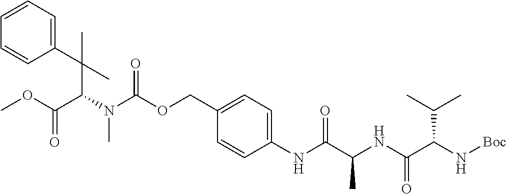 [SMILES: COC(=O)[C@@H](N(C)C(=O)OCc1ccc(NC(=O)[C@H](C)NC(=O)[C@@H](NC(=O)OC(C)(C)C)C(C)C)cc1)C(C)(C)c1ccccc1] | 663(M + Na)⁺/1.280 | G |
| 107 | 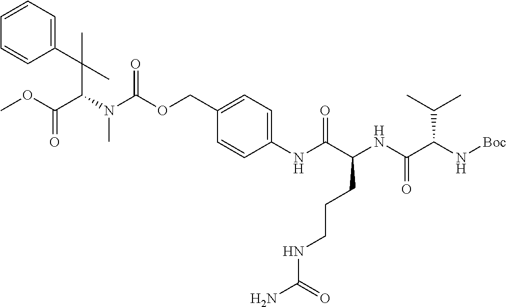 [SMILES: COC(=O)[C@@H](N(C)C(=O)OCc1ccc(NC(=O)[C@H](CCCNC(N)=O)NC(=O)[C@@H](NC(=O)OC(C)(C)C)C(C)C)cc1)C(C)(C)c1ccccc1] | 727(M + H)⁺/1.419 | G |
| 108 | 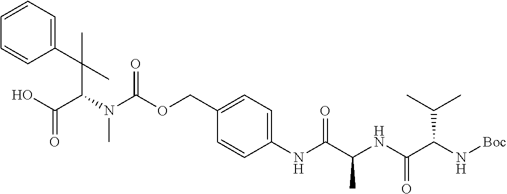 [SMILES: CC(C)[C@H](NC(=O)OC(C)(C)C)C(=O)N[C@@H](C)C(=O)Nc1ccc(COC(=O)N(C)[C@H](C(=O)O)C(C)(C)c2ccccc2)cc1] | 625(M − H)⁻/1.182 | G |
| 109 | 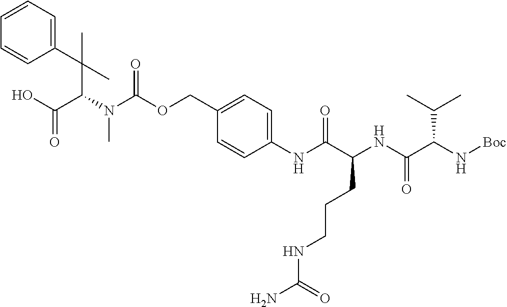 [SMILES: CC(C)[C@H](NC(=O)OC(C)(C)C)C(=O)N[C@@H](CCCNC(N)=O)C(=O)Nc1ccc(COC(=O)N(C)[C@H](C(=O)O)C(C)(C)c2ccccc2)cc1] | 713(M + H)⁺/1.120 | G |
| 110 | 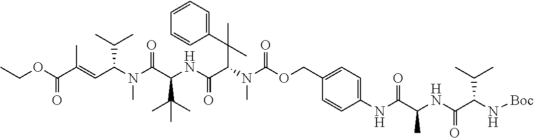 [SMILES: CCOC(=O)/C(C)=C/[C@H](C(C)C)N(C)C(=O)[C@@H](NC(=O)[C@@H](N(C)C(=O)OCc1ccc(NC(=O)[C@H](C)NC(=O)[C@@H](NC(=O)OC(C)(C)C)C(C)C)cc1)C(C)(C)c1ccccc1)C(C)(C)C] | 943(M + Na)⁺/1.468 | G |
| 111 | 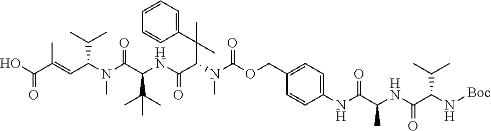 [SMILES: C/C(=C\[C@H](C(C)C)N(C)C(=O)[C@@H](NC(=O)[C@@H](N(C)C(=O)OCc1ccc(NC(=O)[C@H](C)NC(=O)[C@@H](NC(=O)OC(C)(C)C)C(C)C)cc1)C(C)(C)c1ccccc1)C(C)(C)C)C(=O)O] | 893(M + H)⁺/1.315 | G |
| 112 | 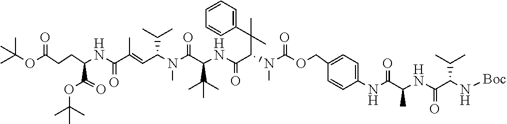 [SMILES: C/C(=C\[C@H](C(C)C)N(C)C(=O)[C@@H](NC(=O)[C@@H](N(C)C(=O)OCc1ccc(NC(=O)[C@H](C)NC(=O)[C@@H](NC(=O)OC(C)(C)C)C(C)C)cc1)C(C)(C)c1ccccc1)C(C)(C)C)C(=O)N[C@H](CCC(=O)OC(C)(C)C)C(=O)OC(C)(C)C] | 1134(M + H)⁺/1.512 | G |
| 113 | 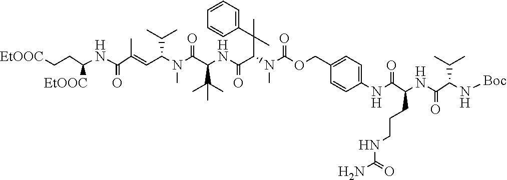 [SMILES: CCOC(=O)CC[C@@H](NC(=O)/C(C)=C/[C@H](C(C)C)N(C)C(=O)[C@@H](NC(=O)[C@@H](N(C)C(=O)OCc1ccc(NC(=O)[C@H](CCCNC(N)=O)NC(=O)[C@@H](NC(=O)OC(C)(C)C)C(C)C)cc1)C(C)(C)c1ccccc1)C(C)(C)C)C(=O)OCC] | 1186(M + Na)⁺/1.302 | G |
| 114 | 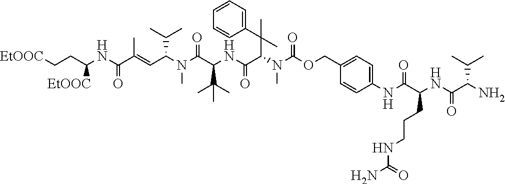 [SMILES: CCOC(=O)CC[C@@H](NC(=O)/C(C)=C/[C@H](C(C)C)N(C)C(=O)[C@@H](NC(=O)[C@@H](N(C)C(=O)OCc1ccc(NC(=O)[C@H](CCCNC(N)=O)NC(=O)[C@@H](N)C(C)C)cc1)C(C)(C)c1ccccc1)C(C)(C)C)C(=O)OCC] | 1086(M + Na)⁺/1.083 | G |
| 115 | 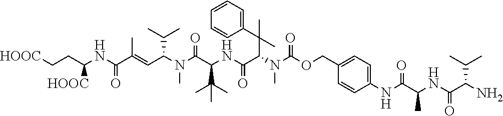 [SMILES: C/C(=C\[C@H](C(C)C)N(C)C(=O)[C@@H](NC(=O)[C@@H](N(C)C(=O)OCc1ccc(NC(=O)[C@H](C)NC(=O)[C@@H](N)C(C)C)cc1)C(C)(C)c1ccccc1)C(C)(C)C)C(=O)N[C@H](CCC(=O)O)C(=O)O] | 922(M + H)+/1.224 | G |
| 116 | 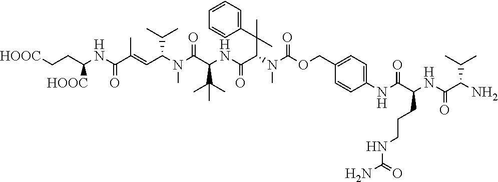 [SMILES: C/C(=C\[C@H](C(C)C)N(C)C(=O)[C@@H](NC(=O)[C@@H](N(C)C(=O)OCc1ccc(NC(=O)[C@H](CCCNC(N)=O)NC(=O)[C@@H](N)C(C)C)cc1)C(C)(C)c1ccccc1)C(C)(C)C)C(=O)N[C@H](CCC(=O)O)C(=O)O] | 1008(M + H)⁺/0.921 | G |

### Reference Examples 117 to 119

The compounds shown in the following table 7 were obtained through the same reaction and treatment as Reference Example 104, using corresponding raw material compounds.

TABLE 7

| | | | LC-MS |
|---|---|---|---|
| Reference | | | Measurement |
| Example | Structural Formula | LC-MS/Rt (min) | Condition |
| 117 | 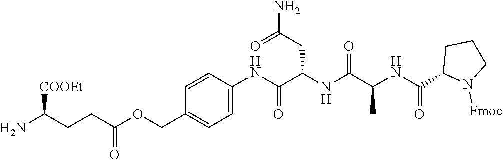 | 785(M + H)⁺/1.043 | B |
| 118 | 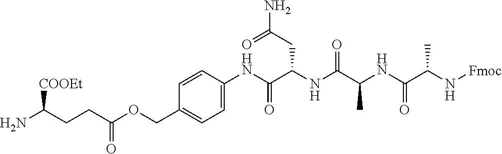 | 759(M + H)⁺/0.948 | B |
| 119 | 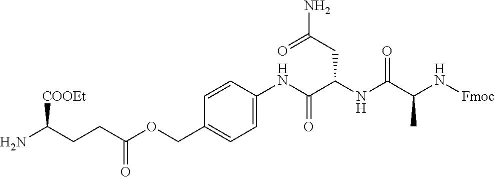 | 688(M + H)⁺/0.781 | E |

### Example 1

### N,β,β-tetramethyl-L-tryptophyl-N-[(3S,4E)-6-{[(1R)-1,3-dicarboxypropyl]amino}-2,5-dimethyl-6-oxohex-4-en-3-yl]-N,3-dimethyl-L-valinamide

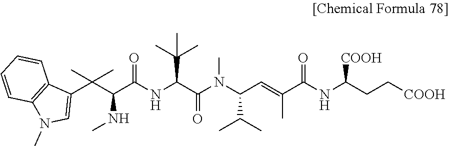

To a solution of Reference Example 3 (100 mg) in chloroform (1.0 mL), trifluoroacetic acid (0.2 mL) was added, and the resultant mixture was stirred at 25° C. for 1 hour. After the reaction ended, the reaction solution was purified by silica gel column chromatography (eluting solvent; methanol:chloroform) to give Example 1 (60 mg).

LC-MS: 656 (M+H)⁺ (1.036 min, Measurement Condition C)

### Example 2

### N,β,β-Trimethyl-L-phenylalanyl-N-[(3S,4E)-6-{[(1R)-1,3-dicarboxypropyl]amino}-2,5-dimethyl-6-oxohex-4-en-3-yl]-N,3-dimethyl-L-valinamide

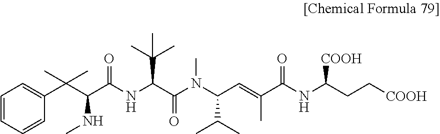 [SMILES: CN[C@H](C(=O)N[C@H](C(=O)N(C)[C@H](/C=C(\C)C(=O)N[C@H](CCC(=O)O)C(=O)O)C(C)C)C(C)(C)C)C(C)(C)c1ccccc1]

To a solution of Reference Example 16 (20 mg) in chloroform (1.0 mL), trifluoroacetic acid (0.2 mL) was added, and the resultant mixture was stirred at 25° C. for 1 hour. After the reaction ended, the reaction solution was purified by reversed phase column chromatography (eluting solvent; acetonitrile with 0.1% TFA:water) to give Example 2 (9.3 mg).

LC-MS: 603 (M+H)⁺ (0.870 min, Measurement Condition D)

### Example 3

### N-[(2E,4S)-2,5-Dimethyl-4-(methyl {3-methyl-N-[(2R)-1-(propan-2-yl) piperidine-2-carbonyl]-L-valyl}amino)hex-2-enoyl]-D-glutamic acid

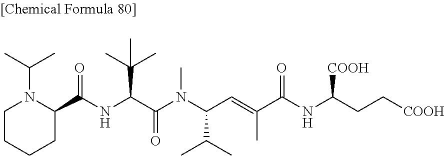 [SMILES: C/C(=C\[C@H](C(C)C)N(C)C(=O)[C@@H](NC(=O)[C@H]1CCCCN1C(C)C)C(C)(C)C)C(=O)N[C@H](CCC(=O)O)C(=O)O]

After a mixed solution of Reference Example 6 (22.1 mg) in tetrahydrofuran (4.0 mL) and water (1.0 mL) was cooled to 0° C., lithium hydroxide (3.12 mg) was added, and thereafter the resultant mixture was stirred at 25° C. for 12 hours. After the reaction ended, the reaction solution was purified by reversed phase column chromatography (eluting solvent; acetonitrile with 0.1% TFA:water) to give Example 3 (5.8 mg).

LC-MS: 567 (M+H)⁺ (0.80 min, Measurement Condition G)

### Examples 4 to 10

The compounds shown in the following table 8 were obtained through the same reaction and treatment as Example 1, Example 2, or Example 3 using corresponding raw material compounds.

TABLE 8

| | | | LC-MS |
|---|---|---|---|
| | | | Measurement |
| Example | Structural Formula | LC-MS/Rt (min) | Condition |
| 4 | 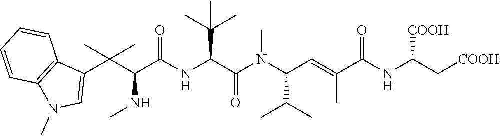 [SMILES: CN[C@H](C(=O)N[C@H](C(=O)N(C)[C@H](/C=C(\C)C(=O)N[C@@H](CC(=O)O)C(=O)O)C(C)C)C(C)(C)C)C(C)(C)c1cn(C)c2ccccc12] | 642(M + H)⁺/0.998 | D |
| 5 | 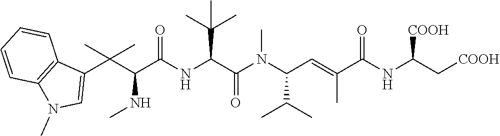 [SMILES: CN[C@H](C(=O)N[C@H](C(=O)N(C)[C@H](/C=C(\C)C(=O)N[C@H](CC(=O)O)C(=O)O)C(C)C)C(C)(C)C)C(C)(C)c1cn(C)c2ccccc12] | 642(M + H)⁺/1.054 | D |
| 6 | 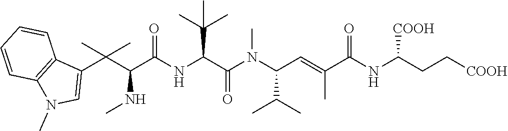 [SMILES: CN[C@H](C(=O)N[C@H](C(=O)N(C)[C@H](/C=C(\C)C(=O)N[C@@H](CCC(=O)O)C(=O)O)C(C)C)C(C)(C)C)C(C)(C)c1cn(C)c2ccccc12] | 656(M + H)⁺/1.038 | D |
| 7 | 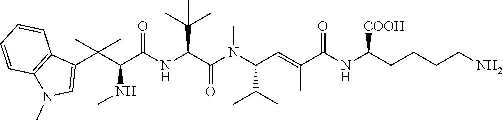 [SMILES: CN[C@H](C(=O)N[C@H](C(=O)N(C)[C@H](/C=C(\C)C(=O)N[C@H](CCCCN)C(=O)O)C(C)C)C(C)(C)C)C(C)(C)c1cn(C)c2ccccc12] | 655(M + H)⁺/0.933 | D |
| 8 | 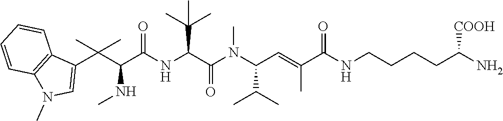 [SMILES: CN[C@H](C(=O)N[C@H](C(=O)N(C)[C@H](/C=C(\C)C(=O)NCCCC[C@@H](N)C(=O)O)C(C)C)C(C)(C)C)C(C)(C)c1cn(C)c2ccccc12] | 655(M + H)⁺/0.854 | D |
| 9 | 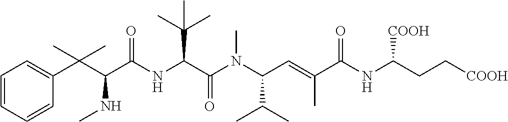 [SMILES: CN[C@H](C(=O)N[C@H](C(=O)N(C)[C@H](/C=C(\C)C(=O)N[C@@H](CCC(=O)O)C(=O)O)C(C)C)C(C)(C)C)C(C)(C)c1ccccc1] | 603(M + H)⁺/1.131 | C |
| 10 | 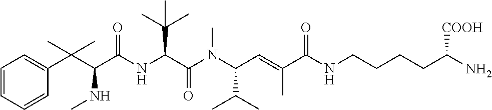 [SMILES: CN[C@H](C(=O)N[C@H](C(=O)N(C)[C@H](/C=C(\C)C(=O)NCCCC[C@@H](N)C(=O)O)C(C)C)C(C)(C)C)C(C)(C)c1ccccc1] | 602(M + H)⁺/0.853 | G |

### Example 11

### N,β,β-Trimethyl-L-phenylalanyl-N-[(3 S,4E)-6-({(5R)-5-carboxy-5-[4-(2,5-dioxo-2,5-dihydro-1H-pyrrol-1-yl)butanamido]pentyl}amino)-2,5-dimethyl-6-oxohex-4-en-3-yl]-N,3-dimethyl-L-valinamide

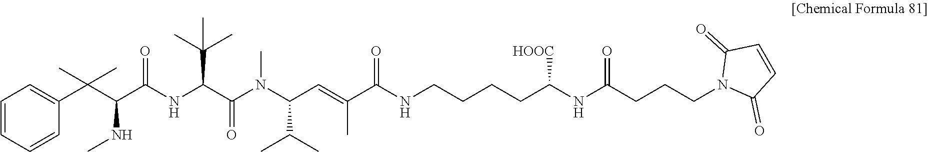 [SMILES: CN[C@H](C(=O)N[C@H](C(=O)N(C)[C@H](/C=C(\C)C(=O)NCCCC[C@@H](NC(=O)CCCN1C(=O)C=CC1=O)C(=O)O)C(C)C)C(C)(C)C)C(C)(C)c1ccccc1]

A mixed solution of ditrifluoroacetate of Example 10 (35.1 mg), N-succinimidyl 4-maleimidebutyrate (17.7 mg), N-diisopropylethylamine (27.3 mg) and N,N-dimethylformamide (1 mL) was stirred at 25° C. for 3 hours. After the reaction ended, the reaction solution was purified by reversed phase column chromatography (eluting solvent; acetonitrile with 0.1% TFA:water) to give Example 11 (9.5 mg).

LC-MS: 767 (M+H)⁺ (0.969 min, Measurement Condition G)

### Examples 12 to 32

The compounds shown in the following table 9 were obtained through the same reaction and treatment as Example 1, Example 2, Example 3 or Example 11 using corresponding raw material compounds.

TABLE 9

| | | | LC-MS |
|---|---|---|---|
| | | | Measurement |
| Example | Structural Formula | LC-MS/Rt (min) | Condition |
| 12 | 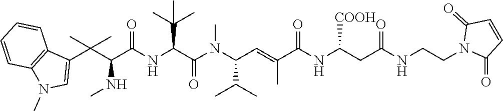 [SMILES: CN[C@H](C(=O)N[C@H](C(=O)N(C)[C@H](/C=C(\C)C(=O)N[C@@H](CC(=O)NCCN1C(=O)C=CC1=O)C(=O)O)C(C)C)C(C)(C)C)C(C)(C)c1cn(C)c2ccccc12] | 764(M + H)⁺/1.108 | G |
| 13 | 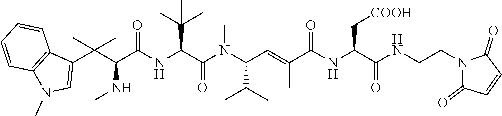 [SMILES: CN[C@H](C(=O)N[C@H](C(=O)N(C)[C@H](/C=C(\C)C(=O)N[C@@H](CC(=O)O)C(=O)NCCN1C(=O)C=CC1=O)C(C)C)C(C)(C)C)C(C)(C)c1cn(C)c2ccccc12] | 764(M + H)⁺/1.023 | G |
| 14 | 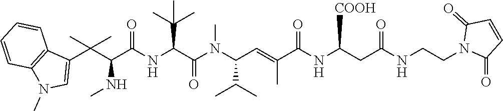 [SMILES: CN[C@H](C(=O)N[C@H](C(=O)N(C)[C@H](/C=C(\C)C(=O)N[C@H](CC(=O)NCCN1C(=O)C=CC1=O)C(=O)O)C(C)C)C(C)(C)C)C(C)(C)c1cn(C)c2ccccc12] | 764(M + H)⁺/1.077 | G |
| 15 | 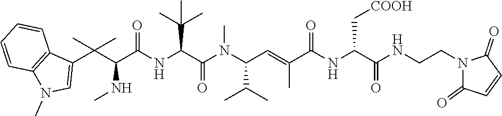 [SMILES: CN[C@H](C(=O)N[C@H](C(=O)N(C)[C@H](/C=C(\C)C(=O)N[C@H](CC(=O)O)C(=O)NCCN1C(=O)C=CC1=O)C(C)C)C(C)(C)C)C(C)(C)c1cn(C)c2ccccc12] | 764(M + H)⁺/1.086 | G |
| 16 | 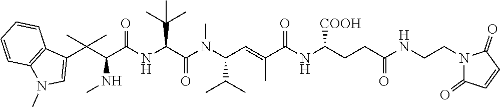 [SMILES: CN[C@H](C(=O)N[C@H](C(=O)N(C)[C@H](/C=C(\C)C(=O)N[C@@H](CCC(=O)NCCN1C(=O)C=CC1=O)C(=O)O)C(C)C)C(C)(C)C)C(C)(C)c1cn(C)c2ccccc12] | 778(M + H)⁺/1.174 | G |
| 17 | 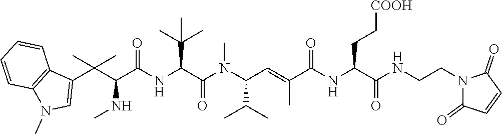 [SMILES: CN[C@H](C(=O)N[C@H](C(=O)N(C)[C@H](/C=C(\C)C(=O)N[C@@H](CCC(=O)O)C(=O)NCCN1C(=O)C=CC1=O)C(C)C)C(C)(C)C)C(C)(C)c1cn(C)c2ccccc12] | 778(M + H)⁺/1.043 | G |
| 18 | 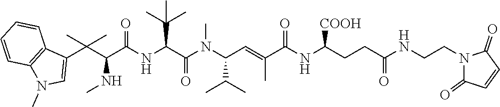 [SMILES: CN[C@H](C(=O)N[C@H](C(=O)N(C)[C@H](/C=C(\C)C(=O)N[C@H](CCC(=O)NCCN1C(=O)C=CC1=O)C(=O)O)C(C)C)C(C)(C)C)C(C)(C)c1cn(C)c2ccccc12] | 778(M + H)⁺/1.081 | D |
| 19 | 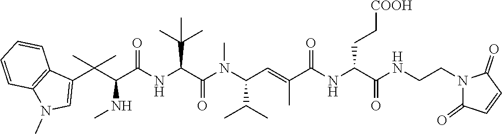 [SMILES: CN[C@H](C(=O)N[C@H](C(=O)N(C)[C@H](/C=C(\C)C(=O)N[C@H](CCC(=O)O)C(=O)NCCN1C(=O)C=CC1=O)C(C)C)C(C)(C)C)C(C)(C)c1cn(C)c2ccccc12] | 778(M + H)⁺/1.081 | G |
| 20 | 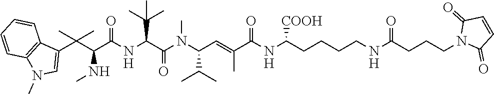 [SMILES: CN[C@H](C(=O)N[C@H](C(=O)N(C)[C@H](/C=C(\C)C(=O)N[C@@H](CCCCNC(=O)CCCN1C(=O)C=CC1=O)C(=O)O)C(C)C)C(C)(C)C)C(C)(C)c1cn(C)c2ccccc12] | 820(M + H)⁺/1.027 | G |
| 21 | 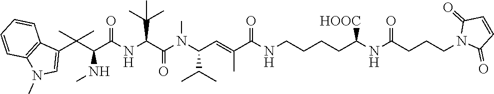 [SMILES: CN[C@H](C(=O)N[C@H](C(=O)N(C)[C@H](/C=C(\C)C(=O)NCCCC[C@H](NC(=O)CCCN1C(=O)C=CC1=O)C(=O)O)C(C)C)C(C)(C)C)C(C)(C)c1cn(C)c2ccccc12] | 820(M + H)⁺/1.041 | D |
| 22 | 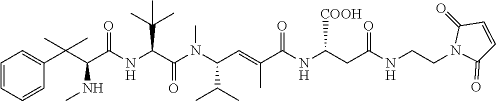 [SMILES: CN[C@H](C(=O)N[C@H](C(=O)N(C)[C@H](/C=C(\C)C(=O)N[C@@H](CC(=O)NCCN1C(=O)C=CC1=O)C(=O)O)C(C)C)C(C)(C)C)C(C)(C)c1ccccc1] | 711(M + H)⁺/0.924 | G |
| 23 | 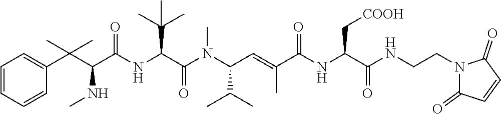 [SMILES: CN[C@H](C(=O)N[C@H](C(=O)N(C)[C@H](/C=C(\C)C(=O)N[C@@H](CC(=O)O)C(=O)NCCN1C(=O)C=CC1=O)C(C)C)C(C)(C)C)C(C)(C)c1ccccc1] | 711(M + H)⁺/0.838 | G |
| 24 | 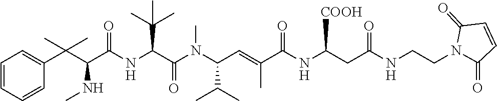 [SMILES: CN[C@H](C(=O)N[C@H](C(=O)N(C)[C@H](/C=C(\C)C(=O)N[C@H](CC(=O)NCCN1C(=O)C=CC1=O)C(=O)O)C(C)C)C(C)(C)C)C(C)(C)c1ccccc1] | 711(M + H)⁺/0.979 | G |
| 25 | 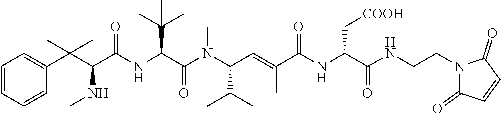 [SMILES: CN[C@H](C(=O)N[C@H](C(=O)N(C)[C@H](/C=C(\C)C(=O)N[C@H](CC(=O)O)C(=O)NCCN1C(=O)C=CC1=O)C(C)C)C(C)(C)C)C(C)(C)c1ccccc1] | 711(M + H)⁺/0.839 | G |
| 26 | 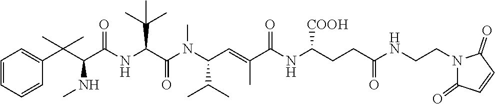 [SMILES: CN[C@H](C(=O)N[C@H](C(=O)N(C)[C@H](/C=C(\C)C(=O)N[C@@H](CCC(=O)NCCN1C(=O)C=CC1=O)C(=O)O)C(C)C)C(C)(C)C)C(C)(C)c1ccccc1] | 725(M + H)+/1.120 | G |
| 27 | 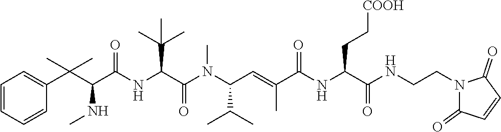 [SMILES: CN[C@H](C(=O)N[C@H](C(=O)N(C)[C@H](/C=C(\C)C(=O)N[C@@H](CCC(=O)O)C(=O)NCCN1C(=O)C=CC1=O)C(C)C)C(C)(C)C)C(C)(C)c1ccccc1] | 725(M + H)+/0.896 | G |
| 28 | 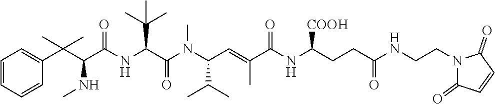 [SMILES: CN[C@H](C(=O)N[C@H](C(=O)N(C)[C@H](/C=C(\C)C(=O)N[C@H](CCC(=O)NCCN1C(=O)C=CC1=O)C(=O)O)C(C)C)C(C)(C)C)C(C)(C)c1ccccc1] | 725(M + H)+/0.821 | D |
| 29 | 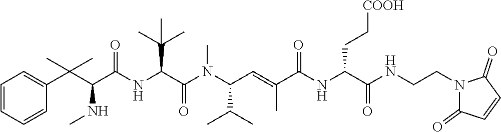 [SMILES: CN[C@H](C(=O)N[C@H](C(=O)N(C)[C@H](/C=C(\C)C(=O)N[C@H](CCC(=O)O)C(=O)NCCN1C(=O)C=CC1=O)C(C)C)C(C)(C)C)C(C)(C)c1ccccc1] | 725(M + H)⁺/0.847 | G |
| 30 | 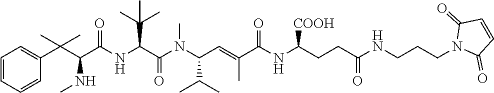 [SMILES: CN[C@H](C(=O)N[C@H](C(=O)N(C)[C@H](/C=C(\C)C(=O)N[C@H](CCC(=O)NCCCN1C(=O)C=CC1=O)C(=O)O)C(C)C)C(C)(C)C)C(C)(C)c1ccccc1] | 739(M + H)⁺/0.852 | G |
| 31 | 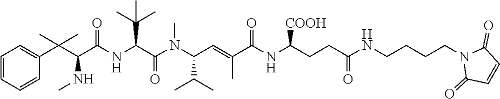 [SMILES: CN[C@H](C(=O)N[C@H](C(=O)N(C)[C@H](/C=C(\C)C(=O)N[C@H](CCC(=O)NCCCCN1C(=O)C=CC1=O)C(=O)O)C(C)C)C(C)(C)C)C(C)(C)c1ccccc1] | 753(M + H)⁺/1.059 | G |
| 32 | 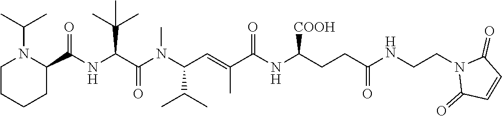 [SMILES: C/C(=C\[C@H](C(C)C)N(C)C(=O)[C@@H](NC(=O)[C@H]1CCCCN1C(C)C)C(C)(C)C)C(=O)N[C@H](CCC(=O)NCCN1C(=O)C=CC1=O)C(=O)O] | 687(M − H)⁻/0.82 | G |

### Example 33

### N-[6-(2,5-dioxo-2,5-dihydro-11H-pyrrol-1-yl)hexanoyl]-L-valyl-N-{4-[(5S,8S,11S,12E,16R)-8-tert-butyl-16,18-dicarboxy-4,10,13-trimethyl-3,6,9,14-tetraoxo-5-(2-phenylpropan-2-yl)-11-(propan-2-yl)-2-oxa-4,7,10,15-tetraazaoctadec-12-en-1-yl]phenyl}-N⁵-carbamoyl-L-ornithine amide

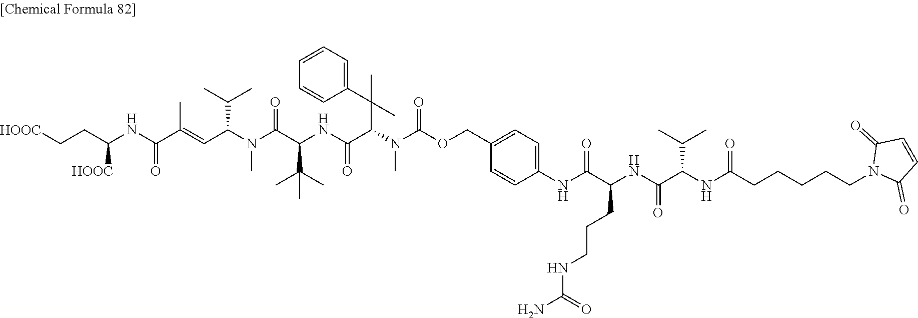 [SMILES: C/C(=C\[C@H](C(C)C)N(C)C(=O)[C@@H](NC(=O)[C@@H](N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)(C)c1ccccc1)C(C)(C)C)C(=O)N[C@H](CCC(=O)O)C(=O)O]

A mixed solution of trifluoroacetate of Reference Example 116 (0.7 mg), N-succinimidyl 6-maleimidehexanoate (0.4 mg), N-diisopropylethylamine (0.16 mg) and N,N-dimethylformamide (0.5 mL) was stirred at 25° C. for 3 hours. After the reaction ended, the reaction solution was purified by reversed phase column chromatography (eluting solvent; acetonitrile with 0.1% TFA:water) to give Example 33 (0.73 mg).

LC-MS: 1201 (M+H)⁺ (1.110 min, Measurement Condition D)

### Examples 34 to 36

The compound shown in the following table 10 was obtained through the same reaction and treatment as Example 33, using a corresponding raw material compound.

TABLE 10

| | | | LC-MS |
|---|---|---|---|
| | | | Measurement |
| Example | Structural Formula | LC-MS/Rt (min) | Condition |
| 34 | 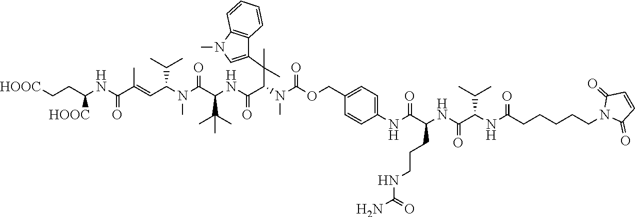 [SMILES: C/C(=C\[C@H](C(C)C)N(C)C(=O)[C@@H](NC(=O)[C@@H](N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)(C)c1cn(C)c2ccccc12)C(C)(C)C)C(=O)N[C@H](CCC(=O)O)C(=O)O] | 1254(M + H)⁺/1.290 | D |
| 35 | 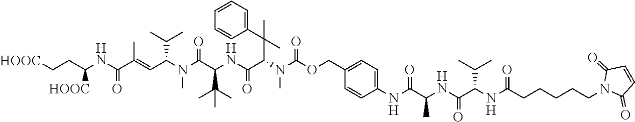 [SMILES: C/C(=C\[C@H](C(C)C)N(C)C(=O)[C@@H](NC(=O)[C@@H](N(C)C(=O)OCc1ccc(NC(=O)[C@H](C)NC(=O)[C@@H](NC(=O)CCCCCN2C(=O)C=CC2=O)C(C)C)cc1)C(C)(C)c1ccccc1)C(C)(C)C)C(=O)N[C@H](CCC(=O)O)C(=O)O] | 1113(M − H)⁻/1.224 | G |
| 36 | 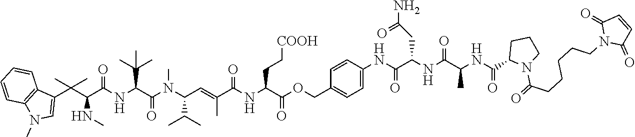 [SMILES: CN[C@H](C(=O)N[C@H](C(=O)N(C)[C@H](/C=C(\C)C(=O)N[C@@H](CCC(=O)O)C(=O)OCc1ccc(NC(=O)[C@H](CC(N)=O)NC(=O)[C@H](C)NC(=O)[C@@H]2CCCN2C(=O)CCCCCN2C(=O)C=CC2=O)cc1)C(C)C)C(C)(C)C)C(C)(C)c1cn(C)c2ccccc12] | 1236(M + H)⁺/3.08 | E |

### Example 37

### (3S,6S,9S,10E,14R)-14-(3-{[2-(3-{[(2R)-2-Amino-2-carboxyethyl]sulfanyl}-2,5-dioxopyrrolidin-1-yl)ethyl]amino}-3-oxopropyl)-6-tert-butyl-8,11-dimethyl-4,7,12-trioxo-3-(2-phenylpropan-2-yl)-9-(propan-2-yl)-2,5,8,13-tetraazapentadec-10-en-15-oic acid

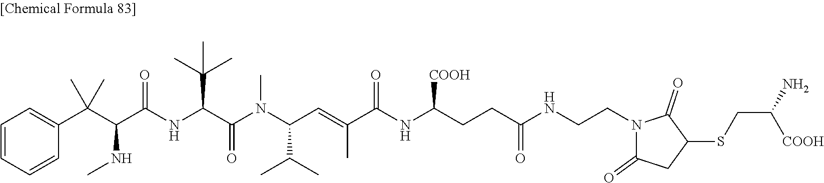 [SMILES: CN[C@H](C(=O)N[C@H](C(=O)N(C)[C@H](/C=C(\C)C(=O)N[C@H](CCC(=O)NCCN1C(=O)CC(SC[C@H](N)C(=O)O)C1=O)C(=O)O)C(C)C)C(C)(C)C)C(C)(C)c1ccccc1]

To an aqueous solution (1.0 mL) of Example 28 (10 mg), cysteine (1.73 mg) was added, and the resultant mixture was stirred at 4° C. for 1 hour. Thereafter, the reaction solution was purified by reversed phase column chromatography (eluting solvent; acetonitrile with 0.1% TFA:water) to give Example 37 (10 mg).

LC-MS: 846 (M+H)⁺, 844 (M−H)⁻ (0.855 min, Measurement Condition B)

### Examples 38 to 59

The compounds shown in the following Table 11 were obtained through the same reaction and treatment as Example 37, using corresponding raw material compounds.

TABLE 11

| | | | LC-MS |
|---|---|---|---|
| | | | Measurement |
| Example | Structural Formula | LC-MS/Rt (min) | Condition |
| 38 | 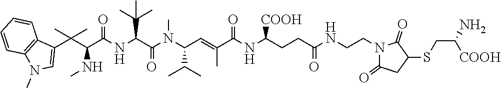 [SMILES: CN[C@H](C(=O)N[C@H](C(=O)N(C)[C@H](/C=C(\C)C(=O)N[C@H](CCC(=O)NCCN1C(=O)CC(SC[C@H](N)C(=O)O)C1=O)C(=O)O)C(C)C)C(C)(C)C)C(C)(C)c1cn(C)c2ccccc12] | 897(M − H)⁻/0.879 | B |
| 39 | 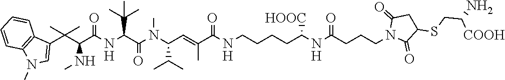 [SMILES: CN[C@H](C(=O)N[C@H](C(=O)N(C)[C@H](/C=C(\C)C(=O)NCCCC[C@@H](NC(=O)CCCN1C(=O)CC(SC[C@H](N)C(=O)O)C1=O)C(=O)O)C(C)C)C(C)(C)C)C(C)(C)c1cn(C)c2ccccc12] | 939(M − H)⁻/1.255 | B |
| 40 | 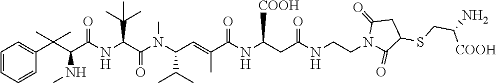 [SMILES: CN[C@H](C(=O)N[C@H](C(=O)N(C)[C@H](/C=C(\C)C(=O)N[C@H](CC(=O)NCCN1C(=O)CC(SC[C@H](N)C(=O)O)C1=O)C(=O)O)C(C)C)C(C)(C)C)C(C)(C)c1ccccc1] | 830(M − H)⁻/0.800 | B |
| 41 | 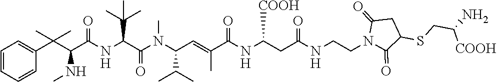 [SMILES: CN[C@H](C(=O)N[C@H](C(=O)N(C)[C@H](/C=C(\C)C(=O)N[C@@H](CC(=O)NCCN1C(=O)CC(SC[C@H](N)C(=O)O)C1=O)C(=O)O)C(C)C)C(C)(C)C)C(C)(C)c1ccccc1] | 830(M − H)⁻/0.823 | B |
| 42 | 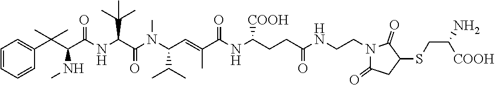 [SMILES: CN[C@H](C(=O)N[C@H](C(=O)N(C)[C@H](/C=C(\C)C(=O)N[C@@H](CCC(=O)NCCN1C(=O)CC(SC[C@H](N)C(=O)O)C1=O)C(=O)O)C(C)C)C(C)(C)C)C(C)(C)c1ccccc1] | 844(M − H)⁻/0.805 | B |
| 43 | 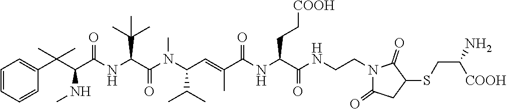 [SMILES: CN[C@H](C(=O)N[C@H](C(=O)N(C)[C@H](/C=C(\C)C(=O)N[C@@H](CCC(=O)O)C(=O)NCCN1C(=O)CC(SC[C@H](N)C(=O)O)C1=O)C(C)C)C(C)(C)C)C(C)(C)c1ccccc1] | 844(M − H)⁻/0.837 | B |
| 44 | 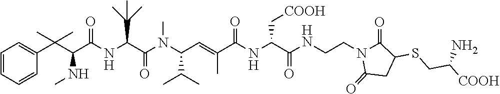 [SMILES: CN[C@H](C(=O)N[C@H](C(=O)N(C)[C@H](/C=C(\C)C(=O)N[C@H](CC(=O)O)C(=O)NCCN1C(=O)CC(SC[C@H](N)C(=O)O)C1=O)C(C)C)C(C)(C)C)C(C)(C)c1ccccc1] | 830(M − H)⁻/0.829 | B |
| 45 | 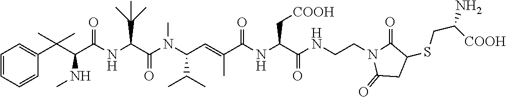 [SMILES: CN[C@H](C(=O)N[C@H](C(=O)N(C)[C@H](/C=C(\C)C(=O)N[C@@H](CC(=O)O)C(=O)NCCN1C(=O)CC(SC[C@H](N)C(=O)O)C1=O)C(C)C)C(C)(C)C)C(C)(C)c1ccccc1] | 830(M − H)⁻/0.830 | B |
| 46 | 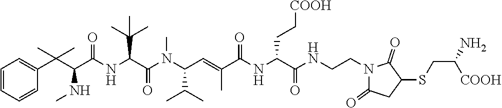 [SMILES: CN[C@H](C(=O)N[C@H](C(=O)N(C)[C@H](/C=C(\C)C(=O)N[C@H](CCC(=O)O)C(=O)NCCN1C(=O)CC(SC[C@H](N)C(=O)O)C1=O)C(C)C)C(C)(C)C)C(C)(C)c1ccccc1] | 844(M − H)⁻/0.808 | B |
| 47 | 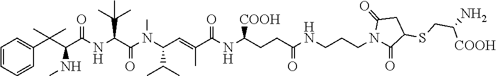 [SMILES: CN[C@H](C(=O)N[C@H](C(=O)N(C)[C@H](/C=C(\C)C(=O)N[C@H](CCC(=O)NCCCN1C(=O)CC(SC[C@H](N)C(=O)O)C1=O)C(=O)O)C(C)C)C(C)(C)C)C(C)(C)c1ccccc1] | 858(M − H)⁻/0.819 | B |
| 48 | 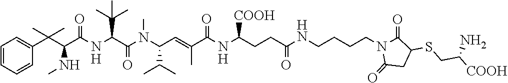 [SMILES: CN[C@H](C(=O)N[C@H](C(=O)N(C)[C@H](/C=C(\C)C(=O)N[C@H](CCC(=O)NCCCCN1C(=O)CC(SC[C@H](N)C(=O)O)C1=O)C(=O)O)C(C)C)C(C)(C)C)C(C)(C)c1ccccc1] | 872(M − H)⁻/0.819 | B |
| 49 | 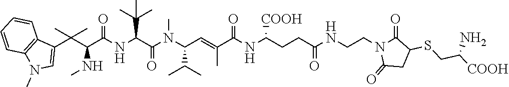 [SMILES: CN[C@H](C(=O)N[C@H](C(=O)N(C)[C@H](/C=C(\C)C(=O)N[C@@H](CCC(=O)NCCN1C(=O)CC(SC[C@H](N)C(=O)O)C1=O)C(=O)O)C(C)C)C(C)(C)C)C(C)(C)c1cn(C)c2ccccc12] | 897(M − H)⁻/0.866 | B |
| 50 | 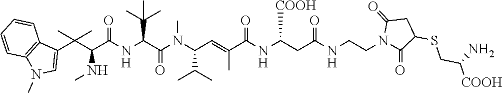 [SMILES: CN[C@H](C(=O)N[C@H](C(=O)N(C)[C@H](/C=C(\C)C(=O)N[C@@H](CC(=O)NCCN1C(=O)CC(SC[C@H](N)C(=O)O)C1=O)C(=O)O)C(C)C)C(C)(C)C)C(C)(C)c1cn(C)c2ccccc12] | 883(M − H)⁻/0.853 | B |
| 51 | 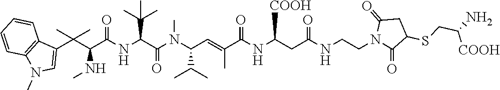 [SMILES: CN[C@H](C(=O)N[C@H](C(=O)N(C)[C@H](/C=C(\C)C(=O)N[C@H](CC(=O)NCCN1C(=O)CC(SC[C@H](N)C(=O)O)C1=O)C(=O)O)C(C)C)C(C)(C)C)C(C)(C)c1cn(C)c2ccccc12] | 883(M − H)⁻/0.843 | B |
| 52 | 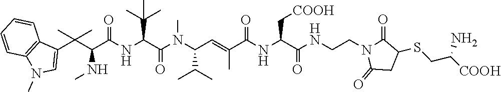 [SMILES: CN[C@H](C(=O)N[C@H](C(=O)N(C)[C@H](/C=C(\C)C(=O)N[C@@H](CC(=O)O)C(=O)NCCN1C(=O)CC(SC[C@H](N)C(=O)O)C1=O)C(C)C)C(C)(C)C)C(C)(C)c1cn(C)c2ccccc12] | 883(M − H)⁻/0.863 | B |
| 53 | 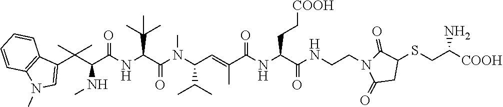 [SMILES: CN[C@H](C(=O)N[C@H](C(=O)N(C)[C@H](/C=C(\C)C(=O)N[C@@H](CCC(=O)O)C(=O)NCCN1C(=O)CC(SC[C@H](N)C(=O)O)C1=O)C(C)C)C(C)(C)C)C(C)(C)c1cn(C)c2ccccc12] | 897(M − H)⁻/0.865 | B |
| 54 | 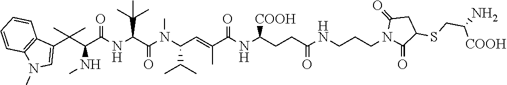 [SMILES: CN[C@H](C(=O)N[C@H](C(=O)N(C)[C@H](/C=C(\C)C(=O)N[C@H](CCC(=O)NCCCN1C(=O)CC(SC[C@H](N)C(=O)O)C1=O)C(=O)O)C(C)C)C(C)(C)C)C(C)(C)c1cn(C)c2ccccc12] | 911(M − H)⁻/0.868 | B |
| 55 | 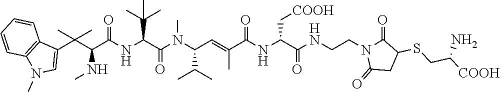 [SMILES: CN[C@H](C(=O)N[C@H](C(=O)N(C)[C@H](/C=C(\C)C(=O)N[C@H](CC(=O)O)C(=O)NCCN1C(=O)CC(SC[C@H](N)C(=O)O)C1=O)C(C)C)C(C)(C)C)C(C)(C)c1cn(C)c2ccccc12] | 883(M − H)⁻/0.876 | B |
| 56 | 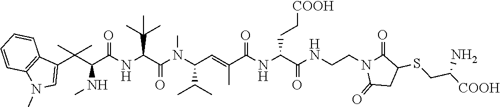 [SMILES: CN[C@H](C(=O)N[C@H](C(=O)N(C)[C@H](/C=C(\C)C(=O)N[C@H](CCC(=O)O)C(=O)NCCN1C(=O)CC(SC[C@H](N)C(=O)O)C1=O)C(C)C)C(C)(C)C)C(C)(C)c1cn(C)c2ccccc12] | 897(M − H)⁻/0.877 | B |
| 57 | 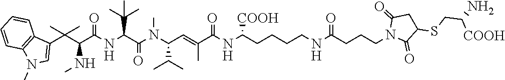 [SMILES: CN[C@H](C(=O)N[C@H](C(=O)N(C)[C@H](/C=C(\C)C(=O)N[C@@H](CCCCNC(=O)CCCN1C(=O)CC(SC[C@H](N)C(=O)O)C1=O)C(=O)O)C(C)C)C(C)(C)C)C(C)(C)c1cn(C)c2ccccc12] | 939(M − H)⁻/1.264 | B |
| 58 | 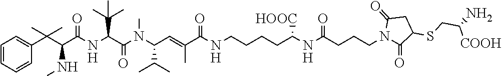 [SMILES: CN[C@H](C(=O)N[C@H](C(=O)N(C)[C@H](/C=C(\C)C(=O)NCCCC[C@@H](NC(=O)CCCN1C(=O)CC(SC[C@H](N)C(=O)O)C1=O)C(=O)O)C(C)C)C(C)(C)C)C(C)(C)c1ccccc1] | 886(M − H)⁻/0.842 | B |
| 59 | 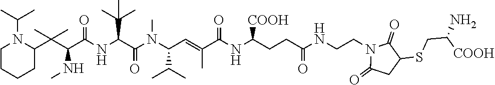 [SMILES: CN[C@H](C(=O)N[C@H](C(=O)N(C)[C@H](/C=C(\C)C(=O)N[C@H](CCC(=O)NCCN1C(=O)CC(SC[C@H](N)C(=O)O)C1=O)C(=O)O)C(C)C)C(C)(C)C)C(C)(C)C1CCCCN1C(C)C] | 808(M − H)⁻/0.741 | B |

An antibody used for producing Example ADCs is commercially available, or may be produced in accordance with the literature shown in the following table.

TABLE 12

| Antibody Name | Reference Literature |
|---|---|
| Brentuximab (Bremtuximab) | Japanese Patent No. 4303964 |

### Example ADC1

Brentuximab—Example 34 Conjugate (Average DAR: 7.41)

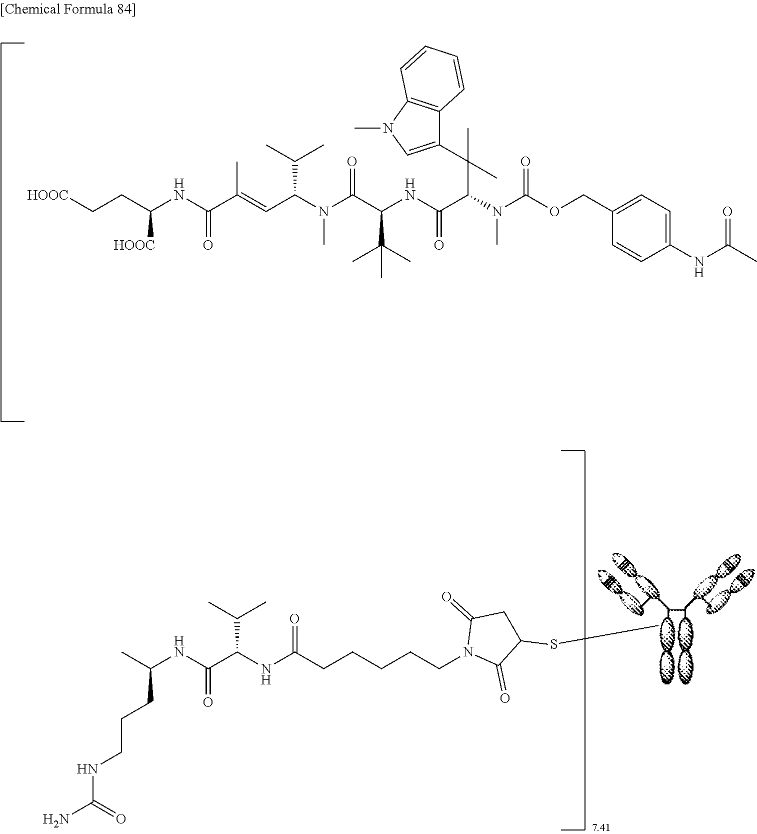

To a phosphate buffered saline solution (3.77 mL, pH 7.4) of brentuximab (91 mg), a trishydroxymethylaminomethane hydrochloride buffered solution (12.2 mL, pH 7.5) of 1 mmol/L tris(2-carboxyethyl)phosphine (TCEP) was added, and the resultant solution was incubated at 37° C. for 45 minutes. After cooling the antibody solution to 0° C., through treatment with a PD-10 desalination column pre-equilibrated with a phosphate buffered saline solution (pH 7.4), a phosphate buffered saline solution (pH 7.4) of the reduced anti-CD30 antibody (brentuximab) was obtained. After cooling this to 0° C., a 1 mmol/L DMSO solution of Example 34 (12.2 mL) 10 times diluted with a phosphate buffered saline solution (pH 7.4) was added as a modifying agent and completely mixed, and the resultant solution was incubated at 4° C. for 16 hours. Thereafter, through purification by a PD-10 desalination column pre-equilibrated with a phosphate buffered saline solution (pH 7.4) and subsequent centrifugal concentration, Example ADC1 (78.2 mg) was obtained.

The average DAR of the ADC thus obtained was measured by reducing or non-reducing SDS-PAGE, or HPLC-HIC. Alternatively, the average DAR may be measured qualitatively or quantitatively by ultraviolet-visible absorption spectroscopy (UV-Vis), reducing or non-reducing SDS-PAGE, HPLC-HIC, SEC, RP-HPLC, LC-MS or the like. These methods are described in Antibody Drug Conjugates, Methods in Molecular Biology vol. 1045, 2013. pp 267-284. L. Ducry, Ed.

When the average drug antibody ratio of an ADC produced with a human IgG₁ antibody is 8, production of the ADC may also be assumed from the results of reducing and non-reducing SDS-PAGE. Specifically, when bands are strongly detected in the vicinity of a molecular weight of 50 kDa and a molecular weight of 25 kDa as a result of SDS-PAGE analysis for Example ADC under disulfide non-reducing conditions, using SeeBlue (R) Plus2 (Thermo Fisher Scientific K.K.) as a marker, this indicates that the modifying agent conjugates to the cysteine residues involved in the disulfide bonds between the light chains and heave chains and of the hinge of the antibody, which means that an ADC with an average drug antibody ratio of 8 is obtained.

The average DAR of Example ADC1, determined from HPLC-HIC analysis, was 7.41.

### Example ADC2

### Brentuximab—Example 34 Conjugate (Average DAR: 3.76)

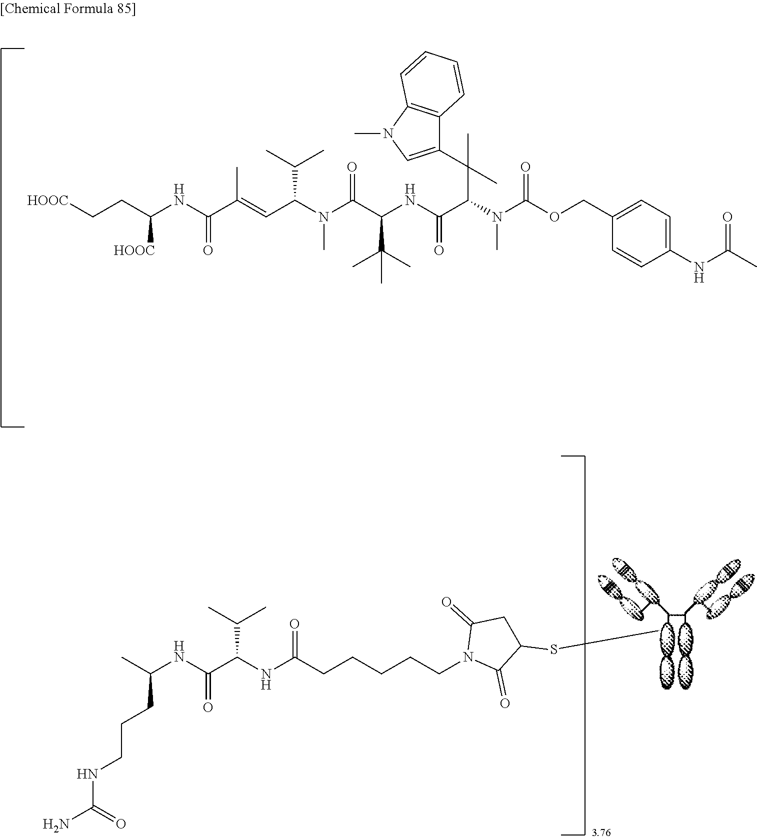

In the protocol of Example ADC 1, by changing the amount of TCEP or the modifying agent to be added, the DAR of the ADC may be adjusted. In accordance with the protocol of Example ADC1, Example ADC 2 was obtained by using TCEP in an amount of 4 molar equivalent.

### Examples ADC3 to 26

The ADCs shown in the following were obtained through the same reaction and treatment as Example ADC1, using corresponding antibodies and modifying agents. In addition, the average DARs of these ADCs were calculated or assumed from UV-Vis, HPLC-HIC or SDS-PAGE analysis in the same manner as Example ADC1. [Chemical Formula 86]

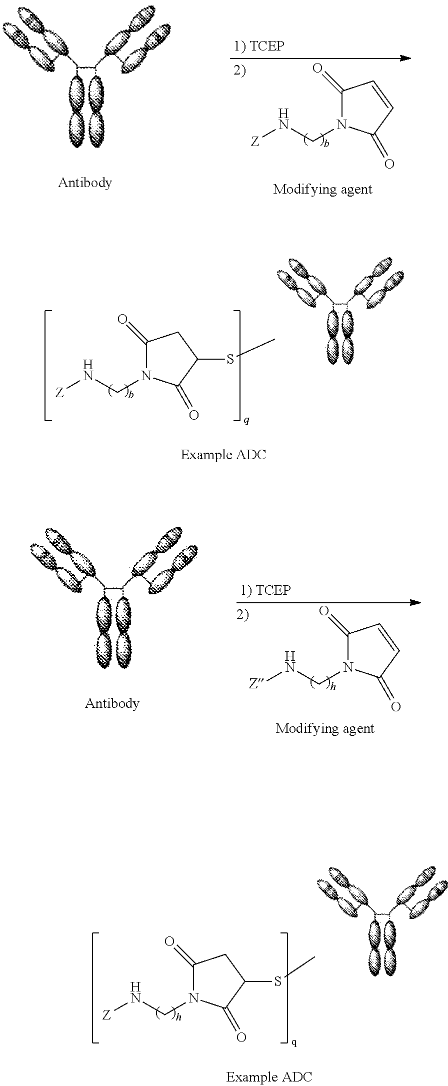

TABLE 13

| | | | Average | HIC | HIC retention |
|---|---|---|---|---|---|
| Example | Antibody | Modifying agent | DAR | condition | time (min) |
| Comparative | Brentuximab | Comparative | 4.12 | I | 9.94 |
| Example | | Example compound | | | |
| ADC1 | | 1 | | | |
| ADC3 | Brentuximab | Example 33 | Production of ADC was confirmed by | | |
| | | | SDS-PAGE | | |
| ADC4 | Brentuximab | Example 18 | 8.00 | I | 5.94 |
| ADC5 | Brentuximab | Example 21 | 6.79 | I | 6.01 |
| ADC6 | Brentuximab | Example 24 | 8 | I | 5.72 |
| ADC7 | Brentuximab | Example 20 | 8 | I | 7.08 |
| ADC8 | Brentuximab | Example 22 | 8 | I | 5.77 |
| ADC9 | Brentuximab | Example 26 | 8 | I | 6.02 |
| ADC10 | Brentuximab | Example 27 | 8 | I | 5.45 |
| ADC11 | Brentuximab | Example 25 | 8 | I | 5.88 |
| ADC12 | Brentuximab | Example 23 | 8 | I | 5.69 |
| ADC13 | Brentuximab | Example 29 | 7.03 | I | 5.89 |
| ADC14 | Brentuximab | Example 30 | 8 | I | 6.03 |
| ADC15 | Brentuximab | Example 31 | 7.76 | I | 6.08 |
| ADC16 | Brentuximab | Example 16 | 7.70 | I | 6.80 |
| ADC17 | Brentuximab | Example 12 | 7.82 | I | 6.56 |
| ADC18 | Brentuximab | Example 14 | 7.73 | I | 6.6 |
| ADC19 | Brentuximab | Example 13 | 7.75 | I | 6.54 |
| ADC20 | Brentuximab | Example 17 | 7.74 | I | 6.57 |
| ADC21 | Brentuximab | Example 15 | 7.64 | I | 6.67 |
| ADC22 | Brentuximab | Example 28 | 4.6 | J | — |
| ADC23 | Brentuximab | Example 28 | 8.00 | I | 4.89 |
| ADC24 | Brentuximab | Example 19 | 7.7 | I | 6.73 |
| ADC25 | Brentuximab | Example 11 | 7.5 | I | 6.79 |
| ADC26 | Brentuximab | Example 32 | 8 | I | 3.80 |

“Production of ADC was confirmed by SDS-PAGE” means that bands were strongly detected in the vicinity of a molecular weight of 50 kDa and a molecular weight of 25 kDa as a result of SDS-PAGE analysis for Example ADC under disulfide non-reducing conditions, using SeeBlue (R) Plus2 (Thermo Fisher Scientific K.K.) as a marker. This indicates that the modifying agent conjugates to the cysteine residues involved in the disulfide bonds between the light chains and heavy chains and of the hinge of the antibody, which means that an ADC is obtained.

The HIC retention time (min) of the Example ADCs in the above Table 13 is that of the peak of ADCs with a DAR of 8, observed by HPLC-HIC analysis. The Rt (min) of the ADC of Comparative Example 1 is that of the peak of ADC with a DAR of 8.

Comparative Example compound 1 in the above Table 13 represents the following compound disclosed in International Publication No. WO 2004/010957. Comparative Example compound 1 is called vedotin.

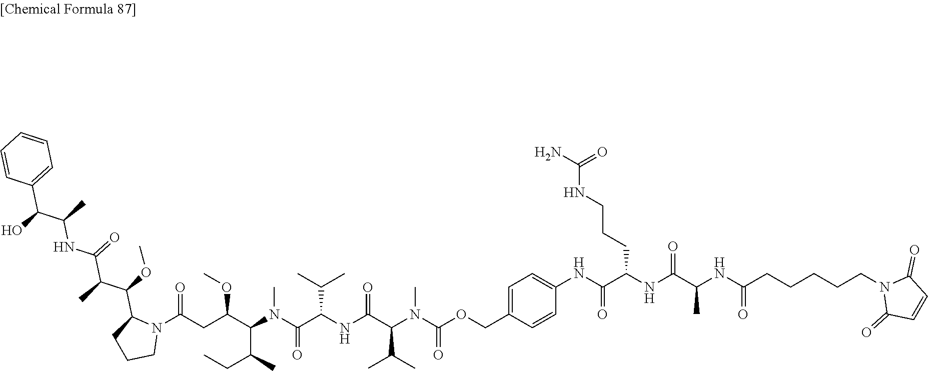 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](C)NC(=O)CCCCCN2C(=O)C=CC2=O)cc1)C(C)C]

Comparative Example ADC1 in the above Table 13 corresponds to brentuximab vedotin, which is an antibody-drug conjugate of Comparative Example compound 1 and brentuximab. Brentuximab vedotin has been pharmaceutically approved in Japan as Adcetris (product name).

Comparative Example compound 2 is monomethyl auristatin (MMAE), and may be purchased as a reagent. Brentuximab vedotin is known to release MMAE through undergoing metabolism in cells (Non Patent Literature 5).

Hereinafter, results of pharmacological tests with respect to particular Examples of antibody-drug conjugate according to the present invention will be shown and its pharmacological actions will be explained, but the present invention is not limited to these Test Examples.

### Test Example 1: Evaluation of Activity for Inhibiting Microtubule Polymerization Using Porcine Tubulins (1)

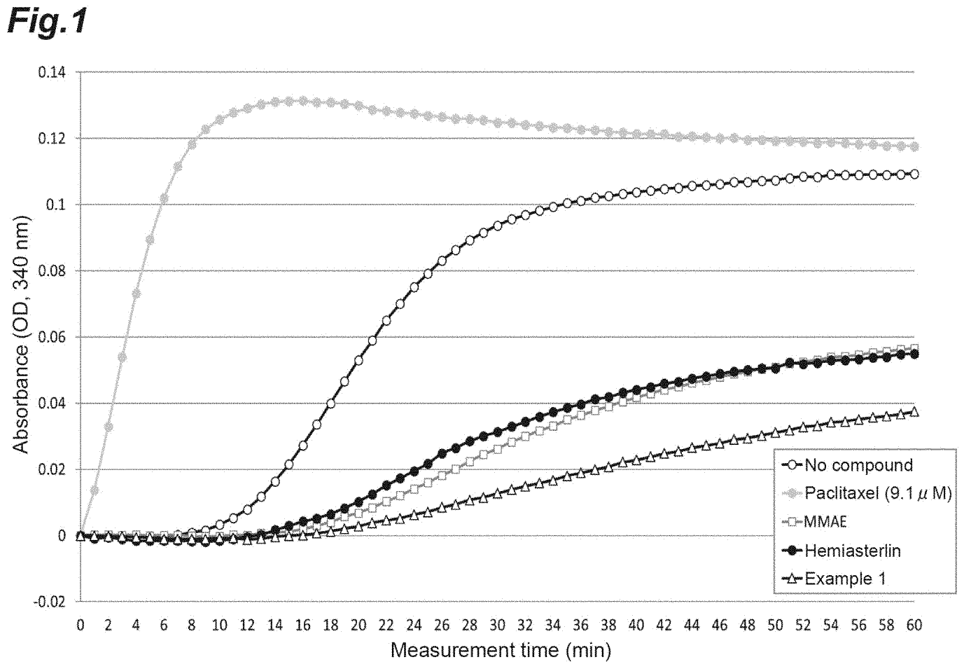

Using a tubulin polymerization inhibition assay kit (catalog number: BK006P) purchased from Cytoskeleton Inc., the polymerization inhibitory activity of compounds of Examples with a concentration of 0.91 μM was evaluated in accordance with the protocol appended to the kit. In summary of the protocol, to a 96 well microplate, 80 mM PIPES pH 6.9, 2 mM MgCl, 0.5 mM EGTA and 5% DMSO buffered solution of the compound to be evaluated was added in an amount of 10 μL for each well, and to these wells, 3 mg/mL porcine tubulin 80 mM PIPES pH 6.9, 2 mM MgCl, 0.5 mM EGTA, 1 mM GTP and 10.2% glycerol solution was added in an amount of 100 μL for each well. In order to examine a state in which tubulins polymerize over time, the absorbance at 340 nm was measured at 37° C., using a microplate reader. As the polymerization of tubulins progresses, the absorbance at 340 nm rises. The results are shown in FIG. 1.

As shown in FIG. 1, monomethyl auristatin (MMAE), hemiasterlin and Example 1 exhibited comparable activities for inhibiting microtubule polymerization in the microtubule polymerization inhibition evaluation test.

### Test Example 2: Evaluation of Activity for Inhibiting Microtubule Polymerization Using Porcine Tubulins (2)

Using a tubulin polymerization inhibition assay kit (catalog number: BK006P) purchased from Cytoskeleton Inc., the polymerization inhibitory activity of compounds of Examples at a concentration of 9.1 μM was evaluated in accordance with the protocol appended to the kit. To a 96 well microplate, 80 mM PIPES pH 6.9, 2 mM MgCl, 0.5 mM EGTA and 5% DMSO buffered solution of the compound to be evaluated was added in an amount of 10 μL for each well, and to these wells, 3 mg/mL porcine tubulin 80 mM PIPES pH 6.9, 2 mM MgCl, 0.5 mM EGTA, 1 mM GTP and 10.2% glycerol solution was added in an amount of 100 μL for each well. In order to examine a state in which tubulins polymerize over time, the absorbance at 340 nm was measured at 37° C., using a microplate reader. As the polymerization of tubulins progresses, the absorbance at 340 nm rises.

The tubulin polymerization inhibitory activity was evaluated based on the proportion of polymerized tubulins 60 minutes after the assay initiation. Specifically, the microtubule polymerization rate (%) was calculated by dividing the absorbance of tubulins that had polymerized at wells to which the compound had been added by the absorbance of tubulins that had polymerized at wells to which the compound had not been added, and multiplying the obtained value by 100. The results are shown in the following Table 14.

TABLE 14

| | Microtubule |
|---|---|
| Compound | polymerization rate (%) |
| Hemiasterlin | 0 |
| Example 2 | 2 |
| Example 3 | 5 |
| Example 6 | 4 |
| Example 9 | 5 |
| Example 37 | 9 |
| Example 39 | 2 |
| Example 42 | 0 |
| Example 46 | 0 |
| Example 48 | 0 |
| Example 49 | 0 |
| Example 54 | 0 |
| Example 58 | 0 |

It is indicated that the lower value the microtubule polymerization rate is, the more strongly the compound inhibits polymerization of microtubules.

As shown from the results of Test Example 1 and Test Example 2, it was revealed that the hemiasterlin derivatives according to the present invention, which are produced through metabolism of an antibody-drug conjugate in target cells and represented by formula (1-1), formula (1-2) and formula (1-3), exhibit tubulin polymerization inhibitory activity.

### Test Example 3: Evaluation of Cytotoxic Activity to iPS Cells

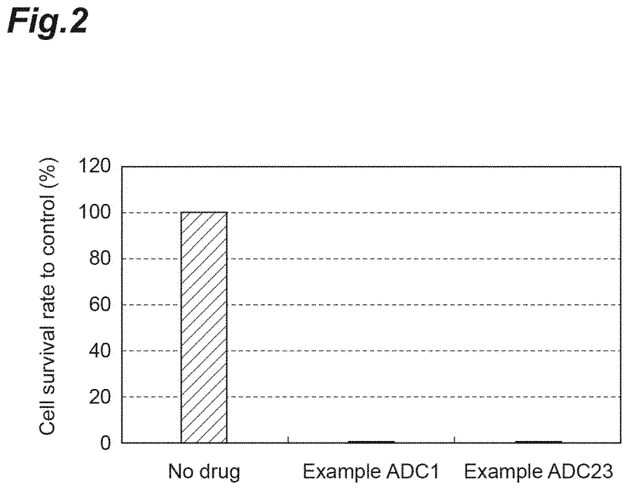

Human iPS cells (201B7) were feeder-free cultured in accordance with the method described in Scientific Reports, 4, 3594 (2014). StemFit medium (AK03N, manufactured by Ajinomoto Co., Inc.) was used as feeder-free culture medium, and iMatrix-511 (manufactured by Nippi, Incorporated) was used as a feeder-free scaffold. Human iPS cells that had become subconfluent were washed with PBS, and then dispersed into single cells by using TrypLE Select (manufactured by Life Technologies). Thereafter, these human iPS cells were seeded on a plastic culture dish coated with iMatrix-511, and feeder-free cultured in StemFit medium in the presence of Y27632 (ROCK inhibitor, 10 μmol/L) at 37° C. under 5% CO₂. At that time, a 12 well plate (manufactured by AGC TECHNO GLASS CO., Ltd., for cell culturing, culture area: 3.8 cm²) was used as the plastic culture dish, and the number of seeded cells of the human iPS cells dispersed into single cells was 0.5×10⁴ cells. One day after seeding, the culture medium was replaced with StemFit medium without Y27632, and culture medium replacement was further performed in the same manner 3 days after seeding. Four days after seeding, the compounds of Example ADC1 and Example ADC23 each dissolved in PBS were added to StemFit medium (AK03, manufactured by Ajinomoto Co., Inc.) with the human iPS cells to reach a final concentration of 200 μg/mL, and culture was performed for 72 hours. After 72 hours, the culture medium was removed, and the cells were washed with PBS, then dispersed into single cells by using TrypLE Select (manufactured by Life Technologies), and stained with 0.4% trypan blue solution (manufactured by Life Technologies) attached to a Countess Automated Cell Counter, and thereafter viable cell counts were measured by the Countess Automated Cell Counter and cell survival rates relative to that of a control were calculated. The results are shown in FIG. 2.

As shown in FIG. 2, strong cytotoxic activity to iPS cells was found for both the compounds of Example ADC1 and Example ADC23 at a concentration of 200 μg/mL. The cell survival rate was 1.2% or less for both cases.

### Test Example 4: Evaluation of Cytotoxic Activity to Differentiated Cells

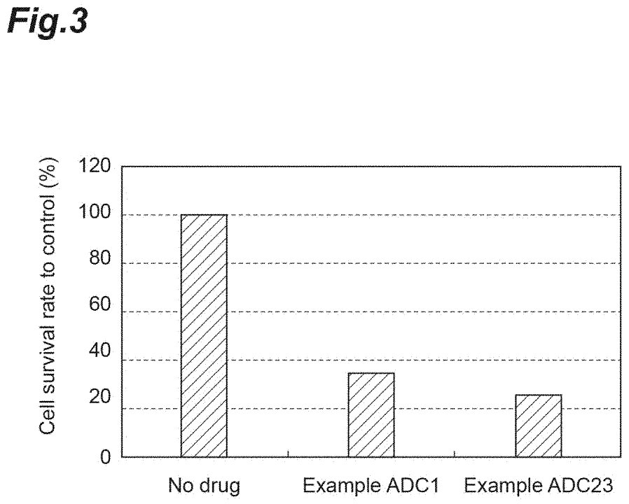

In the same manner as in Test Example 3, feeder-free cultured human iPS cells (201B7) 1 day before becoming subconfluent were dispersed into single cells by using TrypLE Select (manufactured by Life Technologies). Thereafter, these human iPS cells were seeded on a plastic culture dish coated with Vitronectin, truncated recombinant human (manufactured by Life Technologies), and feeder-free cultured in StemFit medium in the presence of Y27632 (ROCK inhibitor, 10 μmol/L) at 37° C. under 5% CO₂. At that time, a 6 well plate (manufactured by AGC TECHNO GLASS CO., Ltd., for cell culturing, culture area: 9.4 cm²) was used as the plastic culture dish, and the number of seeded cells of the human iPS cells dispersed into single cells was 2.5×10⁵ cells. Thereafter, the culture medium was replaced with Definitive Endoderm Induction Medium A attached to a PSC Definitive Endoderm Induction Kit (manufactured by Gibco) and culture was performed for 1 day, and the culture medium was further replaced with Definitive Endoderm Induction Medium B and culture was performed for 1 day. Thereafter, the cells were dispersed into single cells by using TrypLE Select (manufactured by Life Technologies), seeded on a plastic culture dish coated with Hepatocyte Coating attached to a Cellartis Hepatocyte Differentiation Kit (manufactured by Takara Bio Inc.), and cultured at 37° C. under 5% CO₂ for 1 day. At that time, a 24 well plate (manufactured by AGC TECHNO GLASS CO., Ltd., for cell culturing, culture area: 2 cm²) was used as the plastic culture dish, and the number of seeded cells was 2.5×10⁵ cells. Two days after seeding, the compounds of Example ADC1 and Example ADC23 each dissolved in PBS were added to Hepatocyte Progenitor Medium attached to the kit to reach a final concentration of 200 μg/mL, and culture was performed for 72 hours. After 72 hours, the culture medium was removed, and the cells were washed with PBS, then dispersed into single cells by using TrypLE Select (manufactured by Life Technologies), and stained with trypan blue solution (manufactured by Life Technologies) attached to a Countess Automated Cell Counter, and thereafter viable cell counts were measured by the Automated Cell Counter and cell survival rates relative to that of a control were calculated. The results are shown in FIG. 3.

As shown in FIG. 3, weak toxic activity to differentiation-induced cells was found for the compounds of Example ADC1 and Example ADC23 at a concentration of 200 μg/mL. The cell survival rate was 34.7% for Example ADC1, and 24.6% for Example ADC23.

### Test Example 5: Membrane Permeability Test

By the parallel artificial membrane permeability assay (PAMPA), the membrane permeability of compounds of Examples was examined as follows: To the donor plate, System solution (pION inc.) and GIT Lipid-0 (pION inc.) were added in an amount of 200 μL and 4 μL for each well, respectively. To the acceptor plate, Acceptor Sink Buffer (pION inc.) was added in an amount of 200 μL. Both plates were superposed and incubated at 37° C. for 4 hours, and then, UVs in the solutions on the side of acceptor and on the side of donor were measured with a UV plate reader (190 to 500 nm). Compounds with poor UV absorption were measured by LC-MS. The permeability coefficient Pₑ (10⁻⁶ cm/sec) of the drug was calculated by the following formula. The results are shown in Table 15.

$${P}_{e} = - \frac{2.303 {V}_{D}}{A ( t - {τ}_{SS} )} ( \frac{1}{1 + \text{?}} ) \cdot {log}_{10} [ - \text{?} + ( \frac{1 + \text{?}}{1 - R} ) \cdot \frac{{C}_{D} ( t )}{{C}_{D} ( 0 )} ] \\ \text{?} = ( {V}_{D} / {V}_{A} ) {P}_{e}^{( A - D )} / {P}_{e}^{( D - A )} = {r}_{V} {P}_{e}^{( A - D )} / {P}_{e}^{( D - A )} \\ \text{?} = ( {V}_{D} / {V}_{A} ) \\ {V}_{D} = volume of donor well \\ {V}_{A} = volume of acceptor well \\ t = permeation time \\ {τ}^{SS} = steady state time \\ R - retention \\ {C}_{D} and {C}_{A} = concentration in donor and acceptor well \\ \text{?} \text{indicates text missing or illegible when filed} \qquad [ Expression 1 ]$$

TABLE 15

| | Pe(10⁻⁶ cm/sec) |
|---|---|
| Compound | (pH 7.4) |
| MMAE | 15.3 |
| Hemiasterlin | 25.6 |
| Example 1 | <0.1 |
| Example 2 | <0.1 |
| Example 3 | <0.1 |
| Example 5 | <0.1 |
| Example 6 | <0.1 |
| Example 7 | 0.8 |
| Example 9 | <0.1 |
| Example 37 | 0.2 |
| Example 39 | 1.4 |
| Example 40 | 3.0 |
| Example 41 | <0.1 |
| Example 42 | 2.2 |
| Example 46 | 3.5 |
| Example 48 | 5.8 |
| Example 49 | <0.1 |
| Example 54 | <0.1 |
| Example 57 | <0.1 |
| Example 58 | 3.0 |

From the results of Test Example 5, it was revealed that the compounds of Examples represented by formula (1-1), formula (1-2) and formula (1-3) have lower cell membrane permeability than monomethyl auristatin (MMAE) and hemiasterlin.

From the results of Test Examples 1 to 5, it is expected that when any of the antibody-drug conjugates represented by formula (2-1) and formula (2-2) is allowed to act on coexisting iPS cells and differentiated cells, the antibody-drug conjugate exhibits cellular toxicity selectively to iPS cells. The compound produced through metabolism of the antibody-drug conjugate according to the present invention taken up by iPS cells has low cell membrane permeability, and hence the migration into differentiated cells can be hindered. Therefore, the antibody-drug conjugate according to the present invention can be expected to have lower cellular toxicity to differentiated cells than to iPS cells. Accordingly, after inducing differentiation of an iPS cell group, the antibody-drug conjugate according to the present invention allows selective elimination of remaining iPS cells from a heterogeneous state of differentiated cells and remaining iPS cells, and thus differentiated cells can be efficiently obtained.

### INDUSTRIAL APPLICABILITY

As explained above, the antibody-drug conjugate according to the present invention exhibits cytotoxic activity selectively to undifferentiated iPS cells and in contrast has low cellular toxicity to differentiated cells, and therefore, is expected to serve as a cell-specific agent for eliminating a pluripotent stem cell.

1-35. (canceled)

36: A method for eliminating a pluripotent stem cell, comprising:
a step of adding the antibody-drug conjugate that releases a compound represented by formula (1-1) or formula (1-3): 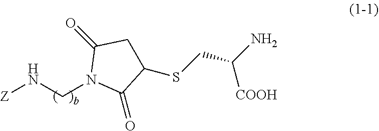
wherein
b represents an integer of 1 to 5; and
Z is a group represented by formula (Z-1) or formula (Z-2): 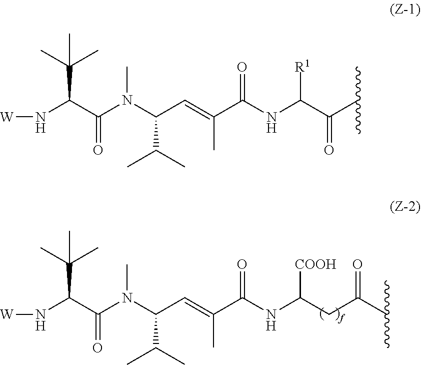
where
W represents a group represented by formula (W-1) or formula (W-2): 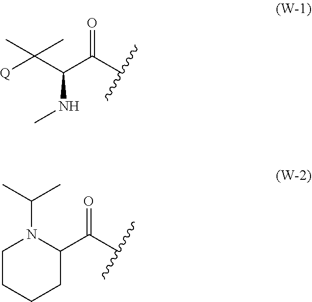
where
Q represents a group represented by formula (Q-1) or formula (Q-2): 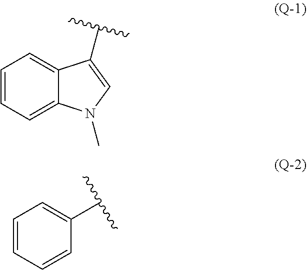 [SMILES: *c1cn(C)c2ccccc12 ; *c1ccccc1]
f represents 1 or 2;
R¹ represents —(CH₂)ᵤ—COOH; and
u represents 1 or 2; 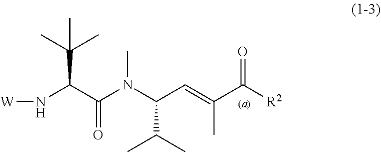
wherein
R² represents a glutamic acid residue (Glu), an aspartic acid residue (Asp) or a lysine residue (Lys);
an N-terminal nitrogen atom of R² forms an amide bond together with carbonyl (a); and
W is a group represented by formula (W-1) or formula (W-2): 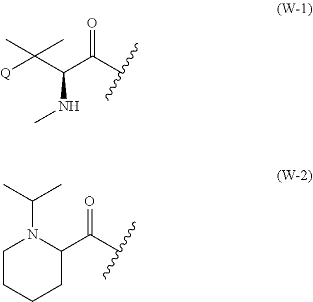
where
Q represents a group represented by formula (Q-1) or formula (Q-2): 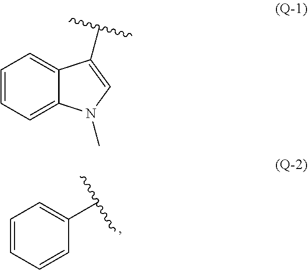 [SMILES: *c1cn(C)c2ccccc12 ; *c1ccccc1]
or a salt thereof to a culture solution containing a pluripotent stem cell.

37: The method for eliminating a pluripotent stem cell according to claim 36, wherein
W is a group represented by formula (W-1).

38: The method for eliminating a pluripotent stem cell according to claim 36, wherein
an antibody-drug conjugate releases the compound selected from the following compounds: 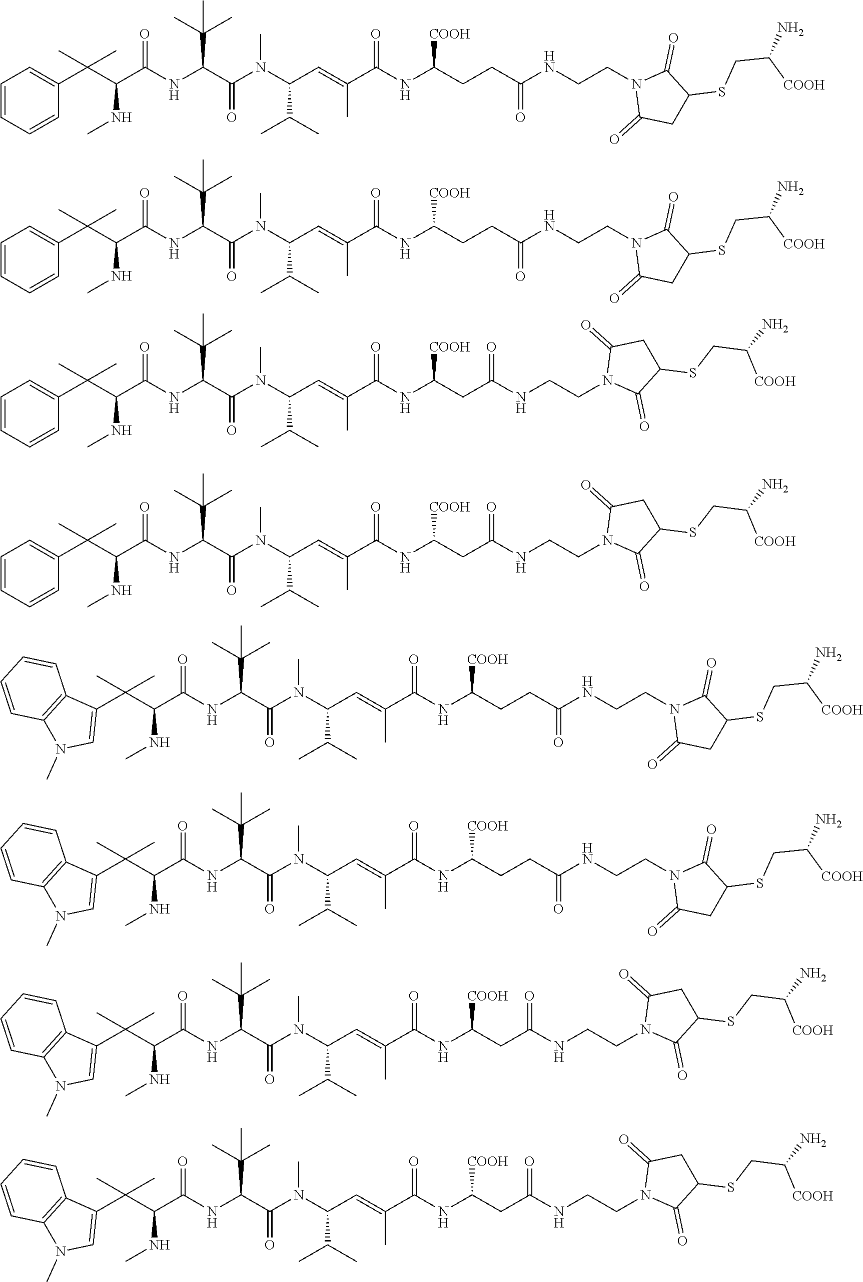
or a salt thereof.

39: The method for eliminating a pluripotent stem cell according to claim 36, wherein
an antibody-drug conjugate releases the compound selected from the following compounds: 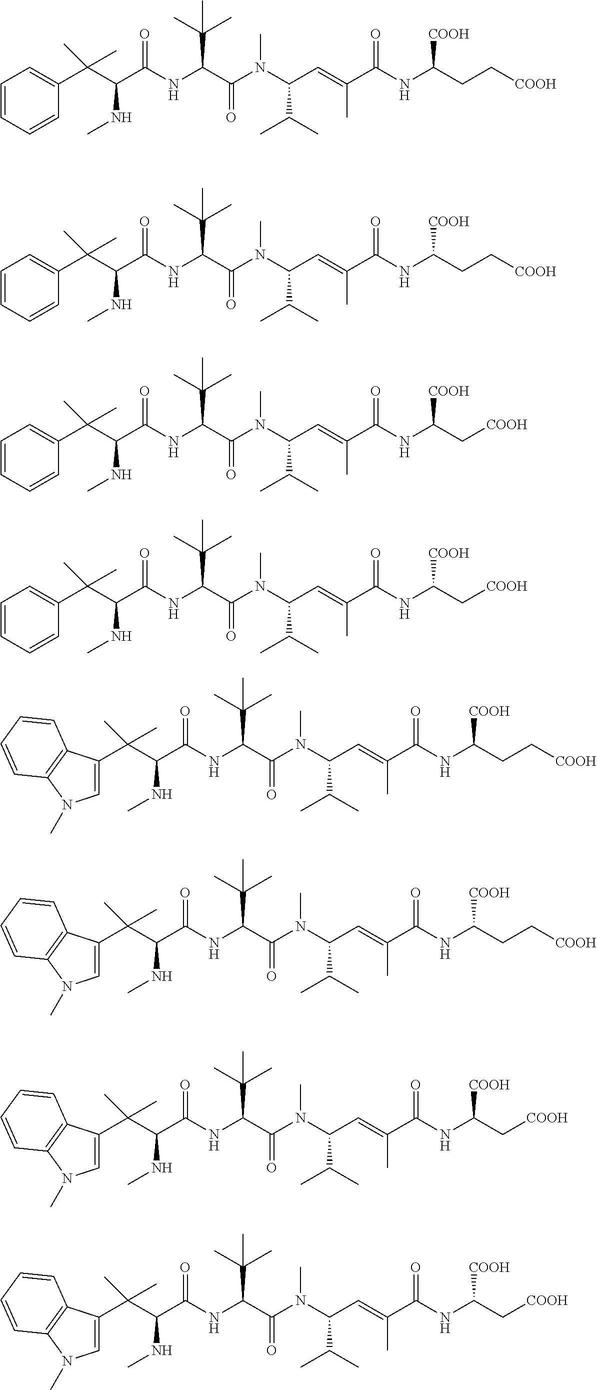 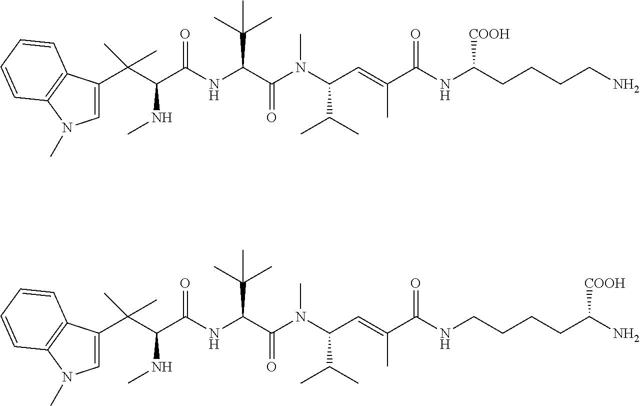 [SMILES: CN[C@H](C(=O)N[C@H](C(=O)N(C)[C@H](/C=C(\C)C(=O)N[C@@H](CCCCN)C(=O)O)C(C)C)C(C)(C)C)C(C)(C)c1cn(C)c2ccccc12 ; CN[C@H](C(=O)N[C@H](C(=O)N(C)[C@H](/C=C(\C)C(=O)NCCCC[C@@H](N)C(=O)O)C(C)C)C(C)(C)C)C(C)(C)c1cn(C)c2ccccc12]
or a salt thereof.

40: The method for eliminating a pluripotent stem cell according to claim 36, wherein the pluripotent stem cell is an ES cell or an PS cell.

41: The method for eliminating a pluripotent stem cell according to claim 36, wherein the pluripotent stem cell is an iPS cell.

42: A method for eliminating a pluripotent stem cell, comprising:
a step of adding the antibody-drug conjugate that releases a compound represented by formula (1-1) or formula (1-3): 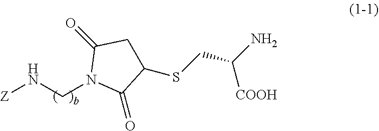
wherein
b represents an integer of 1 to 5; and
Z is a group represented by formula (Z-1) or formula (Z-2): 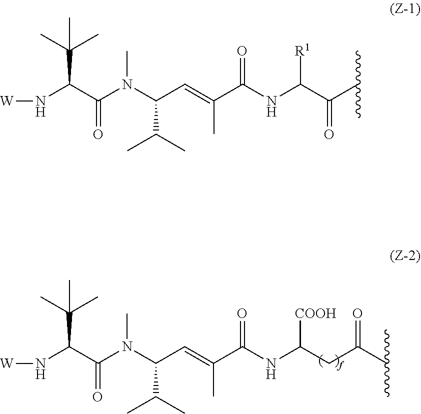
where
W represents a group represented by formula (W-1) or formula (W-2): 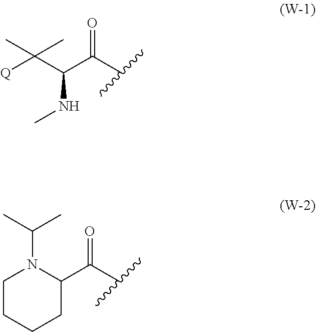
where
Q represents a group represented by formula (Q-1) or formula (Q-2): 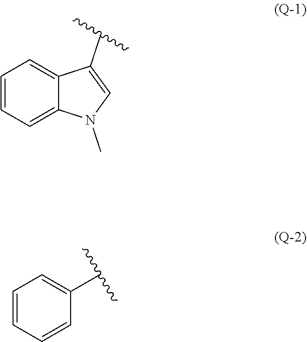 [SMILES: *c1cn(C)c2ccccc12 ; *c1ccccc1]
f represents 1 or 2;
R¹ represents —(CH₂)ᵤ—COOH; and
u represents 1 or 2; 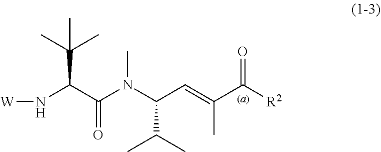
wherein
R² represents a glutamic acid residue (Glu), an aspartic acid residue (Asp) or a lysine residue (Lys);
an N-terminal nitrogen atom of R² forms an amide bond together with carbonyl (a); and
W is a group represented by formula (W-1) or formula (W-2): 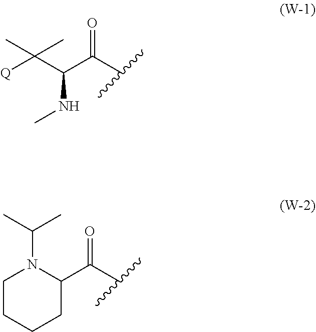
where
Q represents a group represented by formula (Q-1) or formula (Q-2): 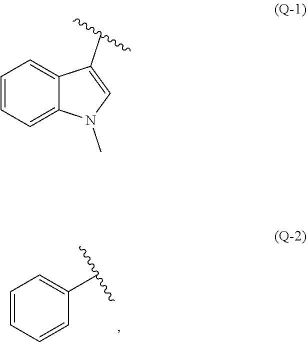 [SMILES: *c1cn(C)c2ccccc12 ; *c1ccccc1]
or a salt thereof to a culture solution containing a cell cluster produced by culturing a pluripotent stem cell.

43: The method for eliminating a pluripotent stem cell according to claim 42, wherein the pluripotent stem cell is an ES cell or an PS cell.

44: The method for eliminating a pluripotent stem cell according to claim 42, wherein the pluripotent stem cell is an iPS cell.

45: A method for producing a cell population including a differentiated cell derived from a pluripotent stem cell with substantially no pluripotent stem cell, comprising:
a step of contacting an antibody-drug conjugate that releases a compound represented by formula (1-1) or formula (1-3): 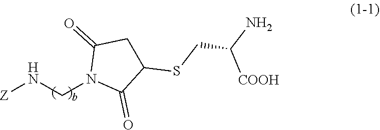
wherein
b represents an integer of 1 to 5; and
Z is a group represented by formula (Z-1) or formula (Z-2): 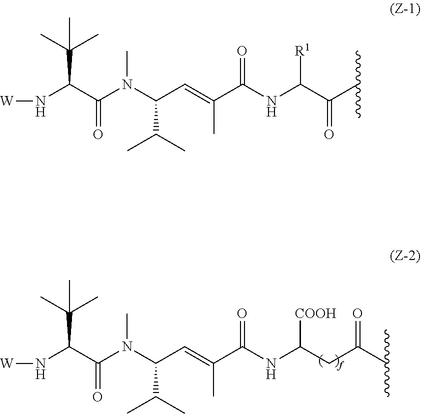
where
W represents a group represented by formula (W-1) or formula (W-2): 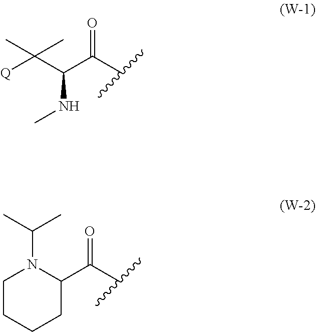
where
Q represents a group represented by formula (Q-1) or formula (Q-2): 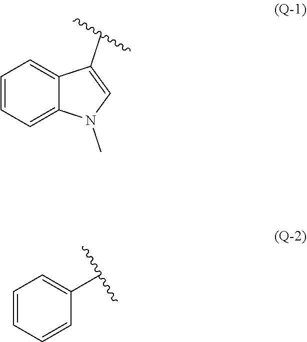 [SMILES: *c1cn(C)c2ccccc12 ; *c1ccccc1]
f represents 1 or 2;
R¹ represents —(CH₂)ᵤ—COOH; and
u represents 1 or 2; 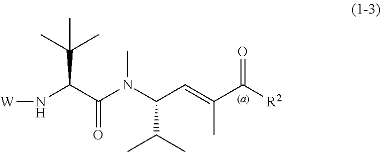
wherein
R² represents a glutamic acid residue (Glu), an aspartic acid residue (Asp) or a lysine residue (Lys);
an N-terminal nitrogen atom of R² forms an amide bond together with carbonyl (a); and
W is a group represented by formula (W-1) or formula (W-2): 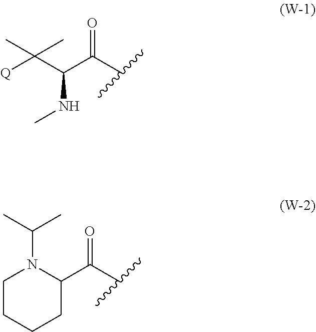
where
Q represents a group represented by formula (Q-1) or formula (Q-2): 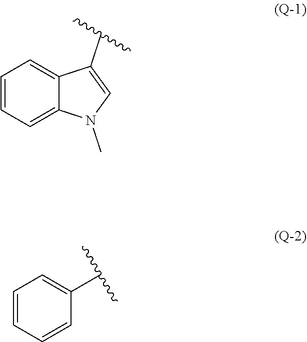 [SMILES: *c1cn(C)c2ccccc12 ; *c1ccccc1]
or a salt thereof and
a cell population including a differentiated cell derived from a pluripotent stem cell.

46: The method for eliminating a pluripotent stem cell according to claim 45, wherein
W is a group represented by formula (W-1).

47: The method for producing a cell population according to claim 45, wherein
an antibody-drug conjugate releases the compound selected from the following compounds: 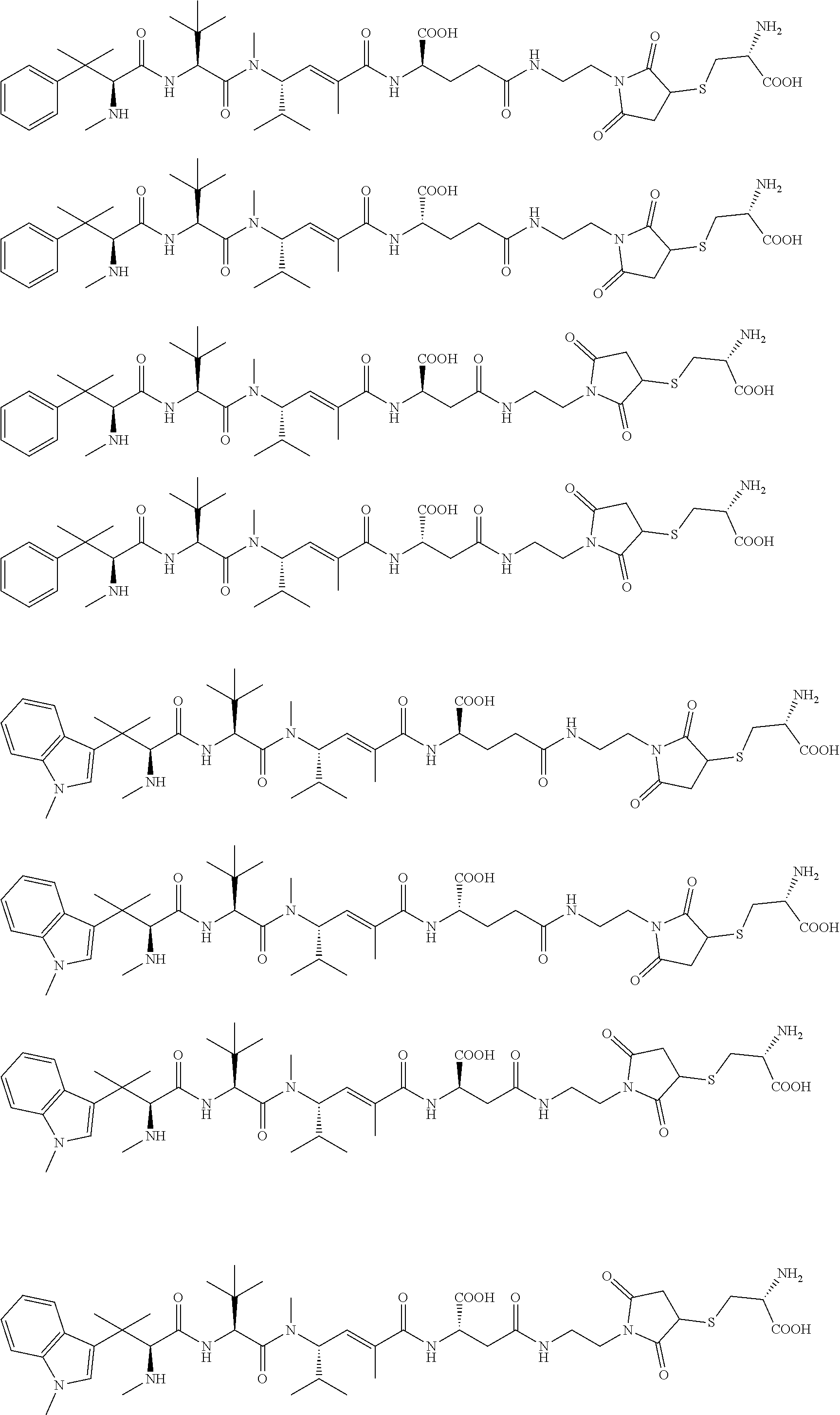
or a salt thereof.

48: The method for producing a cell population according to claim 45, wherein
an antibody-drug conjugate releases the compound selected from the following compounds: 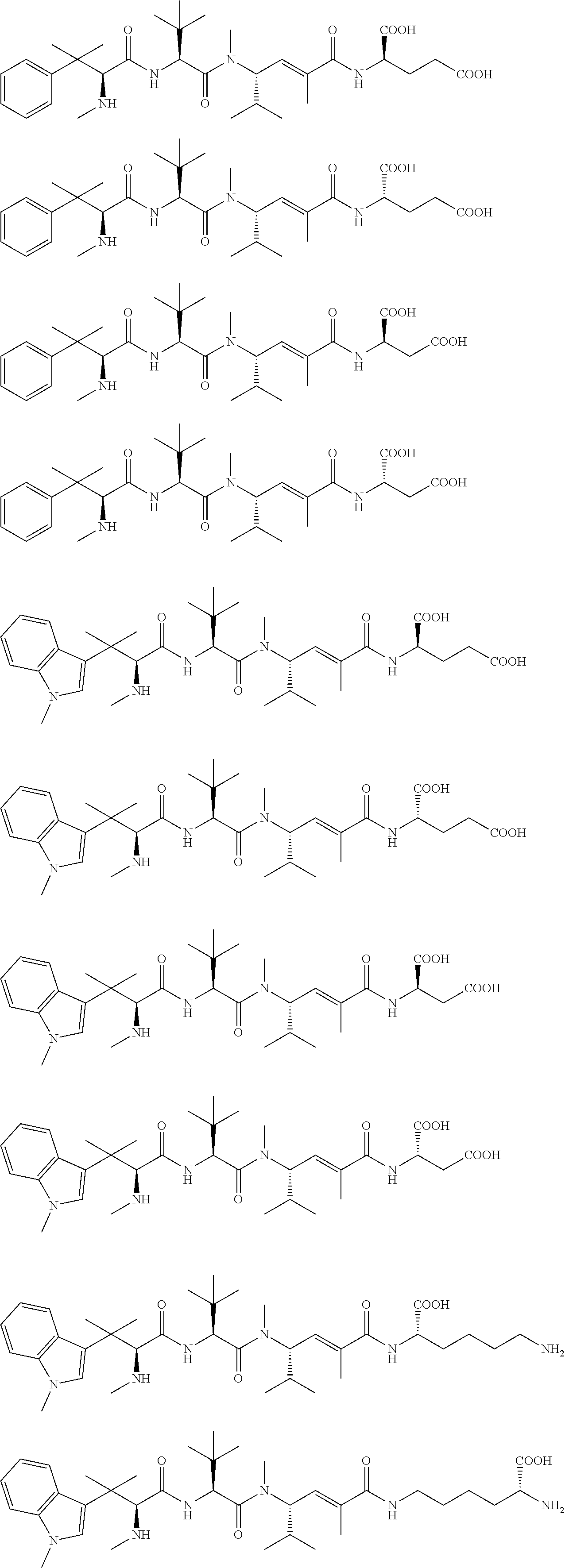
or a salt thereof.

49: A method for producing a cell population including a differentiated cell derived from an PS cell with substantially no PS cell, comprising:
a step of contacting an antibody-drug conjugate that releases a compound represented by formula (1-1) or formula (1-3): 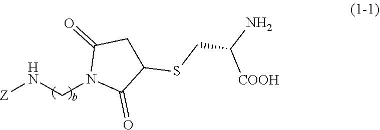
wherein
b represents an integer of 1 to 5; and
Z is a group represented by formula (Z-1) or formula (Z-2): 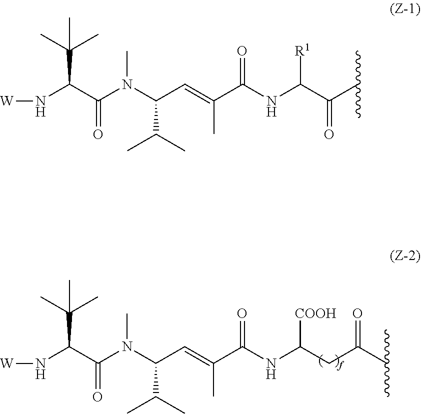
where
W represents a group represented by formula (W-1) or formula (W-2): 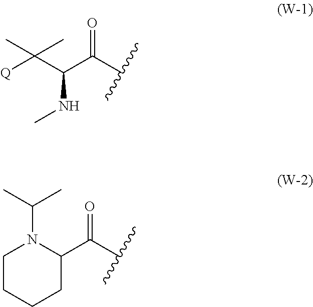
where
Q represents a group represented by formula (Q-1) or formula (Q-2): 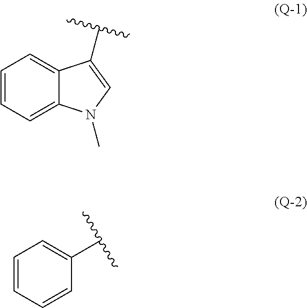 [SMILES: *c1cn(C)c2ccccc12 ; *c1ccccc1]
f represents 1 or 2;
R¹ represents —(CH₂)ᵤ—COOH; and
u represents 1 or 2; 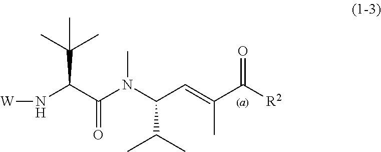
wherein
R² represents a glutamic acid residue (Glu), an aspartic acid residue (Asp) or a lysine residue (Lys);
an N-terminal nitrogen atom of R² forms an amide bond together with carbonyl (a); and
W is a group represented by formula (W-1) or formula (W-2): 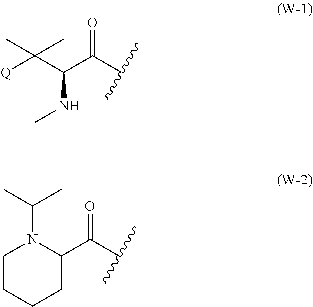
where
Q represents a group represented by formula (Q-1) or formula (Q-2): 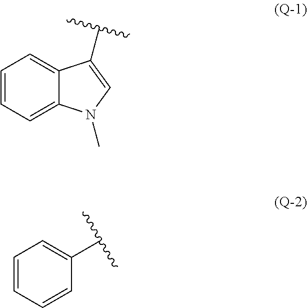 [SMILES: *c1cn(C)c2ccccc12 ; *c1ccccc1]
or a salt thereof and
a cell population including a differentiated cell derived from an PS cell.

50: The method for producing a cell population according to claim 49, wherein
W is a group represented by formula (W-1).

51: The method for producing a cell population according to claim 49, wherein
an antibody-drug conjugate releases the compound selected from the following compounds: 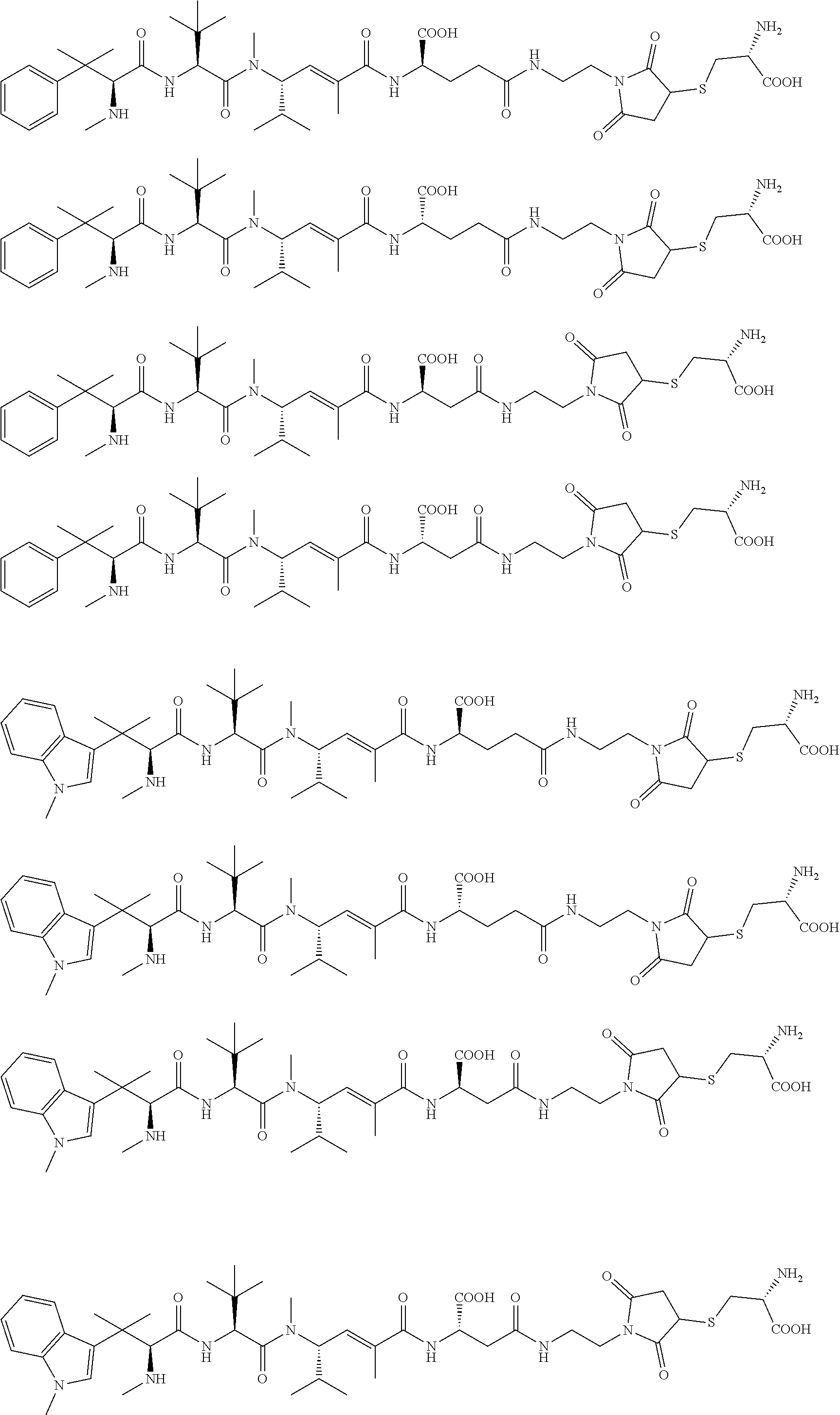
or a salt thereof.

52: The method for producing a cell population according to claim 49, wherein
an antibody-drug conjugate releases the compound selected from the following compounds: 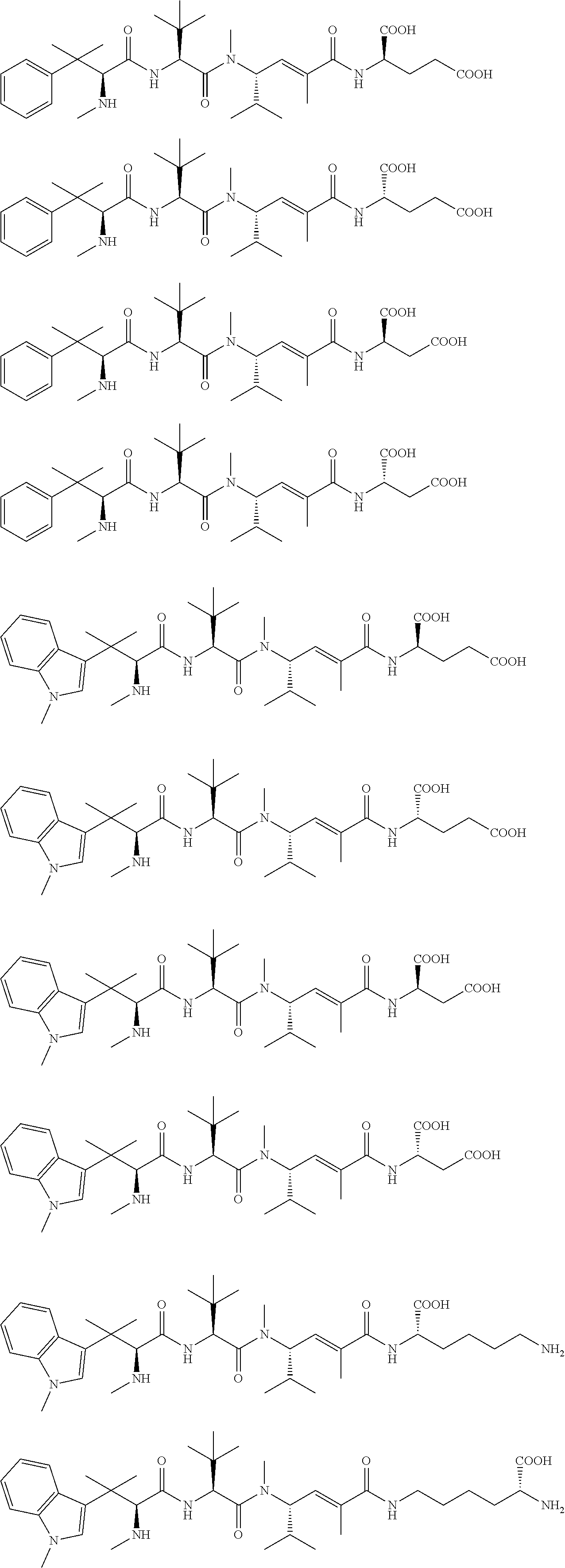
or a salt thereof.

53: The method according to claim 49, comprising:
(1) a step of performing induction of differentiation into a differentiated cell for a cell population including an PS cell; and
(2) a step of contacting the cell population including a differentiated cell obtained in the step (1) with the antibody-drug conjugate or a salt thereof.

54: A method for reducing a pluripotent stem cell, comprising:
a step of adding the antibody-drug conjugate that releases a compound represented by formula (1-1) or formula (1-3): 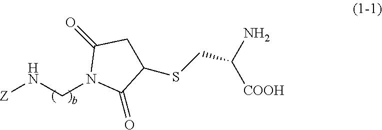
wherein
b represents an integer of 1 to 5; and
Z is a group represented by formula (Z-1) or formula (Z-2): 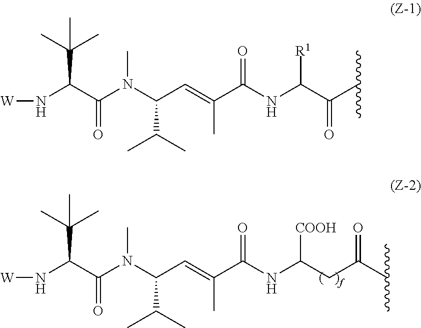
where
W represents a group represented by formula (W-1) or formula (W-2): 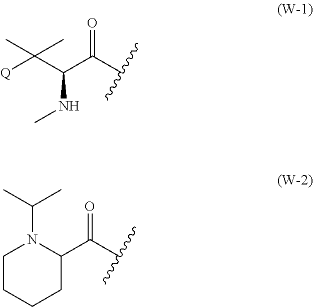
where
Q represents a group represented by formula (Q-1) or formula (Q-2): 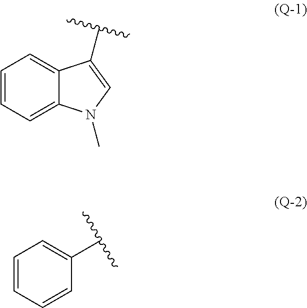 [SMILES: *c1cn(C)c2ccccc12 ; *c1ccccc1]
f represents 1 or 2;
R¹ represents —(CH₂)ᵤ—COOH; and
u represents 1 or 2; 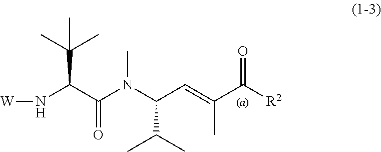
wherein
R² represents a glutamic acid residue (Glu), an aspartic acid residue (Asp) or a lysine residue (Lys);
an N-terminal nitrogen atom of R² forms an amide bond together with carbonyl (a); and
W is a group represented by formula (W-1) or formula (W-2): 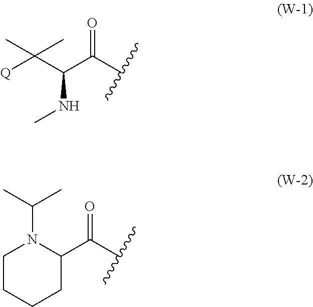
where
Q represents a group represented by formula (Q-1) or formula (Q-2): 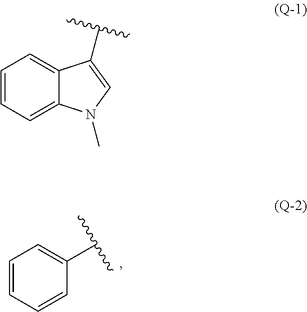 [SMILES: *c1cn(C)c2ccccc12 ; *c1ccccc1]
or a salt thereof to a culture solution containing a pluripotent stem cell.

55: The method for reducing a pluripotent stem cell according to claim 54, wherein the pluripotent stem cell is an ES cell or an PS cell.

56: The method for reducing a pluripotent stem cell according to claim 54, wherein the pluripotent stem cell is an iPS cell.

57: A method for eliminating a pluripotent stem cell, comprising:
a step of adding an antibody-drug conjugate represented by formula (2-1): 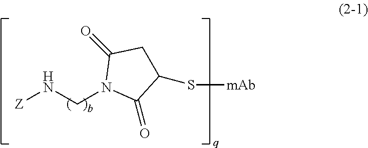
wherein
mAb represents an antibody recognizing an antigen expressed on a surface of a pluripotent stem cell;
q represents an integer of 1 to 20;
b represents an integer of 1 to 5; and
Z is a group represented by formula (Z-1) or formula (Z-2): 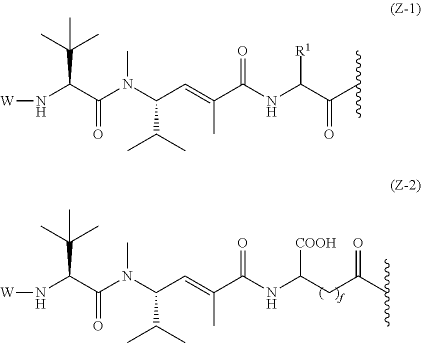
where
W represents a group represented by formula (W-1) or formula (W-2): 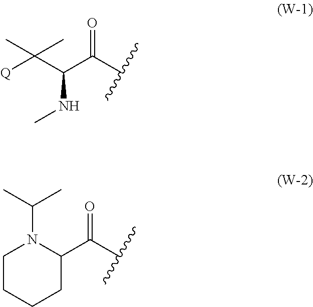
where
Q represents a group represented by formula (Q-1) or formula (Q-2): 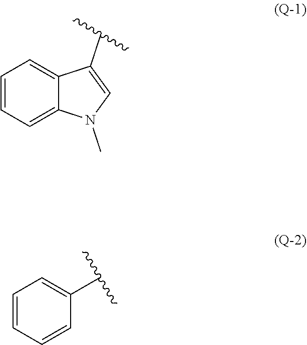 [SMILES: *c1cn(C)c2ccccc12 ; *c1ccccc1]
f represents 1 or 2;
R¹ represents —(CH₂)ᵤ—COOH; and
u represents 1 or 2,
or a salt thereof to a culture solution containing a pluripotent stem cell.

58: The method for eliminating a pluripotent stem cell according to claim 57, wherein
W is a group represented by formula (W-1).

59: The method for eliminating a pluripotent stem cell according to claim 57, wherein
mAb is an anti-CD30 antibody, an anti-TRA1-60 antibody, an anti-TRA1-81 antibody, an anti-SSEA3 antibody, an anti-SSEA4 antibody or an anti-rBC2LCN antibody.

60: The method for eliminating a pluripotent stem cell according to claim 57, wherein
mAb is an anti-CD30 antibody.

61: The method for eliminating a pluripotent stem cell according to claim 57, wherein
q is an integer of 1 to 8.

62: The method for eliminating a pluripotent stem cell according to claim 57, wherein the pluripotent stem cell is an ES cell or an PS cell.

63: The method for eliminating a pluripotent stem cell according to claim 57, wherein the pluripotent stem cell is an iPS cell.

64: A method for eliminating a pluripotent stem cell, comprising:
a step of adding an antibody-drug conjugate represented by formula (2-2): 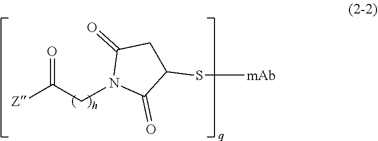
wherein
mAb represents an antibody recognizing an antigen expressed on a surface of a pluripotent stem cell;
q represents an integer of 1 to 20;
h represents an integer of 1 to 5;
Z″ is a group represented by formula (Z-5), formula (Z-6), formula (Z-7), formula (Z-8) or formula (Z-9): 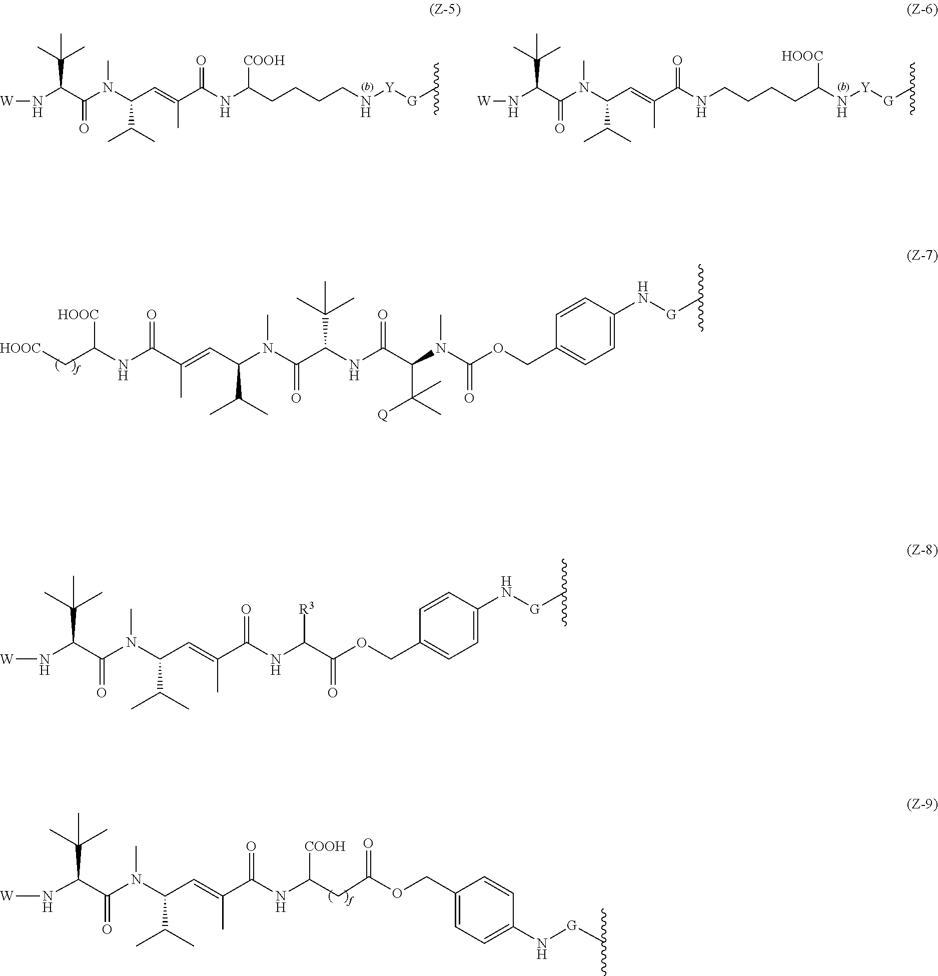
where
W represents a group represented by formula (W-1) or formula (W-2): 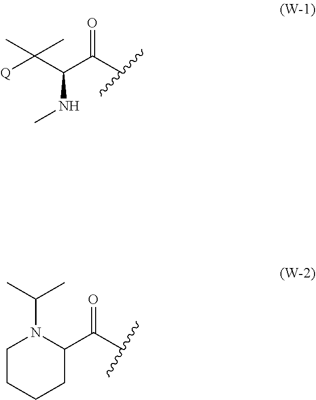
where
Q represents a group represented by formula (Q-1) or formula (Q-2): 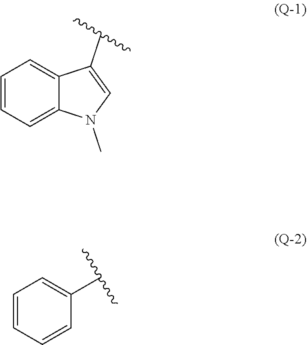 [SMILES: *c1cn(C)c2ccccc12 ; *c1ccccc1]
Y represents a single bond or a group represented by formula (Y-1): 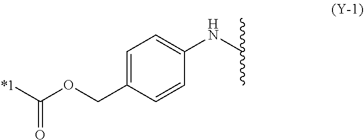
terminus *1 of the group represented by formula (Y-1) represents forming a bond together with amine (b);
G represents a single bond, *2-Gly-, *2-Gly-Gly-, *2-Lys-, *2-Lys-Phe-, *2-Lys-Val-, *2-Lys-Ala-, *2-Cit-Val-, *2-Cit-Phe-, *2-Cit-Leu-, *2-Arg-Phe-, *2-Cit-Ile-, *2-Cit-Trp-, *2-Lys-Phe-Phe-, *2-Lys-Phe-Ala-, *2-Lys-Phe-Gly-, *2-Asn-, *2-Asn-Ala-, *2-Asn-Ala-Ala-, *2-Asn-Ala-Thr-, *2-Asn-Ala-Pro-*2-Asn-Ala-Val-, *2-Asn-Ala-Phe-, *2-Asn-Ala-Tyr-, *2-Asn-Ala-Leu-, *2-Asn-Ala-Gly-, *2-Asn-Thr-Ala-*2-Asn-Thr-Pro-, *2-Asn-Thr-Thr-, *2-Gly-Phe-Gly-Gly-, *2-Gly-Leu-Phe-Gly- or *2-Leu-Ala-Leu-Ala-;
terminus *2 of G represents bonding to Y or —NH—;
f represents 1 or 2;
R³ represents —(CH₂)ᵤ—COOH; and
u represents 1 or 2,
or a salt thereof to a culture solution containing a pluripotent stem cell.

65: The method for eliminating a pluripotent stem cell according to claim 64, wherein
Z″ is a group represented by formula (Z-5) or formula (Z-6);
Y is a single bond; and
G is a single bond.

66: The method for eliminating a pluripotent stem cell according to claim 64, wherein
Z″ is a group represented by formula (Z-5) or formula (Z-6);
Y is a group represented by formula (Y-1); and
G is *2-Cit-Val-, *2-Asn-Ala-, *2-Asn-Ala-Ala- or *2-Asn-Ala-Pro-.

67: The method for eliminating a pluripotent stem cell according to claim 64, wherein
Z″ is a group represented by formula (Z-7), formula (Z-8) or formula (Z-9); and
G is *2-Cit-Val-, *2-Asn-Ala-, *2-Asn-Ala-Ala- or *2-Asn-Ala-Pro-.

68: The method for eliminating a pluripotent stem cell according to claim 64, wherein
W is a group represented by formula (W-1).

69: The method for eliminating a pluripotent stem cell according to claim 64, wherein
mAb is an anti-CD30 antibody, an anti-TRA1-60 antibody, an anti-TRA1-81 antibody, an anti-SSEA3 antibody, an anti-SSEA4 antibody or an anti-rBC2LCN antibody.

70: The method for eliminating a pluripotent stem cell according to claim 64, wherein
mAb is an anti-CD30 antibody.

71: The method for eliminating a pluripotent stem cell according to claim 64, wherein
q is an integer of 1 to 8.

72: The method for eliminating a pluripotent stem cell according to claim 64, wherein the pluripotent stem cell is an ES cell or an PS cell.

73: The method for eliminating a pluripotent stem cell according to claim 64, wherein the pluripotent stem cell is an iPS cell.

74: A method for producing a cell population including a differentiated cell derived from an iPS cell with substantially no iPS cell, comprising:
a step of contacting an antibody-drug conjugate represented by formula (2-1): 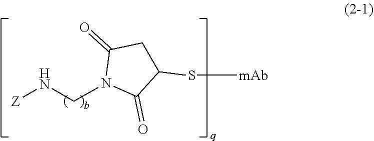
wherein
mAb represents an antibody recognizing an antigen expressed on a surface of a pluripotent stem cell;
q represents an integer of 1 to 20;
b represents an integer of 1 to 5; and
Z is a group represented by formula (Z-1) or formula (Z-2): 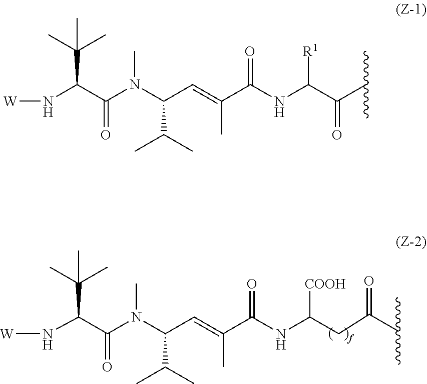
where
W represents a group represented by formula (W-1) or formula (W-2): 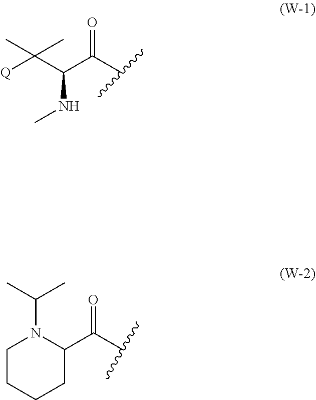
where
Q represents a group represented by formula (Q-1) or formula (Q-2): 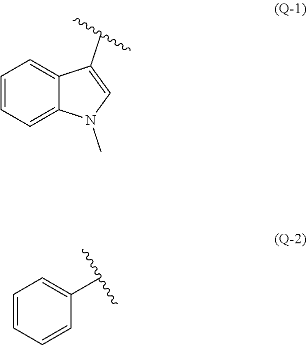 [SMILES: *c1cn(C)c2ccccc12 ; *c1ccccc1]
f represents 1 or 2;
R¹ represents —(CH₂)ᵤ—COOH; and
u represents 1 or 2,
or a salt thereof and
a cell population including a differentiated cell derived from an PS cell.

75: A method for producing a cell population including a differentiated cell derived from an PS cell with substantially no iPS cell, comprising:
a step of contacting an antibody-drug conjugate represented by formula (2-2): 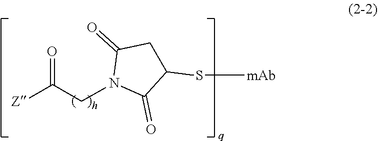
wherein
mAb represents an antibody recognizing an antigen expressed on a surface of a pluripotent stem cell;
q represents an integer of 1 to 20;
h represents an integer of 1 to 5;
Z″ is a group represented by formula (Z-5), formula (Z-6), formula (Z-7), formula (Z-8) or formula (Z-9): 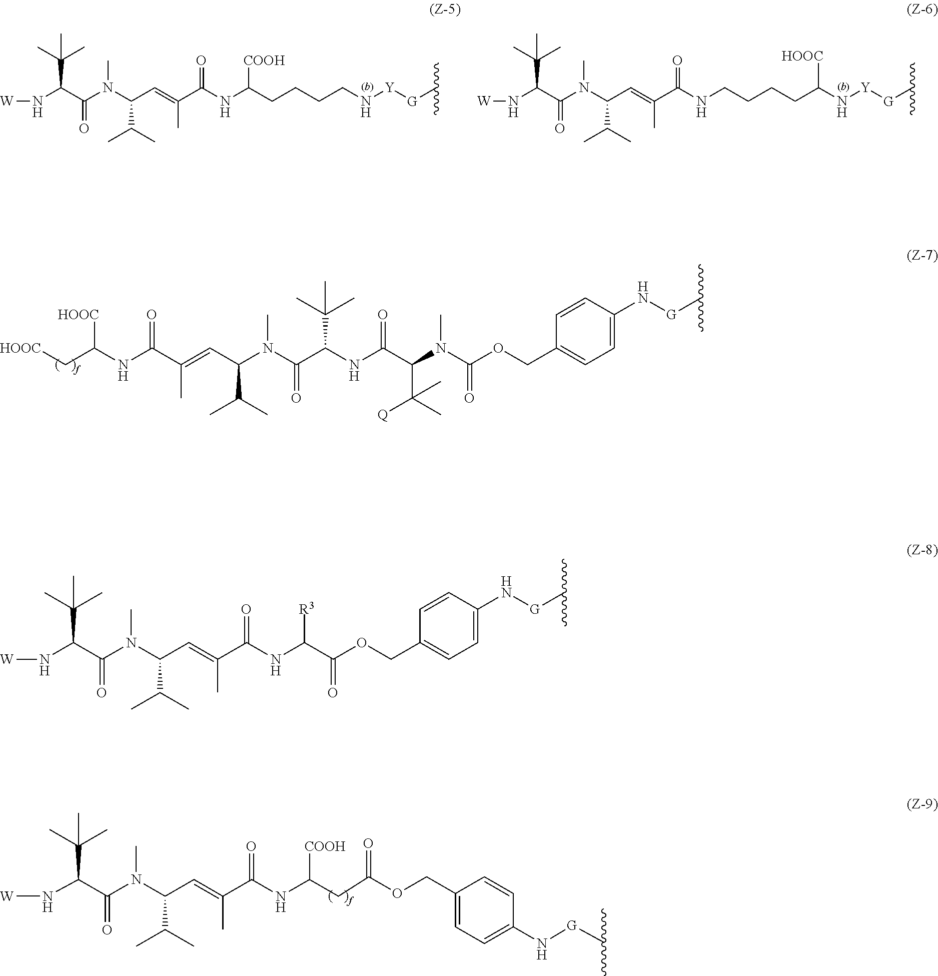
where
W represents a group represented by formula (W-1) or formula (W-2): 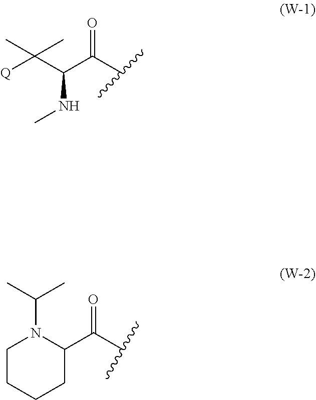
where
Q represents a group represented by formula (Q-1) or formula (Q-2): 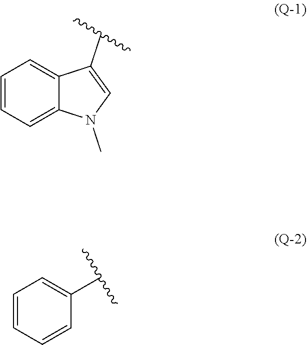 [SMILES: *c1cn(C)c2ccccc12 ; *c1ccccc1]
Y represents a single bond or a group represented by formula (Y-1): 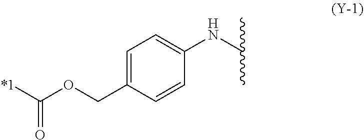
terminus *1 of the group represented by formula (Y-1) represents forming a bond together with amine (b);
G represents a single bond, *2-Gly-, *2-Gly-Gly-, *2-Lys-, *2-Lys-Phe-, *2-Lys-Val-, *2-Lys-Ala-, *2-Cit-Val-, *2-Cit-Phe-, *2-Cit-Leu-, *2-Arg-Phe-, *2-Cit-Ile-, *2-Cit-Trp-, *2-Lys-Phe-Phe-, *2-Lys-Phe-Ala-, *2-Lys-Phe-Gly-, *2-Asn-, *2-Asn-Ala-, *2-Asn-Ala-Ala-, *2-Asn-Ala-Thr-, *2-Asn-Ala-Pro-*2-Asn-Ala-Val-, *2-Asn-Ala-Phe-, *2-Asn-Ala-Tyr-, *2-Asn-Ala-Leu-, *2-Asn-Ala-Gly-, *2-Asn-Thr-Ala-*2-Asn-Thr-Pro-, *2-Asn-Thr-Thr-, *2-Gly-Phe-Gly-Gly-, *2-Gly-Leu-Phe-Gly- or *2-Leu-Ala-Leu-Ala-;
terminus *2 of G represents bonding to Y or —NH—;
f represents 1 or 2;
R³ represents —(CH₂)ᵤ—COOH; and
u represents 1 or 2,
or a salt thereof and
a cell population including a differentiated cell derived from an PS cell.
